(19) United States
(12) Patent Application Publication
(10) Pub. No.: US 2005/0019746 A1
(43) Pub. Date: Jan. 27, 2005

(54) Apoptosis-related kinase/GPCRs

(75) Inventors: Liam Seery, Cork (IE); Ian Hayes, Cork (IE); Finbarr Murphy, Cork (IE)

Correspondence Address: PALMER & DODGE, LLP;KATHLEEN M. WILLIAMS, 111 HUNTINGTON AVENUE, BOSTON, MA, 02199, US

(73) Assignee: EiRx Therapeutics Limited

(21) Appl. No.: 10/781,581

(22) Filed: Feb. 18, 2004

Related U.S. Application Data
(63) Continuation-in-part of application No. 10/764,238, filed on Jan. 23, 2004.
(60) Provisional application No. 60/457,533, filed on Mar. 25, 2003.

(30) Foreign Application Priority Data
Jan. 23, 2003 (GB) 0301566.6

(51) Int. Cl. C12Q 1/00; C12Q 1/68

(52) U.S. Cl. 435/4; 435/6

(57) ABSTRACT

The present invention relates to methods of identifying an agent that modulates the function of an apoptosis-associated polypeptide having a sequence as set out in Table 1B. The invention also relates to methods of modulating apoptosis, diagnostic methods, arrays, kits and compositions based upon the apoptosis-associated polypeptides having sequences as set out in Table 1B and in FIG. 42.

This is a Continuation-in-Part of U.S. application Ser. No. 10/764,238, filed Jan. 23, 2004, which application claims the priority of U.S. Provisional Application No. 60/457,533, filed Mar. 25, 2003, and also claims the foreign priority of United Kingdom Patent Application No. UK 0301566.5, filed Jan. 23, 2003, the entirety of each of which is incorporated herein by reference.

### FIELD OF THE INVENTION

The present invention relates to the identification of a number of human genes, and the proteins they encode, as having a function in the process of apopotosis. The invention also relates to the use of these “apoptosis-associated” genes and proteins in the modulation of apoptosis in cells and methods for identifying modulators of these genes or proteins and hence modulators of apoptosis.

### BACKGROUND TO THE INVENTION

Programmed cell death or apoptosis is a genetically programmed process by which cells die under both physiological and a variety of pathological conditions (Kerr et al, Br. J. Cancer, 26, 239-257, 1972). It serves as the counter-balancing force to mitosis during adult life and is a major contributor to the sculpting of physiological structures during the many processes of development (Wyllie et al, Int. Rev. Cytol, 68, 251-305, 1980). It is characterised by a number of well-defined biochemical hallmarks. These include DNA fragmentation, caused by the activation of an endogenous endonuclease enzyme (Wyllie, Nature, 284, 555-556,1980; Enari et al., Nature, 391, 43-50, 1998). The result is a DNA ladder pattern that can be readily visualised in agarose gels. Coupled with DNA fragmentation is cell shrinkage (Wesselbory et al., Cell Immunol. 148, 234-41, 1993) where water is actively extruded from the cell. The apoptotic cell then undergoes fragmentation into apoptotic bodies that are engulfed by neighbouring cells or cells of the reticuloendothelial system.

A second well-defined characteristic is the exposure of the phospholipid phosphatidylserine to the outside surface of the plasma membrane of the cell as it undergoes apoptosis (Fadok et al., J Immunol. 148, 2207-16, 1992). Normally this lipid is located on the inner side of the membrane lipid bilayer. The underlying mechanism responsible for this lipid flipping is poorly understood at present. Its expression serves as a signal for the recognition and phagocytosis of the apoptotic cell (Fadok et al., J Immunol. 148, 2207-16, 1992)

Under normal physiological conditions apoptosis is tightly regulated. However, there are a number of diseases where the process becomes deregulated, leading to a particular pathology. Examples of where apoptosis is retarded or inhibited include tumour development, a number of inflammatory conditions such as acute respiratory distress syndrome (ARDS) and other related conditions (Matute-Bello et al, Am J Respir Crit Care Med. 56, 1969-77, 1997). Inappropriate or excessive apoptosis occurs under conditions of ischaemia (stroke, myocardial infarction, etc) Linnik et al., Blood. 80, 1750-7, 1992, Gorman et al., J Neurol Sci. 139, 45-52, 1996) a series of neurodegenerative conditions, myelosuppression (Mori et al, Blood. 92, 101-7, 1998) following chemotherapy or irradiation (Lotem et al., Blood. 80, 1750-7, 1992) and a significant number of other diseases where cell death is a key feature of the pathology.

Neutrophils, Inflammation and Phosphatidylinositol Signalling

Neutrophils are recognized as major cellular mediators of inflammation. They play an essential role protecting the host such that they are the first line of defence recruited against invading microorganisms. It is the predominant cell type in the cellular phase of the acute inflammatory response (Edwards S: Biochemistry and Physiology of the Neutrophil. Cambridge, UK, Cambridge University Press, 1994). The importance of the neutrophil in protecting the host can be seen in patients who have severe neutropenia; the consequences of which are recurrent bacterial infections (Boxer L and Dale D C, Neutropenia: causes and consequences. Semin Hematol 2002 39(2) 75-81).

In order to protect the host against invading pathogens, neutrophils have a large arsenal of microbiocidal weapons including a variety of reactive oxygen and nitrogen species which, together with proteases and elastases, are released from their granules into the phagolysosome, following phagocytosis of the invading bacteria. However, some of these damaging agents inevitably leak into the surrounding environment where they can cause tissue damage at the site of inflammation. In addition neutrophils can signal for recruitment of other immune cells due to their ability to synthesize a large array of both pro- and anti-inflammatory cytokines including Interleukin-1α, Interleukin 1β, Interleukin 6, Tumour necrosis factor α (TNFα), IL1 receptor antagonists, as well as the chemokines, Interleukin 8, macrophage inflammatory protein 1α (MIP-1α) and MIP-1β, growth related gene product-α (Cassatell M A: Cytokines Produced by Polymorphonuclear Neutrophils: Molecular and Biological Aspects. Austin, Tex., RG Landes Co., 1996).

Inappropriate activation of these cells and release of proteases and elastases is recognized to contribute to a number of diseases including chronic obstructive pulmonary disease (COPD) and acute respiratory distress syndrome (ARDS) (Nogueraa et al. Thorax 2001;56:432-437; Lamb et al. Crit Care Med September 1999;27(9):1738-44).

One regulatory control mechanism in place in order to limit the extent of adverse activation is priming. Through the action of priming, the level of activation and subsequent responses of the cell can be regulated so that a continuum of activation states can be achieved. One such priming agent that is currently found at sites of inflammation is Granulocyte-Macrophage Colony Stimulating Factor (GM-CSF), a 22 kDa proinflammatory cytokine that is released by several activated cell types including monocytes and T cells. Preincubation of neutrophils with GM-CSF increases the inflammatory process by enhancing (priming) their ability to respond to a number of stimuli including chemotactic and phagocytic factors (Lopez et al. J Clin Invest. 1986;78(5):1220-8). In addition, GM-CSF is known to enhance the biological activities of neutrophils such as increased superoxide production (Weisbart et al. Nature Mar. 28-Apr. 3, 1985;314(6009):361-3), calcium mobilization (Naccache et al. J Immunol May 15, 1985;140(10):3541-6) and arachidonic acid release and synthesis of leukotriene B4 (DiPersio et al. J Immunol. 1988 15;140(12):4315-22).

Granulocyte-macrophage colony stimulating factor activation and priming of neutrophils has previously been shown to be coincidental with stimulation of phosphatidylinositol-3 kinase (al-Shami et al. Blood Feb. 1, 1997;89(3):1035-44). More recent work has positively correlated the regulation of phosphatidylinositol 3-Kinase activity with the enhanced conversion of phosphatidylinositol 4,5-bisphosphate to phosphatidylinositol 3,4,5-triphosphate and increased superoxide anion response (Cadwallader et al. J Immunol. Sep. 15, 2002;169(6):3336-44.).

Furthermore, neutrophils from mice with the regulatory subunit of PI3K-p85 alpha deleted produced comparable levels of superoxide anion in unprimed neutrophils as did wild type mice, however they produced significantly less than the wt post priming, again confirming a role of PI3K in the priming phenomenon for superoxide production (Yasui et al. J Leukoc Biol November 2002;72(5):1020-6). In another knock out study, this time targeting the catalytic subunit of PI3K-p110, investigators found that neutrophils migrated inefficiently to a variety of chemoattractants and that the mice demonstrated a reduced neutrophil oxidative burst, resulting in persistence of viable bacteria in the peritoneal cavity (Hirsch et al. Science Feb. 11, 2000;287(5455):1049-53).

To limit the state of activation, it is known that neutrophils can cycle between unprimed, primed, deprimed and reprimed stated. This phenomenon is thought to be due to controlling the levels of production and degradation of phosphoinositides, particularly phosphatidylinositol 3,4,5, triphosphate. Indeed, neutrophils from mice that deficient in SHIP (SH2-inositol-5-phosphatases) an enzyme that hydrolyses PtdIns (3,4,5)P₃, appear hyper-responsive to priming factors such as GMCSF. Mortality is high in these animals due to a lethal infiltration of the lungs by both macrophages and neutrophils (Helgason et al. Genes Dev Jun. 1, 1998;12(11):1610-20), suggesting that failure to regulate phosphoinositide levels might lead to excessive inflammation.

Neutrophils, Phosphatidylinositol Signaling and Survival

Apart from priming neutrophils, another consequence of the generation PtdIns(3,4,5)P3 is the phosphorylation and activation of AKT, the cellular homologue of the retroviral oncogene v-AKT. In the neutrophil, the biological consequence of AKT activation is inhibition of apoptosis and consequently prolonged survival—a phenomenon that impacts greatly on the number of these otherwise short lived neutrophils.

Studies have shown that inhibition of neutrophil apoptosis following GM-CSF treatment is mediated via PI3K and AKT, since chemical inhibitors of either kinase abrogate survival (Klein et al. J Immunol. 2000 164(8):4286-91). Similarly, other investigators have shown that in genetic knock out mice, inability to activate PI3K resulted in failure of GM-CSF to activate AKT and subsequent inability to delay apoptosis in neutrophils (Yasui et al. J Leukoc Biol November 2002;72(5):1020-6).

Although the direct anti-apoptotic mechanism of AKT activation in the neutrophil is currently unknown, it is known that survival mediated via activation of AKT is likely be due to the ability of AKT to phosphorylate and inactivate a number of pro-apoptotic proteins, including Caspase 9, Bad, and Forkhead Transcription factors. Additionally, AKT can exert a positive effect on NFκB—a transcription factor that promotes survival in response to several apoptotic stimuli—by phosphorylating and activating IκB kinase (IKK), a kinase that induces degradation of the NF-κB inhibitor, IκB.

Phosphatidylinositol Signaling and Cancer

Phosphatidylinositols has been a major focus of cancer research for nearly two decades since it became apparent that the PI3K activity was physically and functionally associated with the transforming activity of viral oncogenes such as the SRC tyrosine kinase and the middle T antigen of the polyoma virus. At the same time, PtdIns(3,4,5)P3, a lipid not previously known to exist, was detected in activated neutrophils (Traynor-Kaplan et al. 1988 Nature 334, 353-356). Since then, the literature on the role of PI3 K, phosphatidylinositols and cancer has increased logarithmically (for review see Vivanco I and Sawyers CL (2002) Nature Reviews Cancer 2 489-501). The oncogenic potential of PtdIns(3,4,5)P3 means that they are kept under tight control such that levels are barely detectable in unstimulated growth owing to the regulation of PI3 K and PIP3 phosphatases (PTEN, SHIP1, SHIP2). The PIP3 phosphatase that is most clearly involved in oncogenesis is PTEN, a 3-position lipid phosphatases that converts PIP3 back to PIP2. Mutations in this phosphatase were originally identified in breast cancers and glioblastomas, but it is now recognized to be one of the most commonly mutated proteins associated with cancer. Along with PTEN, mutations associated with phosphatidylinositol signaling that are also associated with cancer include constitutive PI3 kinase activity and elevated AKT activity.

Neutrophil Model for Apoptosis and for Discovery of Therapeutic Targets

WO 02/04657 describes a neutrophil model of apoptosis. GM-CSF provides a signal which acts through a signal transduction cascade and is associated with significant changes, or patterns of changes, in gene expression in the cell. Thus the inhibition of neutrophil cell death through apoptosis acts by the regulation of ‘effector genes’ that control the process of apoptosis. To date, however, the identities of such ‘effector genes’ and their role in the signalling pathways that lead to the biochemical events of cell death have been incompletely determined. To-date many of the genes found have certain fundamental flaws e.g. they act late in the process, after the cell has committed to a death programme, or they are ubiquitous, that is they are not restricted to a particular cell type. The ideal targets to control apoptosis act early in the process and are restricted to a particular cell type.

The control of apoptosis represents a significant therapeutic target, since many diseases are due to defects in this process. There is, therefore, an acute need to identify the genes that regulate this process. In other words, if one identifies a gene that prevents apoptosis, then this gene/gene product or its function can be blocked by a drug and apoptosis allowed to occur. Similarly, where there is excessive apoptosis, blocking this process is an important therapeutic goal.

It is clear from above that phosphatidylinositol signaling is a fundamental part of the biology of the GM-CSF stimulated neutrophil both with regard to priming the cell for enhanced cellular activity and also for prolonging the survival of the cell by delaying the apoptotic process. Similarly, a link between phosphatidylinositol signaling and tumour survival has also been demonstrated. Clearly, identifying regulated targets associated with phosphatidylinositol signalling in the activated neutrophil would be a valid target for cancer therapy. Therefore it is concluded that neutrophils stimulated with GM-CSF are an excellent model to study for identification of novel therapeutic targets including inflammatory and cancer targets.

### SUMMARY OF THE INVENTION

The present invention identifies a number of genes, “apoptosis-associated” genes, whose expression is correlated with an early stage in the regulation of apoptosis. The identification and role of these genes in apoptosis has been validated using model assays described in WO 02/04657 as described herein and by knocking down gene expression using RNAi and assessing the resultant phenotype for altered apoptosis progression. Accordingly, these genes represent new targets for therapeutic agents. The apoptosis-associated genes disclosed herein are provided in Table 1A (known apoptosis-associated genes), in Table 1B (genes newly identified as apoptosis-associated) and in FIG. 42, which provides polypeptide and amino acid sequences corresponding to the GenBank Accession Numbers provided in Table 1B.

The invention features a method of identifying an agent that modulates the function of an apoptosis-associated polypeptide having a sequence as set out, or identified in Table 1, where the method includes: (a) providing a sample containing an apoptosis-associated polypeptide having a sequence as set out in Table 1, and a candidate agent; and incubating under conditions to permit binding of the candidate (or test) agent to the polypeptide; (b) measuring the binding of the apoptosis-associated polypeptide having a sequence as set out in Table 1B to the candidate agent in the sample; and (c) comparing the binding of the apoptosis-associated polypeptide having a sequence as set out in Table 1B to the candidate agent in the sample with the binding of the polypeptide having a sequence as set out in Table 1B to a control agent, where the control agent is known to not bind to the polypeptide having a sequence as set out in Table 1; where an increase in the binding of the apoptosis-associated polypeptide having a sequence as set out in Table 1B to the candidate agent in the sample relative to the binding of the apoptosis-associated polypeptide of having a sequence as set out in Table 1B to the control agent indicates that the candidate agent modulates the function of the apoptosis-associated polypeptide of having a sequence as set out in Table 1B.

The invention also features a method of detecting the presence in a sample of an apoptosis-associated polypeptide having a sequence as set out in Table 1B, where the method includes: (a) bringing the biological sample containing DNA or RNA into contact with a probe comprising a fragment of at least 15 nucleotides of a nucleic acid having a sequence as set out in Table 1B under hybridizing conditions; and (b) detecting a duplex formed between the probe and nucleic acid in the sample; where detection of a duplex indicates the presence in the sample of an apoptosis-associated polypeptide having a sequence as set out in Table 1B.

Levels of gene expression may be determined in any other appropriate manner. Detecting a decrease in gene expression may be achieved by measuring apoptosis-associated gene expression in treated versus non-treated cells. Preferably, gene expression may be measured by detecting nucleic acid encoding an apoptosis-associated polypeptide such as apoptosis-associated mRNA transcripts, or a fragment thereof. In one embodiment, the method of measuring mRNA transcripts may use an amplification technique as described herein. In another embodiment, apoptosis-associated gene expression may be measured by detecting the apoptosis-associated polypeptide gene product, or fragment thereof, using, for example, agents that bind apoptosis-associated polypeptides. Suitable agents include antibodies.

Accordingly, in another aspect, the invention features a method of detecting the presence in a sample of an apoptosis-associated polypeptide having a sequence as set out in Table 1B, where the method includes: (a) providing an antibody capable of binding to the apoptosis-associated polypeptide having a sequence as set out in Table 1B; (b) incubating a biological sample with said antibody under conditions which allow for the formation of an antibody-antigen complex; and (c) detecting an antibody-antigen complex comprising said antibody; where detection of an antibody-antigen complex indicates the presence in the sample of an apoptosis-associated polypeptide having a sequence as set out in Table 1B.

In another aspect, there is provided a method of modulating apoptosis in a cell comprising the step of increasing, decreasing or otherwise altering the functional activity of an apoptosis-associated polypeptide or the nucleic acid encoding it. In one embodiment, said modulation of apoptosis is inhibition. In another embodiment, said modulation of apoptosis confers survival in a cell.

Suitably, the apoptosis-associated polypeptide has a sequence as identified in Table 1B.

In the context of the present invention the term ‘altered functional activity of an apoptosis associated polypeptide or the nucleic acid encoding it’ includes within its scope increased, decreased or an otherwise altered activity of the apoptosis associated polypeptide as compared with the native protein functioning in its normal environment, that is within a single cell under native conditions. In addition, it also includes within its scope an increased or decreased level of expression and/or altered intracellular distribution of the nucleic acid encoding an apoptosis associated polypeptide, and/or an altered the intracellular distribution of apoptosis associated polypeptide.

In one embodiment, said method involves decreasing apoptosis-associated gene expression. In a preferred aspect, the expression of an apoptosis-associated gene is decreased by greater than 50%, 60%, 70%, 80%, 90%, 100%, 200%, 500% or more of its normal level in untreated cells.

Preferably, a decrease in apoptosis-associated gene expression may be effected by antisense expression. Other means of decreasing apoptosis-associated gene expression will be recognised by those skilled in the art and include introducing dominant negatives, peptides or small molecules including RNA molecules such as siRNA molecules which cause a decrease in gene expression through RNA interference. Suitable siRNA molecules are described in the Examples section herein.

In another embodiment, said method involves increasing apoptosis-associated gene expression. In a preferred aspect, the expression of an apoptosis-associated gene is increased by greater than 50%, 60%, 70%, 80%, 90%, 100%, 200%, 500% or more of its normal level in untreated cells.

Preferably, said method comprises providing an expression vector comprising a nucleic acid sequence encoding an apoptosis-associated polypeptide; introducing the expression vector into the cell and maintaining the cell under conditions permitting expression of the encoded polypeptide in the cell. As defined herein, a nucleic acid encoding an apoptosis-associated polypeptide or an apoptosis-associated polypeptide encompasses fragments thereof.

Accordingly, the invention features a method of modulating apoptosis in a cell, where the method includes: (a) transforming into the cell a double-stranded nucleic acid sequence having a sequence as set out in Table 1B or a complement thereof, where the nucleic acid sequence is operably linked to a regulatory sequence; and (b) culturing the cell under conditions whereby the nucleic acid sequence is expressed; thereby modulating apoptosis in the cell.

The invention also features a method of modulating apoptosis in a cell, where the method includes: (a) transforming into the cell a double-stranded nucleic acid sequence encoding a polypeptide having a sequence as set out in Table 1B, where the nucleic acid sequence is operably linked to a regulatory sequence; and (b) culturing the cell under conditions whereby the nucleic acid sequence is expressed; thereby modulating apoptosis in the cell.

In an additional aspect, the invention features a method of modulating apoptosis in a cell, where the method includes: (a) transforming into the cell a double-stranded nucleic acid sequence encoding a polypeptide having at least 80% sequence identity with a polypeptide having a sequence as set out in Table 1B, where the nucleic acid sequence is operably linked to a regulatory sequence; and (b) culturing the cell under conditions whereby the nucleic acid sequence is expressed; thereby modulating apoptosis in the cell.

Another aspect of the invention features a method of modulating apoptosis in a cell, where the method includes: (a) transforming into the cell an isolated nucleic acid molecule comprising a regulatory sequence operably linked to a nucleic acid sequence that encodes a ribonucleic acid (RNA) precursor, where the precursor comprises: (i) a first stem portion comprising a 15 to 40 nucleotide long sequence that is identical to 15 to 40 consecutive nucleotides of a sequence as set out in Table 1B; (ii) a second stem portion comprising a 15 to 40 nucleotide long sequence that is complementary to 15 to 40 consecutive nucleotides of a sequence as set out in Table 1B, and where the first and second stem portions can hybridize with each other to form a duplex stem; and (iii) a loop portion that connects the two stem portions; (b) culturing the cell under conditions whereby the nucleic acid sequence is expressed; thereby modulating apoptosis in the cell.

Suitably the cell may be a therapeutic target for the treatment of disease. For example, such a cell may be a cancer cell, a cell involved in an inflammatory disorder, a cell involved in an autoimmune disorder or in a neurodegenerative disorder.

In any of the methods described herein, the nucleic acid sequence is a nucleic acid sequence as set out in Table 1B, or a complement thereof. The nucleic acid sequence can encode a polypeptide having a sequence as set out in Table 1B. The nucleic acid sequence can encode a polypeptide having 80% sequence identity to a polypeptide having a sequence as set out in Table 1B.

The methods described herein can be used to decrease or to increase apoptosis.

Another feature of the invention is an RNA precursor encoded by a nucleic acid sequence as set out or identified in Table 1B. Such an RNA precursor can be included in a composition as a biologically active ingredient. Such an RNA precursor or composition can be used for treating a disease or condition characterized by abnormal apoptosis in mammalian tissue, by contacting the tissue with the RNA precursor or composition. Suitably, the disease can be cancer or inflammatory disease.

Another feature of the invention is a host cell transformed by the methods described herein. Such a host cell can contain all or a part of the nucleic acid sequences as set out in Table 1B. Such a host cell can express all or a part of the polypeptide having a sequence as set out in Table 1B.

In a further aspect of the invention, the apoptosis-associated genes as identified in Table 1B may be used to identify downstream targets. For example, proteins which interact with or whose expression is controlled by the apoptosis-associated genes may be identified.

The methods described herein can be used to provide a mammal with an anti-apoptotic protein, where the method includes introducing into the mammal a mammalian cell transformed by the methods described herein.

The modulation of apoptosis can be for therapeutic purposes. Thus, the invention also features a pharmaceutical composition comprising, as an active ingredient, an apoptosis-associated nucleic acid sequence having a sequence as set out in Table 1B, and a pharmaceutically-acceptable carrier.

The invention further features a pharmaceutical composition comprising, as an active ingredient, apoptosis-associated polypeptide having a sequence as set out in Table 1B, and a pharmaceutically-acceptable carrier.

Accordingly, in another aspect of the invention there is provided a method of treatment of disease characterised by abnormal apoptosis comprising administering a modulator of apoptosis-associated gene expression or functional activity to an individual.

Thus, in one embodiment, the invention additionally features a pharmaceutical composition comprising, as an active ingredient, an antibody to apoptosis-associated nucleic acid having a sequence as set out in Table 1B, and a pharmaceutically-acceptable carrier. Such an antibody can be used in a method for diagnosing a disease or condition characterized by abnormal apoptosis in mammalian tissue, the method comprising contacting the tissue with the antibody, and detecting an antibody/antigen complex, wherein said detection is indicative of said disease or condition.

In another aspect, the invention features a pharmaceutical composition comprising, as an active ingredient, an antibody to apoptosis-associated polypeptide having a sequence as set out in Table 1B, and a pharmaceutically-acceptable carrier. Such an antibody can be used in a method for diagnosing a disease or condition characterized by abnormal apoptosis in mammalian tissue, the method comprising contacting the tissue with the antibody, and detecting an antibody/antigen complex, wherein said detection is indicative of said disease or condition.

A further aspect of the invention features a method for treating a disease or condition characterized by abnormal apoptosis in mammalian tissue, the method comprising contacting the tissue with an antagonist of apoptosis-associated polypeptide having a sequence as set out in Table 1B.

An additional aspect of the invention features a method for treating a disease or condition characterized by abnormal apoptosis in mammalian tissue, the method comprising contacting the tissue with an agonist of apoptosis-associated polypeptide having a sequence as set out in Table 1B.

The invention also features a kit for treating a disease or condition characterized by abnormal apoptosis in mammalian tissue, the kit comprising: (a) a polypeptide encoded by a nucleic acid having a sequence as set out in Table 1B; (b) a nucleic acid having a nucleotide sequence as set out in Table 1B; or (c) an antibody recognising an epitope of a polypeptide of (a).

Cells useful in the methods of the invention may be from any source, for example from primary cultures, from established cell lines, in organ culture or in vivo. Cell lines useful in the invention include cells and cell lines of haematopoietic origin. Suitable cells include HeLa, U937 (monocyte), TF1, HEK293 (T), primary cultures of neutrophils or cells having neutrophil characteristics, for example HL60 cells, murine FDCP-1, FDCPmix, 3T3, primary or human stem cells. Other suitable cells include cancer cell lines such as U251, SKOV3, OVCAR3, MCF7 PC3, HCT15, 786-0, HT29, M-14, Molt 4, CEM, K562, Daudi, DU145, NCI-H460 and LnCap.

Where methods are therapeutic, cells may be disease-associated cells such as cancer, inflammatory, autoimmune or neurodegeration-associated cells.

In another aspect, there is provided the use of a modulator of apoptosis-associated gene expression or activity in the manufacture of a medicament for use in the treatment of disease.

Suitably, said modulator is an antisense molecule or an RNA molecule which mediates RNA interference and thus causes a decrease in apoptosis-associated gene expression.

Suitable diseases include cancer, inflammation, autoimmune disease and neurodegenerative disorders.

A number of inflammatory diseases such as asthma, chronic obstructive pulmonary disease (COPD), Cystic Fibrosis (CF), Rheumatoid Arthritis (RA) and Inflammatory bowel disease (IBD) are characterised by a) elevated levels and expression of cytokines and growth factors that act predominantly on myeloid cells, b)prolonged survival of myeloid cells, and c) prolonged activation of myeloid cells.

Thus, increased numbers of activated myeloid cells such as neutrophils are associated with, and strongly implicated in, the pathology of a number of these chronic and acute inflammatory diseases (Williams T J and Jose P J: Novartis Found Symp 2001;234:136-41; discussion 141-8; Barnes P J: Chest February 2000;117(2 Suppl):10S-4S; Nadel J A: Chest February 2000;117(2 Suppl):10S-4S; Ward I et al: Trends Pharmacol Sci December 1999;20(12):503-9; Bradbury J and Lakatos L: Drug Discov Today May 1, 2001;6(9):441-442).

Accordingly, in one embodiment, there is provided the use of an apoptosis-associated gene, or an agent that alters apoptosis-associated gene expression in a cell, in the treatment of inflammatory diseases through the modulation of myeloid cell apoptosis.

In the context of the present invention, the term “myeloid cell” refers to terminally differentiated, non-dividing cells of the myeloid lineage. These cells include neutrophils, eosinophils and monocytes/macrophages. In one embodiment of any aspect of the present invention, the myeloid cell is a neutrophil, eosinophil or monocyte/macrophage.

Inflammatory diseases include, but are not limited to, diseases such as sepsis, Acute Respiratory Distress Syndrome, Pre enclampsia, Myocardial ischemia, reperfusion injury, Psoriasis, Asthma, COPD, bronchiolitis, Cystic Fibrosis, Rheumatoid Arthritis, Inflammatory Bowel Disease, Crohns Disease and Ulcerative colitis.

In another aspect of the invention, there is provided a RNA molecule capable of interferring with expression of an apoptosis associated gene having a sequence as set out in Table 1B. Suitably said RNAi molecule has a sequence as set out in the Examples section herein.

An additional aspect of the invention is an array comprising at least two apoptosis-associated genes having nucleic acid sequences as set out in Table 1B. The nucleic acid sequences can be DNA sequences. The nucleic acid sequences can be RNA sequences.

Another aspect of the invention is an array comprising at least two apoptosis-associated proteins having polypeptides having a sequence as set out in Table 1B.

Apoptosis-associated genes identified herein in Table 1B include genes that 1) are ‘effector’ genes involved in the cells defence mechanisms aimed at preventing apoptosis (anti-apoptotic genes) and thus represent therapeutic targets, 2) make up aspects of the apoptosis and/or GM-CSF signal cascade and thus represent therapeutic targets, 3) initiate the process of apoptosis (pro-apoptotic genes) and thus represent therapeutic targets, and 4) are associated with the processes of apoptosis and defence that will aid in the understanding of key pathways, processes and mechanisms that may subsequently lead to the identification of therapeutic targets.

In particular, the genes identified herein are protein kinases and G-protein Coupled Receptors (GPCRs).

Protein Kinases

Kinases regulate many different cell proliferation, differentiation, and signaling processes by adding phosphate groups to proteins. Uncontrolled signaling has been implicated in a variety of disease conditions including inflammation, cancer, arteriosclerosis, and psoriasis. Reversible protein phosphorylation is the main strategy for controlling activities of eukaryotic cells. It is estimated that more than 1000 of the 10,000 proteins active in a typical mammalian cell are phosphorylated. Phosphorylation occurs in response to extracellular signals (hormones, neurotransmitters, growth and differentiation factors, etc), cell cycle checkpoints, and environmental or nutritional stresses and is roughly analogous to turning on a molecular switch. When the switch goes on, the appropriate protein kinase activates a metabolic enzyme, regulatory protein, receptor, cytoskeletal protein, ion channel or pump, or transcription factor.

The kinases comprise the largest known protein group, a superfamily of enzymes with widely varied functions and specificities (see, for example, Hardie, G. and Hanks, S. (1995) The Protein Kinase Facts Books, Vol I:7-20 Academic Press, San Diego, Calif.).

G-Protein Coupled Receptors

G-protein coupled receptors (GPCRs) constitute a major class of proteins responsible for transducing a signal within a cell. GPCRs have three structural domains: an amino terminal extracellular domain, a transmembrane domain containing seven transmembrane segments, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

GPCR genes and gene-products are potential causative agents of disease (Spiegel et al., J. Clin. Invest. 92:1119-1125 (1993); McKusick et al., J. Med. Genet. 30:1-26 (1993)). Specific defects in the rhodopsin gene and the V2 vasopressin receptor gene have been shown to cause various forms of retinitis pigmentosum (Nathans et al., Annu. Rev. Genet. 26:403-424(1992)), and nephrogenic diabetes insipidus (Holtzman et al., Hum. Mol. Genet. 2:1201-1204 (1993)). These receptors are of critical importance to both the central nervous system and peripheral physiological processes. Evolutionary analyses suggest that the ancestor of these proteins originally developed in concert with complex body plans and nervous systems.

The GPCR protein superfamily can be divided into five families: Family I, receptors typified by rhodopsin and the beta.2-adrenergic receptor and currently represented by over 200 unique members (Dohlman et al., Annu. Rev. Biochem. 60:653-688 (1991)); Family II, the parathyroid hormone/calcitonin/secretin receptor family (Juppner et al., Science 254:1024-1026 (1991); Lin et al., Science 254:1022-1024 (1991)); Family III, the metabotropic glutamate receptor family (Nakanishi, Science 258 597:603 (1992)); Family IV, the cAMP receptor family, important in the chemotaxis and development of D. discoideum (Klein et al., Science 241:1467-1472 (1988)); and Family V, the fungal mating pheromone receptors such as STE2 (Kurjan, Annu. Rev. Biochem. 61:1097-1129 1992)).

There are also a small number of other proteins which present seven putative hydrophobic segments and appear to be unrelated to GPCRs; they have not been shown to couple to G-proteins. Drosophila expresses a photoreceptor-specific protein, bride of sevenless (boss), a seven-transmembrane-segment protein which has been extensively studied and does not show evidence of being a GPCR (Hart et al., Proc. Natl. Acad. Sci. USA 90:5047-5051 (1993)). The gene frizzled (fz) in Drosophila is also thought to be a protein with seven transmembrane segments. Like boss, fz has not been shown to couple to G-proteins (Vinson et al., Nature 338:263-264 (1989)).

G proteins represent a family of heterotrimeric proteins composed of alpha, beta and gamma subunits, that bind guanine nucleotides. These proteins are usually linked to cell surface receptors, e.g., receptors containing seven transmembrane segments. Following ligand binding to the GPCR, a conformational change is transmitted to the G protein, which causes the .alpha.-subunit to exchange a bound GDP molecule for a GTP molecule and to dissociate from the .beta..gamma.-subunits. The GTP-bound form of the alpha.-subunit typically functions as an effector-modulating moiety, leading to the production of second messengers, such as cAMP (e.g., by activation of adenyl cyclase), diacylglycerol or inositol phosphates. Greater than 20 different types of a-subunits are known in humans. These subunits associate with a smaller pool of beta. and gamma. subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

Thus kinases and GPCRs are a major target for drug action and development. Accordingly, it is valuable to the field of pharmaceutical development to identify and characterize previously unknown kinases and GPCRs. The present invention advances the state of the art by identifying a role in apoptosis for certain kinases and GPCRs whose role has not previously been identified.

Accordingly, modulators of the GPCR and Kinase polypeptides identifies herein as apoptosis-associated genes can be indentified using standard assays for GPCR and Kinase activity. Suitable assays are well know to those skilled in the art.

In a preferred embodiment of any aspect of the invention, the apoptosis-associated gene or protein or the gene or protein whose sequence is identified in Table 1B is selected from MAK, GPR86, PCTAIRE protein kinase 3, BAI2, pp125, STK6, ULK1, serine/threonine kinase 16, ribosome S6 protein kinase, TLK2, ethanolamine kinase, MAP kinase-interacting serine/threonine kinase 1, FLJ20559, FLJ13351, NTKL, CDC42-binding protein kinase beta (DMPK-like), RBSK ribokinase, EDG6, DAGK, G protein coupled receptor 12,PRK, mitogen-activated protein kinase kinase 5, phosphatidylinositol 4-kinase, catalytic, beta polypeptide, fins-related tyrosine kinase 4, PSKH1 protein serine kinase H1 and inositol 1,4,5-triphosphate 3-kinase C.

The present invention provides a method of identifying an inhibitor of an apoptosis-associated protein that is encoded by a gene selected from Table 1B, comprising providing a preparation containing said encoded protein; incubating the preparation with a test agent to be screened under conditions to permit binding of the test agent to the protein; determining whether the test agent interacts with the protein by detecting the presence or absence of a signal generated from the interaction of the agent with the protein, and thereby determining whether the test agent inhibits the apoptosis-associated protein.

In the context of this invention, a preparation containing the encoded protein can comprise a cell expressing the protein, a purified protein, including a recombinantly produced protein, or a membrane preparation containing the protein. Furthermore, when used in methods of this invention for identifying inhibitor compounds, the protein preferably comprises the human protein as described herein, but in alternative embodiments, functional variants of this structure can be utilized, including other mammalian homologs (e.g. dog, pig, rat, mouse), fusion proteins (e.g. proteins with sequences added to aid in purification, e.g. GST, glutathione S-transferase sequence), and functional domains or fragments of the protein (e.g. active catalytic domains of protein kinases).

The present invention further provides a method of identifying an inhibitor of an apoptosis-associated protein kinase that is encoded by a protein kinase gene selected from table 1B, comprising providing a preparation containing said encoded protein kinase; incubating the preparation with a test agent to be screened under conditions to permit binding of the test agent to the protein kinase; determining whether the test agent interacts with the protein kinase by detecting a change in the phosphotransferase activity of the protein kinase, and thereby determining whether the test agent inhibits the apoptosis-associated protein kinase.

The present invention further provides a method of identifying an inhibitor of an apoptosis-associated cell surface receptor protein that is encoded by a gene selected from table 1B, comprising providing a cell expressing on its surface a protein that is encoded by cell surface receptor gene selected from table 1B, said protein being associated with a second component capable of providing a detectable signal in response to the binding of an agent to said protein; contacting with an test agent to be screened under conditions to permit binding to the protein; and determining whether the agent binds to and inhibits the protein, by detecting the presence or absence of a signal generated from the interaction of the compound with the protein and thereby determining whether the test agent inhibits the activity of the apoptosis-associated cell surface receptor protein that is encoded by a gene selected from table 1B. In the context of this method the second component capable of providing a detectable signal can be a G-protein, for example a Gi, Go, Gs, G₁₆, G₁₅, Gq or G₁₂₋₁₃ G-protein. In an alternative embodiment the second component can be β-arrestin.

The present invention further provides a process for determining whether a chemical compound specifically binds to and inhibits an apoptosis-associated protein that is encoded by a gene selected from table 1B, which comprises contacting cells producing a second messenger response and expressing the protein that is encoded by a gene selected from table 1B, wherein such cells do not normally express said protein, with the chemical compound under conditions suitable for inhibition of the protein, and measuring the second messenger response in the presence and in the absence of the chemical compound, a change in the second messenger response in the presence of the chemical compound indicating that the compound inhibits the apoptosis-associated protein that is encoded by a gene selected from table 1B.

In the context of this invention, the second messenger response can be selected from, but is not limited to, the following: chloride channel activation, a change in intracellular calcium ion levels, a release of inositol phosphate, a release of arachidonic acid, GTPγS binding, activation of MAP kinase, cAMP accumulation, a change in intracellular potassium ion levels, or a change in intracellular sodium ion levels. The second messenger response can be measured directly by a variety of methods well known in the art, or by a change in reporter gene activity. Such reporter genes include, but are not limited to, secreted alkaline phosphatase, luciferase, and β-galactosidase.

The present invention further provides a method of identifying an activator of apoptosis, comprising providing a preparation containing a protein that is encoded by a gene selected from table 1B; incubating the preparation with a test agent to be screened under conditions to permit binding of the test agent to the protein; determining whether the test agent binds to, and inhibits the protein, by detecting the presence or absence of a signal generated from the interaction of the agent with the protein, and determining whether any test agent that inhibits the protein is an activator of apoptosis.

The present invention further provides a method for identifying an agent that has pro-apoptotic activity, which comprises determining whether a test agent interacts with a protein that is encoded by a gene selected from table 1B in a preparation comprising the encoded protein, and determining whether any agent that interacts with the encoded protein is an activator of apoptosis.

The present invention further provides a method of identifying an inhibitor of tumor cell proliferation, comprising providing a preparation containing a protein that is encoded by a gene selected from table 1B; incubating the preparation with a test agent to be screened under conditions to permit binding of the test agent to the protein; determining whether the test agent binds to, and inhibits the protein, by detecting the presence or absence of a signal generated from the interaction of the agent with the protein, and determining whether any test agent that inhibits the protein is an inhibitor of tumor cell proliferation.

The present invention further provides a method for identification of an agent that inhibits tumor cell proliferation, which comprises determining whether a test agent interacts with a protein that is encoded by a gene selected from table 1B in a preparation comprising the encoded protein, and determining whether any agent that interacts with the encoded protein is an inhibitor of tumor cell proliferation.

In the methods of this invention, determination of whether an agent inhibits tumor cell proliferation can be performed by any of the methods known to one of skill in the art, e.g labeling cells capable of DNA synthesis with BrdU or radiolabeled thymidine.

The present invention further provides a method for identifying an agent that inhibits tumor cell growth, which comprises determining whether a test agent interacts with a protein that is encoded by a gene selected from table 1B in a preparation comprising the encoded protein, and determining whether any agent that interacts with the encoded protein is an inhibitor of tumor cell growth.

The present invention further provides a method for identifying an agent that inhibits tumor growth, which comprises determining whether a test agent interacts with a protein that is encoded by a gene selected from table 1B in a preparation comprising the encoded protein, and determining whether any agent that interacts with the encoded protein is an inhibitor of tumor growth.

The present invention further provides a method for identifying an agent that has pro-apoptotic activity, which comprises determining whether a test agent modulates the activity or expression of a protein that is encoded by a gene selected from table 1B, and determining whether any agent that modulates said activity or expression is an activator of apoptosis.

The present invention further provides a method for identifying an agent that inhibits tumor cell proliferation, which comprises determining whether a test agent modulates the activity or expression of a protein that is encoded by a gene selected from table 1B, and determining whether any agent that modulates said activity or expression is an inhibitor of tumor cell proliferation.

The present invention further provides a method for identifying an agent that inhibits tumor cell growth, which comprises determining whether a test agent modulates the activity or expression of a protein that is encoded by a gene selected from table 1B, and determining whether any agent that modulates said activity or expression is an inhibitor of tumor cell growth.

The present invention further provides a method for identifying an agent that inhibits tumor growth, which comprises determining whether a test agent modulates the activity or expression of a protein that is encoded by a gene selected from table 1B, and determining whether any agent that modulates said activity or expression is an inhibitor of tumor growth.

In the above methods the test agent can be selected from, but is not limited to, a low molecular weight (less than about 5000 Daltons) organic molecule, an antibody or antibody fragment, an antisense oligonucleotide, a small inhibitory dsRNA, or a ribozyme.

The present invention also provides an inhibitor of tumor cell proliferation or tumor growth, identified by the above methods. Such an inhibitor of tumor cell proliferation or tumor growth can be, but is not limited to, a low molecular weight organic molecule (less than about 5000 Daltons), an antibody or antibody fragment, an antisense oligonucleotide, a small inhibitory dsRNA (e.g. as used for RNA interference), or a ribozyme.

The present invention further provides a method of inhibiting tumor growth in a mammal in recognized need of such treatment, said method comprising administering to said mammal in recognized need of such treatment, an inhibitor of the activity or expression of an apoptosis-associated protein that is encoded by a gene selected from table 1B, wherein said administering is in an effective amount to reduce tumor growth in said mammal. In the context of this method, the inhibitor can be selected from, but is not limited to, a low molecular weight organic molecule (less than about 5000 Daltons), an antibody or antibody fragment, an antisense oligonucleotide, a small inhibitory dsRNA and a ribozyme. The small inhibitory dsRNAs described in the Methods section herein for performing RNA intereference experiments to reduce the level of expression of proteins encoded by genes selected from Table 1B are particular examples of inhibitors that can be used in the practice of this method.

The present invention further provides a method of inhibiting tumor cell proliferation in a mammal in recognized need of such treatment, said method comprising administering to said mammal in recognized need of such treatment, an inhibitor of the activity or expression of a protein that is encoded by a gene selected from table 1B, wherein said administering is in an effective amount to reduce tumor cell proliferation in said mammal. In the context of this method, the inhibitor can be selected from, but is not limited to, a low molecular weight organic molecule (less than about 5000 Daltons), an antibody or antibody fragment, an antisense oligonucleotide, a small inhibitory dsRNA and a ribozyme. The small inhibitory dsRNAs described in the Methods section herein for performing RNA intereference experiments to reduce the level of expression of proteins encoded by genes selected from Table 1B are particular examples of inhibitors that can be used in the practice of this method.

The present invention further provides a method of stimulating apoptosis of tumor cells in a mammal in recognized need of such treatment, said method comprising administering to said mammal in recognized need of such treatment, an inhibitor of the activity or expression of a protein that is encoded by a gene selected from table 1B, wherein said administering is in an effective amount to stimulate apoptosis of tumor cells in said mammal. In the context of this method, the inhibitor can be selected from, but is not limited to, a low molecular weight organic molecule, an antibody or antibody fragment, an antisense oligonucleotide, a small inhibitory dsRNA and a ribozyme. The small inhibitory dsRNAs described in the Methods section herein for performing RNA intereference experiments to reduce the level of expression of proteins encoded by genes selected from Table 1B are particular examples of inhibitors that can be used in the practice of this method.

The present invention further provides a method of preparing a composition comprising a chemical compound which specifically binds to and inhibits an apoptosis-associated protein that is encoded by a gene selected from table 1B, which comprises contacting cells expressing the apoptosis-associated protein that is encoded by a gene selected from table 1B, wherein such cells do not normally express the encoded protein, with a test chemical compound under conditions suitable for binding of such a compound to the protein, detecting specific binding to and inhibition of the encoded protein by the test chemical compound, and admixing the test chemical so identified, or a functional analog or homolog of said test chemical, with a carrier, thereby preparing said composition.

The present invention further provides a method of preparing a composition comprising a chemical compound which specifically inhibits an apoptosis-associated protein that is encoded by a gene selected from table 1B, which comprises contacting an apoptosis-associated protein that is encoded by a gene selected from table 1B with a test chemical compound under conditions suitable for binding of such a compound to the protein, detecting specific inhibition of the protein by the test chemical compound, and admixing the test chemical so identified, or a functional analog or homolog of said test chemical, with a carrier, thereby preparing said composition.

The present invention provides a method for identifying neoplasias responsive to treatment with compounds that selectively inhibit neoplasia, comprising exposing a sample of the neoplasia to a compound that has inhibitory activity on an apoptosis-associated protein that is encoded by a gene selected from table 1B, and determining whether the compound inhibits the neoplasia.

The present invention further provides a method for identifying neoplasias responsive to treatment with compounds that selectively inhibit neoplasia, comprising (a) removing a sample of neoplastic tissue from a patient, (b) growing cells from the sample as explants in vitro, (c) contacting a sample of said cells with a compound that has inhibitory activity on an apoptosis-associated protein that is encoded by a gene selected from table 1B, (d) comparing the growth of the cells in the presence of the compound with the growth of cells in the absence of the compound, and (e) determining whether the growth of the neoplasia is sensitive to inhibition by the compound.

The present invention further provides a method for identifying neoplasias responsive to treatment with compounds that selectively inhibit neoplasia, comprising (a) removing a sample of neoplastic tissue from a patient, (b) growing cells from the sample as explants in vitro, (c) contacting a sample of said cells with a compound that has inhibitory activity against an apoptosis-associated protein that is encoded by a gene selected from table 1B, (d) comparing the number of apoptotic cells in the presence of the compound with the number of apoptotic cells in the absence of the compound, and (e) determining whether the compound promotes an increase in apoptosis in the neoplasia.

The present invention further provides a method for identifying neoplasias responsive to treatment with an inhibitor of an apoptosis-associated protein that is encoded by a gene selected from table 1B, comprising determining the level of the protein that is encoded by a gene selected from table 1B in a sample of neoplastic tissue, wherein an elevated level of the apoptosis-associated protein that is encoded by a gene selected from table 1B in the neoplastic tissue, relative to normal tissue, is indicative that the neoplasia has potential for being treated by an inhibitor of the apoptosis-associated protein that is encoded by a gene selected from table 1B.

In one embodiment of this method the determination of the level of the protein that is encoded by a gene selected from table 1B in the neoplastic tissue comprises determining the amount of protein for the protein that is encoded by a gene selected from table 1B in the neoplastic tissue sample.

In another embodiment of this method the determination of the level of the protein that is encoded by a gene selected from table 1B in the neoplastic tissue comprises determining the amount of mRNA encoding for the protein that is encoded by a gene selected from table 1B in the neoplastic tissue sample.

In a further embodiment of this method the determination of the level of the protein that is encoded by a gene selected from table 1B in the neoplastic tissue comprises determining the biochemical activity of the protein that is encoded by a gene selected from table 1B in the neoplastic tissue sample. For example, if the apoptosis-associated protein is a protein kinase, autophosphorylation or phosphorylation of a protein or peptide substrate can be determined. For an apoptosis-associated GPCR protein, determination of the level of activation of downstream signal transduction pathway components can be assessed.

The present invention further provides a method for identifying neoplasias from a patient responsive to treatment with an inhibitor of an apoptosis-associated protein that is encoded by a gene selected from table 1B comprising the steps of: (a) obtaining a suspected neoplasic tissue sample from the patient; (b) contacting the sample with an antibody that is immunoreactive with the apoptosis-associated protein that is encoded by a gene selected from table 1B under conditions effective to allow the formation of immune complexes; and (c) detecting the complexes thus formed, wherein an elevated amount of the apoptosis-associated protein that is encoded by a gene selected from table 1B in the neoplastic tissue, relative to normal tissue, is indicative that the neoplasia has potential for being treated by an inhibitor of the apoptosis-associated protein that is encoded by a gene selected from table 1B.

### BRIEF DESCRIPTION OF THE DRAWINGS

Figures and Tables:

FIG. 1 shows sub G1 analysis of neutrophils cultured overnight either in the presence or absence of GMCSF, prior to analysis for the percentage of cells with a sub-G1 profile. From the profiles it is clear that significantly fewer neutrophils undergo apoptosis when cultured with GM-CSF than when cultured without as determined by the percentage of cells with a sub G1 profile (9.7% with versus 65% without).

FIG. 2 shows that absence of GM-CSF or GM-CSF in the presence of Ly204002 increases the amount of apoptosis, relative to neutrophils cultured in GM-CSF alone.

FIG. 3 shows that absence of GM-CSF or GM-CSF in the presence of an AKT inhibitor (IL-6-Hydroxymethyl-chiro-inositol 2-(R)-2-O-methyl-3-O-octadecylcarbonate) increases the amount of apoptosis, relative to neutrophils cultured in GM-CSF alone.

FIG. 4 shows a comparative analysis of expression data using microarray analysis and QPCR. Dotted bars (dark) are QPCR, hashed bars (light) are Affymetrix.

FIG. 5 shows a “heat map” depicting the relative expression of 12 target genes across a panel of 61 cell lines. Expression is relative to GAPDH.

FIG. 6 shows Forward and Side Scatter analysis of TF1 population by Flow Cytometry. TF1 cells are cultured for 48 h in the presence or absence of GM-CSF (2 ng/ml) prior to acquisition and analysis using a FacsCalibre flow cytometer. The area enclosed in the gate represents the live gate region. From this figure, it is observed that there is a decrease of approx. 50% in the number of cells with side scatter parameters of healthy cells in the factor deprived cells.

FIG. 7 shows cell cycle analysis by flow cytometry. Apoptotic cells have degraded DNA, resulting in an increase in low intensity staining, measured as a sub G1 peak in fluorescent histograms.

FIG. 8 shows a composite of dot plots showing a typical result for TF1 cells treated under the various conditions. Cells are transduced with a retroviral vector containing the full length BCL2 (C) coding sequence or a control vector lacking BCL2 (A and B) and subjected to the indicated conditions. The presence of BCL2 in TF1 cells allows more cells to survive GM-CSF withdrawal, as determined by the percentage of cells in the live gate (enclosed area).

FIG. 9 shows TF1 cells that were either transduced with a retroviral vector containing the full length EDG6 coding sequence or a control vector lacking EDG6 were cultured in the presence or absence of GM-CSF for 48 h prior to analysis for viability using FSC/SSC parameters as described in the text. There were significantly more viable cells expressing EDG6 than those transduced with control vector alone, following GM-CSF withdrawal, indicating that EDG6 protects TF1 cells from apoptosis.

FIG. 10 shows TF1 cells that were either transduced with a retroviral vector containing the full length TLK2 coding sequence or a control vector lacking TLK2 were cultured for 96 h post transduction prior to analysis for viability using FSC/SSC parameters as described in the text. There were significantly more viable cells in the population transduced with control vector alone in contrast to those transduced with TLK2 where less than 1% of cells remained viable, indicating that TLK2 induces apoptosis in TF1 cells.

FIG. 11 shows a determination of the mRNA levels (by QPCR quantification as described) of 2 interferon response genes, OAS1 (NM_{—}002534) and GBP1 (NM_{—}002503), in 7 cell lines (HCT15, A549, SKOV3, DU145, PC3, A498, and U251) post siRNA transfection with MS or GAPDH siRNAs.

FIGS. 12 to 31 show apoptosis as detected by MTT, FSC/SSC and Sub G1 Analysis. The MTT Assay was performed@72 hrs. Optical density/Absorbance correlates positively with cell viability. The FSC/SSC Analysis detects apoptosis by monitoring changes in cell morphology as determined by the cell's forward scatter and side scatter parameters. The % viability positively correlates with the % of the population remaining in the viable cell gate post transfection with gene specific siRNA oligos compared to missense control. The Sub G1 Analysis measures DNA Fragmentation, a characteristic of cells undergoing apoptosis. The % Sub G1 correlates positively with cells undergoing apoptosis. FIG. 25 additionally shows the results of JC-1 and caspase assays as described herein. Apoptosis modulation induced by siRNA oligo is compared to that obtained with control MS, BCL2 and/or Survivin siRNA oligos.

FIG. 32 shows the results of a Wst 1 viability assay.

FIG. 33 shows the results of a BrdU proliferation assay.

FIG. 34 shows apoptosis of transfected cells as measured by Annexin V.

FIG. 35 shows sample results of a clonagenicity assay.

FIG. 36 shows the results of a 14 day clonagenicity assay.

FIG. 37 shows knockdown of PSKH1 in ancorage independent soft agar assay.

FIG. 38 shows knockdown of TLK2 in ancorage independent soft agar assay.

FIG. 39 shows migration of PC3 cells treated with siRNA.

FIG. 40 shows migration of PC3 cells treated with siRNA.

FIG. 41 shows migration of PC3 cells treated with siRNA.

FIG. 42 shows the polynucleotide and polypeptide sequences for the identified genes provided in Table 1B.

Tables 1A and 1B show the list of identified genes and their respective accession numbers.

Table 2 gives the fold change of gene expression of genes identified in Table 1 across treatment groups, referenced to untreated neutrophils.

Table 3 lists a first range of cancer cell lines.

Table 4 lists the QPCR primers for a first set of target genes.

Table 5 shows the results of QPCR in normal/cancer tissues. Expression in cancer tissues is expressed relative to normal cells from the same tissue.

Table 6 shows the results of QPCR in cancer cells.

Table 7 lists a second range of cancer cell lines.

Table 8 lists the QPCR primers for a second set of target genes.

Table 9 shows the sequences of a first batch of siRNA oligonucleotides.

Table 10 demonstrates the efficacy of siRNA in the PC3 prostate cancer cell line.

Table 11 shows the sequences of a second batch of siRNA oligonucleotides. ¹—second, alternative siRNA oligonucleotides are used in Example 7 as indicated in Figures.²—alternative siRNA oligonucleotides used for TLK2 as indicated.

Table 12 demonstrates the efficacy of siRNA for the second batch in several cell lines.

Table 13(a)-(g) shows percentage survival of the indicated cell lines with siRNA relative to missense in a clonogenicity assay over 3 independent experiments.

Table 14 shows the percentage survival of the indicated cell lines post transfection with siRNA oligomers in ancorage independent soft agar assay.

### DETAILED DESCRIPTION OF THE INVENTION

The invention describes human genes involved in apoptosis. Such genes can be used in assays described herein to determine if a candidate substance is an inhibitor of apoptosis. These same assays can be used to determine if the substance is an enhancer of apoptosis.

Such assays include binding assays, where the degree of binding of the substance to the polypeptides described herein is determined. Such assays also include determining if the substance prevents the binding between a polypeptide described herein and another substance known to bind that polypeptide.

The assays also include assays to determine the presence or absence of a polypeptide described herein in a sample, by adding to the sample a substance known to bind that polypeptide, and determining if binding takes place. Alternatively, a nucleic acid probe, the sequence of which is based on the nucleic acid that encodes the polypeptide, can be used to determine the presence or absence in the sample of the nucleic acid encoding the polypeptide. The probe can be added to the sample and incubated under hybridizing conditions to cause binding to the nucleic acid, if it is present in the sample. Such a probe can be labelled to more easily determine if binding has occurred. Alternatively, instead of a nucleic acid probe, an antibody specific for either the nucleic acid of the polypeptide can be used.

In situations where one wishes to know if a mutation has occurred in a nucleic acid encoding a polypeptide described herein, one can obtain a sample containing the nucleic acid, and use a nucleic acid probe specific for the region in which the mutation is believed to occur. Lack of binding, relative to a sample containing the equivalent nucleic acid without the mutation, indicates that the nucleic acid contains a mutation at that location. If it is not known where in the nucleic acid the mutation has occurred, then a sequential series of probes can be used which cover the entire length of the nucleic acid.

These assays can also be used to determine if a polypeptide described herein is being overexpressed in a tissue, e.g., a tumor, or tissue suspected of harboring overproliferating cells. An amount of a known ligand can be added to a sample from the tissue, where the ligand binds the polypeptide. If the amount of bound ligand-polypeptide complex in the sample is greater than that found normally, then the polypeptide is overexpressed. “Normally” can mean the amount of polypeptide found in a sample of another tissue from the same organism, or the equivalent tissue from another organism, or relative to previously-determined baseline levels of expression.

Any of the assays described above can be incorporated into a kit for determining presence/absence of a nucleic acid or polypeptide described herein, or the level of expression of a polypeptide described herein.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N. J.). These documents are incorporated herein by reference.

Definitions

“Apoptosis” or programmed cell death is a controlled intracellular process characterised by the condensation and subsequent fragmentation of the cell nucleus during which the plasma membrane remains intact. It is an active, highly regulated process distinguished by cell shrinkage and packaging of the cell contents into apoptotic bodies that are subsequently engulfed by macrophages, thus avoiding activation of the inflammatory response (for review see Wyllie, Br. Med. Bull. 53:451-465, 1997). Apoptotic death is distinct from other cell processes including necrotic cell death and replicative senescence.

The term “nucleic acid”, as used herein, refers to single stranded or double stranded DNA and RNA molecules including natural nucleic acids found in nature and/or modified, artificial nucleic acids having modified backbones or bases, as are known in the art. A “nucleic acid” of the present invention is a nucleic acid which encodes an apoptosis associated gene or protein as described above. The term moreover includes those polynucleotides capable of hybridising, under stringent hybridisation conditions, to the naturally occurring nucleic acids identified above, or the complement thereof.

An “isolated” polypeptide or nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide. Preferably, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the nucleic acids of the present invention.

“Vector” refers to any agent such as a plasmid, cosmid, virus, autonomously replicating sequence, phage, or linear single-stranded, circular single-stranded, linear double-stranded, or circular double-stranded DNA or RNA nucleotide sequence that carries exogenous DNA into a host cell or organism. The recombinant vector may be derived from any source and is capable of genomic integration or autonomous replication.

A ligand which “binds specifically to” or “specifically recognises” an apoptosis-associated polypeptide of the invention is one which binds to such a polypeptide with a higher affinity than it does to other unrelated polypeptides.

By “modulating apoptosis” is meant that for a given cell, under certain environmental conditions, its normal tendency to undergo apoptosis is changed by at least 10% compared to an untreated cell. For example, blood neutrophils have a defined apoptotic tendency—within a population of cells, greater than 80% will apoptose within the first 24 hours. Modulating the apoptosis of blood neutrophils means changing this normal apoptotic tendency such that apoptosis is increased or decreased relative to the normal rate. Similarly, blood neutrophils in the presence of GM-CSF have a decreased tendency to apoptose. Thus, modulating apoptosis of blood neutrophils in the presence of GM-CSF means increasing or decreasing apoptosis relative to their normal decreased tendency under these conditions. A decreased tendency to apoptose may also be a measurable increase in cell survival and may be the result of an inhibition of apoptosis by inhibiting one or more components of the apoptotic pathway.

The term “expression” refers to the transcription of a genes DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expresssion in the absence of said treatment.

The “functional activity” of a protein in the context of the present invention describes the function the protein performs in its native environment. Altering the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself.

A “reporter gene” is a gene which is incorporated into an expression vector and placed under the same controls as a gene of interest to express an easily measurable phenotype.

The term “myeloid cell” encompasses terminally differentiated, non-dividing (i.e. non-proliferative) cells derived from the myeloid cell lineage and includes neutrophils or polymorphonuclear neutrophils (PMNs), eosinophils and mononuclear phagocytes. The latter cells are known as monocytes when in the blood and macrophages when they have migrated into the tissues. Terminal differentiation is the normal endpoint in cellular differentiation and is usually not reversible.

“Inflammatory disorders” or “inflammatory diseases” are disorders characterised by chronic or acute inflammation. This, in turn, is characterised by elevated levels of cytokines and/or survival factors for myeloid cells. These disorders are characterised by the prolongued survival of myeloid cells including neutrophils, eosinophils and monocytes/macrophages which can be present as a mixture of one or more of these cell types. Accordingly, reference to treatment of inflammatory disorders or diseases includes treatment of the individual cell types or treatment of a mixture of different cell types. The resultant increased numbers of these inflammatory cells is associated with the disease pathology. In chronic inflammation a persistent inflammatory response causes damaging effects such as tissue damage. Chronic Inflammatory Diseases include cystic fibrosis, acute respiratory distress syndrome, chronic obstructive pulmonary disease, inflammatory bowel disease and rheumatoid arthritis. Other inflammatory diseases are known to those skilled in the art and include sepsis, Pre enclampsia, Myocardial ischemia, reperfusion injury, Psoriasis, Asthma, bronchiolitis, Crohns Disease and Ulcerative colitis.

By “polynucleotide” or “polypeptide” is meant the DNA and protein sequences disclosed herein. The terms also include close variants of those sequences, where the variant possesses the same biological activity as the reference sequence. Such variant sequences include “alleles” (variant sequences found at the same genetic locus in the same or closely-related species), “homologs” (a gene related to a second gene by descent from a common ancestral DNA sequence, and separated by either speciation (“ortholog”) or genetic duplication (“paralog”)), so long as such variants retain the same biological activity as the reference sequence(s) disclosed herein.

The invention is also intended to include silent polymorphisms and conservative substitutions in the polynucleotides and polypeptides disclosed herein, so long as such variants retain the same biological activity as the reference sequence(s) as disclosed herein.

Polypeptides

It will be understood that polypeptides identified herein are not limited to polypeptides identified in Table 1B or those polypeptides having the amino acid sequence encoded by the nucleic acid sequences identified in Table 1B or fragments thereof but also include homologous sequences obtained from any source, for example related viral/bacterial proteins, cellular homologues and synthetic peptides, as well as variants or derivatives thereof.

Thus, reference herein to “polypeptides” also includes those sequences encoding homologues from other species including animals such as mammals (e.g., mice, rats or rabbits), especially primates. Particularly preferred polypeptides include homologous human sequences.

The term also covers variants, homologues or derivatives of the amino acid sequences encoded by the nucleic acids identified in Table 1B, as well as variants, homologues or derivatives of the nucleotide sequences coding for the amino acid sequences.

In the context of the present invention, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200, 300, 400 or 500 amino acids with any one of the polypeptide sequences disclosed herein. In particular, homology should typically be considered with respect to those regions of the sequence known to be essential for protein function rather than non-essential neighbouring sequences. This is especially important when considering homologous sequences from distantly related organisms.

Although homology can also be considered in terms of functional similarity (i.e., amino acid residues having similar chemical properties/functions), in the context of the present invention it is preferred to express homology in terms of sequence identity.

Homology comparisons can be conducted by eye, or more usually, with the aid of readily available sequence comparison programs. These publicly and commercially available computer programs can calculate percent homology between two or more sequences.

Percent homology may be calculated over contiguous sequences, i.e., one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (version 2.2.7; see Ausubel et al., 1999 ibid—Chapter 18), FASTA (Altschul et al., 1990, J. Mol. Biol. 215:403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program (e.g., version 10.3).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

The terms “variant” or “derivative” in relation to amino acid sequences includes any substitution of, variation of, modification of, replacement of, deletion of or addition of one (or more) amino acids from or to the sequence providing the resultant amino acid sequence retains substantially the same activity as the unmodified sequence, preferably having at least the same activity as the polypeptides identified in Table 1B or the polypeptides encoded by the nucleic acid sequences identified in Table 1B.

As used herein, the phrase “substantially the same activity” means that a variant polypeptide exhibits at least one of the following relative to the known polypeptide having a sequence described herein: catalyzing the same reaction; inhibiting the same reaction; specifically binding or being bound by at least one molecule specifically bound by the known polypeptide; and serving as substrate or co-factor for the same enzyme.

Polypeptides having the amino acid sequence encoded by the nucleic acid sequences identified in Table 1B, or fragments or homologues thereof may be modified for use in the present invention. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides of the invention. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Polypeptides for use in the invention also include fragments of the full length sequences mentioned above. Preferably said fragments comprise at least one epitope. Methods of identifying epitopes are well known in the art. Fragments will typically comprise at least 6 amino acids, more preferably at least 10, 20, 30, 50 or 100 amino acids.

Proteins are typically made by recombinant means, for example as described below. However they may also be made by synthetic means using techniques well known to skilled persons such as solid phase synthesis. Proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins may also be obtained by purification of cell extracts from animal cells.

Multimeric proteins comprising the apoptosis proteins are also intended to be encompassed by the present invention. By “multimer” is meant a protein comprising two or more copies of a subunit protein. The subunit protein may be one of the proteins of the present invention, e.g., an apoptosis protein as disclosed herein repeated two or more times. Such a multimer may also be a fusion or chimeric protein, e.g., a repeated apoptosis protein may be combined with polylinker sequence, and/or one or more apoptosis peptides, which may be present in a single copy, or may also be tandemly repeated, e.g., a protein may comprise two or more multimers within the overall protein.

Proteins may be in a substantially isolated form. It will be understood that the protein may be mixed with carriers or diluents which will not interfere with the intended purpose of the protein and still be regarded as substantially isolated. A protein for use in the invention may also be in a substantially purified form, in which case it will generally comprise the protein in a preparation in which more than 90%, e.g., 95%, 98% or 99% of the protein in the preparation is a protein as identified herein.

A polypeptide may be labeled with a revealing label. The revealing label may be any suitable label which allows the polypeptide to be detected. Suitable labels include radioisotopes, e.g., ¹²⁵I, enzymes, antibodies, polynucleotides and linkers such as biotin. Labeled polypeptides of the invention may be used in diagnostic procedures such as immunoassays to determine the amount of a polypeptide of the invention in a sample. Polypeptides or labeled polypeptides of the invention may also be used in serological or cell-mediated immune assays for the detection of immune reactivity to said polypeptides in animals and humans using standard protocols.

A polypeptide or labeled polypeptide or fragment thereof may also be fixed to a solid phase, for example the surface of a microarray, an immunoassay well or dipstick. Such labeled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

Immunoassays may be used for detecting polypeptides for examples in detecting a modulation of protein expression or function.

Polypeptides identified herein may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides of the invention may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g., myristolation, glycosylation, truncation, lapidation and tyrosine, serine or threonine phosphorylation) as may be needed to confer optimal biological activity on recombinant expression products of the invention. Such cell culture systems in which polypeptides of the invention are expressed may be used in assay systems to identify candidate substances which interfere with or enhance the functions of the polypeptides of the invention in the cell.

Polynucleotides

Polynucleotides or nucleic acids of the invention include polynucleotides identified in Table 1B or any one or more of the nucleic acid sequences encoding the polypeptides which are encoded by the nucleic acids identified in Table 1B and fragments thereof. Fragments will typically comprise at least 15 consecutive nucleotides of the full-length polynucleotide, more preferably at least 20, 30, 50 or 100 consecutive nucleotides of the full-length polynucleotide. It is straightforward to identify a nucleic acid sequence which encodes such a polypeptide, by reference to the genetic code. Furthermore, computer programs are available which translate a nucleic acid sequence to a polypeptide sequence, and/or vice versa. The disclosure of a nucleic acid and its corresponding polypeptide sequence includes a disclosure of all nucleic acids (and their sequences) which encode that polypeptide sequence.

It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

In preferred embodiments of the invention, nucleic acids of the invention comprise those polynucleotides, such as cDNA, mRNA, and genomic DNA. Such polynucleotides may typically comprise, Homo sapiens cDNA, mRNA, and genomic DNA, etc. Accession numbers are provided in the Examples for the nucleic acid sequences, and the polypeptides they encode can be derived by use of such accession numbers in a relevant database, such as a human sequence database, including a Human Genome Sequence database, may be used to identify such human polynucleotide or polypeptide sequences. Relevant sequences may also be obtained by searching sequence databases such as BLAST with the polypeptide sequences. In particular, a search using TBLASTN may be employed.

Nucleic acids for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence for use in the present invention include any substitution of, variation of, modification of, replacement of, deletion of or addition of one (or more) nucleic acid from or to the sequence. Preferably said variant, homologues or derivatives code for a polypeptide having biological activity.

As indicated above, with respect to sequence homology, preferably there is at least 50 or 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses the use of nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that a nucleic acid is found to hybridize to the nucleic acid having a sequence identified in Table 1B at a level significantly above background. Background implies a level of signal generated by interaction between the test nucleic acid and a non-specific DNA member in a sample which is less than 10 fold, preferably less than 100 fold as intense as the specific interaction observed with the target DNA. The intensity of interaction may be measured, for example, by radiolabelling the probe, e.g., with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below. Conditions for stringency are also described in U.S. Pat. No. 5,976,838, the teachings of which are incorporated herein by reference in its entirety. In particular, examples of highly stringent, stringent, reduced and least stringent conditions are provided in U.S. Pat. No. 5,976,838, in the Table on page 15. Examples of stringency conditions for solutions during and after hybridization are shown, and highly stringent conditions are those that are at least as stringent as, for example, conditions A-F; stringent conditions are at least as stringent as, for example, conditions G-L; and reduced stringency conditions are at least as stringent as, for example, conditions M-R of that table.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 1×SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100 :g/ml denatured salmon sperm DNA at 65° C., or (6) 5×SSC, 5× Denhardt's solution, 50% formamide, 1% SDS, 100 :g/ml denatured salmon sperm DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61(% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 4×SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100 μg/ml denatured salmon sperm DNA at 65° C., or (6) 5×SSC, 5× Denhardt's solution, 50% formamide, 1% SDS, 100 :g/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 50° C., (2) 6×SSC, 50% formamide, 1% SDS, 0.1-2 mg/ml denatured salmon sperm DNA at 40° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 50° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5×SSC, 5× Denhardt's solution, 1% SDS, 100 :g/ml denatured salmon sperm DNA at 50° C., or (6) 5×SSC, 5× Denhardt's solution, 50% formamide, 1% SDS, 100 :g/ml denatured salmon sperm DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

In a preferred aspect, the present invention covers the use of nucleotide sequences that can hybridise to the nucleotide sequence of the present invention under stringent conditions (e.g., 65° C. and 0.1×SSC (1×SSC=0.15 M NaCl, 0.015 M Na₃ Citrate pH 7.0)).

Where the polynucleotide is double-stranded, both strands of the duplex, the use of either individually or in combination, is encompassed by the present invention. Where the polynucleotide is single-stranded, it is to be understood that the use of the complementary sequence of that polynucleotide is also included within the scope of the present invention.

Polynucleotides which are not 100% homologous to the sequences in Table 1B but the use of which falls within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g., rat, mouse, bovine and primate cells), may be obtained and such homologues and fragments thereof in general will be capable of selectively hybridising to sequences identified in Table 1B. Such sequences may be obtained by probing cDNA libraries made from or genomic DNA libraries from other animal species, and probing such libraries with probes comprising all or part of any on of the sequences under conditions of medium to high stringency. The nucleotide sequences of or which encode the human proteins identified in column 3 of Table 1B, may preferably be used to identify other primate/mammalian homologues or allelic variants.

Variants and strain/species homologues may also be obtained using degenerate PCR which will use primers designed to target sequences within the variants and homologues of the sequences of Table 1B. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labeled fragments.

In addition, homologous sequences may be identified by searching nucleotide and/or protein databases using search algorithms such as the BLAST suite of programs.

Alternatively, such polynucleotides may be obtained by site directed mutagenesis of characterised sequences. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. For example, further changes may be desirable to represent particular coding changes found in nucleic acid sequences which give rise to mutant genes which have lost their regulatory function. Probes based on such changes can be used as diagnostic probes to detect such mutants.

Polynucleotides may be used to produce a primer, e.g., a PCR primer, a primer for an alternative amplification reaction, a probe, e.g., labeled with a revealing label by conventional means using radioactive or non-radioactive labels, or the polynucleotides may be cloned into vectors. Such primers, probes and other fragments will be at least 8, 9, 10, or 15, preferably at least 20, for example at least 25, 30 or 40 nucleotides in length, and are also encompassed by the term polynucleotides as used herein.

Polynucleotides such as a DNA polynucleotides and probes for use in the invention may be produced recombinantly, synthetically, or by any means available to those of skill in the art. They may also be cloned by standard techniques. The invention also encompasses a composition comprising one or more isolated polynucleotides encoding an apoptosis protein, e.g., a vector containing a polynucleotide encoding an apoptosis protein, and also host cells containing such a vector. By “host cell” is meant a cell which has been or can be used as the recipient of transferred nucleic acid by means of a vector. Host cells can prokaryotic or eukaryotic, mammalian, plant, or insect, and can exist as single cells, or as a collection, e.g., as a culture, or in a tissue culture, or in a tissue or an organism. Host cells can also be derived from normal or diseased tissue from a multicellular organism, e.g., a mammal. Host cell, as used herein, is intended to include not only the original cell which was transformed with a nucleic acid, but also descendants of such a cell, which still contain the nucleic acid. The exogenous polynucleotide may be maintained as a non-integrated vector, for example, a plasmid, or alternatively, may be integrated into the host genome. The vector can also contain regulatory sequences, e.g., sequences permitting expression of the polynucleotide.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g., of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g., by purifying the reaction mixture on an agarose gel) and recovering the amplified DNA. The primers may be designed to contain suitable restriction enzyme recognition sites so that the amplified DNA can be cloned into a suitable cloning vector.

Polynucleotides or primers for use in the invention may carry a revealing label. Suitable labels include radioisotopes such as ³²P or ³⁵S, enzyme labels, or other protein labels such as biotin. Such labels may be added to polynucleotides or primers of the invention and may be detected by using techniques well known by those in the art.

Polynucleotides or primers for use in the invention or fragments thereof labeled or unlabeled may be used by a person skilled in the art in nucleic acid-based tests for detecting or sequencing polynucleotides of the invention in the human or animal body.

Such tests for detecting generally comprise bringing a biological sample containing DNA or RNA into contact with a probe comprising a polynucleotide or primer of the invention under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO 89/03891 and WO 90/13667.

Tests for sequencing nucleotides for use in the invention include bringing a biological sample containing target DNA or RNA into contact with a probe comprising a polynucleotide or primer of the invention under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing nucleotides identified in Table 1B in a biological sample may be used to determine particular sequences within cells in individuals who have, or are suspected to have, an altered gene sequence, for example within cancer cells including leukaemia cells and solid tumours such as breast, ovary, lung, colon, pancreas, testes, liver, brain, muscle and bone tumours. Cells from patients suffering from a inflammatory disease may also be tested in the same way.

In addition, the identification of the genes described in the Examples will allow the role of these genes in hereditary diseases to be investigated. In general, this will involve establishing the status of the gene (e.g., using PCR sequence analysis), in cells derived from animals or humans with, for example, neoplasms.

The probes for use in the invention may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like.

Homology Searching

Sequence homology (or identity) may be determined using any suitable homology algorithm, using for example default parameters.

Advantageously, the BLAST algorithm is employed, with parameters set to default values. The BLAST algorithm is described in detail at the world wide web site (“www”) of the National Center for Biotechnology Information (“.ncbi”) of the National Institutes of Health (“nih”) of the U.S. government (“.gov”), in the “/Blast/” directory, in the “blast_help.html” file. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8 (see the “blast_help.html” file, as described above) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al. (1994).

Most preferably, sequence comparisons are conducted using the simple BLAST search algorithm provided at the NCBI world wide web site described above, in the “/BLAST” directory.

Preparation of Apoptosis-Associated Polypeptides

Apoptosis-associated polypeptides in accordance with the present invention can be produced by any desired technique, including chemical synthesis, isolation from biological samples and expression of a nucleic acid encoding such a polypeptide. Nucleic acids, in their turn, can be synthesised or isolated from biological sources of _apoptosis-associated proteins.

The invention thus relates to vectors encoding a polypeptide according to the invention, or a fragment thereof. The vector can be, for example, a phage, plasmid, viral, or retroviral vector.

Nucleic acids according to the invention can be part of a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The nucleic acid insert is operatively linked to an appropriate promoter, such as the phage lambda PL promoter, the E. coli lac, trp, phoA and tac promoters, the SV40 early and late promoters and promoters of retroviral LTRs. Other suitable promoters are known to those skilled in the art. The expression constructs further contain sites for transcription initiation, termination, and, in the transcribed region, a ribosome binding site for translation. The coding portion of the transcripts expressed by the constructs preferably includes a translation initiating codon at the beginning and a termination codon (UAA, UGA or UAG) appropriately positioned at the end of the polypeptide to be translated.

As indicated, the expression vectors preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris); insect cells such as Drosophila S2 and Spodoptera Sf9 cells; animal cells such as CHO, COS, 293, and Bowes melanoma cells; and plant cells.

Appropriate culture media and conditions for the above-described host cells are known in the art and available commercially.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK2233, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PA0815 (all available from Invitrogen, Carlsbad, Calif.).

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Sambrook et al., referred to above.

A polypeptide according to the invention can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides according to the present invention can also be recovered from biological sources, including bodily fluids, tissues and cells, especially cells derived from tumour tissue or suspected tumour tissues from a subject.

In addition, polypeptides according to the invention can be chemically synthesised using techniques known in the art (for example, see Creighton, 1983, Proteins: Structures and Molecular Principles, W. H. Freeman & Co., N. Y., and Hunkapiller et al., Nature, 310: 105-111 (1984)). For example, a polypeptide corresponding to a fragment of apoptosis-associated protein can be synthesised by use of a peptide synthesiser.

Antibodies

The invention also provides the use of monoclonal or polyclonal antibodies to polypeptides encoded by the nucleic acids identified in Table 1B or fragments thereof.

Methods for production of antibodies are known by those skilled in the art. If polyclonal antibodies are desired, a selected mammal (e.g., mouse, rabbit, goat, horse, etc.) is immunised with an immunogenic polypeptide bearing an epitope(s) from a polypeptide. Serum from the immunised animal is collected and treated according to known procedures. Various adjuvants known in the art can be used to enhance antibody production. (for further details see for example: Using Antibodies, A Laboratory Manual, edited by Harlow, E. and Lane, D., 1999, Cold Spring Harbor Laboratory Press, (e.g. ISBN 0-87969-544-7)). If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order to generate a larger immunogenic response, polypeptides or fragments thereof maybe haptenised to another polypeptide for use as immunogens in animals or humans.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce single chain antibodies to the apoptosis-associated protein of interest. Antibody-based agents useful in practicing the present invention also include antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to the apoptosis-associated protein of interest.

Techniques for the production and isolation of monoclonal antibodies and antibody fragments are well-known in the art, and are additionally described, among other places, in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against epitopes from polypeptides encoded by the nucleic acids identified in Table 1Bare particularly useful in diagnosis, and those which are neutralising are useful in passive immunotherapy.

Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in detecting apoptosis-associated polypeptides identified herein in biological samples by a method which comprises: (a) providing an antibody of the invention; (b) incubating a biological sample with said antibody under conditions which allow for the formation of an antibody-antigen complex; and (c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues.

Antibodies that specifically bind to the apoptosis proteins can be used in diagnostic methods and kits that are well known to those of ordinary skill in the art to detect or quantify the apoptosis proteins in a body fluid or tissue. Results from these tests can be used to diagnose or predict the occurrence or recurrence of a cancer and other apoptosis-mediated diseases.

The invention also includes use of the apoptosis proteins, antibodies to those proteins, and compositions comprising those proteins and/or their antibodies in diagnosis or prognosis of diseases characterized by proliferative activity. As used herein, the term “prognostic method” means a method that enables a prediction regarding the progression of a disease of a human or animal diagnosed with the disease, in particular, an apoptosis-dependent disease. The term “diagnostic method” as used herein means a method that enables a determination of the presence or type of apoptosis-dependent disease in or on a human or animal.

The apoptosis proteins can be used in a diagnostic method and kit to detect and quantify antibodies capable of binding the proteins. These kits would permit detection of circulating antibodies to the apoptosis proteins which indicates, e.g. enhanced apoptosis in diseased cells.

Antibodies for use in the invention may be bound to a solid support and/or packaged into kits in a suitable container along with suitable reagents, controls, instructions and the like.

When used in a diagnostic composition, an antibody is preferably provided together with means for detecting the antibody, which can be enzymatic, fluorescent, radioisotopic or other means. The antibody and the detection means can be provided for simultaneous, simultaneous separate or sequential use, in a diagnostic kit intended for diagnosis.

The antibodies of the invention can be assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al, eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2,1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognises the primary antibody, e.g., an antihuman antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen.

ELISAs comprise preparing antigen, coating the well of a 96 well microtitre plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

The binding affinity of an antibody to an antigen and the off-rate of an antibody-antigen interaction can be determined by competitive binding assays. One example of a competitive binding assay is a radioimmunoassay comprising the incubation of labelled antigen (e.g., ³H or ¹²⁵I) with the antibody of interest in the presence of increasing amounts of unlabeled antigen, and the detection of the antibody bound to the labelled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labelled compound (e.g., ³H or ¹²⁵I) in the presence of increasing amounts of an unlabeled second antibody.

Methods of Detecting Proteins that Interact with an Apoptosis-Associated Protein

One useful technique for identifying interacting proteins is immunoprecipitation as described above.

Another useful technique for identifying interacting protein is the yeast-two hybrid system described, for example in Bartel et al. (eds.), The Yeast Two-Hybrid System, Oxford University Press (1997) (ISBN: 0195109384) the disclosure of which is incorporated herein by reference.

Protein interactions can also be analysed using protein arrays. These may be generated by a range of different techniques which allow proteins to be deposited on a flat surface at different densities. High density protein arrays can be generated using automated approaches similar to those described for DNA arrays (see below). Proteins interacting with apoptosis-associated proteins may be identified by, for example, using apoptosis-associated proteins to probe an expression array. Positive interactions could then be detected by the presence of, for example, a labelled antibody or by placing a tag on apoptosis-associated. The identity of the interacting protein can be determined by techniques such as mass spectrometry.

Measuring Gene Expression

Levels of gene expression may be determined using a number of different techniques.

a) at the RNA Level

Gene expression can be detected at the RNA level. RNA may be extracted from cells using RNA extraction techniques including, for example, using acid phenol/guanidine isothiocyanate extraction (RNAzol B; Biogenesis), or RNeasy RNA preparation kits (Qiagen).Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U., et al., Science 242:229-237 (1988) and Lewis, R., Genetic Engineering News 10: 1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., (1994), Gynaecologic Oncology, 52: 247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B. (1989) Genomics 4:560. In the Qβ Replicase technique, RNA replicase for the bacteriophage Qβ, which replicates single-stranded RNA, is used to amplify the target DNA, as described by Lizardi et al. (1988) Bio/Technology 6:1197.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., (1992) PNAS (USA) 80:392) begins with a specifically defined sequence unique to a specific target.

Primers suitable for use in various amplification techniques can be prepared according to methods known in the art.

Once the nucleic acid has been amplified, a number of techniques are available for the quantification of DNA and thus quantification of the RNA transcripts present. Methods for detection which can be employed include radioactive labels, enzyme labels, chemiluminescent labels, fluorescent labels and other suitable labels.

The detection of nucleic acids encoding an apoptosis-associated protein can be used, in the context of the present invention, to identify early stage apoptosis in cells.

b) at the Polypeptide Level

Gene expression may also be detected by measuring the apoptosis-associated polypeptides. This may be achieved by using molecules which bind to the apoptosis-associated polypeptide. Suitable molecules/agents which bind either directly or indirectly to apoptosis-associated proteins in order to detect the presence of the protein include naturally occurring molecules such as peptides and proteins, for example antibodies, or they may be synthetic molecules.

Standard laboratory techniques such as immunoblotting as described above can be used to detect altered levels of apoptosis-associated protein, as compared with untreated cells in the same cell population.

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Monitoring the Onset of Apoptosis

A number of methods are known in the art for monitoring the onset of apoptosis. These include morphological analysis, DNA ladder formation, cell cycle analysis, extemalisation of membrane phospholipid phosphatidyl serine and caspase activation analysis. Cell survival may be monitored by a number of techniques including cell cycle analysis and measuring cell viability. Measurements of cell proliferation may be made using a number of techniques including a plaque assay in which adherent cells are plated out in tissue culture plates and left to grow prior to fixing and staining. The number of colonies formed reflects the amount of cell proliferation.

Modifying the Functional Activity of an Apoptosis Protein

The functional activity of an apoptosis protein may be modified by suitable molecules/agents which bind either directly or indirectly to an apoptosis protein, or to the nucleic acid encoding it. Agents may be naturally occurring molecules such as peptides and proteins, for example antibodies, or they may be synthetic molecules. Methods of modulating the level of expression of an apoptosis protein include, for example, using antisense techniques.

Thus, test or candidate agents for use in the present invention can be based on antisense oligonucleotide constructs. Anti-sense oligonucleotides, including anti-sense RNA molecules and anti-sense DNA molecules, would act to directly block the translation of mRNA for the apoptosis-associated protein of interest by binding thereto and thus preventing protein translation or increasing mRNA degradation, thus decreasing the level of the apoptosis-associated protein of interest., and thus activity, in a cell. For example, antisense oligonucleotides of at least about 15 bases and complementary to unique regions of the mRNA transcript sequence encoding the apoptosis-associated protein of interest can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Suitable antisense molecules may be variants, based on these molecules, which have been chemically modified. For example, the antisense nucleic acids can usefully include altered, often nuclease-resistant, internucleoside bonds. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797).

Other modified oligonucleotide backbones are, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Other modified oligonucleotide backbones for antisense use that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Other methods of modulating gene expression are known to those skilled in the art and include dominant negative approaches as well as introducing peptides or small molecules which inhibit gene expression or functional activity.

Small inhibitory RNAs (siRNAs) can also function as test agents for use in the present invention. Gene expression of the apoptosis-associated protein of interest can be reduced by contacting the tumor, subject or cell with a small double stranded RNA (dsRNA), or a vector or construct causing the production of a small double stranded RNA, such that expression of the apoptosis-associated protein of interest is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-25 1; McManus, M. T. and Sharp, P. A. (2002) Nature Reviews Genetics 3:737-747; Bremmelkamp, T. R. et al. (2002) Science 296:550-553; U.S. Pat. No. 6,573,099; WO 01/36646; WO 99/32619; U.S. Pat. No. 6.506,559; WO 01/68836).

Ribozymes can also function as test agents for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hammerhead motif ribozyme molecules that specifically and efficiently catalyze endonucleolytic cleavage of mRNA sequences for the apoptosis-associated protein of interest are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as test agents can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In addition, changes in events immediately down-stream of an apoptosis associated protein, such as expression of genes whose transcription is regulated by expression of an apoptosis-associated protein, can be used as an indication that a molecule in question affects the functional activity of such a protein.

Expressing Apoptosis-Associated Proteins in Cells

Apoptosis-associated proteins may be expressed in cells by introducing vectors encoding the apoptosis-associated polypeptide.

Particularly useful in the present invention are those vectors that will drive expression of polypeptides from the inserted heterologous nucleic acid (“expression vectors”). These will often include a variety of other genetic elements operatively linked to the protein-encoding heterologous nucleic acid insert, typically genetic elements that drive transcription, such as promoters and enhancer elements, those that facilitate RNA processing, such as transcription termination and/or polyadenylation signals, and those that facilitate translation, such as ribosomal consensus sequences. As will be recognised by those skilled in the art, conditions that permit expression of the polypeptide from these vectors will depend on the type of vector and cell expression system chosen.

Vectors for expressing proteins are known for expression in prokaryotic cells, in yeast cells, typically S. cerevisiae and in mammalian cells and each include the specifc genetic elements for expression in the particular cell type.

Vector-drive protein expression can be constitutive or inducible. Inducible vectors include either naturally inducible promoters, such as the trc promoter, which is regulated by the lac operon, and the pL promoter, which is regulated by tryptophan, the MMTV-LTR promoter, which is inducible by dexamethasone, or can contain synthetic promoters and/or additional elements that confer inducible control on adjacent promoters.

Methods for introducing the vectors and nucleic acids into host cells are well known in the art; the choice of technique will depend primarily upon the specific vector to be introduced and the host cell chosen. Plasmid vectors will typically be introduced into chemically competent or electrocompetent bacterial cells. Vectors can be introduced into yeast cells by spheroplasting, treatment with lithium salts, electroporation, or protoplast fusion. Mammalian and insect cells can be directly infected by packaged viral vectors, or transfected by lipid, chemical or electrical means.

Expression vectors can be designed to fuse the expressed polypeptide to small protein tags that facilitate purification and/or visualization.

For example, proteins can be expressed with a tag that facilitates purification of the fusion protein. Suitable tags and their purification means are known and include poly-his/immobilized metal affinity chromatography, glutathione-S-transferase/glutathione affinity resins, Xpress epitope/detectable by anti-Xpress antibody (Invitrogen, Carlsbad, Calif., USA), myc tag/anti-myc tag antibody, V5 epitope/anti-V5 antibody (Invitrogen, Carlsbad, Calif., USA) and FLAG® epitope/anti-FLAG® antibody (Stratagene, La Jolla, Calif., USA).

For secretion of expressed proteins, vectors can include appropriate sequences that encode secretion signals, such as leader peptides. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as those that have a green fluorescent protein (GFP)-like chromophore, fusions to the IgG Fc region, and fusions for use in two hybrid systems.

Apoptosis-associated proteins can be expressed and purified from systems such as these for use in methods for detecting molecules which interact with apoptosis-associated proteins.

Biochemical assay tools and methodologies for use in the above described methods or processes to identify inhibitors of targets proteins are well known in the art. For example, in the case of GPCR receptor proteins, once the gene for a targeted protein, such as a human apoptosis-associated GPCR protein of this invention, is cloned, it is placed into a recipient cell, which then expresses the targeted protein on its surface. This cell, which expresses a single population of the targeted human receptor subtype, is then propagated resulting in the establishment of a cell line. This cell line, which constitutes a drug discovery system, is used in two different types of assays: binding assays and functional assays. In binding assays, the affinity of a compound for both the receptor subtype that is the target of a particular drug discovery program and other receptor subtypes that could be associated with side effects are measured. These measurements enable one to predict the potency of a compound, as well as the degree of selectivity that the compound has for the targeted receptor subtype over other receptor subtypes. The data obtained from binding assays also enable chemists to design compounds toward or away from one or more of the relevant subtypes, as appropriate, for optimal therapeutic efficacy. In functional assays, the nature of the response of the receptor subtype to the compound is determined. Data from the functional assays show whether the compound is acting to inhibit or enhance the activity of the receptor subtype, thus enabling pharmacologists to evaluate compounds rapidly at their ultimate human receptor subtypes targets permitting chemists to rationally design drugs that will be more effective and have fewer or substantially less severe side effects than existing drugs.

Approaches to designing and synthesizing receptor subtype-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor subtype to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

Thus, in one aspect of this invention, activity of an apoptosis-associated GPCR protein of this invention can be monitored by measuring a G-protein-coupled readout. For example, such readouts can be monitored using an electrophysiological method to determine the activity of G-protein regulated Ca²⁺ or K⁺ channels or by using fluorescent dye to measure changes in intracellular Ca²⁺ levels. Other methods that can typically be used to monitor receptor activity involved measuring levels of or activity of GTPγS or cAMP.

Detecting Molecules which Interact with Apoptosis-Associated Proteins

Techniques such as analytical centrifugation, affinity binding studies involving chromatography or electrophoresis can be used to detect molecules which interact directly with apoptosis-associated proteins. Those skilled in the art will appreciate that this list is by no means exhaustive. More specifically, it is possible to use an apoptosis-associated protein as an affinity ligand to identify agents which bind to it; labeling it with a detectable label and using it as a probe to detect apoptotic products in electrophoresis gels; labeling the apoptosis-associated protein target and using it to probe libraries of genes and/or cDNAs; labeling the apoptosis-associated protein target and using it to probe cDNA expression libraries to find clones synthesizing proteins which can bind to the target; performing UV-crosslinking studies to identify agents which can bind to the target; using the apoptosis-associated protein in gel retardation assays which would detect its ability to bind to nucleic acid encoding identified agents; performing footprinting analyses to identify the regions within a nucleic acid to which the target binds. Those skilled in the art will be aware of other suitable techniques and will appreciate that this list is not intended to be exhaustive.

Other techniques that allow the identification of protein-protein interactions include Yeast two hybrid and immunoprecipitation, as described above.

Yeast assays may be used to screen for agents that modulate the activity of an apoptosis-associated GPCR protein of this invention, or variant polypeptides. A typical yeast assay involves heterologously expressing an apoptosis-associated GPCR protein of this invention or a variant polypeptide in a modified yeast strain containing multiple reporter genes, typically FUS1p-HIS-3 and FUS1p-lacZ, each linked to an endogenous MAPK cascade-based signal transduction pathway. This pathway is normally linked to pheromone receptors, but can be coupled to foreign receptors by replacement of the yeast G-protein with yeast/mammalian G-protein chimeras. Strains may also contain further gene deletions, such as deletions of SST2 and FAR1, to potentiate the assay. Ligand activation of the heterologous receptor can be monitored for example either as cell growth in the absence of histidine or with a suitable substrate for beta-galactosidase (lacZ). Such technology is well known in the art. See for example WO 99/14344, WO 00/12704, or U.S. Pat. No. 6,100,042.

Alternatively melanophore assays may be used to screen for modulators of an apoptosis-associated GPCR protein of this invention. An apoptosis-associated GPCR protein can be heterologously expressed in Xenopus laevis melanophores and their activation or inhibition can be measured by either melanosome dispersion or aggregation. Basically, melanosome dispersion is promoted by activation of adenylate cyclase or phospholipase C, i.e. Gs and Gq mediated signalling respectively, whereas aggregation results from activation of Gi-protein resulting in inhibition of adenylate cyclase. Hence, ligand activation of the heterologous receptor can be measured simply by measuring the change in light transmittance through the cells or by imaging the cell response.

Preferably, control experiments are carried out on cells which do not express the apoptosis-associated GPCR protein of the invention in order to establish whether the observed responses are the result of modulation of the apoptosis-associated GPCR protein.

Protein interactions can also be analysed using protein arrays. These may be generated by a range of different techniques which allow proteins to be deposited on a flat surface at different densities. High density protein arrays can be generated using automated approaches similar to those described for DNA arrays (see below). Proteins interacting with apoptosis-associated proteins may be identified by, for example, using these proteins to probe an expression array. Positive interactions could then be detected by the presence of, for example, a labelled antibody or by placing a tag on apoptosis-associated proteins. The identity of the interacting protein can be determined by techniques such as mass spectrometry.

Cells

Cells useful in the method of the invention may be from any source, for example from primary cultures, from established cell lines, in organ culture or in vivo. Cell lines useful in the invention include cells and cell lines of haematopoietic origin. Suitable cells include HeLa, U937 (monocyte), TF1, HEK293 (T), primary cultures of neutrophils or cells having neutrophil characteristics, for example HL60 cells, murine FDCP-1, FDCPmix, 3T3, primary or human stem cells.

Measuring Global Gene Expression

Expression of recombinant apoptosis-associated proteins in cells can be induced using an expression system including any of those described herein.

Determination of expression levels of genes associated with apoptosis-associated proteins will enable the identification of other known or novel genes that play a role in apoptosis.

Regulation of gene activity can be accomplished at a number of levels. Most commonly, regulation is at the transcriptional level—specific transcription factors modulate the expression of subsets of target genes. Post-transcriptional regulation (translational regulation), is determined by the rate and mechanism of RNA processing in the cell, i.e. accumulation, translation and degradation. Subsequent protein-level regulation of genetic activity is accomplished through post-translational modification.

A number of individual gene product types whose expression or function is associated with an apoptosis-associated protein gene expression may be screened for in the present invention. These products include polypeptides and nucleic acids. The expression levels assessed may be absolute levels of production of a particular polypeptide or nucleic acid, or the levels of production of a derivative of any polypeptide or nucleic acid. For example, the invention may be configured to measure the level of expression of a particular mRNA splice variant, or the amount present of a phosphorylated derivative of a particular polypeptide.

Where it is desired to monitor the levels of expression of a known gene product, conventional assay techniques may be employed, including nucleic acid hybridisation studies and activity-based protein assays. Kits for the quantitation of nucleic acids and polypeptides are available commercially.

Where the gene product to be monitored is unknown, however, methods are employed which facilitate the identification of the gene product whose expression is to be measured. For example, where the gene product is a nucleic acid, arrays of oligonucleotide probes may be used as a basis for screening populations of mRNA derived from cells.

a) Arrays

Gene Arrays of oligonucleotides specific to gene sequences archived in public domain databases, such as GenBank, are available commercially from a number of suppliers (such as Incyte Genomics, USA). Examples of such commercial arrays are in the form of either nucleotides spotted onto a membrane filter (such as nitrocellulose), or a solid support (such as glass). Commercial Gene Arrays are used to profile the patterns of gene expression which are associated with the process of apoptosis in neutrophils, and other cell types.

Gene Arrays can additionally be constructed specifically, by spotting nucleotide sequences derived from cDNA clones generated from novel libraries or from cDNA clones purchased commercially. Such arrays allow the expression profiling of proprietary and/or novel nucleotide sequences.

Gene Arrays are additionally constructed by commercial sources (e.g. Genescreen), by spotting nucleotide sequences derived from cDNA clones generated from novel libraries or from cDNA clones purchased commercially. Such arrays allow the expression profiling of proprietary and/or novel nucleotide sequences.

Many of the cDNA sequences or EST (expressed sequence tag) sequences deposited in the public domain databases are derived from a restricted set of tissue types, such as liver, brain and foetal tissue. The cloning of in-house cDNA libraries which are focused to specific cellular events, such as ROS-mediated apoptosis offers the possibility to identify, clone and characterise novel genes which are associated with this process. Similarly, the cloning of in-house cDNA libraries which are focused to specific tissue types, such as the neutrophil, offers the possibility to identify, clone and characterise novel genes whose expression is restricted to this cell type. Libraries (cDNA) constructed using a physical subtraction, such as the ClonTech ‘Select’ SSH method (suppression hybridisation) and novel modifications of such, as described, allow the selective cloning of genes whose expression is differentially regulated in the process or cell type being studied. Gene Array technology is combined with SSH cDNA libraries to identify false-positives and further focus on truly differentially expressed genes. Clones from each SSH library constructed are picked, cultured and archived as glycerol stocks. The cDNA inserts contained within individual plasmid clone are PCR amplified and spotted onto in-house arrays. Differential expression is confirmed using hybridisation with a radiolabelled probe generated from the mRNA used for each reciprocal subtractions.

Arrays of nucleic acids may be prepared by direct chemical synthesis of nucleic acid molecules. Chemical synthesis involves the synthesis of arrays of nucleic acids on a surface in a manner that places each distinct nucleic acid (e.g., unique nucleic acid sequence) at a discrete, predefined location in the array. The identity of each nucleic acid is determined by its spatial location in the array. These methods may be adapted from those described in U.S. Pat. No. 5,143,854; WO90/15070 and WO92/10092; Fodor et al. (1991) Science, 251: 767; Dower and Fodor (1991) Ann. Rep. Med. Chem., 26: 271.

In a preferred aspect of the invention, arrays of nucleic acids may be prepared by gridding of nucleic acid molecules. Oligonucleotides may be advantageously arrayed by robotic picking, since robotic techniques allow the most precise and condensed gridding of nucleic acid molecules; however, any technique, including manual techniques, which is suitable for locating molecules at discrete locations on a support, may be used.

The gridding may be regular, such that each colony is at a given distance from the next, or random. If molecules are spaced randomly, their density can be adjusted to statistically reduce or eliminate the probability of overlapping on the chosen support.

Apparatus for producing nucleic acid microarrays is available commercially, for example from Genetix and Genetic Microsystems. Moreover, pre-prepared arrays of nucleic acid molecules are available commercially, for example from Incyte Genomics Inc. (Human LifeGrid™). Such arrays will comprise expressed sequence tags (ESTs) representative of most or all the genes expressed in a cell or organism, thus providing a platform for the screening of mRNA populations from multiple ROS-treated cells.

Samples for mRNA population analysis may be isolated and purified by any suitable mRNA production method; for example an RNA isolation kit is available from Stratagene.

In addition, where the gene product is a polypeptide, arrays of antibodies may be used as a basis for screening populations of polypeptides derived from cells. Examples of protein and antibody arrays are given in Proteomics: A Trends Guide, Elsevier Science Ltd., July 2000 which is incorporated by reference.

In the context of the present invention, array technology can also be used, for example, in the analysis of the expression of one or more of the apoptosis proteins identified herein. In one embodiment, array technology may be used to assay the effect of a candidate compound on a number of the apoptosis proteins identified herein simultaneously. Accordingly, another aspect of the present invention is to provide microarrays that include at least one, at least two or at least several of the nucleic acids identified in Table 1, or fragments thereof, or protein or antibody arrays.

b) 2D PAGE

For the monitoring of unknown polypeptide gene products, separation techniques such as 2 dimensional gel electrophoresis are employed. 2D PAGE typically involves sample preparation, electrophoresis in a first dimension on an immobilised pH gradient, SDS-PAGE electrophoresis in a second dimension, and sample detection. Protocols for 2D PAGE are widely available in the art, for example at http://www.expasy.ch/ch2d/protocols/, the contents of which as of Nov. 30, 2001 are incorporated herein by reference.

Samples for 2D PAGE may be prepared by conventional techniques. In the case of the present invention, HeLa cells transfected with apoptosis-associated proteins are grown in a suitable medium, such as RPMI 1640 containing 10% foetal calf serum (FCS). The suspension is transferred into a tube and the cells are centrifuged at 1000 g for 5 minutes. Supernatant is discarded and the cells are washed with RPMI 1640 without FCS. After centrifugation and removal of RPMI 1640, 0.8×10⁶ cells are mixed and solubilised with 60 μl of a solution containing urea (8 M), CHAPS (4% w/v), Tris (40 mM), DTE (65 mM) and a trace of bromophenol blue. The whole final diluted HeLa sample is loaded on the first dimensional separation.

The method of the present invention advantageously employs a step of establishing a reference expression level for the gene products being investigated. This can be carried out by using un-transfected HeLa cells to serve as a standard for one or more subsequent assays; or it may be an integral part of every assay. For example mRNA or polypeptide populations from HeLa transfected and untransfected cells may be assessed simultaneously on a nucleic acid array or by 2D PAGE, and changes in expression patterns identified by direct comparison.

Analysis of 2D PAGE results, using appropriate software where necessary, reveals polypeptides of interest which may be isolated, sequenced and used to identify genes encoding them.

Assays

The present invention provides assays that are suitable for identifying substances which bind to polypeptides of the invention and which affect progression of apoptosis as characterised, for example, by caspase activation, DNA fragmentation, cell shrinkage and fragmentation, exposure of phospholipids on the plasma membrane and so forth. In addition assays which are suitable for indentifying modulators of GPCR and protein kinases, including P13 kinases are incorporated.

In general, a substance which inhibits one or more of these aspects of apoptosis either inhibits it completely, or leads to a significant (i.e., greater than 50%) reduction in protein activity at concentrations of 500 mM or less, relative to controls. Preferably, the inhibition is by 75% relative to controls, more preferably by 90%, and most preferably by 95% or 100% relative to controls. A substance which enhances or increases one or more of these aspects of apoptosis leads to a significant (i.e., greater than 50%) increase in protein activity at concentrations of 500 mM or less, relative to controls. Preferably, the increase is by 75% relative to controls, more preferably by 90%, and most preferably by 95% or 100% relative to controls.

In addition, assays can be used to identify substances that interfere with binding of polypeptides of the invention, where appropriate, to components of the apoptotic machinery. Such assays are typically in vitro. Assays are also provided that test the effects of candidate substances identified in preliminary in vitro assays on intact cells in whole cell assays. The assays described below, or any suitable assay as known in the art, may be used to identify these substances.

According to one aspect of the invention, therefore, we provide one or more substances identified by any of the assays described below, viz, apoptosis assays kinase assays, kinase inhibitor assays, GPCR and whole cell assays, as described in further detail below.

Modulator Screening Assays

Methods for inducing apoptosis are well known in the art and include, without limitation, exposure to chemotherapy or radiotherapy agents and withdrawal of obligate survival factors (e.g. GM-CSF, NGF) if applicable. Differences between treated and untreated cells indicates effects attributable to the test compound.

Myeloid cells die spontaneously in culture although with differing time courses depending on the cell type. Neutrophils in culture apoptose within 24 hours although this can be delayed to over 48 hours in the presence of survival factors. Eosinophil apoptosis is observed over 48 hours with a delay to several days in the presence of survival factors. Macrophages are generally much longer lived. Thus, the ability of a compound to modulate myeloid cell apoptosis can be assessed by monitoring the rate of apoptosis in the presence or absence of the test compound and after the withdrawal of obligate survival factors (e.g. GM-CSF, IL-8, IL-5, G-CSF or BAL) if applicable. Differences between treated and untreated cells indicates effects attributable to the test compound.

Compounds having inhibitory, activating, or modulating activity can be identified using in vitro and in vivo assays for apoptosis protein activity and/or expression, e.g., ligands, agonists, antagonists, and their homologs and mimetics. Modulator screening may be performed by adding a putative modulator test compound to a tissue or cell sample, and monitoring the effect of the test compound on the function and/or expression of an apoptosis protein. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds.

A substance that inhibits apoptosis as a result of an interaction with a polypeptide of the invention may do so in several ways. For example, if the substance inhibits a PI3K or GPCR signalling pathway, it may directly disrupt the binding of a polypeptide of the invention to a component of the singnalling pathway by, for example, binding to the polypeptide and masking or altering the site of interaction with the other component. A substance which inhibits a PI3K or GPCR signalling pathway may do so by inhibiting the phosphorylation or de-phosphorylation of proteins involved. Candidate substances of this type may conveniently be preliminarily screened by in vitro binding assays as, for example, described below and then tested, for example in a whole cell assay as described below. Examples of candidate substances include antibodies which recognise a polypeptide of the invention.

A substance which can bind directly to a polypeptide of the invention may also inhibit its function in apoptosis by altering its subcellular localisation and hence its ability to interact with its normal substrate. The substance may alter the subcellular localisation of the polypeptide by directly binding to it, or by indirectly disrupting the interaction of the polypeptide with another component.

These substances may be tested using, for example the whole cells assays described below. Non-functional homologues of a polypeptide of the invention may also be tested for inhibition of apoptosis since they may compete with the wild type protein for binding to components of the apoptotic machinery whilst being incapable of the normal functions of the protein or block the function of the protein bound to the apoptotic machinery. Such non-functional homologues may include naturally occurring mutants and modified sequences or fragments thereof.

Alternatively, instead of preventing the association of the components directly, the substance may suppress the biologically available amount of a polypeptide of the invention. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

Suitable candidate substances include peptides, especially of from about 5 to 30 or 10 to 25 amino acids in size, based on the sequence of the polypeptides described in the Examples, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for a polypeptide of the invention. Furthermore, combinatorial libraries, peptide and peptide mimetics, defined chemical entities, oligonucleotides, and natural product libraries may be screened for activity as inhibitors of binding of a polypeptide of the invention to the apoptotic machinery. The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of any of the stages of apoptosis.

Other suitable test or candidate substances which can be tested in the above assays include combinatorial libraries, defined chemical entities, peptide and peptide mimetics, oligonucleotides and natural product libraries, and display (e.g. phage display libraries) and antibody products. Assays may also be carried out in the presence of ligands or activators of the G-protein coupled receptors. Test substances may be used in an initial screen of, for example, 10 substances per reaction, and the substances of these batches that show inhibition or activation tested individually. Test substances may be used at a concentration of from 1 nM to 1000 μM, preferably from 1 μM to 100 μM, more preferably from 1 μM to 10 μM.

Cells expressing an apoptosis-associated GPCR protein of this invention may be used to screen for ligands using functional assays. The same assays, in the presence or absence of a ligand, may be used to identify agonists or antagonists of the apoptosis-associated GPCR protein that may be employed for a variety of therapeutic purposes. It is well known to those in the art that the over-expression of a GPCR can result in the constitutive activation of intracellular signaling pathways. In the same manner, over-expression of the apoptosis-associated GPCR protein in any cell line as described above, can result in the activation of the functional responses described below, and any of the assays herein described can be used to screen for both agonist and antagonist ligands of the apoptosis-associated GPCR protein.

A wide spectrum of functional assays can be employed to screen for modulators of apoptosis-associated GPCR proteins. These assays range from traditional measurements of total inositol phosphate accumulation, cAMP levels, intracellular calcium mobilization, and potassium or sodium currents, for example; to systems measuring these same second messengers but which have been modified or adapted to be of higher throughput, more generic and more sensitive; to cell based assays reporting more general cellular events resulting from receptor activation such as metabolic changes, differentiation, cell division or proliferation.

In the preceding methods or processes of this invention for the identification of inhibitors of apoptosis-associated proteins, the type of compounds acting on these proteins, or the biochemical pathways that they modulate in cells, that are detectable, include antagonists, inverse agonists, partial inverse agonists, allosteric or allotopic antagonists, agonists, partial agonists, and allosteric or allotopic agonists. Assays for the detection of such compounds can be performed in the presence or absence of an activating ligand or other activating mechanism, e.g. binding of an activating protein or factor, or modification by phosphorylation or another reversible covalent modification mechanism.

In the practice of this invention, in order to detect interaction of a test agent with a protein kinase of this invention, any of the commonly used immunoassay techniques may be used for quantitation of the phosphotransferase activity of protein kinase apoptosis-associated proteins of this invention, including immunoprecipitation, immunoblotting (Western blotting), and ELISA assays. In one embodiment, an anti-kinase antibody is used for isolation of the kinase protein, for example by immunoprecipitation, and change in activity of the protein is quantitated either by using a labeled anti-phosphoamino acid antibody to assess autophosphorylation of the protein, or by the protein's phosphotransferase activity on a peptide or protein substrate. In a further embodiment, an ELISA assay is used in which the kinase is initially captured using an anti-kinase antibody, and autophosphorylation then assessed in a second step using a labeled anti-phosphoamino acid antibody.

For ELISA assays, specific binding pairs can be of the immune or non-immune type. Immune specific binding pairs are exemplified by antigen-antibody systems or hapten/anti-hapten systems. Examples of such include fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art (e.g. see Using Antibodies, A Laboratory Manual, edited by Harlow, E. and Lane, D., 1999, Cold Spring Harbor Laboratory Press, (e.g. ISBN 0-87969-544-7)). Such methods involve immunizing an animal with the antigen member of the specific binding pair. If the antigen member of the specific binding pair is not immunogenic ( e.g. a hapten) it can be covalently coupled to a carrier protein to render it immunogenic.

Furthermore, in the case of apoptosis-associated protein kinases of this invention, a detection method for activity changes in such a kinase, particularly when assay of purified proteins, or a protein in a membrane preparation is contemplated, is an assay for protein or peptide phosphotransferase activity, whereby activity is monitored by the incorporation of radiophosphorus into the protein through autophosphorylation with P³² phosphate, or by incorporation of radiophosphorus into an alternative substrate protein or peptide. Alternatively, many non-radioactive assay formats are available for assessment of kinase activity in purified preparations of enzymes, e.g. ELISAs, scintillation proximity, electrophoretic, immunoprecipitation, bead-based and Western blot. Detection methodologies include the use of radioactive tracers, colorimetric or chemiluminescent substrates, time-resolved fluorescence, resonance energy transfer (FRET), and fluorescence polarization. Many of these assay formats are readily available as commercial kits.

A substance is identified as a modulator of apoptosis activity when it is found to inhibit, decrease, increase, enhance, or activate such activity. In general, a substance which inhibits one or more of these aspects of apoptosis either inhibits it completely, or leads to a significant (i.e., greater than 50%) reduction in protein activity at concentrations of 500 mM or less, relative to controls (i.e., substance known to not modulate one or more aspects of apoptosis). Preferably, the inhibition is by 75% relative to controls, more preferably by 90%, and most preferably by 95% or 100% relative to controls. The inhibition may prevent apoptosis, or may simply delay or prolong apoptosis. A substance which enhances or increases one or more of these aspects of apoptosis leads to a significant (i.e., greater than 50%) increase in protein activity at concentrations of 500 mM or less, relative to controls. Preferably, the increase is by 75% relative to controls, more preferably by 90%, and most preferably by 95% or 100% relative to controls.

Polypeptide Binding Assays

One type of assay for identifying substances that bind to a polypeptide of the invention involves contacting a polypeptide of the invention, which is immobilised on a solid support, with a non-immobilised candidate substance determining whether and/or to what extent the polypeptide of the invention and candidate substance bind to each other. Alternatively, the candidate substance may be immobilised and the polypeptide of the invention non-immobilised.

The binding of the substance to the apoptosis-associated polypeptide can be transient, reversible or permanent. Preferably the substance binds to the polypeptide with a Kd value which is lower than the Kd value for binding to control polypeptides (i.e., polypeptides known to not be apoptosis-associated polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

In a preferred assay method, the polypeptide of the invention is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads (Smith and Johnson, 1988; Gene 67(10):31-40). As a control, binding of the candidate substance, which is not a GST-fusion protein, to the immobilised polypeptide of the invention is determined in the absence of the polypeptide of the invention. The binding of the candidate substance to the immobilised polypeptide of the invention is then determined.

This type of assay is known in the art as a GST pulldown assay. Again, the candidate substance may be immobilised and the polypeptide of the invention non-immobilised.

It is also possible to perform this type of assay using different affinity purification systems for immobilising one of the components, for example Ni—NTA agarose and histidine-tagged components.

Binding of the polypeptide of the invention to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Other in Vitro Assays

Other assays for identifying substances that bind to a polypeptide of interest are also provided. The binding of the substance to the apoptosis-associated polypeptide can be transient, reversible or permanent. Preferably the substance binds to the polypeptide with a Kd value which is lower than the Kd value for binding to control polypeptides (i.e., polypeptides known to not be apoptosis-associated polypeptides). Preferably the Kd value of the substance is 2 fold less than the Kd value for binding to control polypeptides, more preferably with a Kd value 100 fold less, and most preferably with a Kd 1000 fold less than that for binding to the control polypeptide.

Substances which affect kinase activity are of interest.

Substances which inhibit or affect kinase activity may be identified by means of a kinase assay as known in the art, for example, by measuring incorporation of ³²P into a suitable peptide or other substrate in the presence of the candidate substance. Similarly, substances which inhibit or affect proteolytic activity may be assayed by detecting increased or decreased cleavage of suitable polypeptide substrates.

Assays for these and other protein or polypeptide activities are known to those skilled in the art, and may suitably be used to identify substances which bind to a polypeptide of interest and affect its activity.

Whole Cell Assays

Candidate substances may also be tested on whole cells for their effect on apoptosis. Preferably the candidate substances have been identified by the above-described in vitro methods. Alternatively, rapid throughput screens for substances capable of inhibiting or enhancing apoptosis may be used as a preliminary screen and then used in the in vitro assay described above to confirm that the affect is on a particular polypeptide of interest.

The candidate substance, i.e., the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance identified by the method of the invention on apoptosis comprises administering the candidate substance to a cell and determining whether the substance inhibits apoptosis. Techniques for measuring apoptosis in a cell population are well known in the art. The extent of apoptosis in treated cells is compared with the extent apoptosis in an untreated control cell population to determine the degree of inhibition, if any. For example, an inhibitor of apoptosis may be assayed by measuring the proportion of cells in a sub-G1 population and comparing this to the proportion of cells in an untreated population.

The concentration of candidate substances used will typically be such that the final concentration in the cells is similar to that described above for the in vitro assays.

A candidate substance is typically considered to be an inhibitor of apoptosis if the proportion of cells undergoing apoptosis is reduced to below 50%, preferably below 40, 30, 20 or 10% of that observed in untreated control cell populations.

Suitably a polypeptide of interest in the context of the above assays is a polypeptide encoded by any nucleic acid sequence identified in Table 1B.

Methods for determining whether compounds that interact with an apoptosis-associated protein are inhibitors tumor growth, tumor cell growth or tumor cell proliferation, or activators of tumor cell apoptosis, are well known in the art.

The effects of compounds on tumor growth can be monitored by any of the many methods known to those of ordinary skill in the art, or modifications thereof. For example, the efficacy of the compounds can be tested in animal models. The efficacy of the compound alone or in combination with conventional anti-tumor agents such as cytotoxic/anti-neoplastic agents and anti-angiogenic agents can be compared to the conventional agents alone. Typically, a tumor of a given size is present in a rat or mouse. The mouse is treated with the agent and the size of the tumor is measured over time. The mean survival time of the animals can also be measured. The compounds modulating the activity of an apoptosis-associated protein that are used must actively bind the receptor in the animal which is to be tested. Xenografts can be implanted into the animal to test the ability of species specific compounds to inhibit. Suitable test animals include, but are not limited to, inbred rats such as Fischer 344 and Lewis rats, and athymic NCR-NU mice.

The effects of compounds on tumor cell growth or tumor cell proliferation can be monitored by any of the many methods known to those of ordinary skill in the art, or modifications thereof (e.g. see Methods section herein).

The effects of compounds on apoptosis can be monitored by any of the many methods known to those of ordinary skill in the art, or modifications thereof (e.g. see Methods section herein). Specific examples of apoptosis assays are also exemplified in the following references. Assays for apoptosis in lymphocytes are disclosed by: Li et. al., (1995) Science 268:429-431; Gibellini et. al. (1995) Br. J. Haematol. 89:24-33; Martin et al. (1994) J. Immunol. 152:330-42; Terai et al., (1991) J. Clin Invest. 87:1710-5; Dhein et al. (1995) Nature 373:438-441; Katsikis et al. (1995) J. Exp. Med. 1815:2029-2036; Westendorp et al. (1995) Nature 375:497; and DeRossi et al. (1994) Virology 198:234-44. Assays for apoptosis in fibroblasts are disclosed by: Vossbeck et al. (1995) Int. J. Cancer 61:92-97; Goruppi et al. (1994) Oncogene 9:1537-44; Fernandez et. al. (1994) Oncogene 9:2009-17; Harrington et al. (1994) EMBO J., 13:3286-3295; and Itoh et al., (1993) J. Biol. Chem. 268:10932-7. Assays for apoptosis in neuronal cells are disclosed by: Melino et al. (1994) Mol. Cell Biol. 14:6584-6596; Rosenblaum et al. (1994) Ann. Neurol. 36:864-870; Sato et. al. (1994) J. Neurobiol. 25:1227-1234; Ferrari et al. (1995) J. Neurosci. 1516:2857-2866; Talley et. al. (1995) Mol. Cell Biol. 1585:2359-2366; Talley et al. (1995) Mol. Cell. Biol. 15:2359-2366; and Waikinshaw et al. (1995) J. Clin. Invest. 95:2458-2464. Assays for apoptosis in insect cells are disclosed by: Clem et al. (1991) Science 254:1388-90; Crook et al. (1993) J. Virol: 67:2168-74; Rabizadeh et al. (1993) J. Neurochem. 61:2318-21; Birnbaum et al. (1994) J. Virol. 68:2521-8, 1994; and Clem et al. (1994) Mol. Cell. Biol. 14:5212-5222.

Therapeutic Uses

Many disease conditions are associated with defects in abnormal apoptosis. Such conditions include cancer, inflammation, autoimmune disease and neurodegenerative disordes. One therapeutic approach to treating such diseases may therefore be to modulate apoptosis. Such an approach may also be used for therapy of any proliferative disease in general. Thus, since the polypeptide of the invention appears to be required for normal apoptosis, it represents a target for modulation of its functions, particularly in diseased cells including tumour cells and inflammatory cells.

Cancer or proliferative diseases/disorders include malignant and pre-neoplastic disorders. The present invention is especially useful in relation to treatment or diagnosis of adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breast cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

One possible approach is to express anti-sense constructs directed against polynucleotides of the invention, preferably selectively in diseased cells, to inhibit or otherwise modulate gene function and induce or inhibit apoptosis. Another approach is to use non-functional variants of polypeptide of the invention that compete with the endogenous gene product for cellular components of the apoptotic machinery, resulting in inhibition of function. Alternatively, compounds identified by the assays described above as binding to a polypeptide of the invention may be administered to diseased cell to prevent or enhance the function of that polypeptide. This may be performed, for example, by means of gene therapy or by direct administration of the compounds. Suitable antibodies of the invention may also be used as therapeutic agents.

Alternatively, double-stranded (ds) RNA is a powerful way of interfering with gene expression in a range of organisms that has recently been shown to be successful in mammals (Wianny and Zernicka-Goetz, 2000, Nat Cell Biol 2000, 2, 70-75). Double stranded RNA corresponding to the sequence of a polynucleotide according to the invention can be introduced into or expressed in diseased cells of an individual to interfere with normal apoptosis.

In general, a disease is defined as being “treated” if the abnormal apoptosis associated with the disease or condition is significantly inhibited (i.e., by 50% or more) relative to controls. Preferably, the inhibition is by 75% relative to controls, more preferably by 90%, and most preferably by 95% or 100% relative to controls. The inhibition may prevent apoptosis, or may simply delay or prolong apoptosis.

Administration

Substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components (or antisense constructs) for use in inhibiting apoptosis may be administered directly as a naked nucleic acid construct. They may further comprise flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. It is particularly preferred to use polynucleotides/vectors that target specifically tumour or proliferative cells, for example by virtue of suitable regulatory constructs or by the use of targeted viral vectors.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide, polypeptide, compound or vector described here may be conjugated, joined, linked, fused, or otherwise associated with a membrane translocation sequence.

Preferably, the polynucleotide, polypeptide, compound or vector, etc described here may be delivered into cells by being conjugated with, joined to, linked to, fused to, or otherwise associated with a protein capable of crossing the plasma membrane and/or the nuclear membrane (i.e., a membrane translocation sequence). Preferably, the substance of interest is fused or conjugated to a domain or sequence from such a protein responsible for the translocational activity. Translocation domains and sequences for example include domains and sequences from the HIV-1-trans-activating protein (Tat), Drosophila Antennapedia homeodomain protein and the herpes simplex-1 virus VP22 protein. In a highly preferred embodiment, the substance of interest is conjugated with penetratin protein or a fragment of this. Penetratin comprises the sequence RQIKIWFQNRRMKWKK and is described in Derossi et al., 1994, J. Biol. Chem. 269:10444-50; use of penetratin-drug conjugates for intracellular delivery is described in WO 00/01417. Truncated and modified forms of penetratin may also be used, as described in WO 00/2927.

In the context of this invention, the carrier used in preparing a composition is preferably a pharmaceutically acceptable carrier. For therapeutic use, preferably the composition is comprised of a pharmaceutically acceptable carrier and a non-toxic therapeutically effective amount of a compound that inhibits the apoptosis-associated protein (or a pharmaceutically acceptable salt of such a compound).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of amount of a compound that inhibits the apoptosis-associated protein. Methods of preparing pharmaceutical compositions for therapeutic application of a compound that inhibits an apoptosis-associated protein are well known in the art, e.g. Remington's Pharmaceutical Sciences, Mack Publishing Company, Easton, Pa., 18th edition (1990).

The pharmaceutical compositions of the present invention may comprise an inhibitor compound as active ingredient, a pharmaceutically acceptable carrier and optionally other therapeutic ingredients or adjuvants. Other therapeutic agents may include cytotoxic, chemotherapeutic or anti-cancer agents, or agents which enhance the effects of such agents. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the inhibitor compounds of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an inhibitor compound composition may also be administered by controlled release means and/or delivery devices. The composition may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an inhibitor compound of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an inhibitor compound may also be prepared in powder or liquid concentrate form.

Dosage levels for the compounds of the combination of this invention will be approximately as described herein. It is understood, however, that the specific dose level for any particular patient will depend upon a variety of factors including the age, body weight, general health, sex, diet, time of administration, route of administration, rate of excretion, drug combination and the severity of the particular disease undergoing therapy.

To determine whether a patient has a type of neoplasia that is likely to respond to treatment with an inhibitor of an apoptosis-associated protein that is encoded by a gene selected from table 1B, a neoplastic tissue sample from the patient is exposed to such an inhibitor (e.g. a compound identified by the screening methods of this invention; a small inhibitory dsRNA, for example as described in Methods section) and is tested to determine whether the neoplastic tissue sample exhibits sensitivity to treatment with the inhibitor.

For example, in a patient with a solid tumor, a suspected neoplastic tissue sample is obtained, processed, and cultured in appropriate tissue culture medium and conditions in the presence and absence of an inhibitor to determine whether the neoplastic tissue sample is sensitive to treatment with such an inhibitor of an apoptosis-associated protein that is encoded by a gene selected from table 1B. Sensitivity to an inhibitor can be characterized by growth inhibition or by an increase in apoptosis in the neoplastic cells treated with the inhibitor, relative to the untreated tissue sample.

In one embodiment, the diagnostic method of this invention involves determining whether a neoplastic tissue sample is responsive to treatment with an inhibitor of an apoptosis-associated protein that is encoded by a gene selected from table 1B by exposing the neoplastic tissue sample to such an inhibitor and determining whether such treatment reduces the growth of tumor cells in vitro. Briefly, suspected neoplastic tissue samples are removed from a patient and grown as explants in vitro. The tissue samples are grown in the presence and absence of an inhibitor of an apoptosis-associated protein that is encoded by a gene selected from table 1B. After being grown in culture, cells can be fixed by the addition of cold trichloroacetic acid, and protein levels measured using the sulforhodamine B (SRB) colorimetric protein stain assay as previously described by Skehan, P., et al, New Colorimetric Assay For Anticancer-Drug Screening, J. Natl. Cancer Inst. 82: 1107-1112, 1990. Alternatively, a number of other methods are available to measure growth inhibition and can be used instead of the SRB assay. These methods include counting viable cells following trypan blue staining, labeling cells capable of DNA synthesis with BrdU or radiolabeled thymidine, neutral red staining of viable cells, or MTT or WST-1 cell viability assays. Inhibition of cell growth indicates that the neoplasia in question is sensitive to the inhibitor of an apoptosis-associated protein that is encoded by a gene selected from table 1B. Inhibition of cell growth is indicative that the patient would be an appropriate candidate for treatment with such an inhibitor.

In another aspect of the diagnostic method of this invention, sensitivity of a neoplastic tissue to treatment with an inhibitor of an apoptosis-associated protein that is encoded by a gene selected from table 1B is tested with an apoptosis assay. For example, a suspected neoplastic tissue sample is processed and exposed to such an inhibitor. Sensitivity to the inhibitor is characterized by an increase in apoptosis in the neoplastic tissue sample treated with the inhibitor relative to the untreated tissue sample. Inappropriate regulation of apoptosis is thought to play an important role in many pathological conditions including cancer, AIDS, Alzheimer's disease, etc. Patients with neoplasias that exhibit an increase in cell death through apoptosis after treatment with an inhibitor of an apoptosis-associated protein that is encoded by a gene selected from table 1B are candidates for treatment with such an inhibitor.

In one type of apoptosis assay, suspected neoplastic cells are removed from a patient. The cells are then grown in culture in the presence or absence of an inhibitor of an apoptosis-associated protein that is encoded by a gene selected from table 1B. Apoptotic cells are measured by combining both the attached and “floating” compartments of the cultures. Suitable detailed protocols for assessing apoptosis in tumor cell cultures treated with pro-apoptotic compounds have been described in the literature (e.g. see Piazza, G. A., et al., Cancer Research, 55:3110-16, 1995). Alternatively, other apoptosis assays as described herein, or in references quoted herein, may be used, e.g. morphological or DNA fragmentation assays.

As an alternative to determination of inhibition of cell growth or an increase in apoptosis in response to exposure to an inhibitor compound in the above methods of this invention, various surrogate markers can be evaluated that would indicate the effectiveness of the compound at promoting apoptosis or inhibiting cell growth, and thus inhibiting tumor growth. For example, if the apoptosis-associated protein is a protein kinase, inhibition of autophosphorylation or phosphorylation of a cellular substrate protein can be determined, and would be predictive of an effect on apoptosis or cell growth. For an apoptosis-associated GPCR protein, determination of inhibition of downstream signal transduction pathways would be predictive of an effect on apoptosis or cell growth.

The invention is further described, for the purposes of illustration only, in the following examples.

### EXAMPLES

### Example 1

### The Neutrophil Model of Apoptosis

A model system for the identification of early-regulated genes in apoptosis of human primary neutrophils is described in WO 02/04657. WO 02/04657 describes the isolation of human primary neutrophils, the dose responsive effect of GM-CSF on neutrophil survival and the effect of the fungal metabolite, gliotoxin, on GM-CSF. Thus, from this model, parameters of neutrophil apoptosis are established which allow apoptosis-associated genes to be identified.

In addition, neutrophil apoptosis can be measured by DNA fragmentation as follows:

Neutrophils are isolated from the blood as described in WO 02/04657 and resuspended at a concentration of 2×10⁶/ml. Five hundred microlitres are pipetted into wells of a 24 well plate and incubated in the presence or absence of survival factors.

After this incubation, the neutrophils are carefully resuspended by gentle agitation and the total contents of the well are placed into an eppendorf and centrifuged@2800 rpm for 2 min@RT in a minifuge. The cell pellet is carefully resuspended in 300 μl of ice cold hypotonic fluorochrome solution (50 μg/ml Propidium Iodide, 0.1% Sodium Citrate and 0.1% Triton X-100) and placed in a fridge for 24 h@4° C.

Prepared samples are analysed with a FacsCalibre flow cytometer (Becton Dickinson) using double discrimination to ensure single cell suspension with Log Forward and Side Scatter Parameter acquisition.

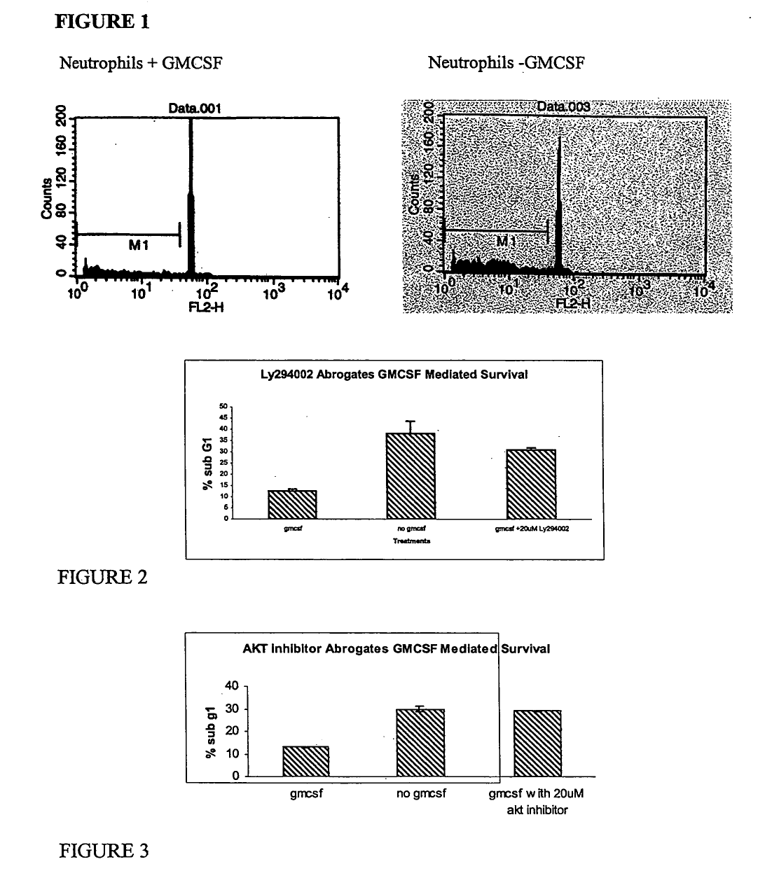

As seen in FIG. 1, healthy cells (neutrophils cultured overnight in the presence of GM-CSF) yield a single G1 peak (since neutrophils are non-cycling), with few cells in the sub-G1 peak. In contrast, neutrophils cultured in the absence of GM-CSF have increased apoptosis, as detected by a reduction in the amount of cells in the G1 peak and enhanced number in the sub-G1 peak (indicative of DNA fragmentation).

Furthermore, we confirm that the PI3 Kinase/AKT pathway has a role in GM-CSF mediated survival signal as follows:

Neutrophils are seeded at 2×10⁶/ml in a 24 well plate in full culture medium (1 ml/well). A PI3K inhibitor (Ly294002) is added to the neutrophils to a final concentration of 20 μM prior to the addition of GM-CSF (50 units/ml). Control wells received either no GM-CSF or F GM-CSF (50 units/ml) in the absence of inhibitor. Neutrophils are cultured for 18 h at 37° C. prior to harvest and prepared for sub G1 analysis, as previously described.

Inhibition of PI3 Kinase with Ly294002 is represented in FIG. 2. As can be seen, culturing neutrophils in the absence of GM-CSF for 18 h increases the number of cells undergoing apoptosis relative to those cultured in the presence of GMCSF, as determined by sub G1 analysis. In contrast, when neutrophils are cultured in the presence of both GM-CSF and PI3K inhibitor, survival is abrogated, indicative that GM-CSF is mediating survival via the PI3K pathway.

We also demonstrate a role for AKT in GM-CSF mediated survival as follows:

Neutrophils are seeded at 2×10⁶/ml in a 24 well plate in full culture medium (1 ml/well). An AKT inhibitor ([1L-6-Hydroxymethyl-chiro-inositol 2-O-methyl-3-O-octadecylcarbonate] (Calbiochem)) is added to the neutrophils to a final concentration of 20 μM and incubated for 2 h prior to the addition of GM-CSF (50 units/ml). Control wells receive either no GM-CSF or GM-CSF (50 units/ml) in the absence of inhibitor. Neutrophils are cultured for 18 h at 37° C. prior to harvest and prepared for sub G1 analysis, as previously described.

Inhibition of AKT with [1L-6-Hydroxymethyl-chiro-inositol 2-O-methyl-3-O-octadecylcarbonate] (Calbiochem), is represented in FIG. 3. As can be seen, culturing neutrophils in the absence of GM-CSF for 18 h increases the number of cells undergoing apoptosis relative to those cultured in the presence of GMCSF, as determined by sub G1 analysis. In contrast, when neutrophils are cultured in the presence of both GM-CSF and AKT inhibitor, survival is abrogated, indicative that GM-CSF is mediating survival via the PI3K pathway.

### Example 2

### Analysis of Gene Expression in the Neutrophil Model of Apoptosis Identifies Regulators of Apoptosis

Commercial microarrays are used to measure global gene expression associated with neutrophil apoptosis, GM-CSF inhibition of neutrophil apoptosis, and the inhibition of this effect using the fungal metabolite Gliotoxin. In control experiments, an inactive analogue of Gliotoxin, Methyl Gliotoxin is used. Analysis of such microarray results identifies genes whose expression pattern changes (either up-regulation or down-regulation) in an association with a measurable apoptotic phenotype.

This model discovery assay is configured to target the ‘early’ regulatory events occurring in apoptosis and, in particular, in the inhibition of apoptosis by GM-CSF. When apoptosis by GM-CSF is itself inhibited by a drug, such as gliotoxin, then changes, or patterns of changes can be targeted by clustering those changes that are common and both increase and/or decrease depending on the treatment. For example, a change that is a ‘decrease’ following induction of apoptosis is a candidate target gene, however, a change that is additionally an ‘increase’ following inhibition of apoptosis by GM-CSF has a higher probability of being a target gene because its regulation shows increased correlation with the process. Likewise, a change that is further a ‘decrease’ following inhibition of GM-CSF inhibitory effect has a yet higher probability of being a target gene because its regulation shows increased correlation with the process.

This example describes the use of the Affymetrix Human U95 microarray chipset obtained from Affymetrix (USA). This chipset contains oligonucleotide probes representing approximately 50,000 human mRNAs.

Isolation and Purification of Neutrophil RNA for Affymetrix Gene Expression Profiling

Primary human neutrophils are isolated and purified from peripheral blood of 15 normal healthy individuals using standard techniques. Each sample divided into the following treatment groups
1) T0 untreated control
2) Spontaneous apoptosis T4 (incubated in serum for 4 hours at 37° C.)
3) GM-CSF (50 U) and gliotoxin (10 μM) treated (incubated for 4 hours at 37° C.)
4) GM-CSF (50 U) and methylgliotoxin (10 μM) treated (incubated for 4 hours at 37° C.)

Total RNA is then prepared using two sequential acid phenol/guanidine isothiocyanate extractions as per manufacturers instructions (RNAzol B; Biogenesis), and subsequently purified using RNeasy RNA preparation kits (Qiagen) as per manufacturers instructions. The samples were pooled according to treatment.

Reverse Transcription and Second Strand Synthesis of Neutrophil RNA for Affymetrix Profiling

The Superscript Choice system (Gibco) was used to perform reverse transcription and second strand synthesis reactions on the neutrophil RNA samples according to the protocol in the Affymetrix GeneChip Expression manual using the T7-(dT) oligomer provided by Molecular Diagnostic Laboratory, Aarhus.

The quality of the second strand cDNA was confirmed by performing PCR using primers designed to target the 5′, middle and 3′ ends of GAPDH cDNA. This enabled a comparison to be made of the relative amounts of product and give an indication of the proportion of full-length cDNA. The cDNA was then sent to Molecular Diagnostic Laboratory, Dept. Clinical Biochemistry, Aarhus University Hospital, Skejby Sygehus, DK 8200 Aarhus N Denmark for profiling against the Affymetrix Hu95A, B, C, D and E chips.

Bioinformatic Analysis of Differential Gene Expression Across Treatment Groups Identifies of Regulators of Apoptosis

A variety of measurement outputs are provided by the Affymetrix Genechip software analysis. These are used to compute the significance of differential gene expression. The following parameters are determined:

Positive: the number of probe pairs scored positive. A probe pair is considered Positive when the following two criteria are met:

1) The perfect match intensity (PM) minus the mismatch intensity (MM)>statistical Difference Threshold (SDT, also referred to as Difference Threshold).*
PM−MM>SDT

2) After background subtraction, the PM/MM>the Statistical Ratio Threshold (SRT, also referred to as Ratio Threshold).*
PM/MM>SRT

*The user sets the SDT and the SRT.

Negative: the number of probe pairs scored negative. A probe pair is considered Negative when the following two criteria are met:

1) The MM minus the PM>Statistical Difference Threshold
MM−PM>SDT

2) After background subtraction, the MM/PM>the Statistical Ratio Threshold
MM/PM>SRT

This eliminates from analysis those probe pairs for which the difference between the PM and the MM is insignificant. Some probe pairs may not meet either set of requirements and therefore will not be scored as Positive or Negative.

Pairs: The total number of probe pairs for this gene on the probe array.

Pairs Used: This is the number of probe pairs per gene used in the analysis. This can be either the total number of probes per gene or the chip or the number of probe pairs in a pre-selected subset, which can be designated by a probe-mask file.

Pairs in average: This is the number of probe pairs used in the computation of Log Avg and Avg Diff. There are 3 modes of determining the Pairs in Avg:

All-inclusive: All probe pairs for a gene are used in the computation. This is the default when the number of probe Pairs for the gene is less than 8.

Olympic scoring: Olympic scoring excludes the probe pairs with the maximum and minimum values for the intensity difference (PM−MM). This method is used if the number of probe Pairs is between 9 and the # of probes threshold (NPT*, default=15).

Super Olympic scoring: Only data within 3 standard deviations (STP*) of the mean of the intensity difference which is computed with both the maximum and minimum excluded, are used. The exclusion of maximum and minimum is mandatory, that is even if they fall within the specified range, they are excluded. This method is used if the number of probe Pairs for the gene is greater than the # of probes threshold.

Positive fraction: This is the ratio of probe pairs scored Positive divided by the number of probe pairs used. The Pos Fraction is a good indicator of whether or not a transcript is present. If a majority of the probe pairs are positive, probe usage is good and the transcript is probably present.

Log Avg (Log Average Ratio): An intensity ratio (PM/MM) for each probe pair used is calculated where the intensity of the perfect match is divided by the intensity of the mismatch. An average of the intensity ratios is calculated for each gene from the Pairs in Avg and multiplied by 10.
Log Avg=10×[S log(PM/MM]/(#probe Pairs In Avg)

Note: Log Avg=0, indicates random cross hybridization. The higher the Log Avg, the more confident one is that the gene transcript is present.

PM Excess (Perfect match excess): The number of probe pairs for which the intensity ratio exceeds a given value (ratio limit).

MM Excess (Mismatch excess): The number of probe pairs for which the reciprocal of the intensity ratio exceeds a given value (ratio limit).

The numbers shown in PM excess and MM excess show how many of the PM/MM or MM/PM ratios exceeded the defined value. For example, 5 means that 5 of the total PM/MM or MM/PM (ratios) were greater than 10 (the default value).

Pos/Neg (Positive/Negative): This is the ratio of Positive scoring probe pairs to the Negative scoring probe pairs.

Avg Diff (Average Difference): An intensity difference (PM/MM) for each probe pair used is calculated where the intensity to the mismatch is subtracted from the intensity of the perfect match. An average of the intensity differences is calculated for each gene from the probe Pairs In Avg.
S(PM−MM)/(# probe Pairs In Avg)

Abs Call (Absolute Call): There are 3 possible outcomes in Abs Call: “P” (present), “A” (absent), and “M” (marginal). The call is based on the values of Pos Fraction, Log Avg, and Pos/Neg.

Inc (Increase): for each gene, the number of probe pairs for which PM minus MM is significantly greater in the experimental data than in the baseline data. To be considered significant, two conditions must be met:
(PM−MM)experimental−(PM−MM) baseline>Change Threshold (CT)* 1)
[(PM−MM)experimental−(PM−MM) baseline]/(PM−MM)baseline>Percent Change Threshold (PCT)*/100 2)

Dec (Decrease): For each gene, the number of probe pairs for which PM−MM is significantly greater in the baseline data than in the experimental data. To be considered significant, two conditions must be met:
(PM−MM)baseline−(PM−MM)experimental>CT 1)
[(PM−MM)baseline−(PM−MM)experimental]/(PM−MM)baseline>PCT/100 2)

Pos Fraction (Positive fraction): This is the ratio of probe pairs scored Positive divided by the number of probe Pairs used. The Pos Fraction is a good indicator of whether or not a transcript is present. If a majority of the probe pairs are positive, probe usage is good and the transcript is probably present

Log Avg (Log Average Ratio): An intensity ratio (PM/MM) for each probe pair used is calculated where the intensity of the perfect match is divided by the intensity of the mismatch. An average of the intensity ratios is calculated for each gene from the Pairs in Avg and multiplied by 10.
Log Avg =10×[S log(PM/MM)]/(#probe Pairs In Avg)

Note: Log Avg=0, indicates random cross hybridization. The higher the Log Avg, the more confident one is that the gene transcript is present.

Avg Diff (Average Difference): An intensity difference (PM/MM) for each probe pair used is calculated where the intensity to the mismatch is subtracted from the intensity of the perfect match. An average of the intensity differences is calculated for each gene from the probe Pairs In Avg.
S(PM−MM)/(# probe Pairs In Avg)

Avg Diff Change (Average Difference Change): The difference between the experimental and baseline average differences (Avg Diff).
Avg Diff Experimental−Avg Diff Baseline

Inc Ratio (Increase Ratio): For each gene, the ratio of the number of probe pairs identified as increased (Inc) to the number of probe Pairs Used in the analysis.

Dec Ratio (Decrease Ratio): For each gene, the ratio of the number of probe pairs identified as decreased (Dec) to the number of probe Pairs Used in the analysis.

Max Inc & Dec Ratio (Maximum Increase Ratio and Decrease Ratio): the greater of Inc Ratio or Dec Ratio.

Pos Change (Positive Change): The difference between the number of probe pairs scored positive in the experimental file and the baseline file for each gene.

Neg Change (Negative Change): The difference between the number of probe pairs scored negative in the experimental file and the baseline file for each gene.
Inc/Dec Increase/Decrease: Ratio of the number of probe pairs that increased over the number of probe pairs that decreased, refer to Inc and Dec.

Diff Call (Difference Call): There are 5 possible outcomes for difference call. The level of expression of an RNA is increased (I), decreased (D), marginally increased (MI), marginally decreased (MD) or there is no detectable change in expression level (NC). The difference between all decisions is based on the values of Inc/Dec, Inc Ratio (or Dec Ratio), Dpos-Dneg Ratio and Log Avg Ratio Change

Fold Change: The fold change is the ratio of the average differences (Avg Diff) between the experimental and baselines files. This number reflects the magnitude of the change, but not the direction of the change (refer to Diff Call). For example, if the Avg Diff Experimental is 3000 and the Avg Diff Baseline is 300 then the fold change is 10. If the Avg Diff Experimental is 300 and the Avg Diff Baseline is 3000 then the fold change is also 10. If either Avg Diff experimental or Avg Diff Baseline is <20, the value is set at “20” for the purpose of this calculation.

Sort Score: Sort Score is based on both Fold Change and Avg Diff Change. This score can be used to evaluate the differences in the expression of genes between the experimental and baseline files. The larger numeric value for the Sort Score, the more “significant” or reliable is the measured difference in expression of a gene between the experimental and baseline files. For example, a Fold Change of 5 has a greater Significance value when the Avg Diff Change value goes from 200 to 1000 than when it goes from 20 to 100.

According to the principles of the model screening approach already outlined, here and in WO 02/04657, we have used the following patterns of differential gene expression across treatment groups to identify apoptotic regulators;
1. Down-regulated in Untreated (T=4 hours) compared with Untreated (T=0 hours) and (T4:T0)
2. Down-regulated in GM-CSF+Gliotoxin (T=4 hours) compared with Untreated (T=0 hours) (G:T0) and
3. Up-regulated or unchanged in GM-CSF+Methylgliotoxin (T=4 hours) compared with Untreated (T=4 hours) (M:T4) and
4. Up-regulated or unchanged in GM-CSF+Methylgliotoxin (T=4 hours) compared with GM-CSF+Gliotoxin (T=4 hours) (M:G) OR
1. Up-regulated in Untreated (T=4 hours) compared with Untreated (T=0 hours) (T4:T0) and
2. Up-regulated in GM-CSF+Gliotoxin (T=4 hours) compared with Untreated (T=0 hours) (G:T0) and
3. Down-regulated or unchanged in GM-CSF+Methylgliotoxin (T=4 hours) compared with Untreated (T=4 hours) (M:T4) and
4. Down-regulated or unchanged in GM-CSF+Methylgliotoxin (T=4 hours) compared with GM-CSF+Gliotoxin (T=4 hours) (M:G)

Using these parameters, we identified a number of differentially regulated genes, including those listed in Tables 1A and 1B.

Table 2 gives the fold change of gene expression across treatment groups, referenced to untreated neutrophils according to the treatment patterns listed above.

Bioinformatic gene function analysis, using the Gene Ontology database (www.geneontology.org) is used to further characterise the differentially regulated genes identified. Those genes identified in Tables 1A and 1B were determined to share homology with kinases and G-protein coupled receptors

Confirmation of Array Data Using QPCR

Quantitative PCR (QPCR) offers high precision level detection among gene expression profiling techniques. Furthermore, real time analysis can complement microarray analysis by offering high sensitivity and accurate quantification. Additionally, due to it requiring gene specific primers, there is heightened specificity of target genes. In the current example, a number of genes that were identified by microarray analysis as being regulated in apoptosis are further profiled using QPCR across the various treatment groups and referenced to untreated neutrophils.

Neutrophil RNA is isolated and purified from the various treatments as listed above. Similarly, Reverse Transcription and second Strand Synthesis is performed as above. The conditions that Quantitiative PCR is performed under are described in detail in Example 3.

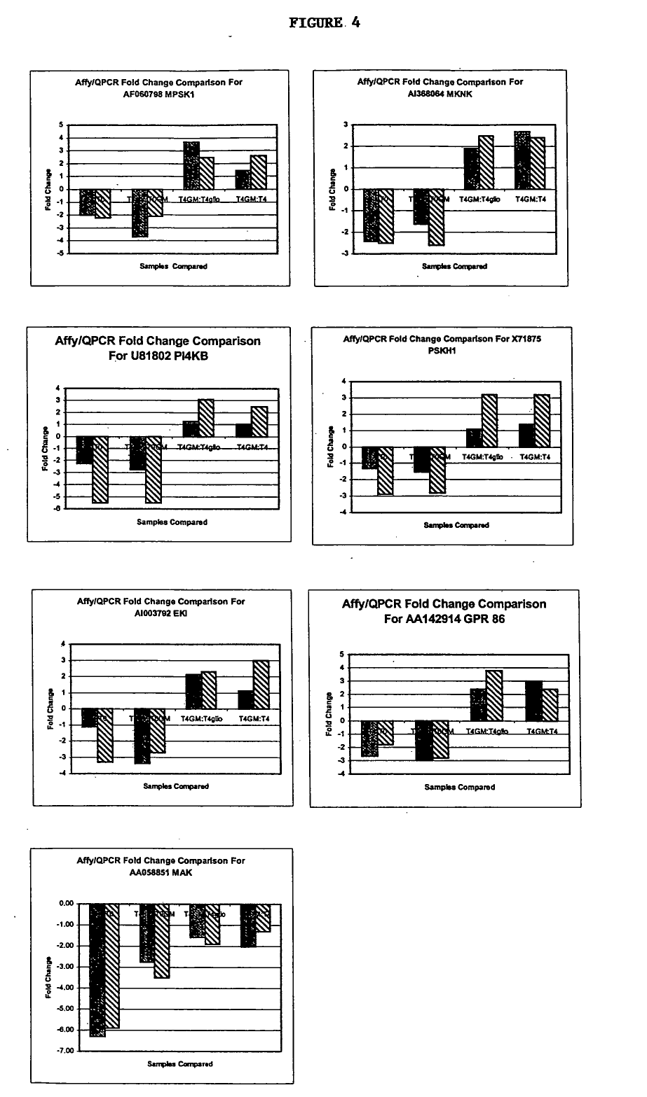

The results of QPCR analysis for a number of target genes are given in FIG. 4. For the seven genes that were compared, there is excellent correlation between the results of the microarray analysis and QPCR over the various treatment groups.

This result therefore confirms the described regulation of target genes as determined by microarray.

Descriptions of Some Apoptosis Modifiers

Table 1A lists differentially regulated genes encoding kinases and GPCRs which are well-characterised examples of regulators of apoptosis. These are therapeutic targets for cancer and inflammatory disease. These include the following known genes whose functions are described.

1) Protein Kinase C Zeta (PKCζ)

Protein kinase C's are serine-threonine kinases which play a central role in the signal transduction pathways involved in cell regulation, cancer and apoptosis. The eleven isoforms identified are structurally related but have different co-factor requirements. They constitute three families: (i) the classical isoforms (cPKC-α, βI,βII, and γ) are DAG-activated and Ca2+ dependent (ii) the novel isoforms (n PKC-δ, ε, η, θ, and μ) are DAG activated but Ca2+-independent; and (iii) the atypical isoforms (PKC-ζ and l/λ) are both DAG unresponsive and Ca2+ independent. Isoforms are translocated from cytosol and membrane fractions, upon activation.

Protein kinase ζ has been implicated in several signal transduction pathways regulating differentiation, malignant transformation and apoptosis of mammalian cells. Two of the major pathways where PKCζ is implicated directly are the sphingomyelinase/ ceramide pathway and the PI3/AKT pathway, where signaling through either in not mutually exclusive (Calcerrada et al, FEBS Lett 2002 13; 514 (2-3) 361-5.

The fact that PKCζ can be activated by Phosphatidylinositol 3,4,5-triphosphate (Nakanishi et al J Biol Chem 1993; 268 (1): 13-6), suggests that it functions in the PI3K/AKT survival pathway. Full activation of AKT enzyme requires phosphorylation of a threonine and a serine amino acid. The phosphorylation of the threonine is due to PDK1, however the mechanism leading to the serine phosphorylation by PDK2, is less understood. Recent evidence suggests that PDKζ may act as an adapter, by binding PDK2 and AKT, it facilitates the serine phosphorylation of AKT leading to full activation (Hodgkinson C P et al 2002 Biochemistry 412(32): 10351-9).

In addition murine erythroleukemia cells (Friend) respond to high dose ionizing radiation with the activation and nuclear translocation of p85alpha subunit of phosphatidylinositol-3-kinase (PI-3-kinase) that results in downstream activation and nuclear translocation of PKCζ. Following treatment with wortmannin, a relatively specific inhibitor of PI-3-kinase, both an increased number of apoptotic cells and the inhibition of protein kinase C zeta translocation is detected. This result suggests that a potential role of the PI-3-kinase/PKC zeta pathway in protecting Friend cells from ionizing radiation-induced apoptosis offering PKC zeta for consideration as possible target of pharmacological treatments (Cataldi et al Int J Oncol January 2003;22(1):129-35).

The product of the par-4 gene, expression of which correlates with apoptosis, specifically interacts with the regulatory domain of PKCζ to inhibit its activity. Overexpression of par-4 induces apoptosis in NIH-3T3 cells, however this can be abrogated if PKCζ (but not its kinase inactive mutant) is overexpressed suggesting the PKCζ can control survival (Ddiaz-Meco et al, Cell 1996 86(5):777-86). Another example where PKCζ protects cells is highlighted by de Thonel et al (Blood 2001, 98(13) 3770-7) who showed that the mechanism of Fas resistance in acute myeloid leukemia (AML) was due to PKCζ expression. Chemical inhibition or overexpression of par-4 restored Fas-induced apoptosis.

Similarly, inhibition of PKCζ by overexpression of the kinase dead, dominant negative PKCζ in the U937 human leukemic cell line resulted in a decrease in BCL2 expression, with a simultaneous increase in Bax expression. Moreover PKCζ inhibition accelerated the occurrence of apoptosis in leukemic cells exposed to etoposide and TNFα, suggesting its inhibition sensitized the cells to these reagents. Inhibition of PKCζalso increased the sensitivity of tumor cells grown in nude mice to etoposide. Consequently, the authors conclude that inhibition of PKCζ is an attractive target for chemosensitzation of tumour cells (Filomenko et al Cancer Res 62(6): 1815-21).

One consequence of activation of PKCζ is the activation of NFκB by indirectly inducing the phosphorylation IκK, allowing NFκB to migrate into the nucleus to transcribe anti apoptotic genes (Diaz-Meco et al EMBO J 1994 13(12): 2842-8). Mice with targeted inactivation of PKCζ by homologous recombination, though grossly normal appeared to have phenotypic alterations in their secondary lymphoid organs. Moreover these mice had impaired activation of the NFκB pathway, particularly in the lung where IκK activation was inhibited (Leitges et al Mol Cell October 2001;8(4):771-80).

Protein Kinase Cζ has also been implicated in generation of reactive oxidants in endothelial cells stimulated with TNFα. Treatment of endothelial cells with TNFα resulted in the phosphorylation of p47 and gp91(NADPH oxidase subunits), a necessary event for oxidant production. Inhibition of PKCζ prevented the TNF-alpha-induced phosphorylation of p47(phox), and its translocation to the membrane, with subsequent inhibition of oxidant generation (Frey et al, Circ Res May 17, 2002;90(9): 1012-9).

Contrary to the antiapoptotic examples listed above, PKCζ has also been suggested to be proapoptotic in neutrophils treated with TNFα. Exposure of TNFα dose dependently increased expression of PKCζ which correlated with death (Das et al, Mol Cell Biochem 1999;197(1-2):97-108.

2) 3′ Phosphoinositide Dependent Kinase 1 (PDK1)

PDK1 plays a central role in activating the AGC protein kinases Akt, S6K and RSK, MSK and SGK. In particular, PDK1 plays an important role in regulating cell survival due to its ability to phosphorylate and activate Akt. Though for full activation, Akt needs to be phosphorylated on threonine −308 and serine −473 residues, threonine phosphorylation alone is sufficient for partial activation. Subsequent to activation, Akt survival is mediated by a number of events including phosphorylation and inhibition of Bad, Caspase 9 and Forkhead transcription factors.

Overexpression studies have demonstrated the PDK1 has a role in mammary gland oncogenesis. Retrovirally transduced COMMA-1D mouse mammary epithelial with PDK1 exhibited a high degree of transformation (as measured by anchorage-independent growth in soft agar) that was associated wit hthe activation of Akt and protein kinase C. The authors conclude that activation of PDK1 can lead to mammary tumorigenesis, and suggest that PDK1 expression may be an important target in human breast cancer (Zeng et al Cancer Res 2002 62(12): 3538-43).

In agreement with this hypothesis, Arico et al (J Biol Chem 2002 277(31) 27613-21) demonstrated that the antineoplastic effect of Celecoxib, a COX-2 specific inhibitor, previously shown to reduce the number of adenomatous colorectal polyps, might be mediated by inhibition of PDK1. Celecoxib induced apoptosis in the colon cancer cell line HT-29 by inhibiting PDK-1, which resulted in inhibition of phosphorylation of Akt. Expression of a constitutively active form of Akt had a low protective effect toward celecoxib-induced cell death where-as expression of a constitutive active mutant of PDK1 was potent at inhibiting apoptosis, confirming that Celecoxib antineoplastic activity was through PDK-1 inhibition. Similarly, UCN-01 (7-hydroxystaurosporine), a drug now in clinical trials and with a unique fingerprint pattern, induced dephosphorylation and inactivation of Akt, resulting in the turn-off of the survival signals and the induction of apoptosis. Further analysis revealed that UCN-01-mediated Akt inactivation was caused by inhibiting upstream Akt kinase PDK1 (IC50=33 nM) both in vitro and from cells, but not by suppressing Akt itself or phosphatidylinositide-3-OH kinase. UCN-01-induced PDK1 inhibition was also observed in in vivo murine and human tumor xenografts. Overexpression of active form of Akt diminished the cytotoxic effects of UCN-01, suggesting that UCN-01 may in part exert its cytotoxicity by inhibiting PDK1-Akt survival pathway. Because UCN-01 has already proved to have potent anti-tumor activity in vivo, PDK1-Akt survival pathway is a new, attractive target for cancer chemotherapy (Sato et al. Oncogene Mar. 7, 2002;21(11):1727-38).

In one further example demonstrating the pivitol role that PDK1 plays in cancer, Ballif et al (J Biol Chem 2001 276 (15) 12466-75) demonstrated that the anti-tumorigenic and anti-proliferative agent n-alpha-tosyl-1-phenylalanyl chloromethyl ketone (TPCK) prevented phosphorylation of the PDK1 regulatory sites in S6K1 and Akt. In turn this resulted in a decrease in the phosphorylation status of the proapoptotic protein BAD, resulting in cell death of the IL3 dependent 32D cells.

In knock out studies, Lawlor et al (EMBO J 2002 21(14) 3728-38), generated mice that either lack PDK1 or possess PDK1 hypomorphic alleles, expressing only 10% of the normal level of PDK1. PDK1 (−/−) embryos die at embryonic day 9.5, displaying multiple abnormalities. In contrast, hypomorphic PDK1 mice are viable, though these mice are 40-50% smaller than control animals. Similarly, organ and cell volume are reduced proportionately. The authors conclude that PDK1 is essential for mouse embryonic development and regulates cell size.

3) Jak3

STATs constitute a family of DNA-binding proteins that reside in the cytoplasm until they are activated by tyrosine phosphorylation. Phosphorylation of STATs is catalyzed by members of the Janus family of tyrosine kinases, including JAK3. However, there appears to be a more generalized role for the Jak family than activation of gene transcription via the Stat family. In at least one example of cell activation, Jak3 has been shown to associate with the regulatory subunit of PI3K and regulate IL-7-induced P13 kinase activation by mediating tyrosine phosphorylation of the p85 subunit. Specific inhibition of IL-7-induced Jak kinase activity ablates p85 tyrosine phosphorylation, subsequent P13 kinase activation, and, ultimately, proliferation (Sharfe et al, Blood 1995 86(6) 2077-85).

The importance of Jak 3 in apoptosis and survival is seen in inhibitor studies. WHI-P131, a specific inhibitor of Jak3 inhibited the clonogenic growth of JAK3-positive leukemia cell lines DAUDI, RAMOS, LC1;19, NALM-6, MOLT-3, and HL-60 (but not JAK3-negative BT-20 breast cancer, M24-MET melanoma, or SQ20B squamous carcinoma cell lines) in a concentration-dependent fashion. On these findings the authors conclude that potent and specific inhibitors of JAK3 such as WHI-P131 may provide the basis for the design of new treatment strategies against acute lymphoblastic leukemia, the most common form of childhood cancer (Sudbeck et al; Clin Cancer Res 1999, 5(6) 1569-820. Furthermore inhibitors of Jak3 WHI-P131 and WHI-p159 potent inhibitors of glioblastoma cell adhesion and migration (Narla et al, Clin Cancer Res 4(10)2463-71).

Jak3-deficient mice display vastly reduced numbers of lymphoid cells. Thymocytes and peripheral T cells from Jak3-deficient mice have a high apoptotic index, suggesting that Jak3 provides survival signals. Wen et al (Mol Cell Biol 2001 21(2) 678-89) reported that that Jak3 regulates T Iymphopoiesis at least in part through its selective regulation of Bax and BCL2. Jak3-deficient thymocytes express elevated levels of Bax and reduced levels of BCL2 relative to those in wild-type littermates. They conclude by suggesting that Jak 3 regulates cell survival through their selective and cell context-dependent regulation of pro- and antiapoptotic BCL2 family proteins and that Bax and BCL2 play distinct roles in T-cell development.

4) Phosphofructokinase, liver (PFKL)

Phosphofructokinase (PFK; ATP:D-fructose-6-phosphate-1-phosphotransferase is a tetramer formed by the random association of the products of two separate gene loci to form the five possible tetramers. PFKs of muscle and liver are homotetramers of the M and L subunits, respectively. Red cells have all five isozymes: M4, M3L, M2L2, ML3, and L4.Liver phosphofructokinase; functions in glycolysis, phosphorylates fructose-6-phosphate to fructose-1,6-bisphosphate

6-Phosphofructokinase (PFK) plays a central role in the regulation of glycolysis in both normal and neoplastic cells. Since PFK also mediates the Pasteur effect, it coordinates the two modes of energy production in most cell systems, i.e., glycolysis and respiration. The energy production in the cancer cell is characterized by a predominance of aerobic glycolysis (the Warburg effect) and a diminution or lack of the Pasteur effect.

Staal et al (Cancer Res 1987 47(19) 5047-51) demonstrated that there was an increase in the PFKL levels in glioma tissue relative to normal brain tissue and that this corresponded with a decrease in levels of normal PFK. Other investigators have reported that there was a significant increase in PFK activity in carcinomas in comparison with phosphofructokinase in normal thyroid tissue. Specific activities of follicular adenomas are rather heterogeneous. When these tumors were divided into three groups of increasing proliferative activity as judged by histopathological criteria, highest specific activities of phosphofructokinase were found in the group with the highest proliferative activity. The latter group resembles the enzyme activities found in carcinomas. All three isozymes of PFK, M-(muscle)type, L-(liver)type and P-(platelet)type, were present (van der Heijden et al; Tumour Biol 1986; 7(1) 7-9.

Further investigation into the isozymic profile of PFK from malignant cells of human leukemias, lymphomas, virus-transformed cell lines, and established malignant cell lines of lymphoid, myeloid, erythroid, and fibroblastic origin and their normal counterparts revealed that there was an increase in levels of PFKL in all malignant tissue (Vora, Cancer Res 1985 45(7) 2993-30001).

Accordingly, amongst the genes identified using the screening approach described herein there are a significant number of are well characterised regulators of apoptosis which are recognised therapeutic targets for cancer and inflammatory disease. Thus, the genes which are identified herein for the first time as regulators of apoptosis are also candidate therapeutic targets.

### Example 3

### Differential Expression of Identified Gene Targets in Normal Versus Transformed Tissue Samples and Their Expression in Cancer Cell Lines

This example describes screening normal and transformed tissue for expression levels of target genes. In addition the expression of these genes is also examined across a broad range of cancer cell lines isolated from various tissues. Expression is measured using real time QPCR.

A) Differential Expression in Normal and Transformed Tissue and in Cancer Cell Lines

Source of Normal and Tumour Tissue RNA

A panel of tumour and matched normal adjacent tissue (NAT) RNA was obtained from Ambion for the tissues indicated in the following tissues. Each sample is derived from a non-pooled, individual human tumour and normal adjacent tissue, and tumour classification is as follows:

| Tissue | Tumour type |
|---|---|
| Colon | Invasive, moderately differentiated adenoma |
| Kidney | Renal cell carcinoma |
| Lung | Adenocarcinoma |
| Breast | Adenocarcinoma |
| Ovary | Adenocarcinoma |

Cancer Cell Lines

The cancer cell lines listed in Table 3 were obtained from the National Cancer Institute and maintained in RPMI with 10% FCS. Cells are cultured at 37° C. in a humidified atmosphere containing 5% CO₂ and harvested by trypsinisation when confluent for subsequent isolation of total cellular RNA for QPCR Analysis.

Isolation of Total Cellular RNA

Cell lines, as indicated below, are grown in logarithmic growth. RNA is prepared using the RNeasy kit (Qiagen) according to the manufacturer's instructions. For each cell line 5×10⁶ cells are harvested and the cell pellets lysed in 600 μl buffer RLT containing β-mercaptoethanol. DNA is sheared by passing the lysate through a KIAshredder column (Qiagen) prior to loading the sample onto an RNeasy column. DNA is removed by on-column digestion with RNase-free DNase followed by buffer wash and elution in RNase-free H₂O.

cDNA Preparation for QPCR Analysis

RNA (1-2 μg) primed with an oligo (dT) primer is reverse transcribed to first strand cDNA in a reaction containing Superscript II (Life Sciences), RT buffer and dNTPs as per manufacturers instructions. Reactions are incubated at 42° C. for 2 hours and stored until use at −20° C.

Performing Quantitative RT PCR

The Qiagen QuantiTect SYBR Green PCR kit provides a master mix containing Hot-start Taq polymerase and an optimised concentration of SYBR Green together with a specialized buffer. The buffer contains a balanced combination of KCl and (NH₄)₂SO₄, which promotes a high ratio of specific to non-specific primer binding during the annealing, step of each PCR cycle. This makes it unnecessary to optimise the MgCl₂ concentration. This kit is used in accordance with the manufacturer's instructions.

Opticon Real-Time PCR Machine (NJ Reseach) and Settings:

Experimental parameters are defined for each run by creating a master file. A master file has 3 components:
1 Plate file
2 Protocol file
3 Options file

Plate file: sets out the layout of the plate where the wells are assigned as sample, standard, blank(no template control) or empty well

Protocol file: Sets the PCR parameters and plate read step and allows the inclusion of melting curve profile

Options file: sets options for data processing and quantitation including;
1. Apply norms and zeros: normalization for variation in signal between wells by applying a normalization constant to data from each well
2. Subtract blanks: data from wells designated as blanks can be subtracted from sample data
3. Subtract base line signal
4. Threshold: sets the C(T) cycle threshold line for use in quantitation. Can be set manually or automatically to a multiple of standard deviations above the mean over a defined cycle range.

Option settings can be adjusted during post-run data analysis.

Quantitation

The relationship between starting copy number and the number of cycles preceding detection can be used to calculate the initial quantity of template in a sample. First the position of the threshold or C(T) line must be defined on a graph of fluorescence vs. cycle number. This is usually set to a point where the samples exceed background noise and begin to increase. The threshold cycle for an individual sample is then defined as the cycle at which the fluorescence trace crosses the C(T) line. By including quantitation standards, a graph of a standard curve of log quantity vs. C(T) cycle can be plotted. The quantity of initial template in unknown samples can then be calculated by applying the sample's threshold cycle to the standard curve.

In order to correct for differences in the efficiency of the RT reaction, quantitative PCR is performed using primers directed to a reference, housekeeping gene (e.g. GAPDH or RPS13) and the results expressed as a ratio of target to reference gene template amount.

Standards

Both RNA and DNA standards are commonly used in quantitative PCR. For these experiments DNA standards were used which consist of cloned PCR products in a pMSCV vector (Clontech). In this way large amounts of reference material can be produced which can be quantified by spectrophotometry. Plasmids are linearised since this conformation more closely simulates the amplification efficiency of cDNA. Ten fold dilutions of standards from 100 pg/ml to 1 fg/ml are included to establish a range that encompasses that of test cDNA results.

To summarise QPCR procedure: The cDNA templates are amplified with target-specific QPCR primers using QuantiTectSYBR Green PCR kit (Qiagen 204143) on a DNA Engine Opticon System (MJ Research). The amplification conditions include a 95° C. step for 15 min for initial activation of HotStarTaq DNA polymerase, followed by 35 cycles of (15 s at 94° C., 30 s at 60° C., 30 s at 72°). The fluorescence of the samples at 521 nm is read between the annealing and extension steps of the protocol. The melting curves are calculated at the end of the 35 cycles and confirm product homogeneity.

A standard curve is plotted using the log [template quantity] of the RPS13 control template dilutions (as above) versus cycle number at which the fluorescence intensity measured in the well exceeds the level specified in the cycle threshold parameters (the C(T) value). An estimate of the quantity of initial template in treatment samples is determined from this plot and the amount of target mRNA present in each sample is normalized across samples by calculating the ratio of target to RPS13 for each sample.

PCR Primers

Primers for target genes are designed using Genetool software (Biotools Inc) and sequences are given in Table 4.

Differential Expression of Targets Between Normal and Cancer Tissue

There is differential expression of the target genes across a range of matched normal and cancer tissues as determined by QPCR. Table 5 represents the fold differences between the matched pairs. Results presented demonstrate that identified genes (e.g. GPR86, GRAF, EKI, NTKL, CDC42, RBSK, EDG6, PRK/CNK, FLT4 and PSKH1) are differentially expressed and/or correlated with cancer as compared to adjacent normal tissue. This indicates an association between deregulated expression of these genes and manifestation of a cancer phenotype.

These examples demonstrate that the apoptosis associated genes identified are positively associated with cancer phenotype.

Gene Targets are Expressed in a Range of Cancer Cell Lines

Table 6 shows the differential expression of candidate genes across a broad range of cancer cell lines derived from a variety of cancer types. Absolute levels were measured using QPCR and normalized against a house keeping gene (RPS13). Expression of the target genes is detected in some or all of a broad range of cancer cell lines, isolated from various locations in the body. Levels of expression vary considerably depending on cancer cell type.

B) Further Expression Profiling

Expression profiling was extended using an alternative QPCR method on a further range of cancer cell lines identified in Table 7.

Purification of Total Cellular RNA from Cell Lines

The cell lines, as indicated in Table 7, are grown in logarithmic growth. RNA is prepared substantially as described above using the RNeasy midi kit (Qiagen). DNA is sheared by homogenisation with a rotorstator homogeniser prior loading the sample onto an RNeasy column. RNA is eluted in a total of 400 μl RNase-free H₂O. Contaminating genomic DNA is removed by treatment with DNA-free DNase (Ambion) for 15 minutes and DNase activity removed by resin-based inactivation according to manufacturer's instructions. RNA was ethanol precipitated and washed in 70% ethanol prior to suspension in THE RNA storage buffer (Ambion).

cDNA Preparation for QPCR Analysis

RNA (10 μg) primed with random hexanucleotide primers is reverse transcribed to first strand cDNA using a cDNA Archive Kit (ABI) in a reaction containing reverse transcriptase, RT buffer and dNTPs. Reactions are incubated at 25° C. for 10 minutes and 37° C. for 2 hours and stored until use at −70° C.

Quantitative PCR

Quantitative PCR (QPCR) profiling of gene expression is performed using SYBR Green PCR 2× master mix kit (Abgene) in accordance with manufacturer's instructions. PCR primers are designed using either Primer Express software (ABI) or web-based Gene Tools software (Stratagene). All primers are designed to generate amplicons of between 90 bp and 120 bp at an annealing temperature of 60° C. and are tested at 50 nM, 100 nM and 200 nM in standard QPCR reactions to identify concentration at which amplification of the target sequence is optimum and specific. Primer sequences and optimum determined concentration is shown in Table 8.

Amplification is performed using an ABI7000 sequence detection system following the manufacturer's instructions. Each PCR comprised cDNA derived from 50 ng RNA with appropriate primers and 1×SYBR Green master mix in a total volume of 25 μl. Gene expression levels are expressed relative to the GAPDH house keeping gene using the comparative Ct (cycle threshold) method according to standard methodology. Briefly, for each cell line sample, the Ct value derived from QPCR of GAPDH is subtracted from the target gene Ct to give a value referred to as ΔCt. An internal control on each plate allows each ΔCt to be normalised to eliminate variation between QPCR experiments, giving a value referred to as ΔΔCt. Expression of the target gene relative to GAPDH in each sample is calculated using the equation; relative expression=2^{-ΔΔCt}.

Representative Data Showing Expression of Target Genes in Cancer Cell Lines.

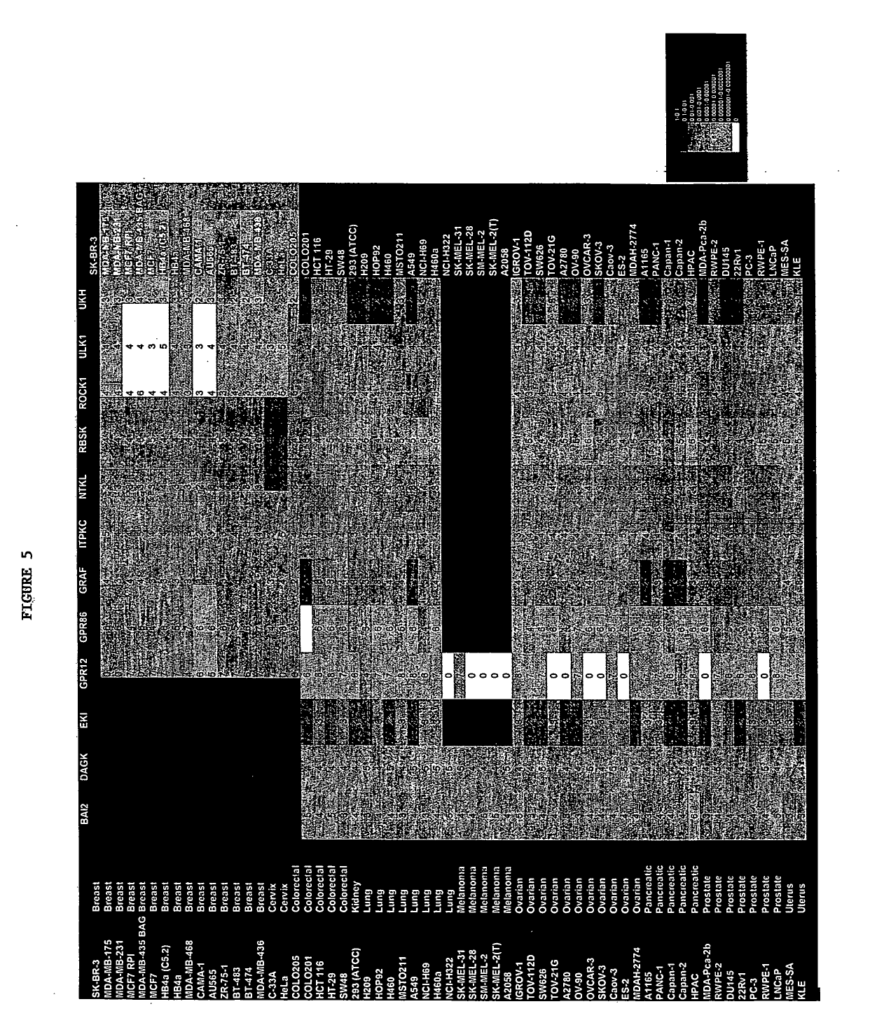

The expression profiling data is depicted in FIG. 5 as a “heat map”, where different shades represent the expression level of each target gene. The expression of each gene is described relative to the expression of the GAPDH housekeeping gene.

Summary of Data

This expression profiling data shows that the twelve identified target genes are expressed throughout a range of cell lines representing various types of human tumours. BAI2 is expressed in all cell lines tested with a 3-log range relative to GAPDH, with no noted prevalence in a particular cancer type. This is also true for the expression of DAGK-theta and EKI. The expression of GPR12 mRNA is detectable in two thirds of the cell line panel, with the expression level showing a large 5-log range. It is notable that GPR12 expression is present mainly in cell lines representing breast, colorectal, lung, pancreatic and prostate cancers. This is in contrast to melanoma and ovarian cell lines, which express low or undetectable levels of GPR12. Of the cell lines expressing GPR12, three breast lines, a single lung line and two pancreatic lines express higher than average levels of GPR12 mRNA and may be deemed to be overexpressing the gene. The GPR86 gene has a wide range of expression, encompassing a 5-log range relative to GAPDH throughout the panel of cancer cell lines, with no prevalence in any cancer cell type. The GRAF gene is expressed in all cell lines tested, with a narrow distribution of expression within a 3-log range. While GRAF is expressed at relatively high levels throughout the cell line panel, it is notable that GRAF expression is particularly prevalent in cell lines representing colorectal and pancreatic cell lines. The expression of ITPKC is detectable in all cell lines tested, with expression levels distributed over a 4-log range. ITPKC expression is particularly high in cell lines derived from breast and prostatic cancers. The profiling of NTKL expression demonstrates that this gene is expressed at a relatively high level across the entire panel of cancer cell lines with a particularly narrow distribution, spanning only a 2-log range. RBSK is expressed throughout the cell line panel, across a 4-log range. It is notable that the majority of breast, colorectal and prostate cells lines express relatively high levels of RBSK compared to average expression across the entire panel. ROCK1 and ULK1 genes are expressed throughout the cell line panel, with no noted prevalence in any particular cancer types. UKH is expressed at relatively high levels across a 3-log range throughout the panel of cancer cell lines. Notably, UKH is expressed at very high levels in a number of cells lines representing breast, lung and ovarian cancers.

### Example 4

### Overexpression of Identified Genes Functionally Modulates Apoptosis

The erythroleukaemic TF1 cell line is growth factor dependent, requiring GM-CSF for survival in culture. Withdrawal of GM-CSF causes TF 1 cells to undergo spontaneous apoptosis. The impact of candidate regulators of the apoptotic process, such as EDG6 and TLK2, can be determined in these cells by measuring the extent of apoptosis following introduction of the candidate gene to the cells.

Analysis of TF1 Apoptosis by Light Scatter Analysis

Light Scatter Analysis takes advantage of the fact that by using the laser beam of a flow cytometer one can determine the size (Forward Scatter) and granularity (Side Scatter) of a cell. The morphological changes associated with apoptosis, such as decreased size (shrinkage) and granularity affect these parameters. As a consequence, cells undergoing apoptosis will move to the left and slightly down, from the parameters of a healthy population.

TF1 cells are plated into 24 well plates (2×10⁵/ml) and are cultured for 48 h in the presence or absence of GM-CSF (2 ng/ml). Cells are then harvested by centrifugation (1000 rpm, for 10 min) and washed in PBS. The pellet is resuspended in PBS (2×10⁵ cells/ml) and acquired by a FacsCalibre. Forward and Side scatter parameters are assessed using Cell Quest software.

Analysis of TF1 Apoptosis by Sub G1 Analysis

TF1 cells are cultured for 48 h in the presence or absence of GM-CSF (2 ng/ml). Cells are then harvested by centrifugation (1000 rpm, for 10 min) and washed in PBS. The pellet is resuspended in PBS (2×10⁵ cells/ml) and 1 ml is then added in a dropwise fashion to ice cold 70% EtoH whilst continuously vortexing. Cells are then permeabilized by incubating at −20° C. for 24 h. To stain DNA, cells are centrifuged and washed in PBS. Cells are resuspended in 500 μl Propidium Iodide Buffer (Propidium iodide (50 μg/ml); RNAse A (50 μg/ml) for 15 min in the dark before flow cytometric analysis using a FacsCalibre (Becton Dickinson). Analysis is performed using Cell Quest software.

GM-CSF Withdrawal Decreases the Percentage of Cells in Live Gate

Culturing TF1 cells in the absence of GM-CSF induces cell death. This death induces morphological changes in the Forward and Side Scatter parameters that can be detected by FacsCalibre analysis. Consequently, by observing the percentage of cells in the live gate (which is an arbitrary region pre-set on a healthy population of TF1 cells grown in the presence of GM-CSF (2 ng/ml)), one observes that there is a decrease in the percentage of cells with these light scatter parameters, when cytokine is withdrawn.

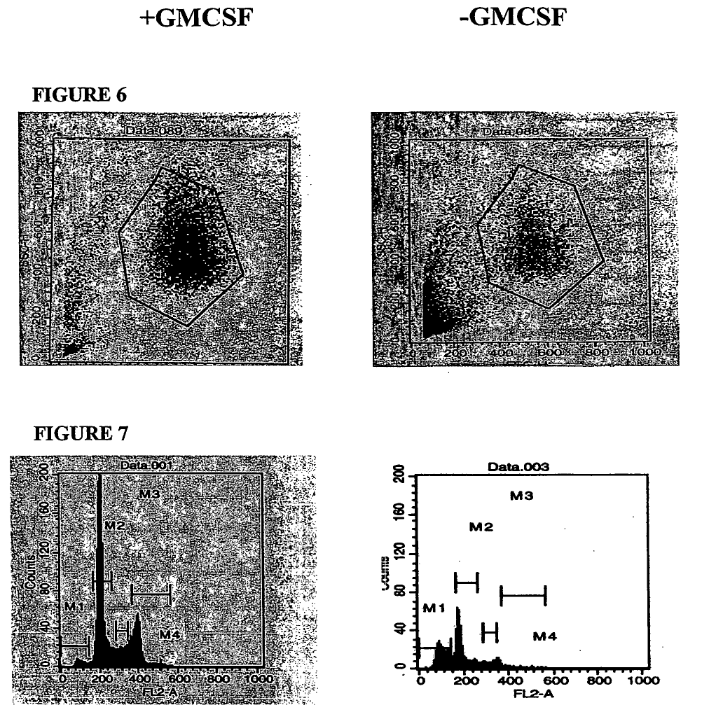

FIG. 6 shows the percentage of cells in the live gate decreases from 77% to 35% upon factor withdrawal for 48 h, representing a decrease of approx. 50% over the time period.

FIG. 7 shows TF1 cells grown in the absence of GM-CSF for 48 h have an increase of cells with sub-G1 profiles from 5% to approximately 55%, indicative of the cells undergoing apoptosis.

Expression of the Control Apoptosis-Regulator BCL2 Inhibits the Apoptosis of TF1 Cells

A number of gene products have been previously shown to regulate the effect of apoptotic stimuli on cells. The archetypical example of this is BCL2 protein expression where overexpression of this gene, has previously been shown to inhibit apoptosis in a number of cellular systems. (Hockenbery D, Nunez G, Milliman C, Schreiber R D, Korsmeyer S J, Nature 348(6299):334-6, 1990). We have transduced the human BCL2 gene into TF1 cells to examine its effect on survival following GM-CSF withdrawal.

To assess the antiapoptotic effect of BCL2 over-expression in TF1 cells, cells are transduced with either retrovirus expressing full length BCL2 coding sequence or control vector lacking BCL2. The cells are then induced to undergo apoptosis by withdrawal of GM-CSF for 48 h. The percentage of transduced cells remaining in the live gate (an arbitrary region pre-set on a healthy population of TF1 cells grown in the presence of GM-CSF (2 ng/ml)) is recorded. An index of antiapoptotic activity is calculated by computing the difference in samples ±GMCSF.

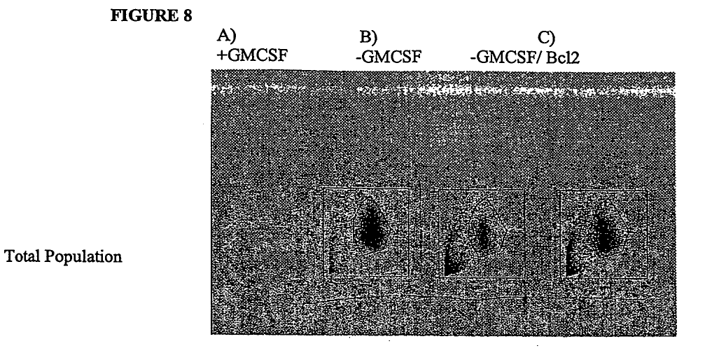

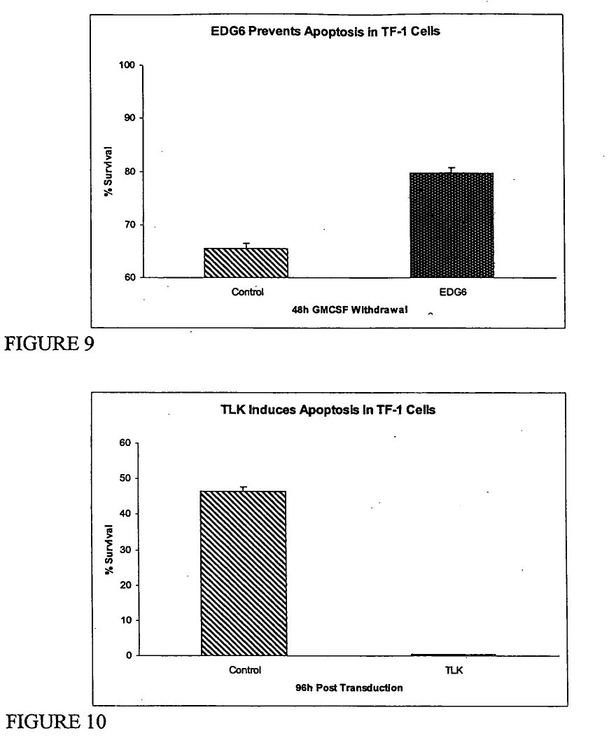

As can be seen in FIG. 8, removal of GM-CSF from the culture medium induces apoptosis of control cells, as determined by the number of cells moving out of the live gate (indicated by the gate). In contrast, expression of BCL2 reduces the number of cells moving from the live gate. The ability of BCL2 to prevent GM-CSF withdrawal induced apoptosis has been peviously reported (Ito, Overexpression of BCL2 suppresses apoptosis in the human leukemia cell line TF1, Rinsho Byori 1997, 45(7) 628-37).

These data illustrate the appropriate nature of this assay for determination of apoptotic or survival activity.

Overexpression of Identified GPCR EDG6 Decreases Apoptosis in TF1 Cells Which is Induced by Withdrawal of GM-CSF

To assess the apoptotic activity of EDG6, TF1 cells are transduced with either a retrovirus expressing the EDG6 coding sequence or a control vector lacking EDG6. The EDG6 coding sequence encoding the amino acid sequence described in Table 1B is cloned into the retroviral expression vector pMSCV (Clontech). Plasmids are transfected into the retroviral packaging cell line Phoenix 293 (Standford) using the CalPhos Mammalian Transfection Kit (Clontech) according to the manufacturer's instructions. Retrovirus is harvested 72 hours later. Retrovirus is transduced to TF1 cells by incubation for 18 hours. Media is changed and cells are incubated for an additional 48 hours.

The transduced cells are cultured for a further 48h in the absence or presence of GM-CSF (2 ng/ml) for a further 48 h prior to examining for viability using forward scatter/side scatter parameters. FIG. 9 shows that withdrawal of GM-CSF in control TF1 cells reduces viability to 65% whereas those cells over-expressing EDG6 had a viability of greater than 79%, indicating that EDG6 expression confers survival in TF1 cells.

Overexpression of Identified Kinase TLK2 Induces Apoptosis in TF1 Cells.

To assess the apoptotic activity of TLK2, TF1 cells are transduced with either a retrovirus expressing the TLK2 coding sequence or a control vector lacking TLK2. The TLK2 coding sequence encoding the amino acid sequence set out in Table 1B is cloned into the retroviral expression vector pMSCV (Clontech). Plasmids are transfected into the retroviral packaging cell line Phoenix 293 (Standford) using the CalPhos Mammalian Transfection Kit (Clontech) according to the manufacturer's instructions. Retrovirus is harvested 72 hours later. Retrovirus is transduced to TF1 cells by incubation for 18 hours. Media is changed and cells are incubated for an additional 96 hours.

The cells are examined for viability after 96 hours using forward scatter/side scatter parameters. FIG. 10 shows the control cell population is still viable (46.33%) while the TLK2 transfected cell population are almost all dead (0.48% viable cells). These data demonstrate that TLK2 mediates a strong pro-apoptotic effect in TF1 cells.

### Example 5

### Knockdown of Identified Genes in Cancer Cell Lines

The functional impact of silencing these genes is examined by using small interfering RNA oligomers. The effect of this silencing is measured using viability and apoptosis assays as described in Examples 6 to 8.

To evaluate the ability of each siRNA oligonucleotide to knock down its intended target, each siRNA was validated in cell lines known to express the target. The efficacy of knockdown was measured using QPCR in two separate experiments, A) and B).

A)

Design of siRNA Oligonucleotides.

The coding sequences of the novel identified genes, kinases/GPCRS, are screened for AAN19TT siRNA target sequences with a GC content of 40-55% (http://www.ambion.com/techlib/misc/siRNA_finder.html). Candidate oligonucleotides are subject to a BLAST search against the Genbank database to ensure that the selected sequences share no significant homology with any other human genes. In addition, the siRNA for a subset of genes were designed by Ambion using their proprietary design algorithms. The oligonucleotides selected are depicted in Table 9. The siRNA duplexes are chemically synthesised as N19(RNA)+TT(DNA) by Qiagen and purified to the Qiagen HPP standard. The oligos are supplied annealed, and are resuspended to a concentration of 20□M in siRNA buffer (100 mM potassium acetate, 30 mM HEPES-KOH, pH 7.4, 2 mM magnesium acetate) then incubated for 1 min at 90° C. followed by 1 hour at 37° C. The siRNA designed by Ambion is chemically synthesised by Ambion and purified by HPLC. These siRNA duplexes are resuspended in H₂O at 20 μM. Duplex siRNAs are stored at −20° C. until required.

Validation of siRNA Efficacy in Silencing Gene Expression.

To evaluate the ability of each siRNA oligonucleotides to knock down its intended target, each siRNA was validated in a cell line known to express the target, in this example the genes were tested PC3 cells, which were shown by expression profiling to express all target genes. The efficacy of knockdown was measured using QPCR. Cells are plated at a density of 2×10⁴ cells in each well of a 24 well plate and incubated at 37° C. overnight. The siRNA is transfected into cells using RNAiFect reagent (Qiagen) with siRNA at a concentration of 50 nM according to manufacturer's instructions. Additional wells of cells are transfected with 50 nM of a non-silencing control (Qiagen). The latter sample serves as a negative control for RNAi and also demonstrates the specificity of the knock down by the siRNA. Transfected cells are harvested at 24 hours post transfection, and RNA extracted using RNeasy Miniprep columns with an on-column DNase digestion step. The RNA is eluted in 30 μl of RNase-free H₂O. cDNA is synthesised from 12 μl of this RNA using an anchored oligo(dT) primer (Sigma) and Transcriptor reverse transcriptase (Roche) at 55° C. in a total volume of 20 μl following the manufacturer's instructions. The resulting cDNA is diluted to a total of 200 μl with H₂O and 10 μl used in each QPCR reaction. The levels of transcript are compared by QPCR, relative to the non-silencing control. QPCR is performed using primers, conditions and methodology identical to those used for expression profiling, see Table 8. All reactions were performed in duplicate and the average Ct values calculated. For each sample, the Ct of each target gene in normalised to GAPDH. Relative expression between cells transfected with target or non-silencing siRNA, normalised to GAPDH expression, is determined by the comparative Ct method. Table 10 shows the percentage knockdown in PC3 cells of target genes with reference to the control cells (in this case cells transfected with non-silencing siRNA).

B)

A second batch of siRNAs were generated substantially as described above and chemically synthesised as N19(RNA)+TT(DNA) by Eurogentec (Belgium). The purity of the siRNA Oligos is described by a Synthesis Scale of 0.211mol and are PAGE purified. The oligos are annealed at a concentration of 20 μM in annealing buffer (100 mM potassium acetate, 30 mM HEPES-KOH, pH 7.4, 2 mM magnesium acetate) for 1 min at 90° C. followed by 1 hour at 37° C. Duplex siRNAs are stored at −20° C. until required. Table 11 shows the sequences of the oligonucleotides selected.

Validation of siRNA Efficacy in Silencing Gene Expression

These siRNA oligos are initially tested for efficacy in a cell line in which the gene is expressed for example in the Hela, U251, TF1 or DU145 cell lines. Cells are plated at a density of between 7.5-10×10⁴ in each well of a 6 well plate and incubated at 37° C. overnight in DMEM culture medium (1× Dulbeccos modified Eagles medium (Sigma D2554), 0.004% folic acid, 4 mM L-glutamine, 0.37% sodium bicarbonate, 0.1 mM sodium pyruvate) containing 10% fetal calf serum (FCS). 2 μg of duplex siRNA is transfected into cells using DMRIE-C reagent (Invitrogen 10459-014) according to methods described in U.S. Patent Application No. 60/498,488. In brief, to prepare 1 ml of a transfection solution 2 μg of siRNA duplex in added to 1 ml of Optimem (Gibco), to which 8 μl of DMRIE C reagent is added. Transfection mix is added directly to the cells and incubated for 4 hrs at 37° C. Post this 4 hr incubation, transfection solution is removed and cells are refed with fresh culture medium and incubated for a further 24 hrs. An additional medium change is required at this point to minimise toxicity of the transfection procedure. Cells are harvested at 24 or 48 hrs post transfection for analysis of mRNA expression. Additional wells of cells are transfected with 2 μg of a missense control as described above.

In the case of TF1 cells, 2×10⁵ cells/ well are transfected with 2 μg of siRNA in each well of a 24 well plate using 8 ∞l DMRIE-C reagent (InVitrogen) per ml of Optimem (Gibco). Cells are simultaneously transfected in parallel with a control missense siRNA. Recombinant GM-CSF at 2 ng/ml is added to the OPTI-MEM media during transfection. The transfection media is replaced after four hours with fresh RPMI 1640 containing 2 μg/ml GMSCF and 10% FCS and incubated at 37° C. Twenty-four hours post-transfection, the media are changed; RPMI 1640+10% FCS containing 2 μg/ml GMSCF is added to 3 wells and RPMI 1640+10% FCS without GMSCF is added to the other 3 wells. These cells are harvested 48 and 72 hours post transfection for QPCR and phenotypic assays. Control TF1 cells, which are not transfected, are similarly treated. Samples are labeled T48+G, T48−G, according to the time of harvesting post-transfection and the presence (+G) or absence (−G) of GM-CSF in the media. RNA is isolated, cDNA is reverse transcribed and QPCR is performed as described above. Short Term (72 hrs post transfection) Cell Viability/Apoptosis assays employed are discussed below.

The missense control used serves as a negative control demonstrating that activation of the RNAi machinery itself is not toxic to the cells. Missense controls also illustrate the specificity of the knock down induced by siRNA oligomers. The sequences of the missense control siRNA oligos used are:

| | |
|---|---|
| MS | |
| Sense: | UGAGAAUGUGAUGCGCGUCTT |
| Antisense: | GACGCGCAUCACAUUCUCATT |

The missense control outlined above is designed by introducing 4 base pair mismatch substitutions into the siRNA oligo sequence for ELAV-1 (Accession number xm_{—}008947; gi 1475941). It was confirmed that these substitutions render the siRNA oligo incapable of decreasing the expression ELAV-1 and thus validate the MS siRNA as a true negative control.

Additional genes which have previously been described in the literature to be associated with apoptosis were selected as control genes in RNAi assays. For example Survivin is a known key apoptosis modulator whose expression is disregulated in cancer and is a known drug target in development for cancer. When its expression (which is maintaining survival and the cancer phenotype) is reduced by an siRNA oligo specific to Survivin the cancer cells die (as illustrated in the cell viability assays discussed below). Thus Survivin knockdown provides a positive control for inducing cytotoxicity and allows the function and the potential role of any of the target genes identified herein in potentiating a cancer phenotype to be determined by analogy, if, on knockdown, a target gene also induces a cytotoxic response in cancer cells. These controls and the sequence of the siRNA oligomers used against them are illustrated below.

Survivin (Survivin B, SurB, SURB, SUR)

| | |
|---|---|
| Sense: | GAACUGGCCCUUCUUGGAGtt |
| Antisense: | CUCCAAGAAGGGCCAGUUCtt |
| PI3KR1 | |
| Sense: | AUGAUCGAUGUGCACGUUUtt |
| Antisense: | AAACGUGCACAUCGAUCAUtt |
| BCL2 | |
| Sense: | GUACAUCCAUUAUAAGCUGtt |
| Antisense: | CAGCUUAUAAUGGAUGUACtt |
| c-Raf (CRAF) | |
| Sense: | UAGUUCAGCAGUUUGGCUAtt |
| Antisense: | UAGCCAAACUGCUGAACUAtt |

After DMRIE transfections with siRNA Oligos against the apoptosis associated genes identified herein, or controls, cells are harvested at 24 or 48 hours post transfection as stated previously. RNA is then extracted using RNeasy Miniprep columns (Qiagen 74104) after lysis on KIAshredder columns (Qiagen 79654). RNA is quantified by spectrophotometric analysis and 1 μg is reverse transcribed into cDNA using SuperScriptII RNAseH-Reverse Transcriptase (Invitrogen 18064-014). The levels of transcript are compared by QPCR as described in detail in Example 3, relative to missense controls. QPCR primers are designed to amplify a PCR product of approximately 100 to 300 nucleotides in length from the CDS, following the guidelines outlined in the Quantitect SYBR Green PCR handbook from Qiagen.

QPCR Primers selected are those outlined in Table 4 as described above.

Two template standards are used for quantitative PCR assessment of mRNA levels:
1. A control RPS13 i.e. Ribosomal Protein Sub unit 13 (NM_{—}001017) amplified PCR product diluted to [1000,100,10,1 and 0.1 fg] respectively, used to quantify the level of transcript;
2. RPS13 amplified from sample templates as separate PCR reactions for normalisation purposes.

The cDNA templates from appropriate cell lines transfected with either gene specific, positive control e.g. Survivin or missense control siRNA are amplified with gene-specific QPCR primers using QuantiTectSYBR Green PCR kit (Qiagen 204143) on a DNA Engine Opticon System (MJ Research) (as described in detail in Example 3). The amplification conditions include a 95° C. step for 15 min for initial activation of HotStarTaq DNA polymerase, followed by 35 cycles of (15 s at 94° C., 30 s at 60° C., 30 s at 72°). The fluorescence of the samples at 521 nm is read between the annealing and extension steps of the protocol. The melting curves are calculated at the end of the 35 cycles and confirm product homogeneity.

A standard curve is plotted using the log [template quantity] of the RPS 13 control template dilutions (as above) versus cycle number at which the fluorescence intensity measured in the well exceeds the level specified in the cycle threshold parameters (the C(T) value). An estimate of the quantity of initial template in treatment samples is determined from this plot and the amount of target mRNA present in each sample is normalized across samples by calculating the ratio of target to RPS13 for each sample.

The normalised expression values are used to estimate percentage knockdown of target with reference to the control, transfected, cells (in this case cells transfected with missense siRNA).

siRNA Oligonucleotides are Effective at Reducing Target Gene Expression

Targeting specific genes with siRNA oligonucleotides effectively reduces the expression of the targeted gene's mRNA. This is measured by comparing the relative levels of specific mRNA present in the cells before and following treatment with the respective siRNA oligo and also by comparing the level of the specific mRNA in cells transfected with the missense control oligos. The level of knockdown achieved varies for each oligo, across the range of cells lines examined.

Table 12 gives the percentage decrease of the target genes following treatment of four different cell types with siRNA.

siRNA Does Not Induce the Interferon Response

Inhibiting gene expression using siRNA has been associated with non-specific induction of interferon from target cells (Sledz C A et al. Nat Cell Biol. September 2003; 5(9): 834-9. 2003). Since induction of interferon may possibly affect biological assays, we control for the effect of our siRNA oligonucleotides on inducing interferon. This is examined by quantitating message RNA levels for two interferon responsive genes, namely OAS1, and GBP1, both before and after siRNA transfection.

QPCR Primers, designed by MWG Biotech as described previously, are as follows.

| | |
|---|---|
| Forward Primer | Reverse Primer |

| | | |
|---|---|---|
| Gene | (5′-3′) | (5′-3′) |

| | | |
|---|---|---|
| OAS1 | GCGCCCCACCAAGCTCAAG | GTCCGAAATCCCTGGGCTGT |
| (NM_00253 | A | GTT |
| 4) | | |
| GBP1 | TATGGTGGTGGTGGCAATT | ACGGCCAGGGCGAAGATCC |
| (NM_00250 | GTGG | |
| 3) | | |

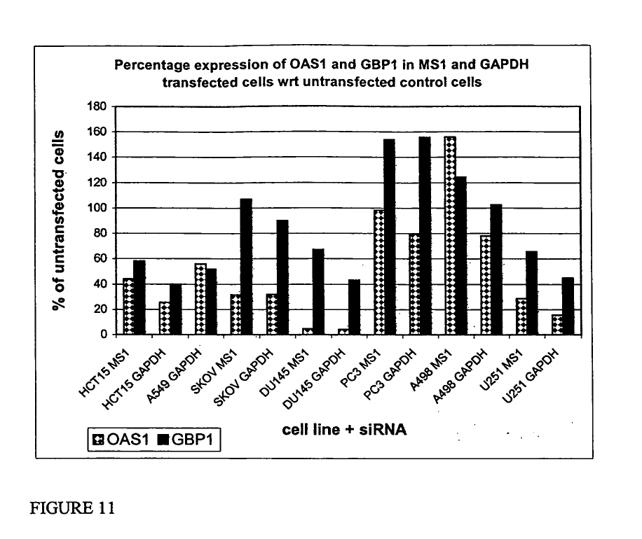

FIG. 11 shows a determination of the mRNA levels (by QPCR quantification as described) of 2 interferon response genes, OAS1 (NM_{—}002534) and GBP1 (NM_{—}002503), in 7 cell lines (HCT15, A549, SKOV3, DU145, PC3, A498, and U251) post siRNA transfection with MS or GAPDH siRNAs. This demonstrates that in HCT15, A549, SKOV3, DU145 and U251 cells transfection of siRNA does not increase mRNA levels of interferon response genes as compared to untransfected cells. Some increase in mRNA of interferon response genes was noted for PC3 and A498 cells but this is not considered of a sufficient magnitude to influence biological assays particularly in view of the results using a missense control (MS).

The levels of expression of OAS1 and GBP1 in siRNA-transfected cells are expressed as a percentage of that in untransfected control cells.

### Example 6

### Knockdown of Identified Genes in Cells Functionally Modulates Apoptosis as Determined by Short Term Assays

The present example demonstrates that the identified ‘novel’ genes play a fundamental role in the apoptosis pathway in a variety of cell types. We demonstrate that disruption of the functional expression of these genes modulates apoptosis and cell viability across a broad range of cancer cell lines over a 72-hour period.

Following confirmation of siRNA activity, the siRNA duplexes are introduced into cancer cell lines to test for modulation of apoptosis/cell viability and other characteristics associated with a cancer phenotype (discussed in Examples 7 and 8), on knockdown of the target gene's mRNA expression. Test cell lines include Hela, TF1, U251, SKOV3, OVCAR3, MCF7, PC3, HCT15, 786-0, HT29, M-14, H460, LnCap, DU145, A549, A498, HCT 116, PanC1, MDA-MB-23 land MDA-MB-468,

Except where indicated, the methodology employed for transfection and subsequent analysis by QPCR or Cell Viability/apoptosis assays, only varies depending on the scale of experiment employed and the cell line utilised. In brief, test cell lines are seeded 24 hours prior to transfection at varying densities depending on the cells line and scale of experiment necessary, but such that they are 40-70% confluent on transfection. Cells are transfected with 2 μg of siRNA per ml of Optimem (Gibco) plus 8 μl of DMRIE C reagent (InVitrogen). Cells are simultaneously transfected in parallel with a control missense siRNA. The transfection media is replaced after four hours with fresh media containing 10% FCS and incubated for 24 hours at 37° C. An additional medium change is required post this 24 hour incubation so as to minimise toxicity associated with the transfection procedure. These cells are harvested at 24 or 48 hours post transfection for QPCR Analysis, RNA is isolated, cDNA is reverse transcribed and QPCR is performed as described previously. Cells are harvested at 48 or 72 hours post transfection for our short term cell viability/apoptosis and phenotypic assays and at 10-14 days post transfection for our long term viability assays (as discussed in Examples 7 and 8).

TF1 cells are cultured as described above. Cell Viability/Apoptosis assays employed are discussed below.

The sensitivity of the Survival assays for detecting apoptosis induction/decreased cell viability due to RNAi knockdown of specific genes is validated by including siRNA oligos against Survivin and/or BCL2 (apoptosis/viability controls) and the missense negative control oligo in each experiment as discussed in detail previously.

Determination of the Effect of Target Knockdown on Cell Survival Over a 72 h Period i.e Short Term Cell Viability Assays

To determine the effect of knocking down the expression of the target genes by RNAi, the viability of cells where the target mRNA is reduced, is examined using a number of assays as described below. Furthermore, to demonstrate that decreased viability occurred through apoptosis, a number of apoptosis assays are performed.

Cell Viability and Apoptosis Assays Used Include:

1. MTT High Throughput Screen (MTT HTS)

The MTT assay measures the ability of cells to convert the soluble MTT salt into an insoluble blue formazan crytal. This conversion requires the activity of mitochondrial succinate dehydrogenase in viable cells. Thus, dead cells are not able to convert MTT. Consequently, the amount of colour change is directly proportional to the level of viability of the cell population.

The number of cells alive 72 hours post transfection with siRNA oligos against our target genes is compared to the number of viable cells remaining post transfection with the missense siRNA oligo and a % cytotoxicity is ascertained for each of our target genes in the cell lines examined.

Cells are seeded into a 96-well plate and allowed to adhere overnight at 37° C. in a humidified atmosphere containing 5% CO₂ in air. Wells are seeded with an appropriate cell number that is cell line dependent. Cells are next transfected with siRNA Oligomers, at a concentration of 2 μg, against the genes of interest. Transfection mix consists of 2 μg of siRNA Duplex in 1 ml of Optimem containing 8 μl of DMRIE-C Reagent as described in detail above. Transfection continues for 4 hrs after which the transfection solution is removed and cells are refed with fresh medium and allowed incubate overnight. An additional media change at 24 hrs post transfection is essential to minimise toxicity of the transfection reagent. Cells are subsequently incubated for a further 48 hr period post transfection at a 37° C. incubation as described above, following which MTT quantification of cell number is performed.

Cell viability is determined using an MTT assay. Briefly, 10 μgl of a 5 mg/ml MTT solution in PBS (Sigma) is added to 100 μl aliquots of cells, mixed thoroughly and incubated for 2.5-4 hours at 37° C. Mitochondrial succinate dehydrogenase of viable cells converts the soluble MTT salt to an insoluble blue formazan crystal. Medium is subsequently aspirated off the wells and formazan crystals are solubilised by the addition of 100 ul of DMSO. Cell viability is correlated to MTT conversion to Formazan crystals that generates a deep purple coloured solution on solubilization with DMSO. Plates are read at 570 nm on a Molecular Devices Emax precision micro plate reader.

2. Forward Scatter/Side Scatter (FSC/SSC) Analysis

FSC/SSC Analysis utilised in this example represents an apoptosis assay that can distinguish accurately between cells that have undergone apoptosis or necrosis from viable cells. This is based on the characteristics changes in cell size and granularity associated with viable, apoptotic and necrotic cell morphology. To ascertain the characteristic cell size and granularity of a cell population, cells transfected with a missense control are analysed utilising the flow cytometer. The position of this cell population on the FSC and SSC scale are noted by isolating the population with a gate. The influence of siRNA oligo transfection against our target genes on cell viability is ascertained by observing the movement of the cell population from the viable cell gate previously recorded. Necrotic cells increase in size before disintegrating and therefore are noted to shift up and to the right of the viable cell population initially while next shifting completely into the debris section of the FSC/SSC plot (bottom left) when disintegrated. Apoptotic cells, however simply move to the left of the viable cells population gradually reducing in cell size, and then shift gradually down the SSC scale as the granularity of the cells increases in late stage apoptosis.

Therefore due to the cell size and granularity changes associated with apoptosis, the change in the FSC/SSC parameters can be used to identify cells undergoing apoptosis post siRNA transfection. The percentage of cells with Forward Scatter/Side Scatter (FSC/SSC) profiles of viable cells is determined 24-72 hrs post transfection of model cells with missense siRNA oligonucleotides as described above. Transfection procedure is carried out exactly as previously described. Cells are harvested at these time points, centrifuged (1000 rpm, for 10 min) and washed in PBS (Sigma). The pellet is resuspended in PBS or medium and cells acquired immediately by a FacsCalibre (HP Biosciences). Forward and Side scatter parameters are assessed using Cell Quest software. The position of cell populations transfected with siRNA oligos against our target genes as determined by the FSC/SSC parameters are compared to the position of cells transfected with the missense control siRNA as already stated. The % of cell remaining within the viable cell gate post transfection with target siRNA oligos is ascertained for each gene target across the cell lines examined.

3, Sub G1 Analysis of Apoptosis

Quantification of the % of Cells in each phase of the cell cycle is achieved by permeabilising the cell suspension and staining the DNA in the nucleus of each cell with Propidium Iodide. This is a fluorescent stain and therefore the intensity of the fluorescence detected by the flow cytometer correlates with the amount of DNA within that cell. By analysing cells transfected with our missense siRNA oligos the % of the cell population expected in each phase of the cell cycle in a viable cell population is ascertained. Cells in G1 have a diploid amount of DNA or ‘2n’, those with a DNA quantity>2n are in the synthesis or ‘S’ phase of the cell cycle, those with ‘4n’ are in the G2M phase of the cell cycle and are about to divide to give 2 cells. However cells undergoing apoptosis have <2n or a Sub G1 amount of DNA as its has been fragmented and has leaked out of the nucleus of the cell, therefore by quantifying the % of cells with a sub G1 amount of DNA, one can quantify the % of apoptotic cell is the population. Quantification of this population is performed at 24-72 hours post transfection. Therefore, the percentage of cells within the Sub G1 phase of cell cycle represents a measure of apoptosis in the cell population post transfection of an siRNA oligomer to a novel identified gene, compared to cells transfected with the missense control. Cell Cycle Analysis/SubG1 parameters are obtained by resuspending cells in a buffer, (0.1% Sodium Citrate, 0.1% TritonX-100, 200 μl of Propidium Iodide at 5 mg/ml made up to 20 mls in PBS) and staining the cells for 7-24 hours at 4° C. in the dark. The PI stained cells are then acquired by the flow cytometer. Analysis of FL2 fluorescence is performed on Cell Quest software to allow quantification of the Sub-G1 phase of the cell.

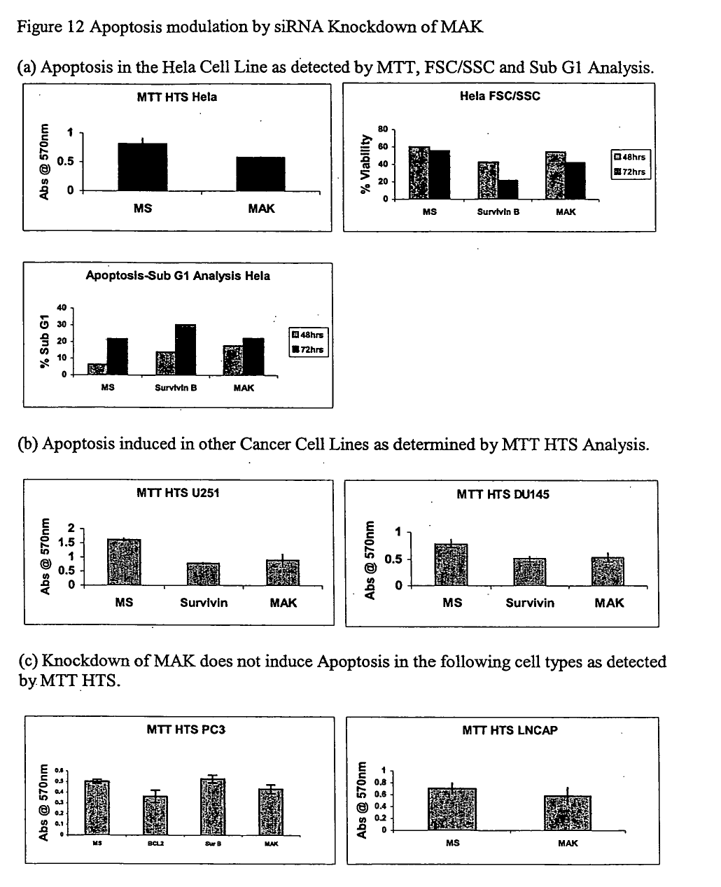

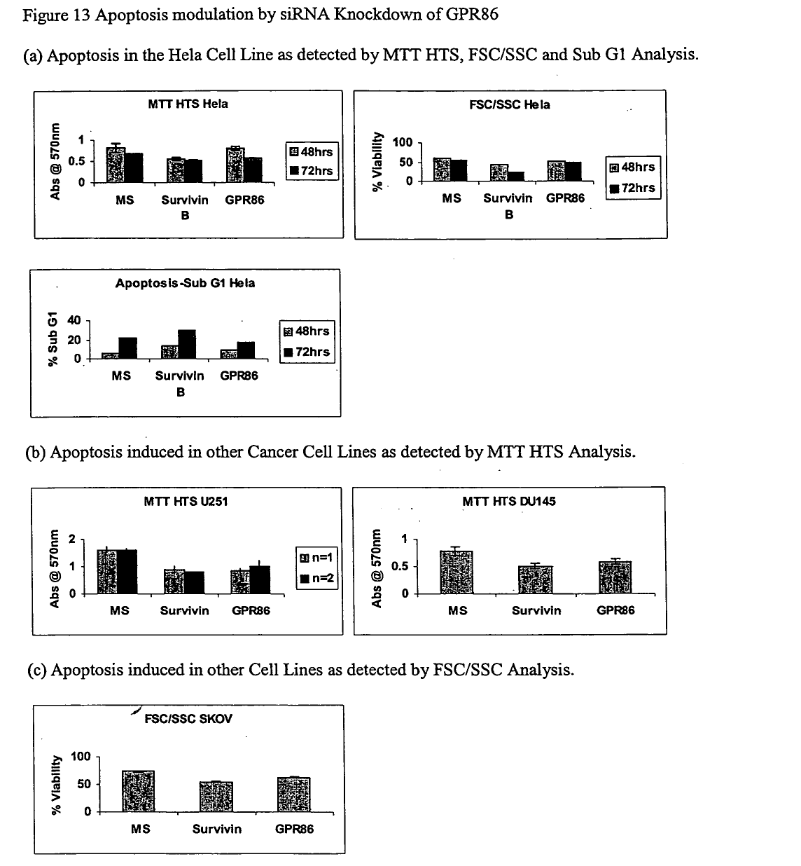

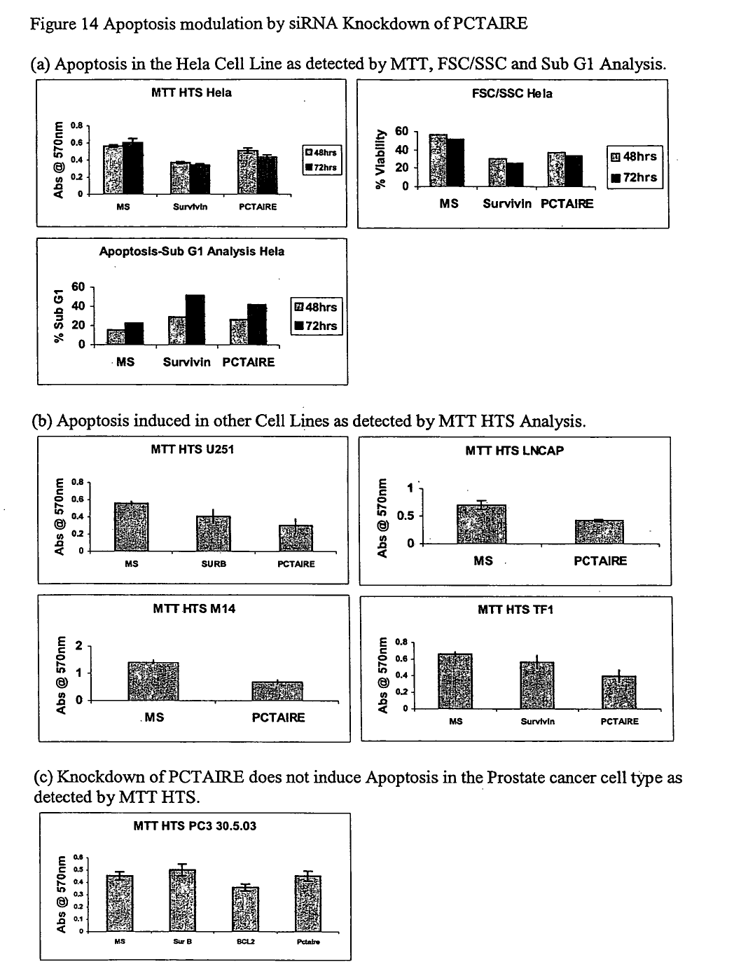

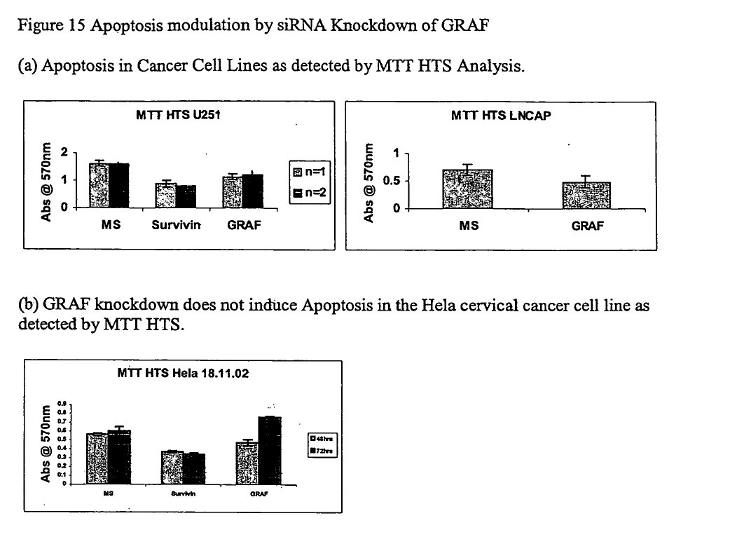

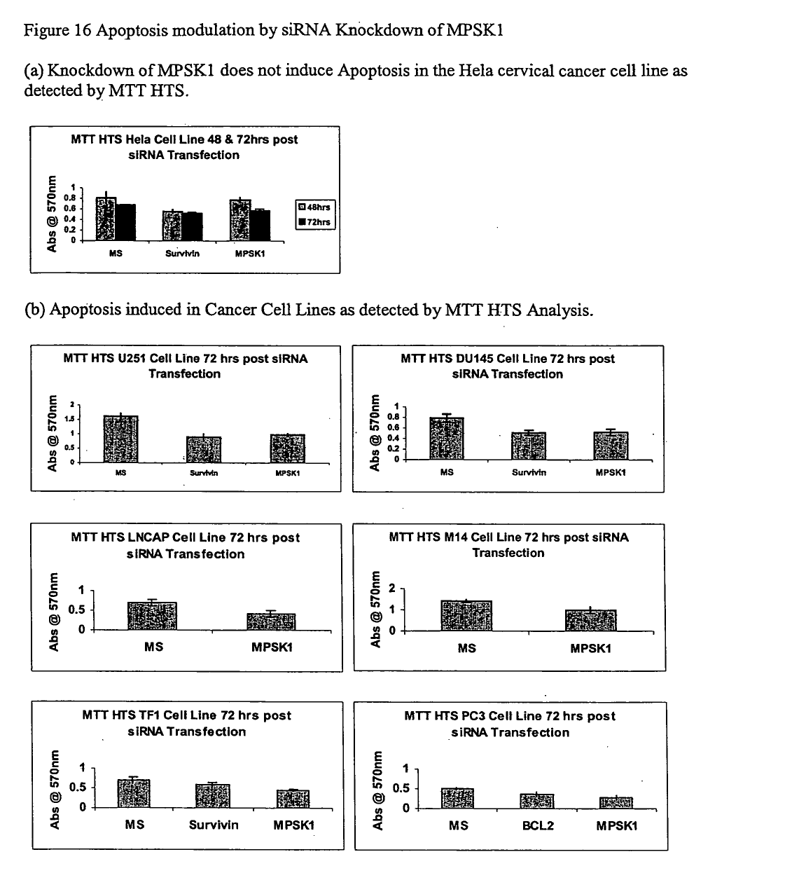

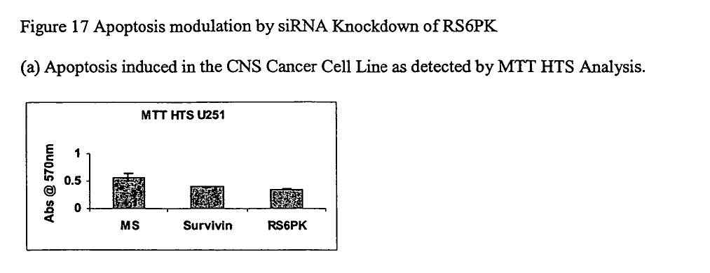

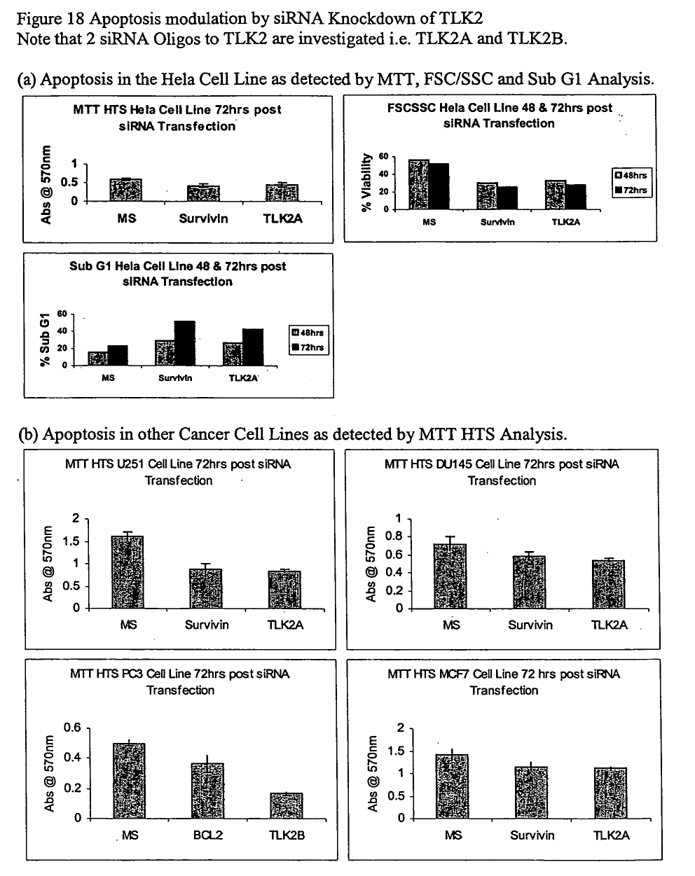

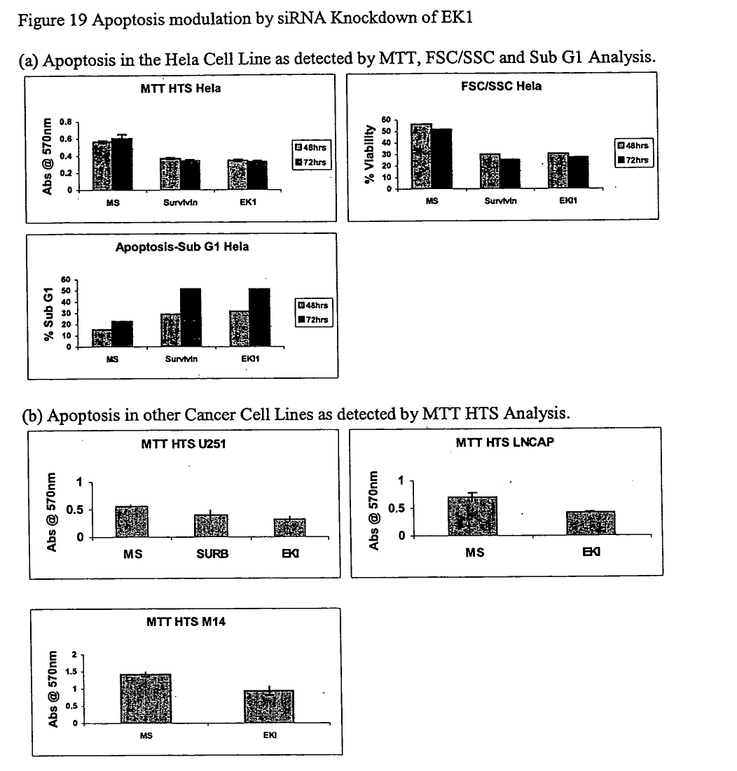

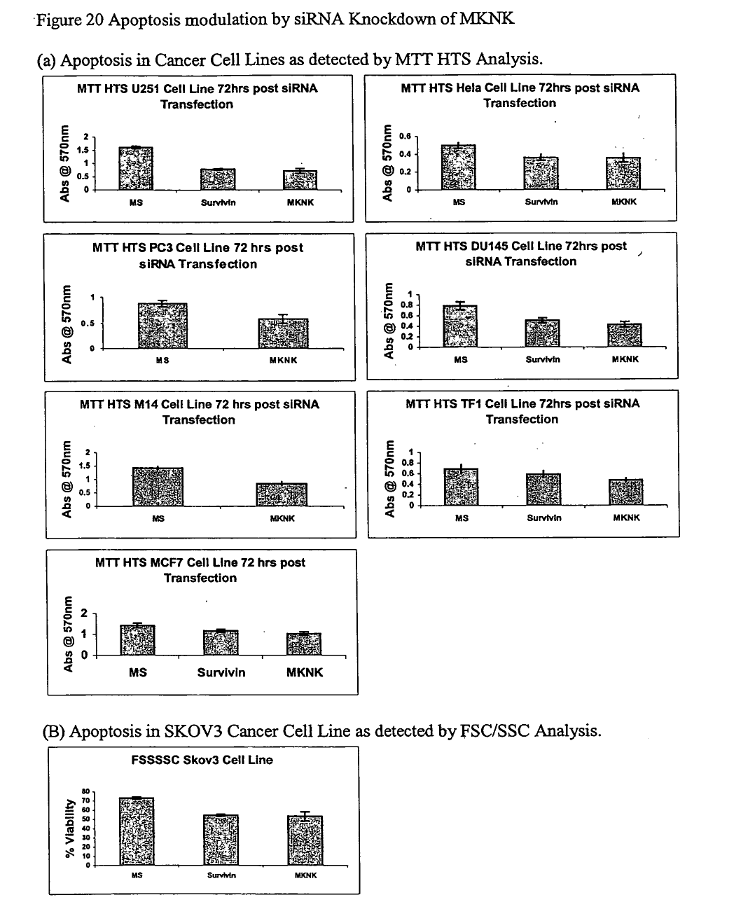

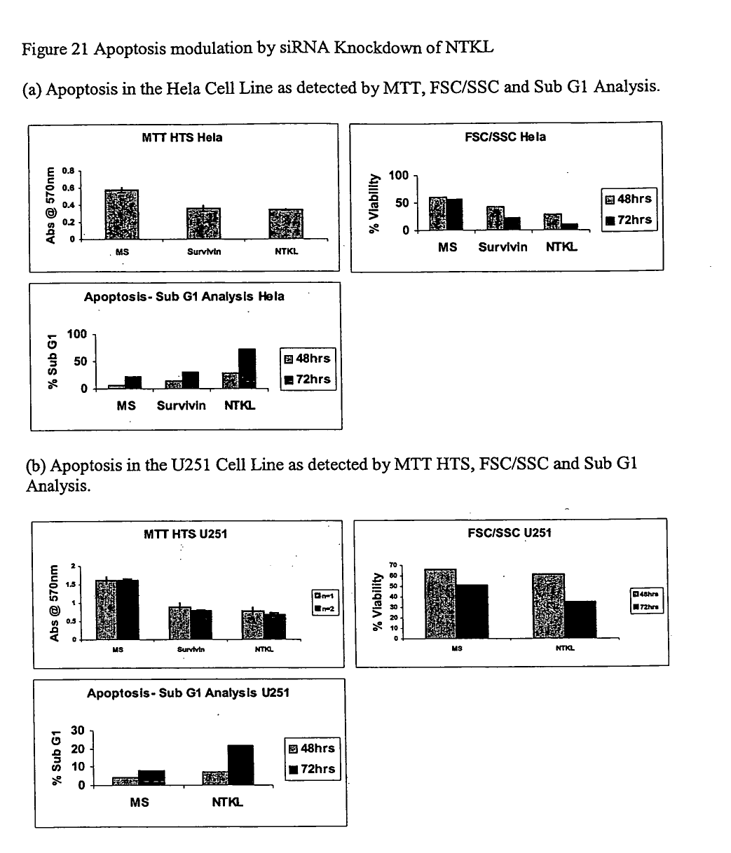

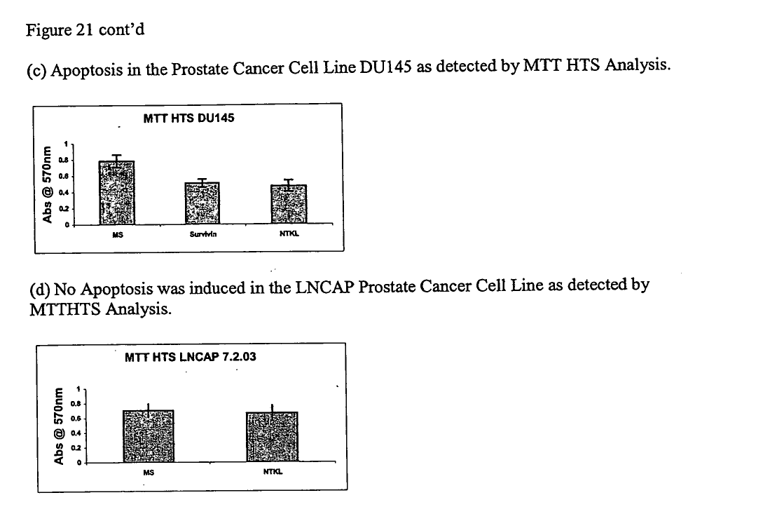

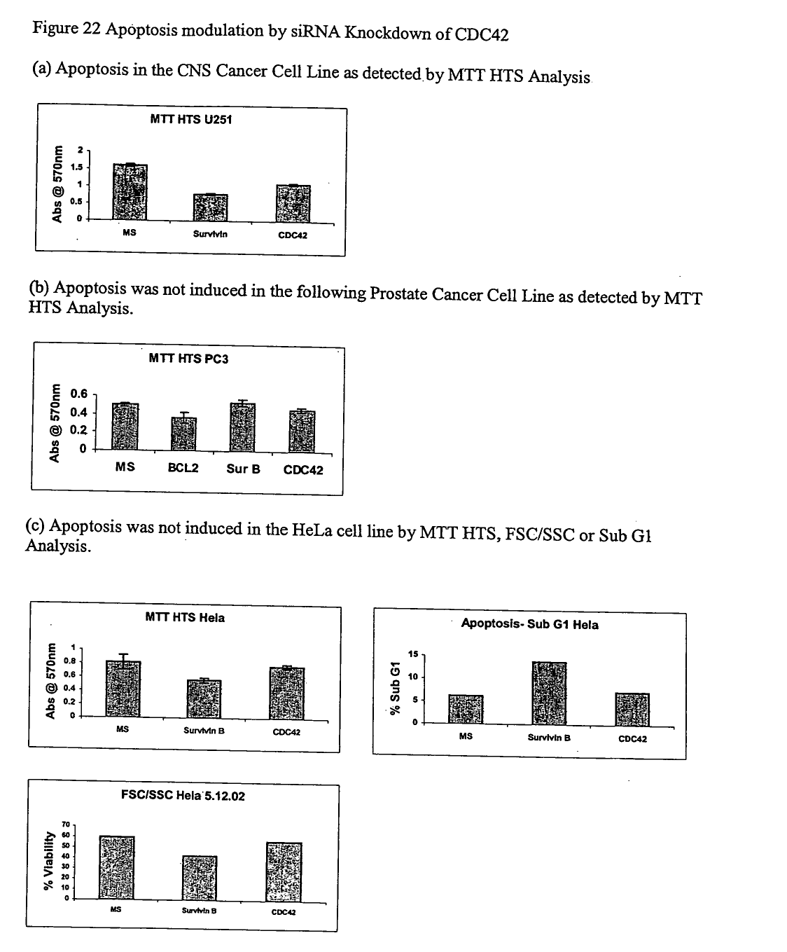

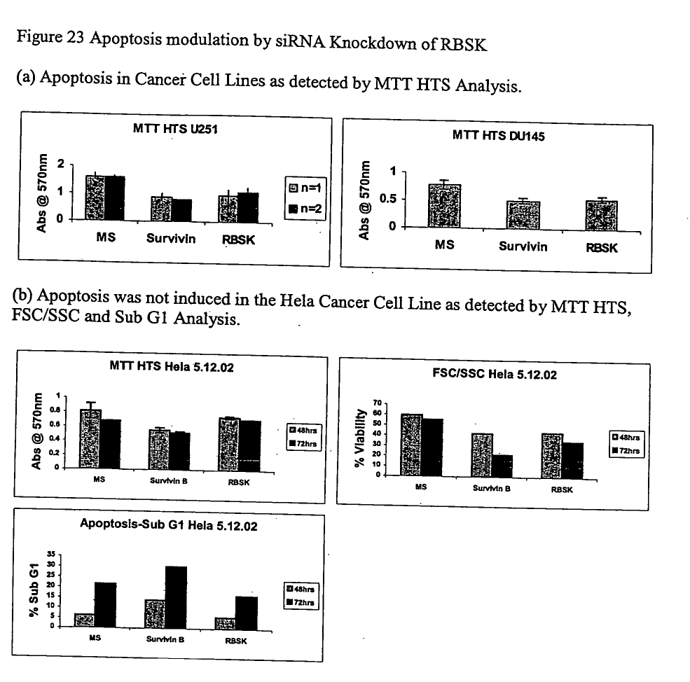

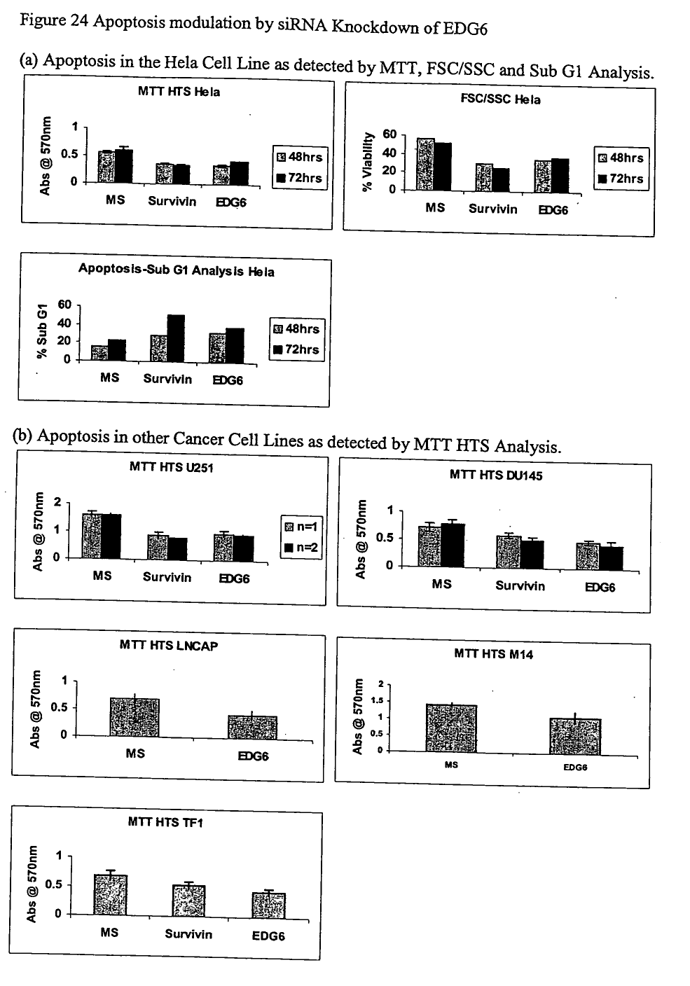

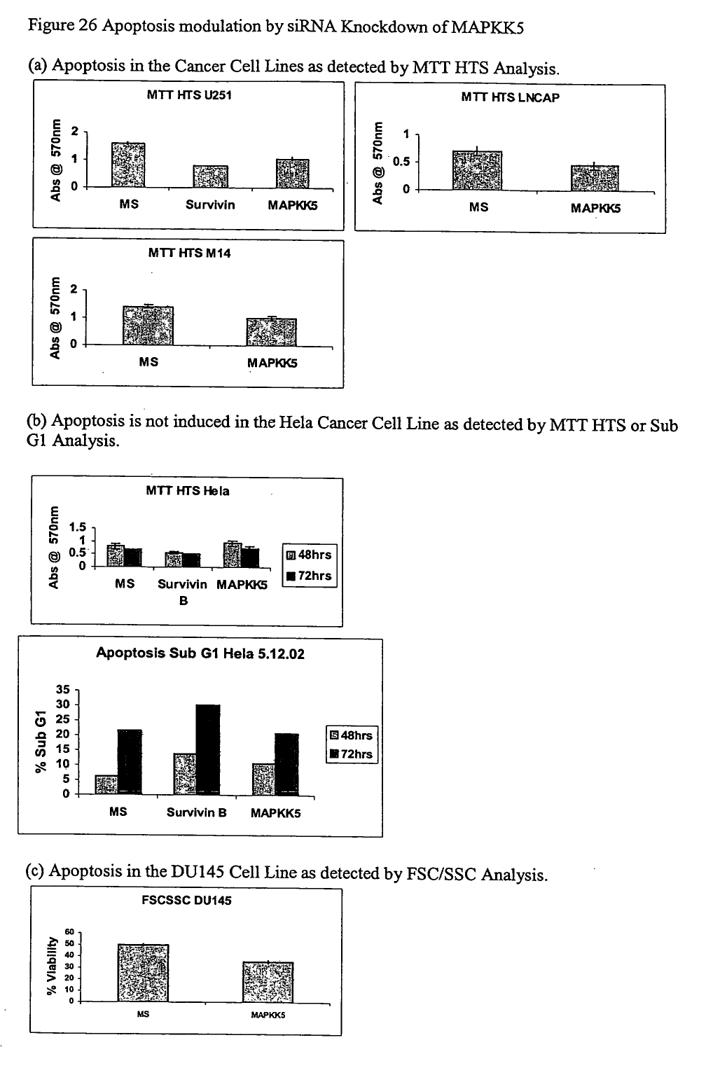

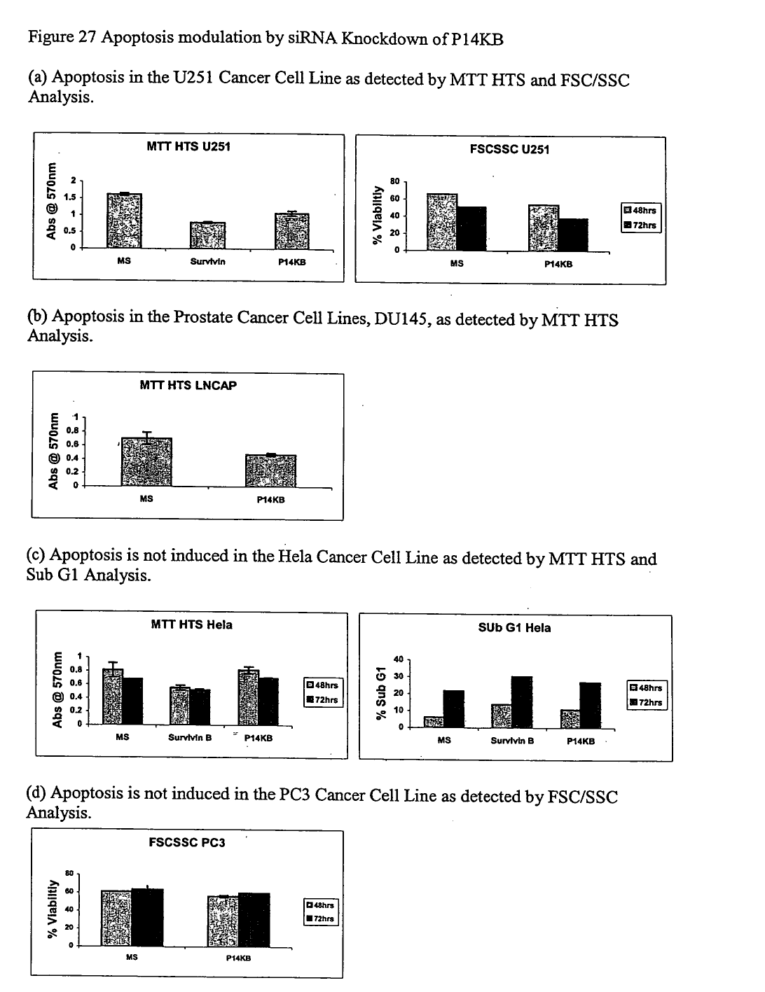

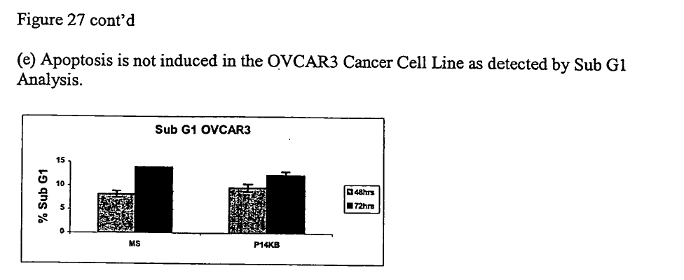

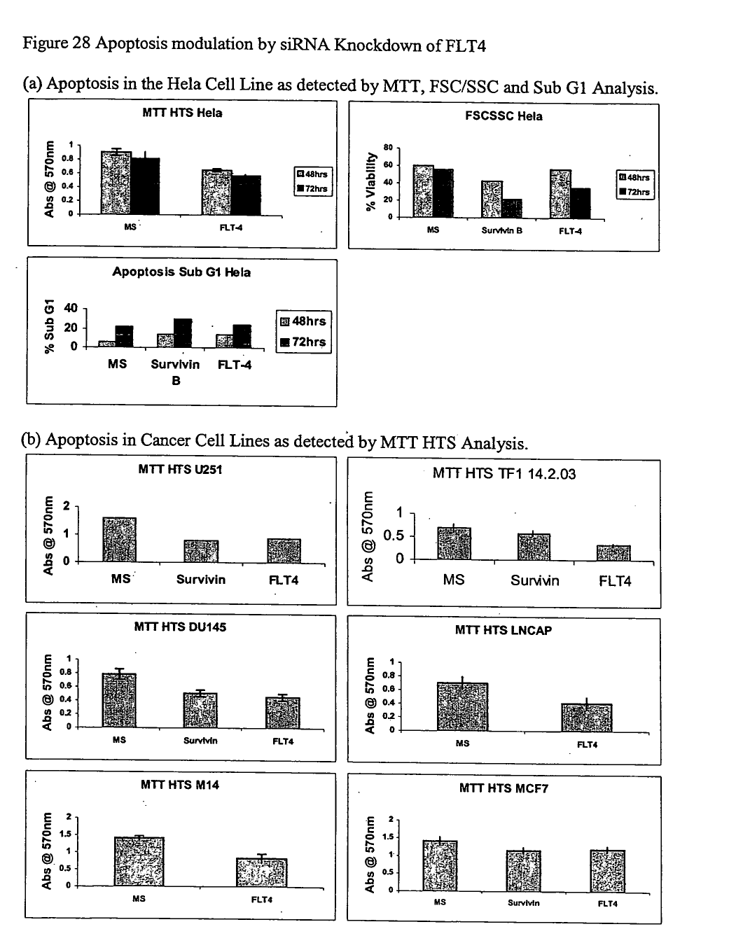

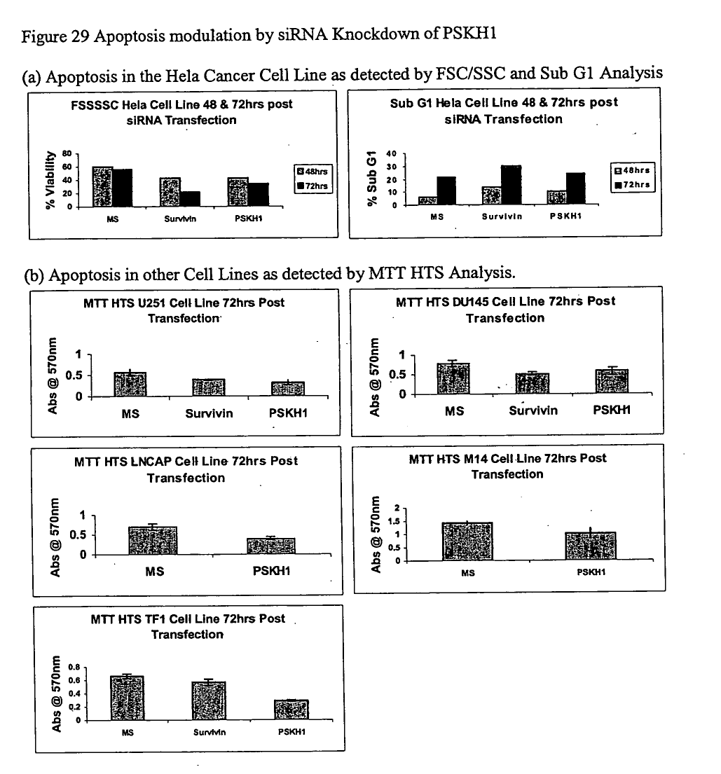

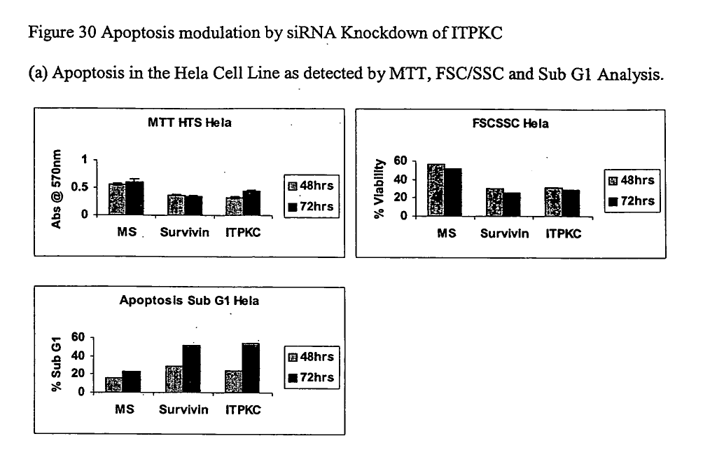

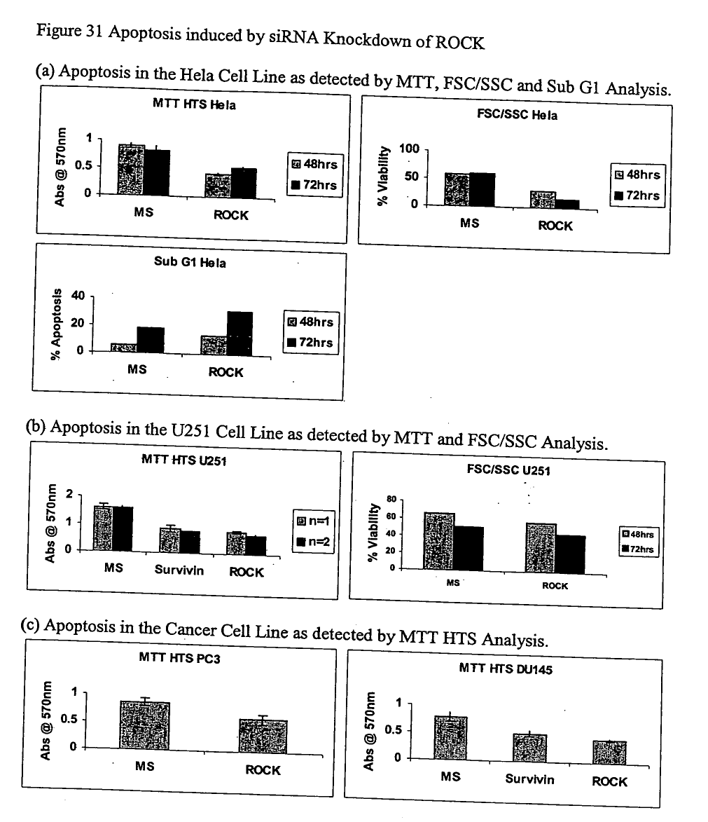

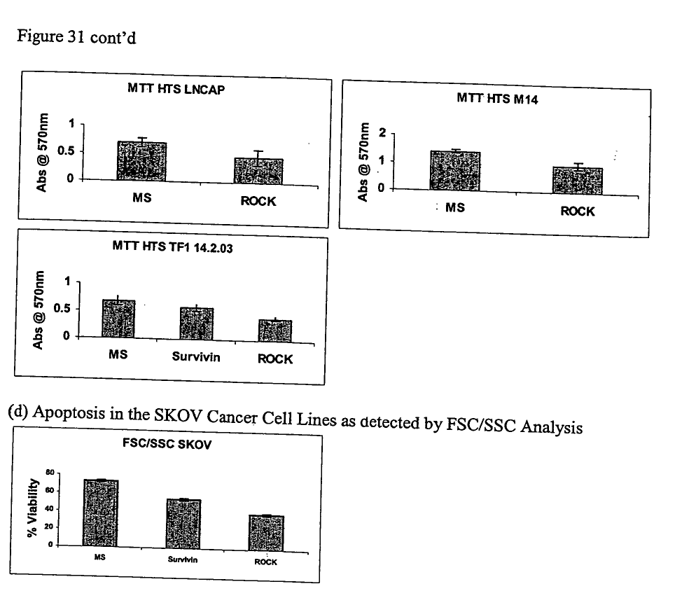

FIGS. 12 to 31 demonstrates that knockdown of target genes by RNAi modulates apoptosis as detected by MTT HTS, FSC/SSC and Sub G1 Analysis. These figures demonstrate the differential induction of apoptosis by target genes, compared to the Missense (MS), BCL2 and/or Survivin Controls, across the range of cell lines examined for each gene utilising these apoptosis assays.

4. JC-1 Analysis of Mitochondrial Membrane Depolarisation

Analysis of the Mitochondrial membrane potential of a cell population determines the % of Apoptotic and viable cells within a population. Mitochondrial Membrane Potential is quantified utilising the JC-1 Dye acquired from Molecular Probes and used according to manufacturers instructions. Cells are transfected with target specific siRNA oligomers, our positive control i.e. Survivin and a negative control i.e. Missense (refered to as MS) as described previously. Briefly, cells are harvested and incubated with the JC1 dye 48-72 hrs post transfection with siRNA oligonucleotide. When this dye is incubated with viable cells its remains in an aggregate form that fluoresces red on activation with an argon laser. However when apoptosis is occurring the JC-1 dye shifts to a predominantly monomer form that fluoresces green on activation. Therefore a shift from a red to a red/green or green fluorescent population indicates an apoptotic population. Analysis of fluorescence is performed on Cell Quest software to allow quantification of the apoptosis occurring in the population of cells examined. The % of cells undergoing apoptosis post transfection with target specific siRNA oligos is compared directly to apoptosis induced by the siRNA missense oligo.

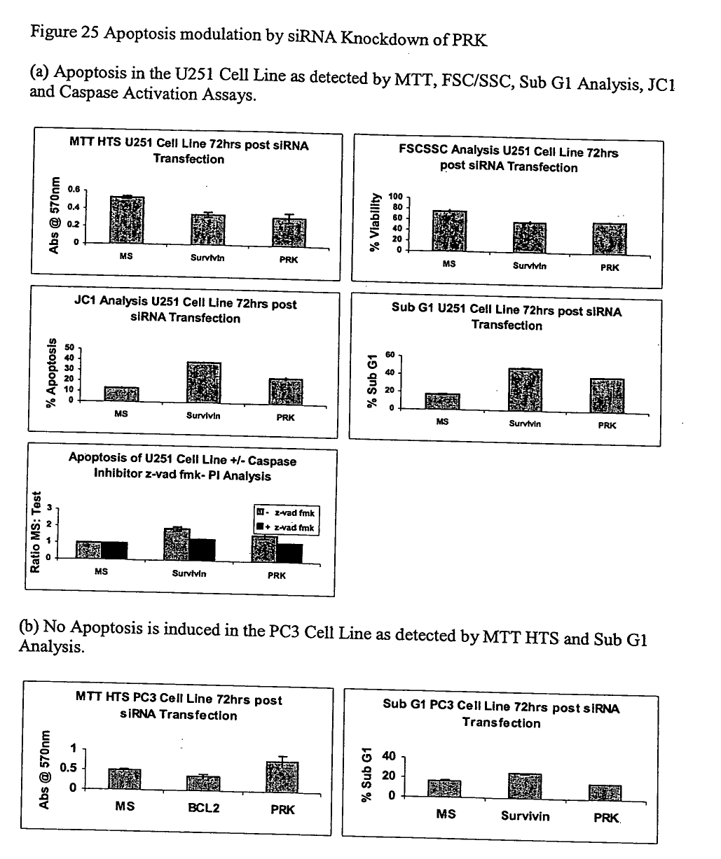

FIG. 25 Demonstrates that knockdown of target genes by RNAi modulates apoptosis as detected by JC-1 Analysis. This demonstrates the differential induction of apoptosis by a target gene, compared to the Missense (MS), BCL2 and/or Survivin Controls, in the U251 cell line.

5. Analysis of the Role of Caspase Activation and Apoptosis Utilising the Caspase Inhibitor z-vad fmk

To determine whether apoptosis induced by transfection of siRNA oligomers against novel identified genes and detected by analysis of the Sub G1 population, is Caspase Dependent, transfection experiments and the subsequent 72 hr incubation is performed±z-vad fink. The general Caspase Inhibitor reduces apoptosis only if it is induced via a caspase dependent pathway.

FIG. 25 Demonstrates that knockdown of target genes by RNAi that induces apoptosis as detected by Sub G1 Analysis may depend on caspase activation in a gene and cell line specific manner. This example demonstrates the differential induction of apoptosis in a caspase dependent or independent pathway by a target gene, compared to the Missense (MS), BCL2 and/or Survivin Controls, in the U251 cell line.

6. WST-1 Viability Screen

The Wst1assay measures the ability of cells to convert the wst1 salt into soluble blue formazan. This conversion requires the activity of mitochondrial succinate dehydrogenase in viable cells. Thus, dead cells are not able to convert wst-1. Consequently, the amount of colour change is directly proportional to the level of viability of the cell population.

Cells (Panc1, DU145, PC3, MDA-MB-231, MDA-MB-468, HCT116) are plated at a density of 3000 cells/well in 100 μl medium in 96-well plates and incubated overnight at 37° C. in a humidified atmosphere containing 5% CO₂. The adherent cells are then transfected with siRNA at a concentration on 25 nM or 50 nM using RNAiFect reagent (Qiagen) following the manufacturers instructions. Briefly, 1 μg of siRNA is added to a total volume of 100 μl EC-R buffer (Qiagen) and an appropriate volume of RNAiFect added to the cells, which is cell line-dependent. After a 15 minute incubation at room temperature, the siRNA-RNAiFect complexes are diluted to 275 nM or 550 nM with EC-R buffer. The diluted siRNA complexes are then added to cells so as to give a final concentration of 25 nM (MDA-MB-231 and MDA-MB-468) or 50 nM (Panc1, DU145, PC3, HCT116). After a period of 4 hours, the transfection solution is removed from the cells by inversion and fresh growth medium added. All transfections are performed in triplicate wells and include a mock transfection (transfection reagent only), a negative control siRNA (Qiagen non-silencing siRNA) and positive controls targeting BCL2, Survivin and C-Raf.

The Wst1 cell viability assay is performed 72 hours post-transfection. Wst1 reagent (Roche) is added to each well of transfected cells so as to give a 11× dilution (10 μl of Wst1 in 100 μl cell growth medium), the 96-well plate mixed thoroughly and incubated at 37° C. for a period of time that is cell line dependent. During this incubation, the tetrazolium salt WST-1 (4-[3-(4-iodophenyl)-2(4-nitrophenyl)-2H-5tetrazolio]-1,3-benzene disulphonate) is converted to formazan by the mitochondrial activity of viable cells. The formazan dye released, proportional to viable cell number, is measured using a scanning multiwell spectrophotometer at 450 nM. The relative number of viable cells in each transfected cell population is expressed as the percentage decrease of absorbance derived from mock-transfected cells.

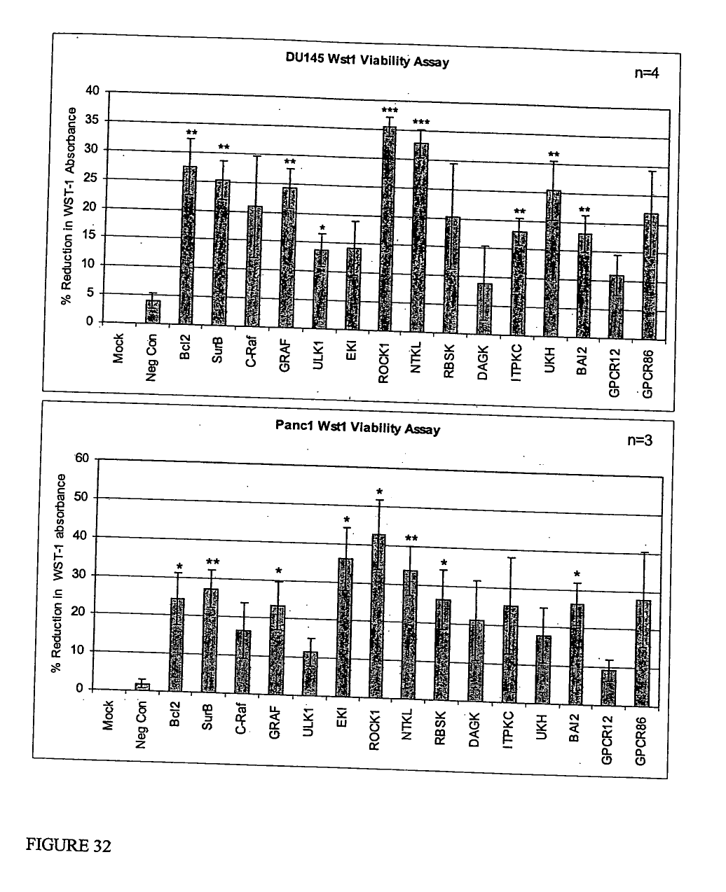

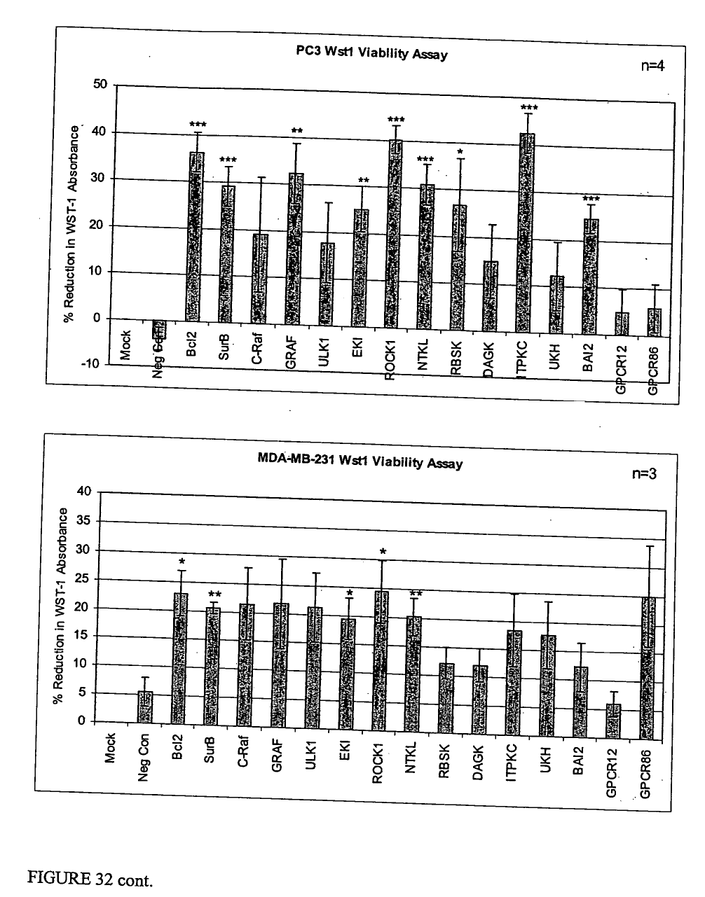

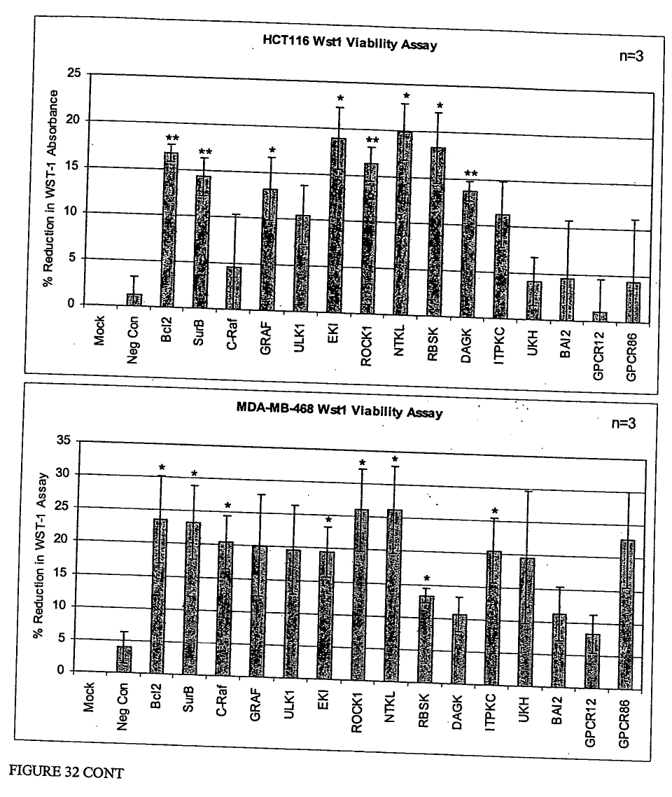

As can be seen in FIG. 32, knockdown of the target genes by siRNA modulates cell death as detected by Wst-1 conversion. This example demonstrates the differential induction of cell death by knocking down our target genes, compared with Missence, and Negative Control. The consequences of targeting other well characterised apoptotic genes (BCL2, c-Raf and Survivin) are also indicated.

FIG. 32 shows the reduction of cell viability of cancer cell lines transfected with siRNA silencing target genes. Each graph shows the mean reduction in WST-1 absorbance relative to mock transfected cells from an n=3 or n=4 independent experiments as stated. Error bars show the standard error. Significance testing was performed using a Student's T-test comparing each gene to the negative control where *P<0.05, **P<0.01 and ***P<0.001. Statistical analysis is performed on all samples. However due to the limited sample size and the inclusion of sample outliers which increase the standard error, not all genes were recorded as causing a significant effect; however these genes may cause significant biological effect should the sample size be increased.

7. Cell Proliferation Assay (BrdU)

Proliferating cells can incorporate bromodeoxyuridine into their DNA during DNA synthesis. The BrdU assay can measure the incorporation of bromodeoxyuridine into the DNA of cells undergoing DNA synthesis, and therefore quantitate the relative number of viable cells undergoing S-phase in a cell population.

Cells are transfected as above for the WST-1 assay, but using white-walled clear-bottomed 96-well plates. The BrdU assay is performed 72 hours post-transfection using a BrdU Chemilluminescent Proliferation Assay Kit (Roche) following the manufacturer's instructions. Briefly, BrdU labelling solution is added to each well of transfected cells and the cells incubated for a period of 2-4 hours depending on the cell line. Medium is removed from the cells by inversion and the cells fixed in FixDenat solution for 30 minutes before adding anti-BrdU-POD solution for a further 60 minutes. Antibody solution is removed by inversion and the wells rinsed three times with washing buffer. After washing, substrate solution is added to each well and, following a 3 minute incubation, the relative light units measured by a microplate luminometer. The relative number of proliferating cells in each transfected cell population is expressed as the percentage decrease in relative light units derived from mock-transfected cells.

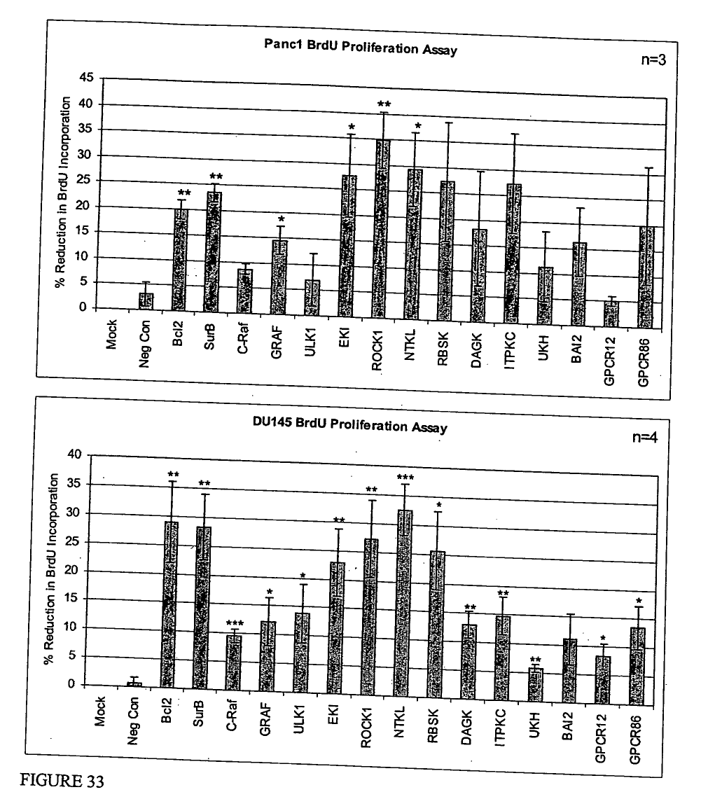

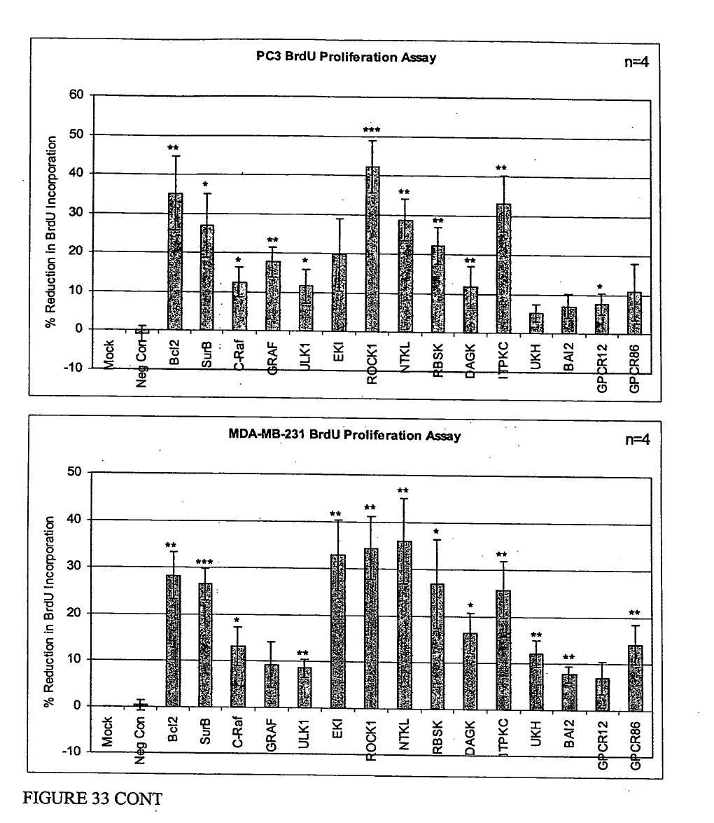

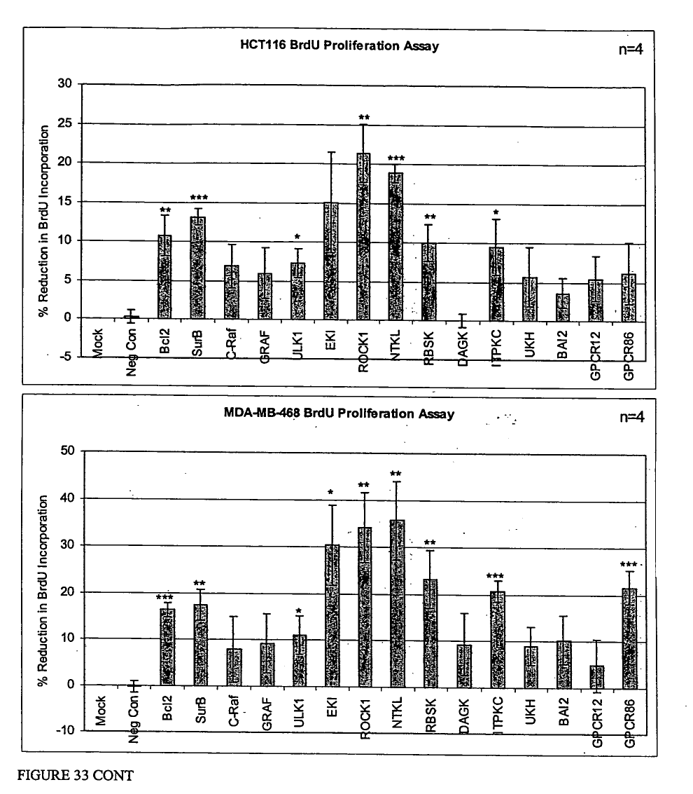

FIG. 33 demonstrates that knockdown of our target genes by siRNA modulates cell proliferation as detected by bromodeoxyuridine uptake. The BrdU assay measures the number of cells that are viable and attached to the tissue culture surface that are undergoing DNA synthesis within a defined 2 hour period. This example demonstrates the differential reduction in cell death by knocking down our target genes, compared with Missence, and Negative Control. The consequences of targeting other well characterised apoptotic genes (BCL2, c-Raf and SSurvivin) are also indicated.

FIG. 33 shows the reduction of cell proliferation of cancer cell lines transfected with siRNA silencing target genes. Each graph shows the mean reduction in relative light units, derived from the incorporation of BrdU, relative to mock transfected cells from an n=3 or n=4 independent experiments as stated. Error bars show the standard error. Significance testing was performed using a Student's T-test comparing each gene to the negative control where *P<0.05, **P<0.01 and ***P<0.001. Statistical analysis is performed on all samples. However due to the limited sample size and the inclusion of sample outliers which increase the standard error, not all genes were recorded as causing a significant effect; however these genes may cause significant biological effect should the sample size be increased.

8. Annexin V Apoptosis Assay.

To demonstrate that the loss of cell viability is due to the induction of apoptosis, the apoptotic status of cells in which the target had been silenced is examined using an assay measuring the binding of Annexin V to an apoptotic marker on the cell surface. Apoptosis or programmed cell death is an important and active regulatory pathway of cell growth and proliferation. Cells respond to specific induction signals by initiating intracellular processes that result in characteristic physiological changes. Among these are externalisation of phosphotidylserine (PS) to the cell surface, cleavage and degradation of specific cellular proteins, compaction and fragmentation of nuclear chromatin and loss of membrane integrity (in late stages). Annexin V is a calcium-dependant phospholipid binding protein with a high affinity for phosphotidyl serine (PS), a membrane component normally localised to the internal face of the cell membrane. Early in the apoptotic pathway molecules of PS are translocated to the outer surface of the cell membrane where Annexin V can readily bind to them.

Annexin V assays are performed using the Guava Nexin Assay, which utilises Annexin V-PE to detect PS on the external membrane of apoptotic cells. The cell impermeant dye 7-AAD is included in the assay as an indicator of membrane structural integrity as it is excluded from live and early apoptotic cells but permeates late stage apoptotic and dead cells. Assays are performed using a Guava Technologies Personal Cytometer and Guava Nexin Kit (catalogue no. 4500-0010, Guava Technologies Inc) using a modification of the manufacturer's protocol, where cells are transfected with siRNA in 24-well plates and assays performed at 24, 48 and 72 hours post-transfection.

Cells (DU145, PC3 & HCT116) are plated at a density of 20000 cells/well in 500 μl medium in 24-well plates and incubated overnight at 37° C. in a humidified atmosphere containing 5% CO₂. The adherent cells are then transfected with siRNA (EKI, ROCK1, NTKL and RBSK) at a concentration of 50 nM using RNAiFect reagent (Qiagen) following the manufacturer's instructions. Briefly, 1 μg of siRNA is added to a total volume of 100 μl EC-R buffer (Qiagen) and an appropriate volume of RNAiFect added to the cells, which was cell line-dependent. After a 15 minute incubation at room temperature, the siRNA-RNAiFect complexes are diluted to 550 nM with EC-R buffer. The diluted siRNA complexes are then added to cells so as to give a final concentration of 50 nM. After a period of 4 hours, the transfection solution is removed from the cells and 500 μl fresh growth medium added. All transfections included a mock transfection (transfection reagent only) and a negative control siRNA (Qiagen non-silencing siRNA).

The 500 μl of media is decanted from the cells and combined with 150 μl of PBS used to wash them. 100 μl trypsin is used to detach the cells and this is neutralised and the cells resuspended with 500 μl of the media/PBS mix. The cells are spun and washed with 500 μl 1× Nexin Buffer and resuspended in buffer at an concentration of 2-10×10⁵ cells/ml. To 40 μl of the cell suspension, 2.5 μl Annexin V-PE and 2.5 μl 7-AAD is added and the cells incubated on ice for 20 minutes shielded from light. Then, 450 μl 1× Nexin Buffer is added to the cells and the cell population analysed using the Guava Personal Cytometer following the manufacturer's instructions. Three populations of cells can thus be distinguished using this assay: (i) non-apoptotic cells (Annexin V (−) and 7-AAD (−)) (ii) early apoptotic cells (Annexin V (+) and 7-AAD (−)) (iii) Late apoptotic and dead cells (Annexin V (+) and 7-AAD (+)). The total number of early and late apoptotic cells is measured and the relative number of apoptotic cells calculated.

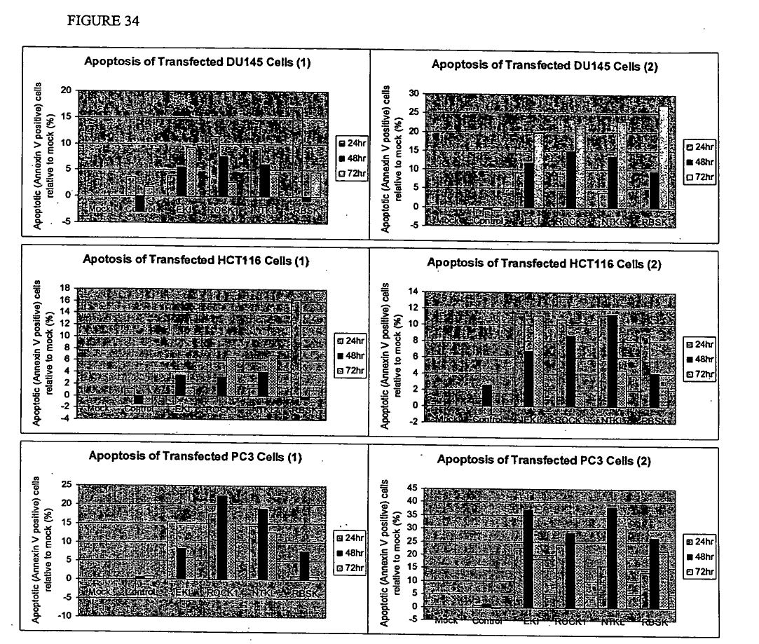

FIG. 34 demonstrates that knockdown of target genes by siRNA induces apoptosis in various cell lines as determined by the increase in phosphotidylserine positive cells. This example demonstrates the induction of apoptosis by knocking down our target genes, compared with Missence, and Mock Control (transfection reagent alone).

FIG. 34 shows cells transfected with siRNA silencing the EKI, ROCK1, NTKL and RBSK genes undergo apoptosis. Each graph shows the percentage of transfected DU145, HCT116 or PC3 cells staining with the apoptosis-specific marker Annexin V, relative to the mock transfected (transfection reagent only) cell population. The results from two independent experiments are shown.

### Example 7

### Knockdown of Identified Genes in Cells Functionally Modulates Apoptosis as Determined by Long Term Viability Assays

In this example, the siRNA Oligomers and siRNA Efficiency Studies performed for each siRNA oligomer are described in Example 5.

Inhibition of Target Gene Expression in Cancer Cell Lines by siRNA Modulates Long-Term Cancer Cell Viability, as Measured by the Clonagenicity Assay

The Clonagenicity Assay represents a Cell Viability Screen that allows assessment of a cytotoxic insult at a variety of seed densities of cancer cell lines over an extended period of logarithmic growth, such that the definitive effect of the test substance can be accurately quantified. In this instance, the ‘cytotoxic insult’ to be tested involves the transfection of siRNA Oligomers to knockdown the expression of novel target Kinases and GPCRs as outlined previously. Cell number is quantified 10-14 days post siRNA transfection by MTT conversion as described in detail in Example 6.

The Clonagenicity Assay

This example measures the effect of knocking down expression of a target gene, on a range of cancer cells over a 14-day period. Model cell lines include Hela, DU145, TF1, U251, SKOV3, OVCAR3, MCF7, PC3, HCT15, 786-0, HT29, M-14, H460, LnCap, PC3, A498 and A549.

The Clonagenicity Assay is optimised for each cell line examined with respect to seed number. The seed number varies for each cell line and accounts for the differences in cell size, growth rate and sensitivity to transfection-induced toxicity for each cell line pursued. Cells lines given as models for this example include HCT15, MCF7, A549, SKOV3, DU145, U251, PC3, A498 and HeLa. The siRNA oligo to Survivin serves as a positive control for toxicity in this assay and the missense control oligomer acts as a negative control as described in detail in Examples 5 and 6. The c-Raf, BCL2 and PI3K siRNA oligomers serve as additional controls as previously mentioned. The Clonagenicity assay is performed as outlined below.

Method for 10-14 Day Clonagenicity Assay/Long Term Cytotoxicity Screen

Cells are seeded into a 96-well plate as described below and allowed to adhere overnight at 37° C. in a humidified atmosphere containing 5% CO₂ in air. Wells are seeded with an appropriate cell number (cell line dependent) in Column 1 of the 96 well plates and this cell suspension is diluted 1 in 2 across the 96 well plates from Column 1 through to 6 (represented on the x-axis in results). Cells are next transfected with siRNA Oligomers, at a concentration of 2 μg/ml, against the genes of interest. Transfections continue for 4 hrs after which the transfection solution is removed and cells are refed with fresh medium and allow to incubate overnight. An additional media change@24 hrs post transfection is made to minimise toxicity of the transfection reagent. Cells are subsequently incubated for a 10-14 Day period post transfection at a 37° C. incubation as described above. Medium is renewed weekly following which MTT quantification is performed as described in Example 5.

Inhibition of Target Gene Expression in Cancer Cell Lines by siRNA Modulates Long Term Cancer Cell Viability, as Measured by Growth in the Anchorage Independence/Soft Agar Assay

The Anchorage Independence/Soft Agar Assay allows assessment of the influence of modulation of gene expression on clonal tumouroid formation by observing and quantifying tumouroid morphology and colony number over an extended period of logarithmic growth thereby ascertaining any cell death is induced.

The ability pf cancer cells to form tumouroids in semi solid agar is characteristic of an aggressive tumour cell phenotype. In the present example, the functional consequence of knocking down the target gene is examined in this anchorage independent cellular assay.

Prior to instigating siRNA experiments, the ability of each of cancer cells lines for anchorage independent tumouroid formation in soft agar is assessed. Visual observation of the tumours formed post a 6-day incubation period indicates that the PC3, U251, Hela, MCF7, HCT15 and A549 cell lines are capable of forming well-defined tumours in this assay and act as appropriate models for this cancer phenotype assay. However all the cancer cell lines outlined above may serve as models for this cancer cell characteristic as they are highly competent in RNAi.

The ability of this assay to successfully identify cytotoxicity on knockdown of gene expression by RNAi is validated by identification of siRNA against Survivin as a cytotoxic agent in the cell lines examined. Therefore the effect of reducing expression of our Kinases/GPCRS of interest with specific siRNA Oligomers on tumouroid formation is assessed and results compared to Survivin induced cytotoxicity utilising the Anchorage Independence/Soft Agar Assay. The Soft Agar Assay is performed as outlined below.

Method for the Soft Agar Assay

Cells are seeded into a 6-well plate and allowed to adhere overnight at 37° C. in a humidified atmosphere containing 5% CO₂ in air. Cells are next transfected with siRNA Oligomers, at a concentration of 2 μg, against the genes of interest. Transfections continued for 4 hrs after which the transfection solution is removed and cells are refed with fresh medium and allowed incubate overnight. Transfected cells are next harvested for seeding into soft agar assay as described below. Cells are seeded at 5×10⁴ cells per well of a 6 well plate and incubated over a 12/14-Day period whilst proliferating in a 0.05% Agar/Medium cell suspension at 37° C. in a humidified atmosphere containing 5% CO₂ in air. Colonies formed are subsequently stained with Crystal Violet, their morphology is assessed and colonies formed are enumerated at a 4× magnification.

The cytotoxic effect of each gene is determined by a reduction in colony number compared to our MS control, but any influence on the morphology of the colony is also noted.

Decreased Expression of Target Genes by siRNA decreases Viability of Cancer Cells Over a 10/14-Day Clonagenicity Assay—Part I

This example demonstrates that reducing mRNA expression of target genes can induce cell death over a 10/14-day clonagenicity assay as determined by MTT analysis, with reference to apoptosis/viability controls Survivin, BCL2, c-Raf, PI3K and a missense (MS) control. The control gene Survivin induces a significant decrease in the viability/clonagenicity of the cell lines examined in this assay compared to the MS Control.

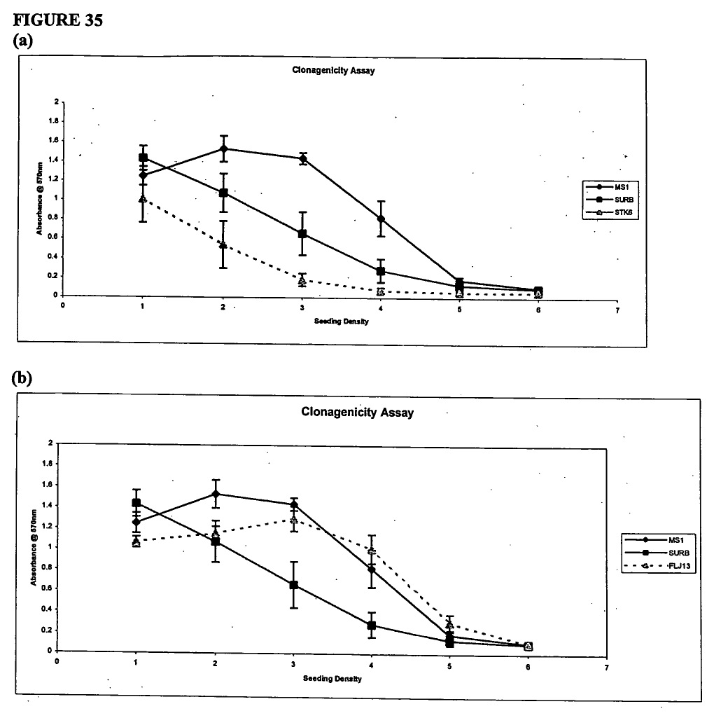

An example of results/clonagenicity profiles obtained from the Clonagenicity Assay for a gene with a cytotoxic effect on knockdown (STK6) and one that does not induce cytotoxicity on knockdown (FLJ13351) is illustrated for the U251 Cell Line below in FIG. 35.

Targeting a number of the genes appeared to cause toxicity across the panel of cell lines used whereas; other genes appeared to induce cytotoxicity in a cell type specific manner. The maximum cytotoxicity induced by the knockdown of each gene across 3 independent experiments is determined from the clonagenicity profiles as described below and then summarised in Table 13(a-g) for each cell line.

FIG. 35 shows (a)Transfection of the siRNA Oligo against STK6 decreases viability in the U251 cell line as determined by the Clonagenicity Assay. Note seed density decreases from 3.5 to 0.1×10³ cells on the x-axis and the y-axis represents cell number as quantified by MTT conversion, 10-14 days post transfection. This decrease is substantially greater that with the apoptosis gene, Survivin. (b)Transfection of the siRNA Oligo against FLJ13351 does not induce cytotoxicity in the U251 cell line as determined by the Clonagenicity Assay. Note seed density decreases from 3.5 to 0.1×10³ cells on the x-axis and the y-axis represents cell number as quantified by MTT conversion, 10-14 days post transfection.

Note:

To be considered cytotoxic, the siRNA Oligo against the gene target must induce a decreased viability in a cell line across at least 2 seed densities in the clonagenicity assay, such that a significant difference between the target gene and MS is observed i.e. no crossing of the Standard Error bars is detected. The average percentage viability, relative to missense, is recorded for the dilution point at which Maximum Toxicity is induced is recorded in Table 13.

Table 13 Clonagenicity Assay—This table gives the average percentage survival of the indicated cell lines relative to the missense in a clonagenicity assay, over 3 independent experiments. As an internal reference, the known survival gene ‘Survivin’, BCL2, c-Raf and PI3KR1 was also targeted by siRNA. When no Cytotoxic response is detected ‘-’ is recorded.

These examples demonstrate that knockdown of target gene mRNA results in the functional modulation of apoptosis and cell death in certain cancer cells while not inducing apoptosis in cells derived from alternate tumour types. The cancer cells types affected by down regulation of gene expression varies for each ‘novel’ identified apoptosis regulator. This example therefore demonstrates a direct therapeutic application for inhibition of the same identified gene for the therapeutic treatment of specific cancers, since inhibition results in cytotoxicity of particular cancer cell types.

Decreased Expression of Target Genes by siRNA Decreases Viability of Cancer Cells Over a 14-day Clonagenicity Assay—Part II

The effect of silencing target genes is measured over a 14-day period using a clonagenicity assay and where clonagenicity is measured by counting colonies.

Cells (MDA-MB-231, MDA-MB-468, Panci, A549 and HCT116) are plated at a density of 20000 cells/well in 500 μl medium in 24-well plates and incubated overnight at 37° C. in a humidified atmosphere containing 5% CO₂. The adherent cells are then transfected with siRNA at a concentration of 50 nM using RNAiFect reagent (Qiagen) following the manufacturer's instructions. Briefly, 1 μg of siRNA is added to a total volume of 100 μl EC-R buffer (Qiagen) and an appropriate volume of RNAiFect added to the cells, which was cell line-dependent. After a 15 minute incubation at room temperature, the siRNA-RNAiFect complexes are diluted to 550 nM with EC-R buffer. The diluted siRNA complexes are then added to cells so as to give a final concentration of 50 nM. After a period of 4 hours, the transfection solution is removed from the cells and 500 μl fresh growth medium added. All transfections included a mock transfection (transfection reagent only), a negative control siRNA (Qiagen non-silencing siRNA) and positive control siRNA (targeting BCL2, Survivin and C-Raf).

At a time of 24 hours post-transfection, the transfected cells are removed from the tissue culture plate by trypsinization and neutralisation in cell growth medium. The density of viable cells was determined using a ViaCount Assays in combination with a Guava Personal Cytometer following the manufacturer's instructions. Cells are diluted to a density of 2500 cells/ml and 50 μl aliquots of this cell suspension is added to triplicate wells of a 6-well plate containing 2.5 ml of cell growth medium, so that 125 cells are seeded per well. Cells are incubated for a period of 14 days. The cells are fixed and stained by removing the medium and adding 0.5% (w/v) methylene blue in 50:50 ethanol:water so that the cells are immersed. The cells are incubated for 30 minutes and the staining solution is removed by immersion of the plate in water. Colonies are counted.

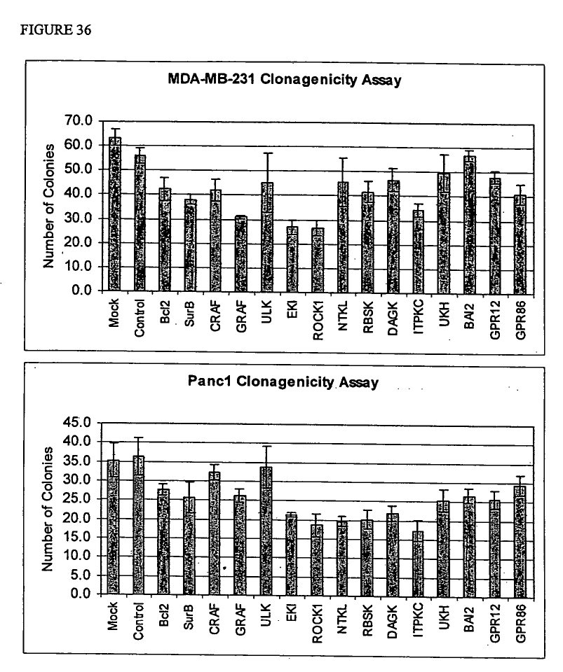

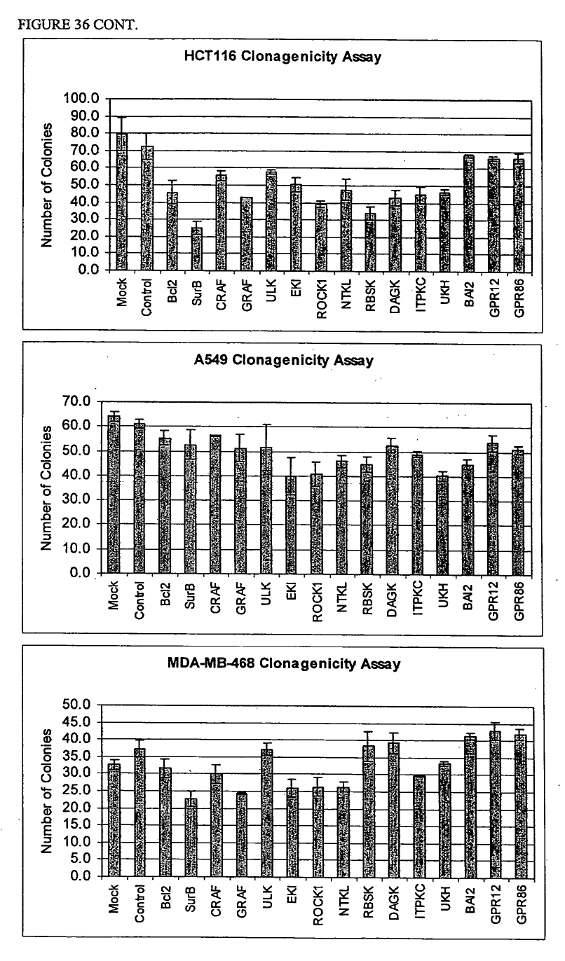

FIG. 36 shows cells transfected with siRNA silencing target genes have decreased viability over a 14-day clonagenicity assay. Each graph shows the mean number of colonies derived from cells transfected with siRNA. The data represents triplicate wells from a single experiment. Error bars show the standard error.

Soft Agar Assay

Soft Agar Assay demonstrates that decreased expression of target genes by RNAi induces Apoptosis/cell death in Cancer Cell Lines as determined by a decreased ability of cancer cells to form tumouroids

Examples of genes with a cytotoxic effect on knockdown as detected by the soft agar assay are outlined in Table 14. Table 14 gives the percentage survival of the indicated cell lines post transfection with siRNA oligomers to target genes relative to that of the missense in an anchorage independent assay. As an internal reference, the known survival gene Survivin was also targeted by siRNA. This table illustrates the magnitude of cytotoxicity induced by the knockdown of each target gene compared to the MS control i.e. the number of colonies formed are stained with Crystal violet and enumerated visually as discussed.

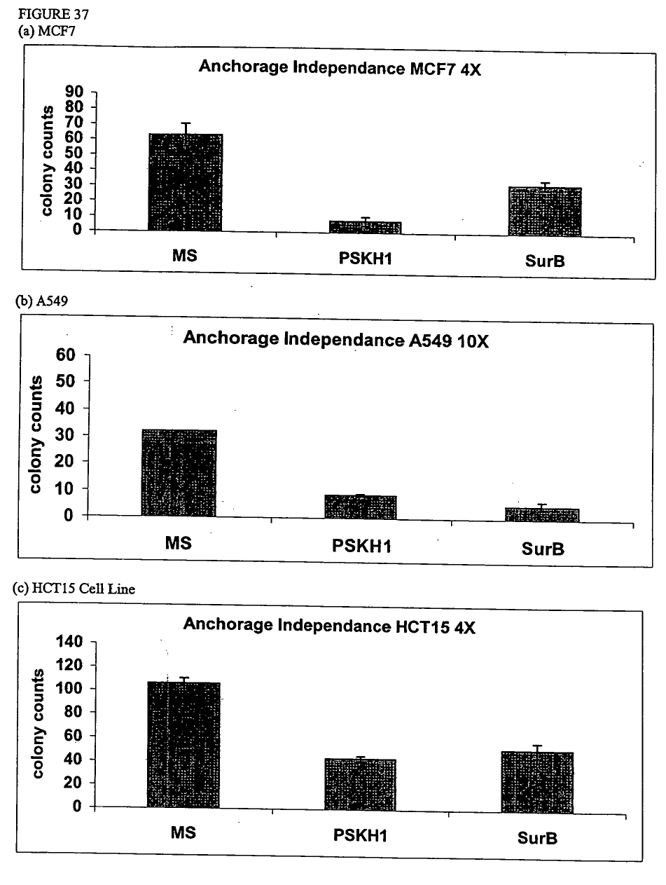

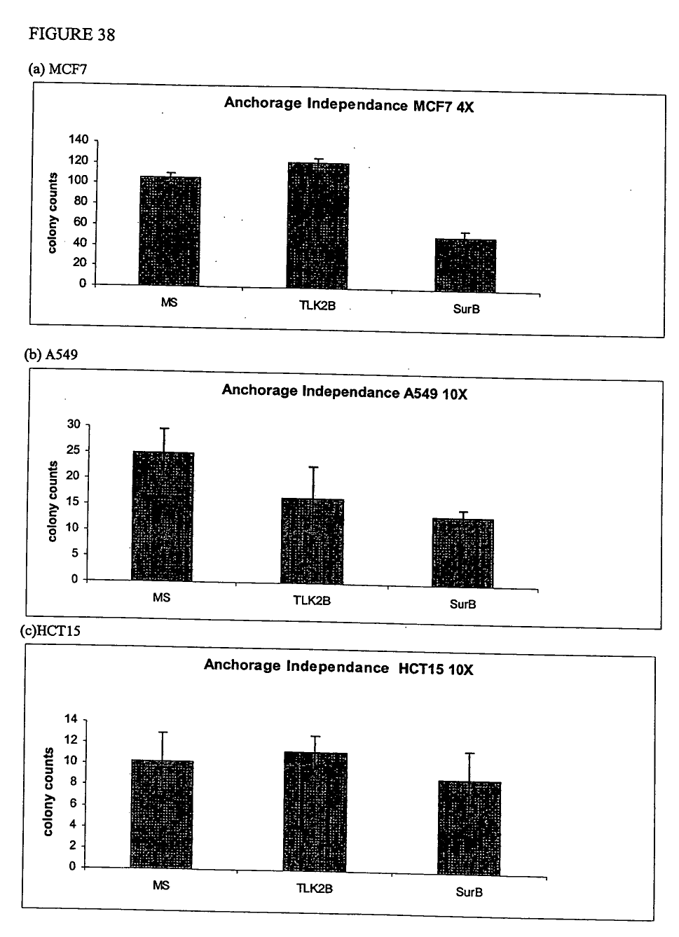

Examples of the Soft Agar Results for the PSKH1 and TLK2 genes are also illustrated individually in FIGS. 37 and 38 to illustrate the graphs from which percentage cytotoxic response noted in Table 14 was determined. Together these results demonstrate a decrease in viability following knockdown of target gene expression in a range of cancer cell types.

FIG. 37 shows Knockdown of the PSKH1 gene by RNAi modulates apoptosis in cancer cell lines as determined by the Anchorage Independence Soft Agar Assay. FIG. 38 shows Knockdown of the TLK2 gene by RNAi does not modulate apoptosis in cancer cell lines as determined by the Anchorage Independence Soft Agar Assay.Cytotoxicity is quantified as a decrease in the number of tumouroids/colonies formed by the cancer cell lines post transfection with the PSKH1 siRNA oligo over a 14-day period of logarithmic growth. As an internal reference, the known survival gene ‘Survivin’ was also targeted by siRNA.

Of the targets shown, MPSK1 and PSKH1 are the most cytotoxic of the kinases examined as determined by the soft agar assay inducing significant cell death in all cell lines. MKNK induced cell death consistently in 2 of the cell lines examined and in the HCT15 cell line but only in one experiment. PRK/CNK induced toxicity consistently in the HCT15 cell line and in the A549 cell line but again only in one experiment. Survivin induced cell death in all cell lines and acted as a reproducible control for this experiment whereas c-Raf only induced cell death in the A549 and HCT15 cell lines.

This example demonstrates that a number of the target genes are able to induce death in the indicated cell lines over a 14-day anchorage independent assay. These examples demonstrate that knockdown of ‘novel’ identified ‘apoptosis regulators’ mRNA results in the functional modulation of apoptosis in certain cancer cells while not inducing apoptosis in cells derived from alternate tumour types. The cancer cells types affected by down regulation of gene expression varies for each ‘novel’ identified apoptosis regulator. This example therefore demonstrates a direct therapeutic application for inhibition of the same identified gene for the therapeutic treatment of specific cancers, since inhibition results in cytotoxicity of particular cancer cell types.

Note on Colony Morphology:

The cell lines illustrated in this example form tumouroids of varying size and shape in the soft agar assay. However it is noted that when knockdown of a particular gene is impacting on cell viability the morphology of the tumouroids formed by all cell lines, but most especially the A549 and HCT15, altered. The tumouroids transfected with MS depict a dispersed/scattered morphology and the majority of the tumouroids in these samples are dispersed however when a toxic gene is transfected the morphology changed often quiet dramatically (e.g. MKNK and MPSKI in the A549) from a dispersed morphology to solid, rounded tumours which are generally smaller in size. These solid, rounded tumouroids are predominant when cytotoxicity is induced by the siRNA transfected.

RNAi Knockdown of Target Gene Expression Decreases the Long Term Viability of Cancer Cells

Assessment of the long-term viability of cancer cells utilising the combination of the Soft Agar Assay and the Clonagenicity Assay, demonstrates that particular genes have potential as candidate drug targets (See Summary Section). These examples demonstrate that knockdown of ‘novel’ identified ‘apoptosis regulators’ mRNA results in the functional modulation of apoptosis in cells over a number of cell generations, in combination with the added stresses of cell cloning and anchorage independent growth. Additionally, this example demonstrates a direct therapeutic application for inhibition of the same identified genes for the therapeutic treatment of cancer, since inhibition results in cytotoxicity of a particular cancer cell.

### Example 8

### Knockdown of Identified Genes in Cells Functionally Modulates the Migration of Cancer Cells

Migration of cancer cells is one of the most pernicious characteristics of a tumour. In the following example we demonstrate that by inhibiting expression of the indicated target gene, we can prevent migration. In this example we use a commercially available Cell Migration assay.

Cell Migration Assay

Cells are seeded into a 6-well plate and allowed to adhere overnight at 37° C. in a humidified atmosphere containing 5% CO₂ in air. Cells are next transfected with siRNA oligonucleotides, at a concentration of 2 μg/ml, against the genes of interest, as described in detail in Example 5. Transfections are continued for 4 hrs after which the transfection solution is removed and cells are refed with fresh medium and allowed incubate overnight. Transfected cells are next harvested for seeding into migration assay as described below.

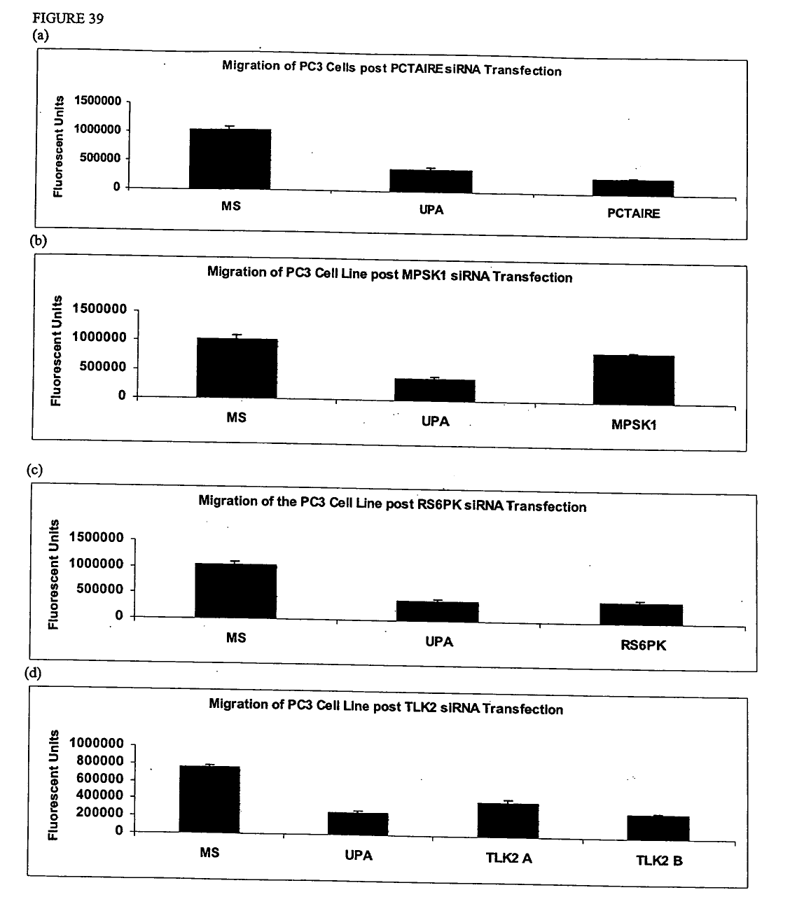

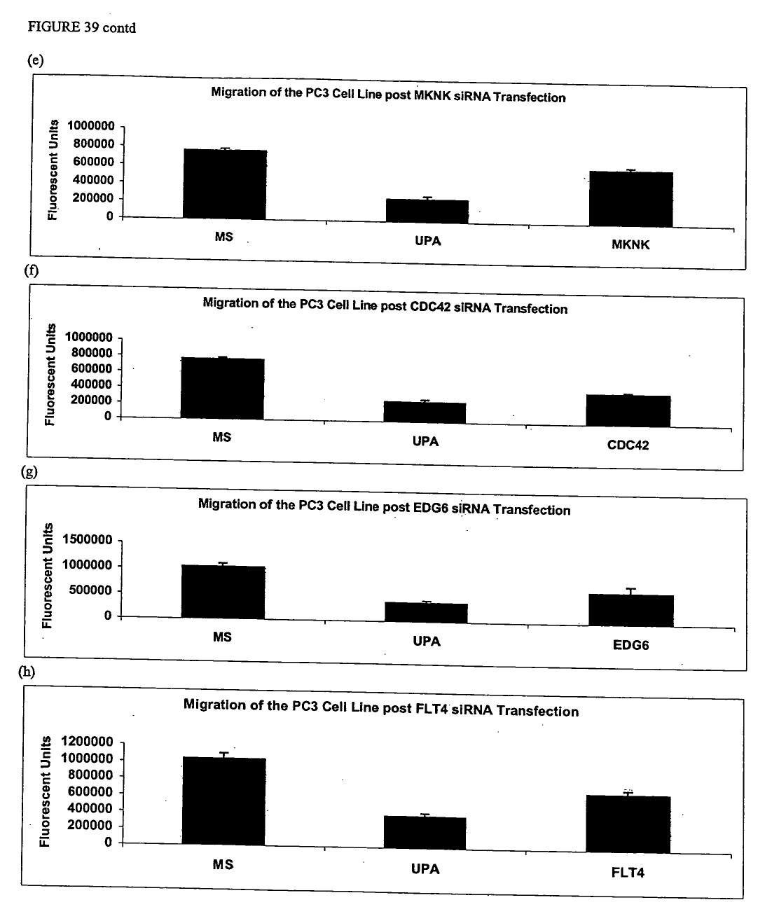

The Chemicon Cell Migration Assay (ECM550) is used to quantify the number of PC3 cells that successfully migrate across a Boyden Chamber towards a chemoattractant over a 24-hour incubation period. Assessment of PC3 cell migration is performed routinely 48 hours post siRNA Transfection. The effect of reducing the expression of genes of interest is compared to that of a missense control oligomer and to the influence of UPA knockdown, which is reported in the literature to decrease PC3 cell migration. The Migration Assay is performed as per manufacturers inductions. Results are illustrated in FIGS. 39 to 41.

FIG. 39 shows Knockdown of target gene by siRNA in PC3 cells decreases their ability to migrate.

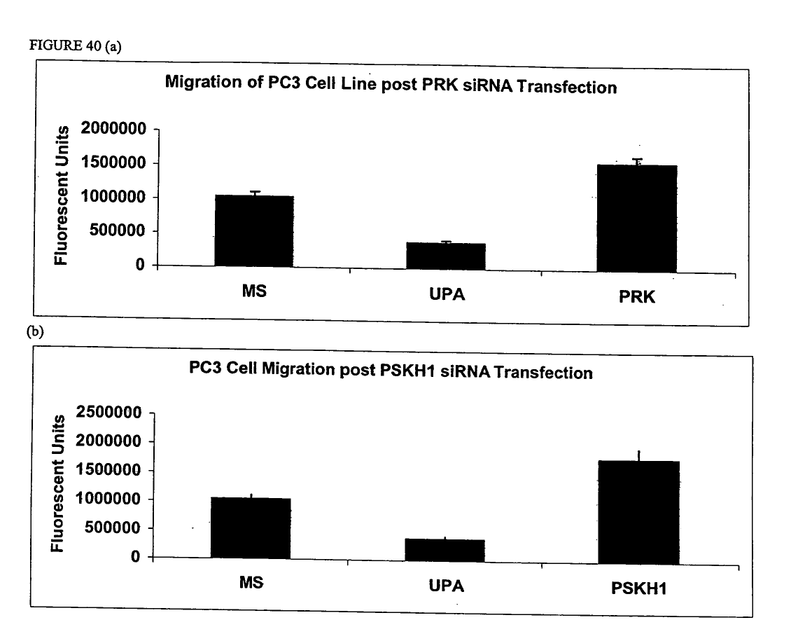

FIG. 40 shows Knockdown of target gene by siRNA in PC3 cells increases their ability to migrate.

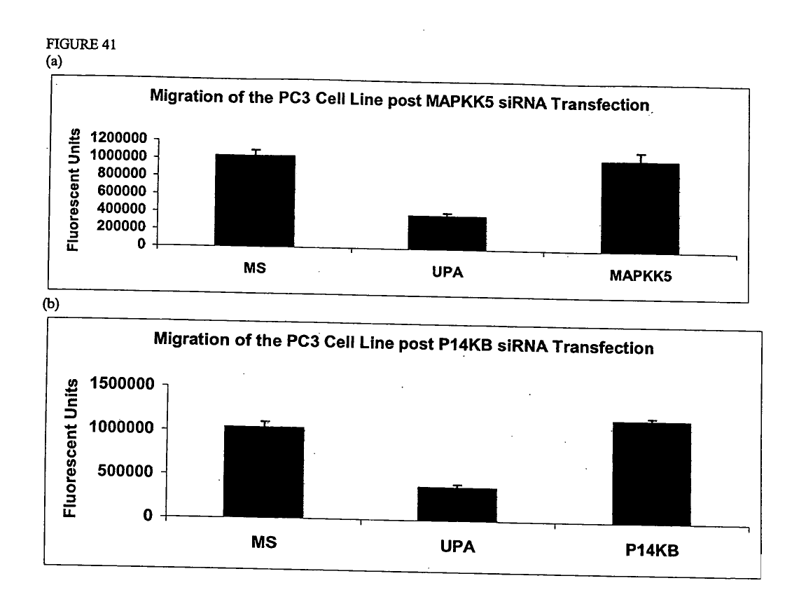

FIG. 41 shows Knockdown of target gene by siRNA in PC3 cells does not modulate their ability to migrate.

Cells were plated and transfected with the indicated siRNA as described above. Migration of PC3 cells was tracked using a fluorescent dye. Consequently increases in fluorescent units correlates positively with increased migration. The effect of targeting the specific gene was compared with targeting either missense control or Urokinase Plasminogen Activator (UPA); a gene previously demonstrated to modify migration in PC3 cells.

Targeted Knockdown of Target Genes Modulates Migration in the PC3 Prostate Cancer Cell Line

In the current example the prostate cell line PC3 is used to measure the effect of targeting the various kinases and GPCRs by siRNA. The PC3 cell line is traditionally used in such a chemotaxis assay.

Decreasing the expression of PCTAIRE, MPSK1, RS6PK, TLK2, MKNK, CDC42, EDG6 and FLT4 results in varying degrees of inhibition of migration of the prostate cancer cells across the 8 μm porous filter of the Boyden Chamber compared to both the missense and Urokinase Plasminogen Activator (UPA) control genes.

In contrast, decreasing the expression of MAK, PRK/CNK and PSKH1 by siRNA in the PC3 Cell Line by RNAi increases the ability of cancer cells to migrate.

Altering the expression of MAPKK5 and P14KB by RNAI had no influence on the migration of the cell line and therefore these genes must not play a role in the locomotion/chemotaxis of these prostate cancer cells.

These examples demonstrate that knockdown of ‘novel’ identified apoptosis regulators' mRNA, results in the functional modulation of the cancer phenotype of Prostate Cancer Cells. By decreasing the expression of these genes, we influence the capability of these aggressive cells for chemotaxis and therefore affect the metastatic potential of the cancer, a property that contributes greatly to cancer recurrence, resistant to therapy and mortality. Therefore this example further demonstrates a direct therapeutic application for inhibition of the same identified genes for the therapeutic treatment of cancer since inhibition may result in decreased metastasis of cancer cells.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### Table 1

### Identified Genes—Genbank Accession and Gen Identifiers. A. Known Regulators of Apoptosis and Cell Survival B. Novel Apoptotic Regulators

TABLE 1A

| Accession | | | Gene | Protein |
|---|---|---|---|---|
| (Array | | GenBank | Identification | Identification |
| identifier) | Unigene Title | Accession | Number | number |
| AI274392 | 3-phosphoinositide | NM_002613 | 4505694 | 4505695 |
| | dependent protein kinase-1 (PDK1) | | | |
| AI684957 | Protein kinase C, | Z15108 | 35500 | 35501 |
| | zeta (PKCζ) | | | |
| U09607 | Janus kinase 3 (a protein | U09607 | 508730 | 508731 |
| | tyrosine kinase, leukocyte; JAK3) | | | |
| X15573 | Phosphofructokinase, | X15573 | 35430 | 35431 |
| | liver (PFKL) | | | |

TABLE 1B

| Accession | | | Gene | Protein |
|---|---|---|---|---|
| (Array | | GenBank | Identification | Identification |
| identifier) | Unigene Title and Acronyms (as used herein) | Accession | Number | number |
| AI125216 | Rho-associated, coiled-coil containing protein kinase 1 (ROCK1/ROCK) | NM_005406 | 4885582 | 4885583 |
| AA058851 | MAK (Male germ cell-associated kinase; MAK) | NM_005906 | 13699865 | 11496279 |
| AA142914 | G protein-coupled receptor 86 (GPR86, GPCR86) | AF295368 | 12711484 | 12711485 |
| AA535884 | PCTAIRE protein kinase 3 (PCTAIRE) | X66362 | 297101 | 297102 |
| AB005298 | G-protein coupled receptor (BAI2) | NM_001703 | 4502356 | 4502357 |
| AB014521 | GTPase regulator associated with the focal adhesion kinase pp125 (GRAF) | NM_015071 | 7662207 | 7662208 |
| AF011468 | Serine/threonine kinase 6 (STK6) | NM_003600 | 4507274 | 4507275 |
| AF045458 | Serine/Threonine kinase (ULK, ULK1) | NM_003565 | 4507830 | 4507831 |
| AF060798 | Serine/threonine kinase 16 (MPSK1) | NM_003691 | 4505836 | 4505837 |
| AF090421 | Ribosome S6 protein kinase (RS6PK) | NM_004755 | 4759051 | 4759052 |
| AF162667 | Tousled-like kinase 2 (TLK2, TLK2A, TLK2B) | AF162667 | 6063018 | 6063019 |
| AI003792 | Ethanolamine kinase (EKI/EKI 1) | NM_018638 | 21071078 | 10092615 |
| AI368064 | MAP kinase-interacting serine/threonine kinase 1 (MKNK) | NM_003684 | 21361100 | 21361101 |
| AI400203 | Homo sapiens cDNA (FLJ20559, UK, UKH) | NM_017881 | 8923529 | 8923530 |
| AI400203 | Homo sapiens cDNA (FLJ13351, FLJ13) | AK023413 | 10435341 | NA |
| AI741642 | N-terminal kinase-like (Telomerase associated; NTKL) | AF225424 | 9963850 | 9963851 |
| AI761729 | CDC42-binding protein kinase beta (DMPK-like; CDC42, CDC42BPK) | NM_006035 | 16357473 | 16357474 |
| AI823980 | Ribokinase (RBSK) | AJ404857 | 10799802 | 10799803 |
| AJ000479 | G-protein-coupled receptor (EDG6) | NM_003775 | 4503458 | 4503459 |
| L38707 | Diacylglycerol kinase (DAGK) | L38707 | 606756 | 606757 |
| U18548 | G protein-coupled receptor 12 (GPR12, GPCR12) | U18548 | 604499 | 604500 |
| U56998 | Cytokine inducible kinase (CNK, PRK) | U56998 | 1488262 | 1488263 |
| U71087 | mitogen-activated protein kinase kinase 5 (MAPKK5) | U71087 | 1616778 | 1616779 |
| U81802 | phosphatidylinositol 4-kinase, catalytic, beta polypeptide (PI4KB/T3 PI4KB) | U81802 | 1894946 | 1894947 |
| X69878 | fms-related tyrosine kinase 4 (FLT4) | X69878 | 297049 | 297050 |
| X71875 | Protein serine kinase H1 (PSKH1) | XM_043047 | 22067477 | 14776113 |
| Y11999 | inositol 1,4,5-trisphosphate 3-kinase C (ITPKC) | Y11999 | 1914774 | 1914775 |

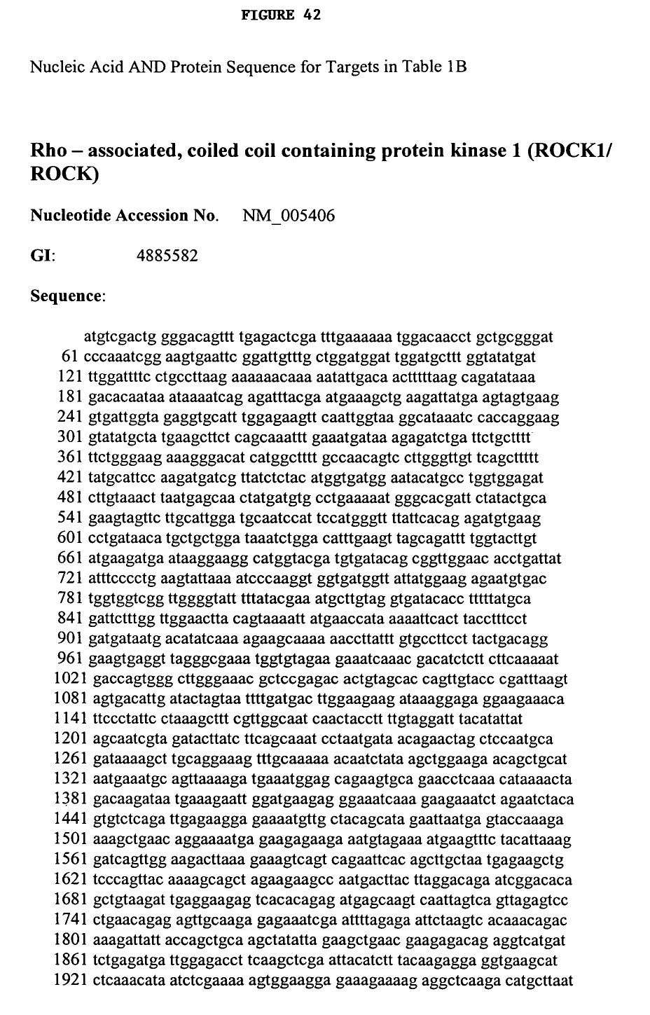

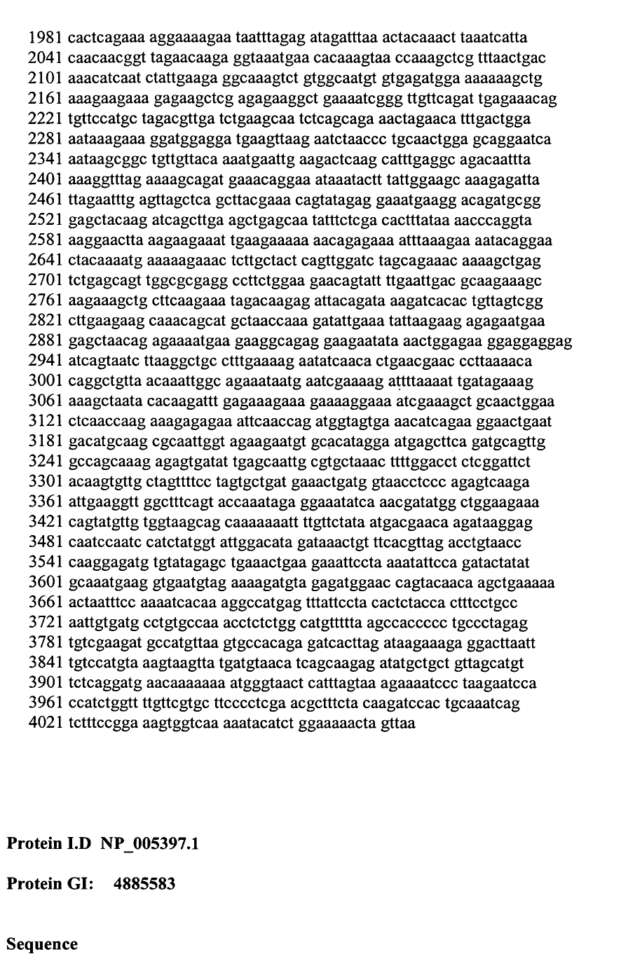

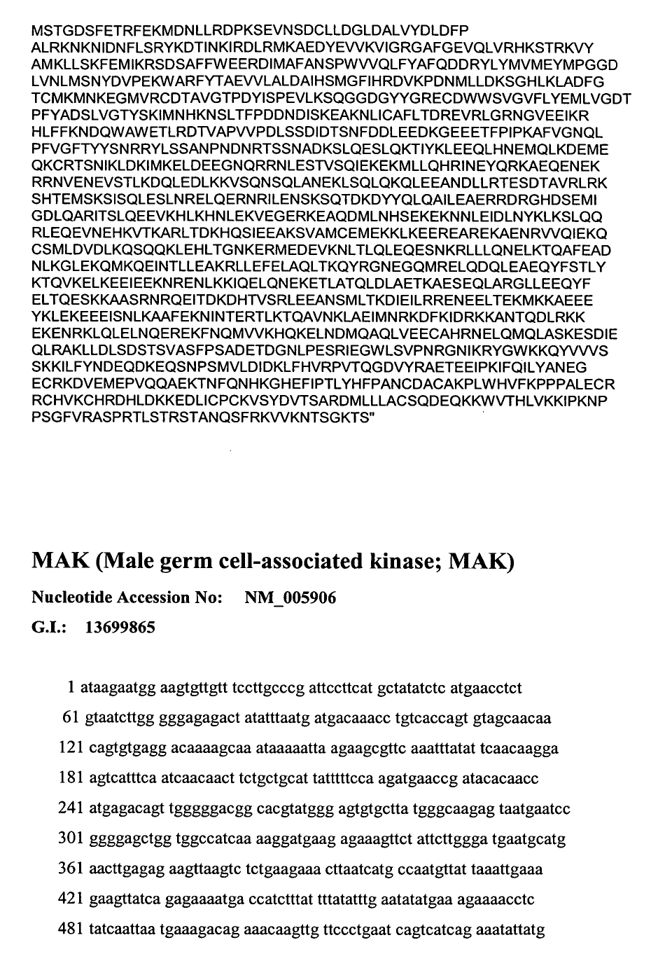

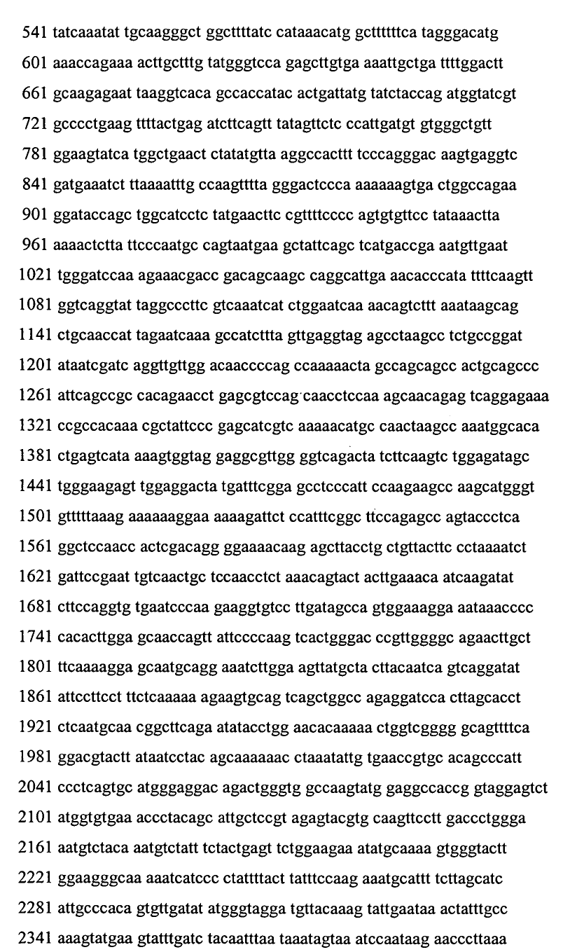

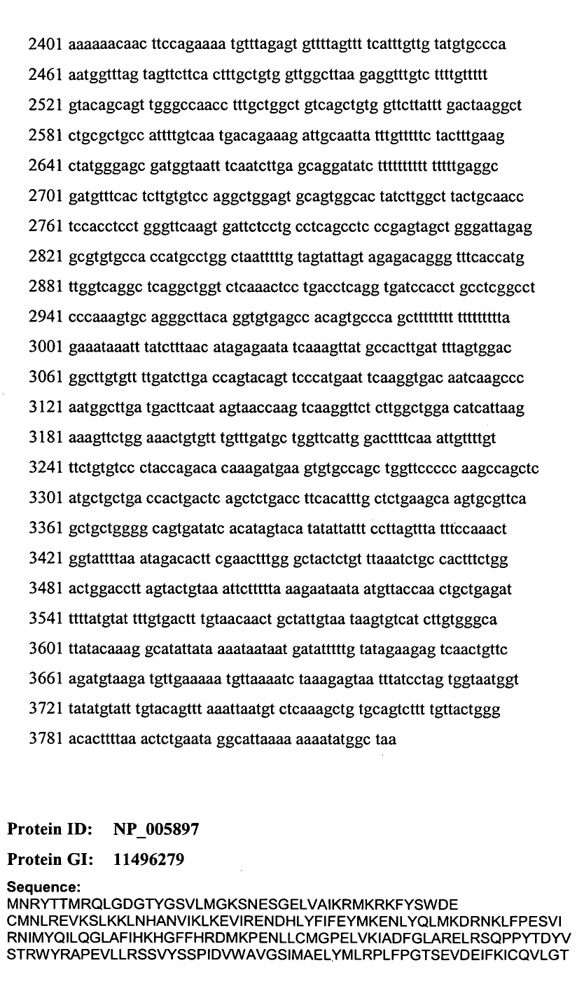

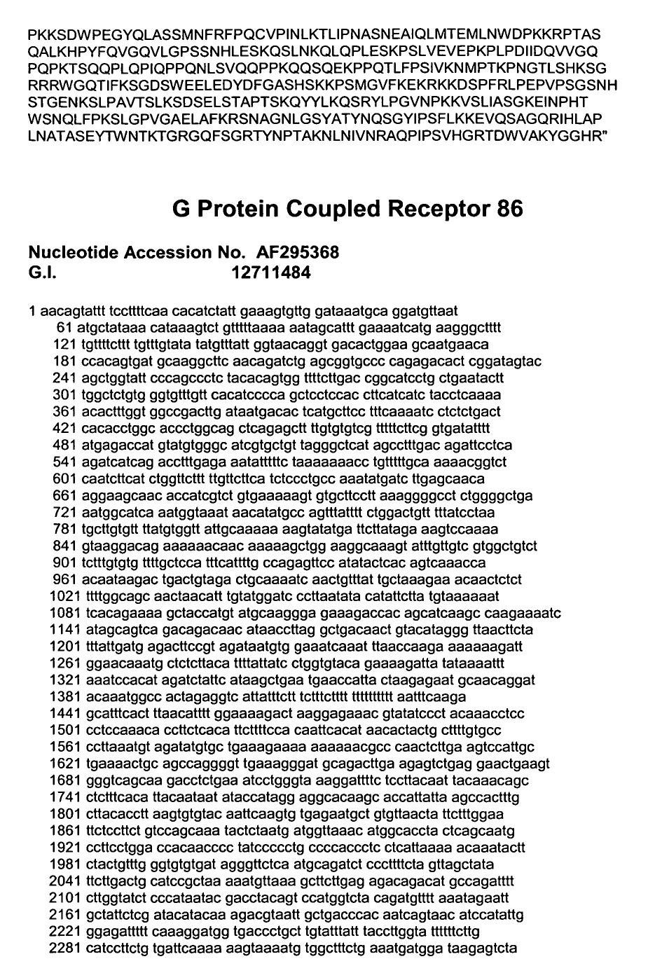

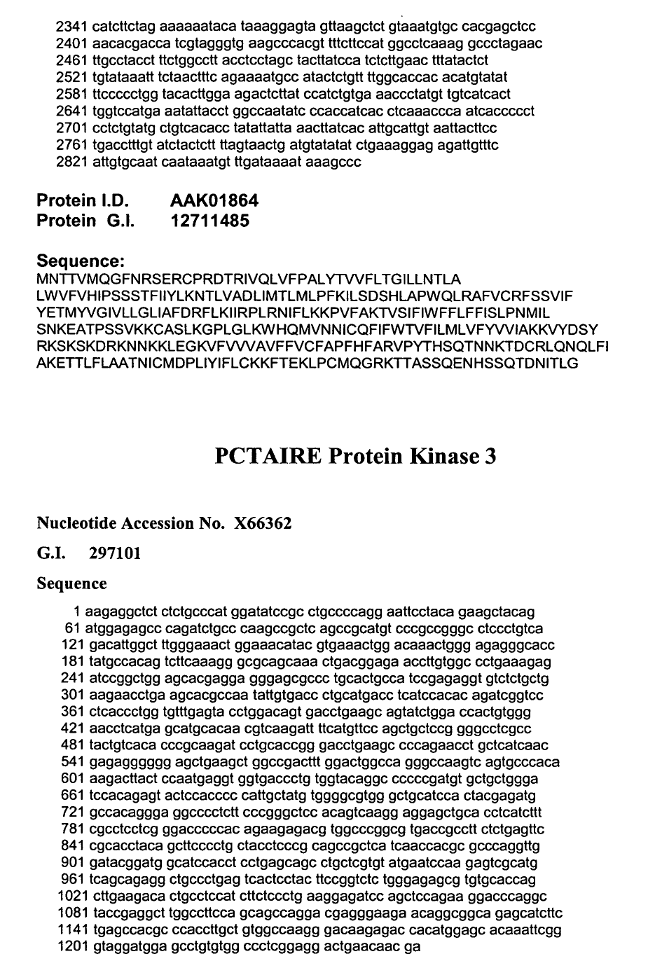

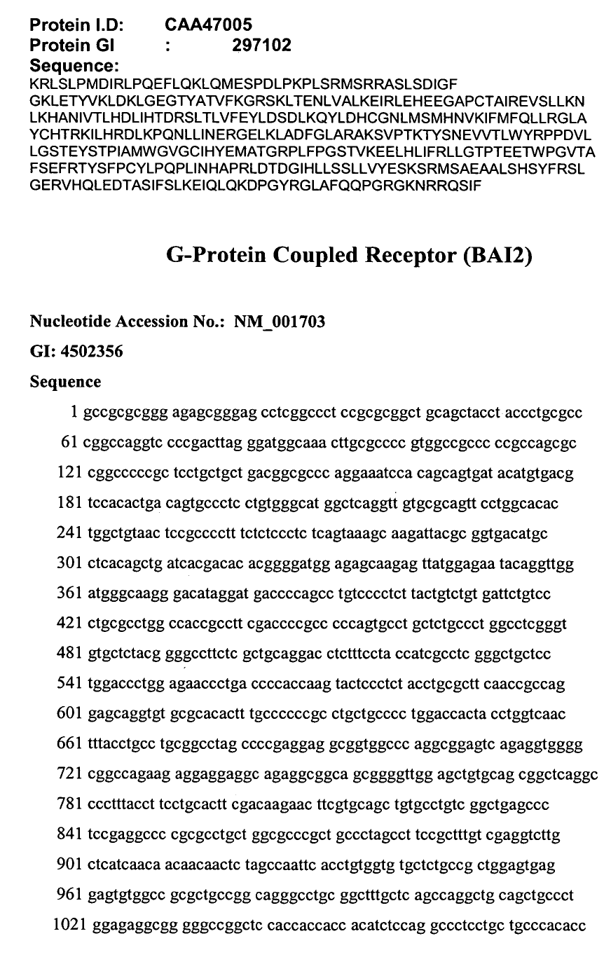

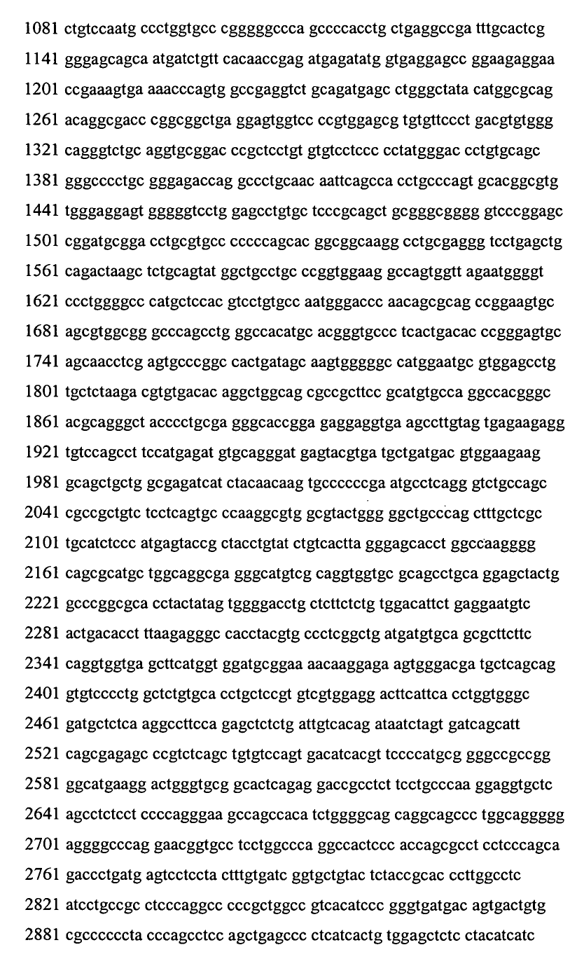

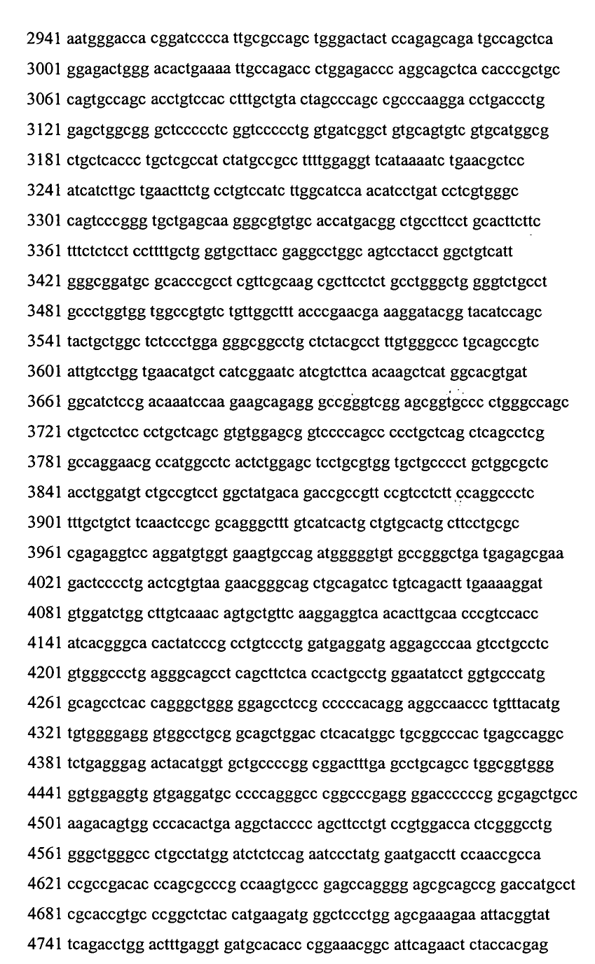

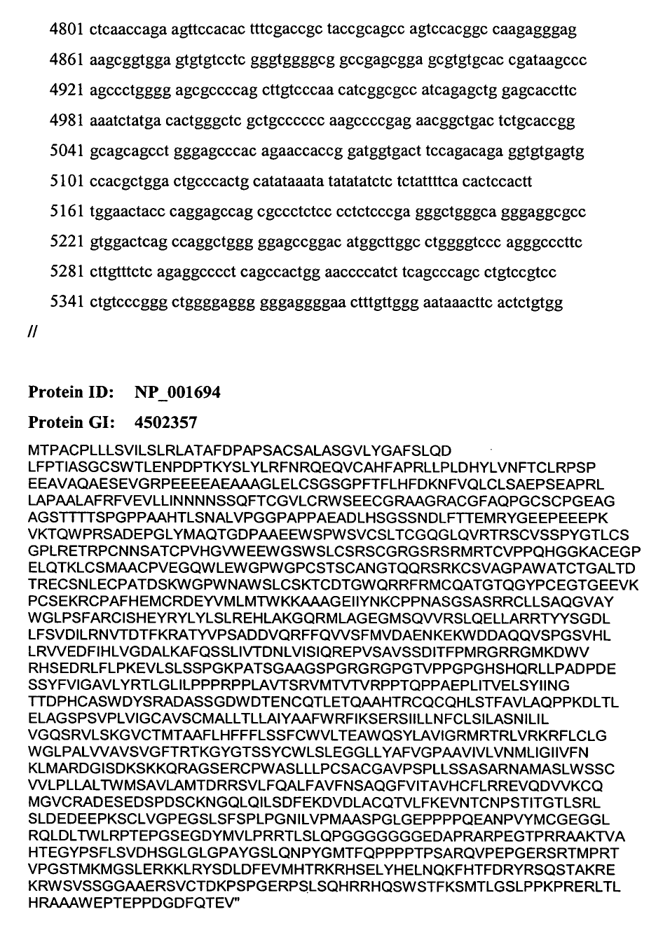

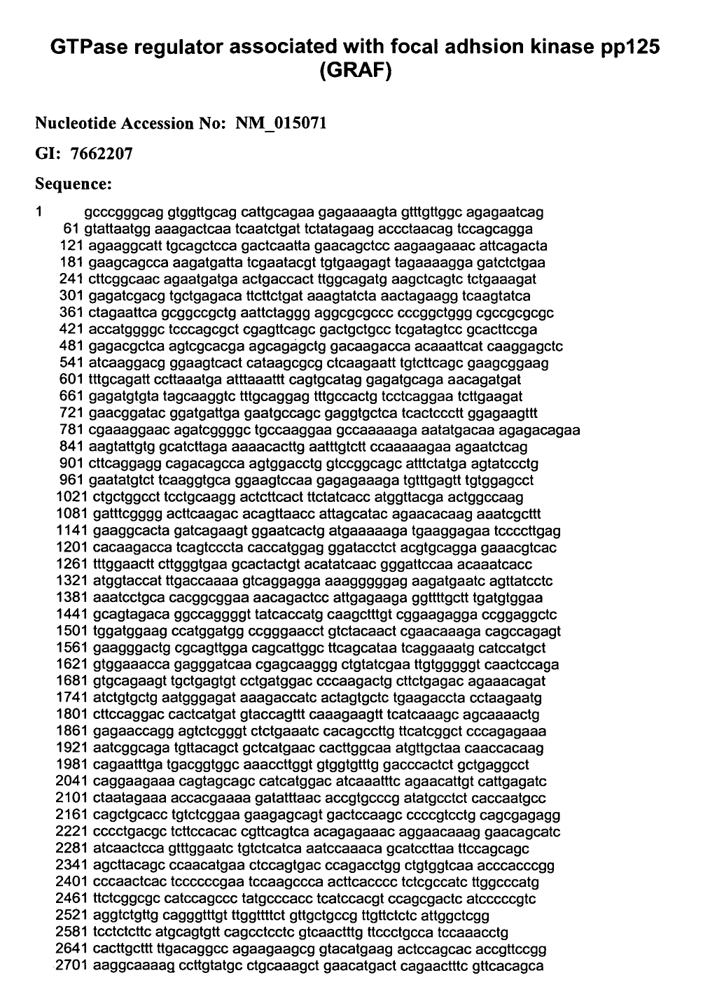

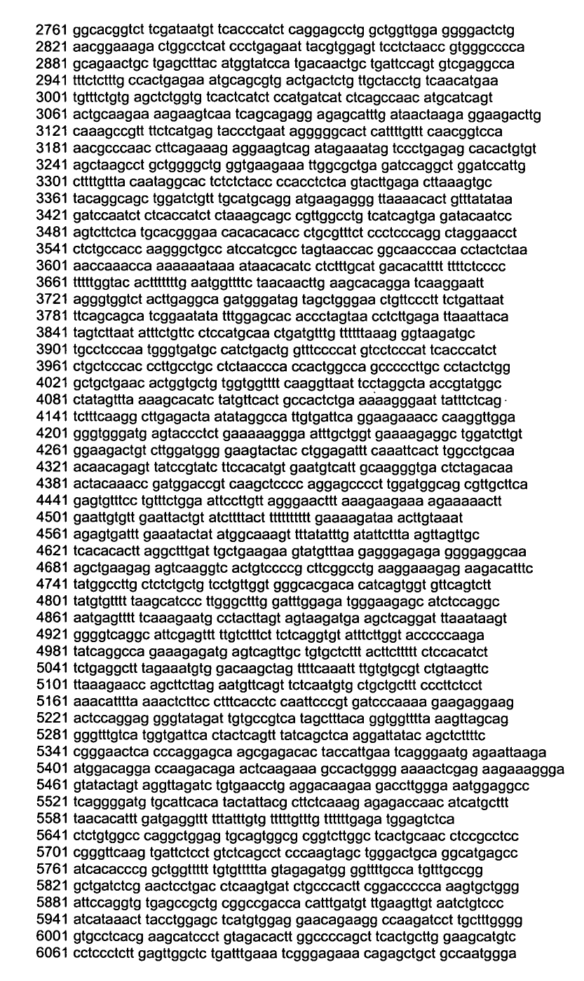

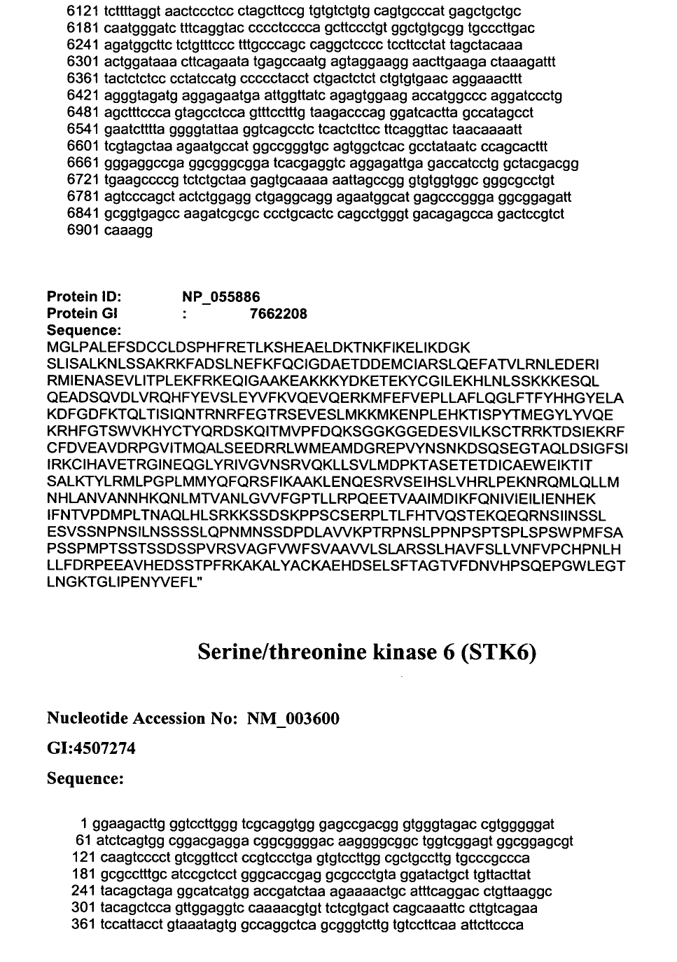

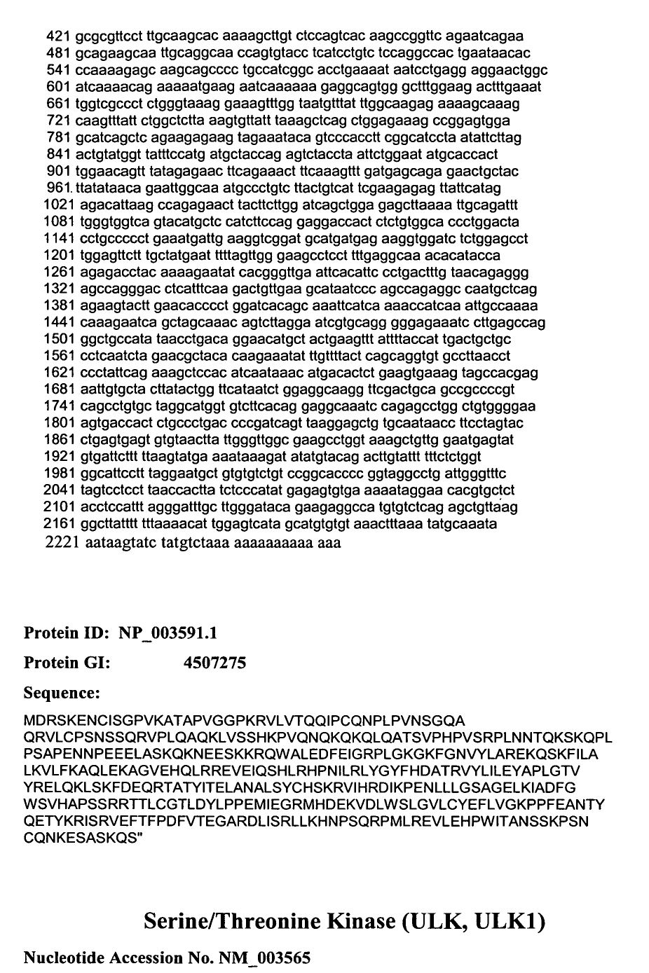

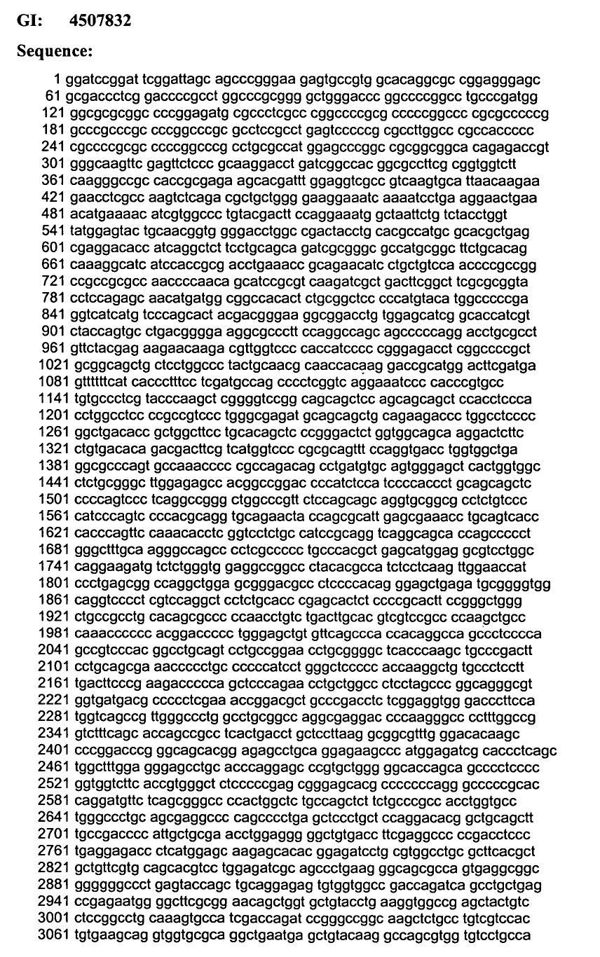

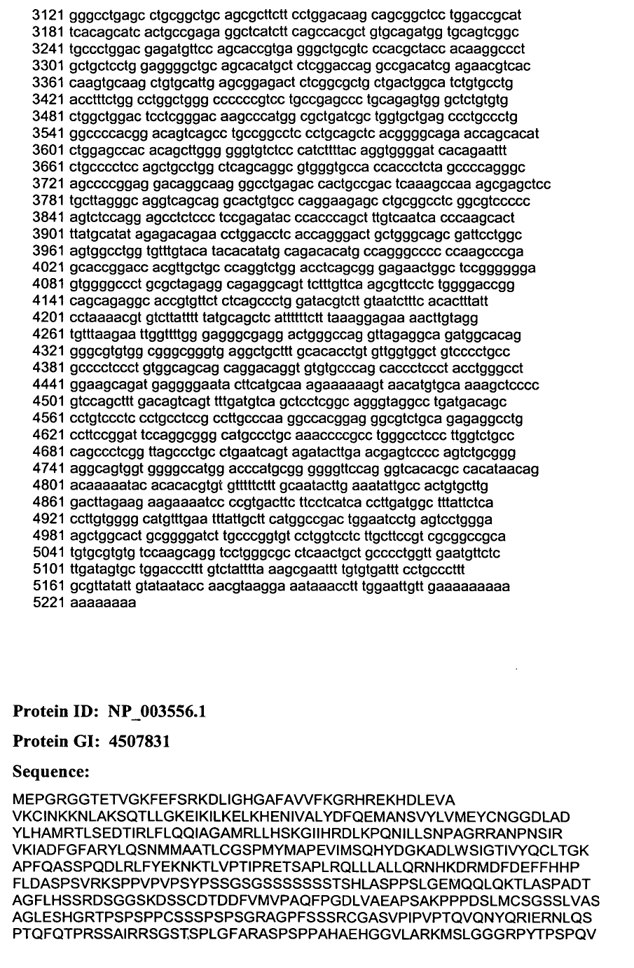

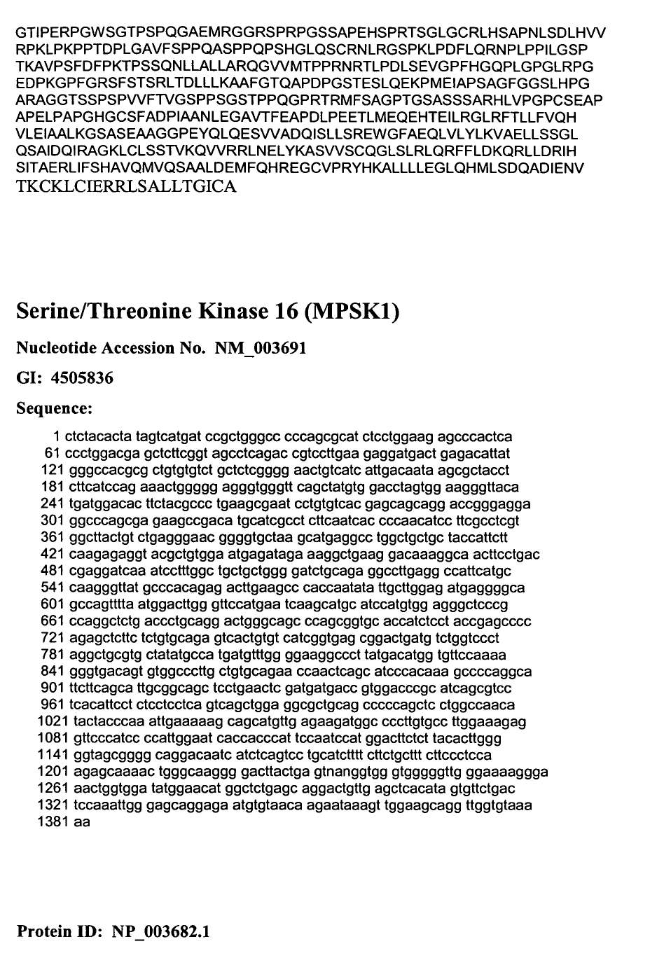

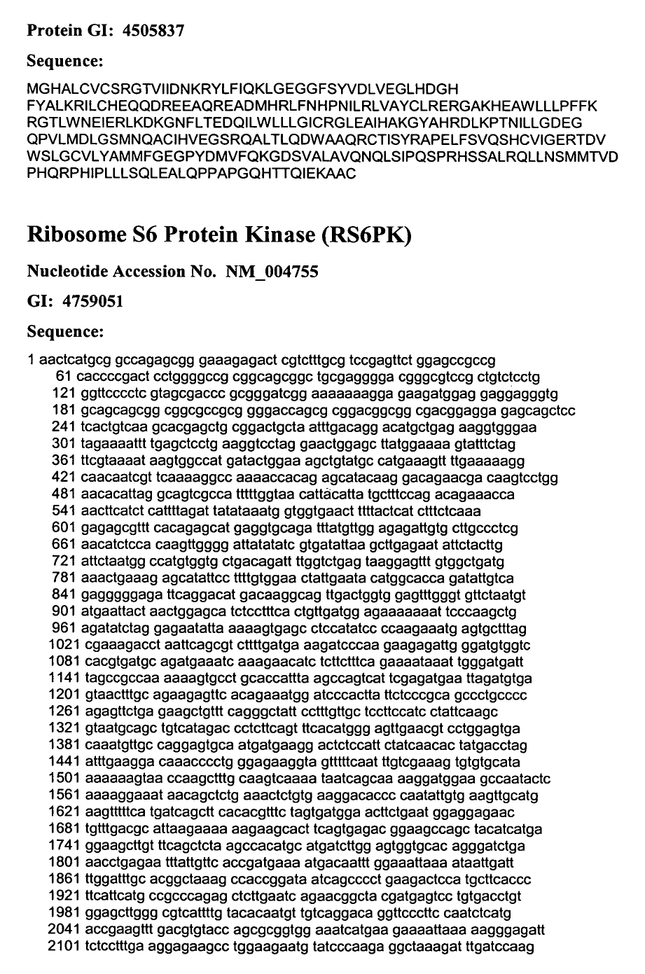

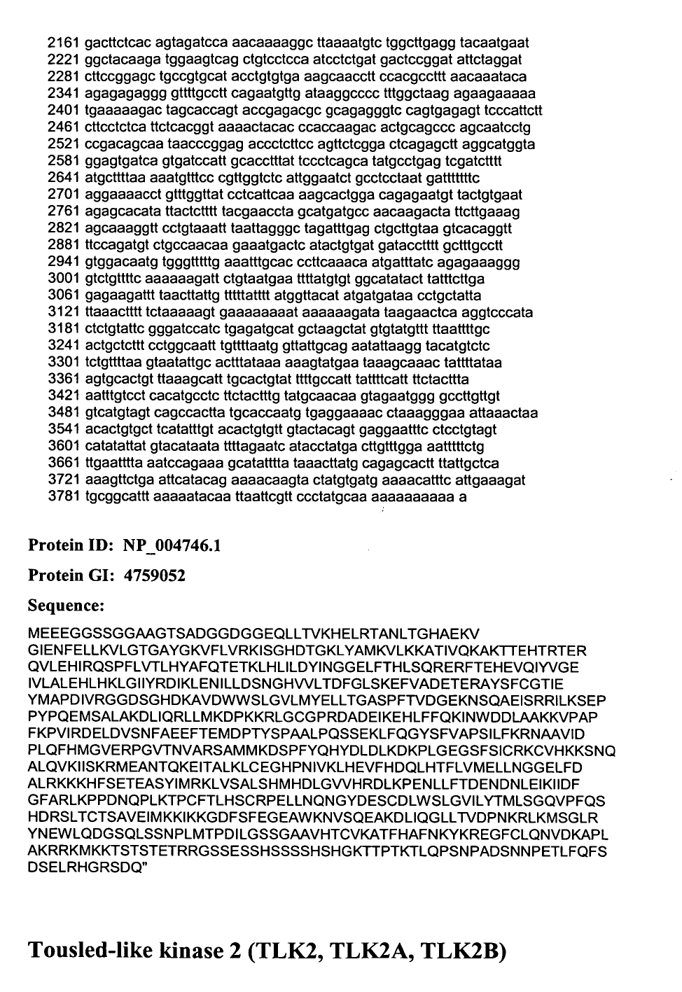

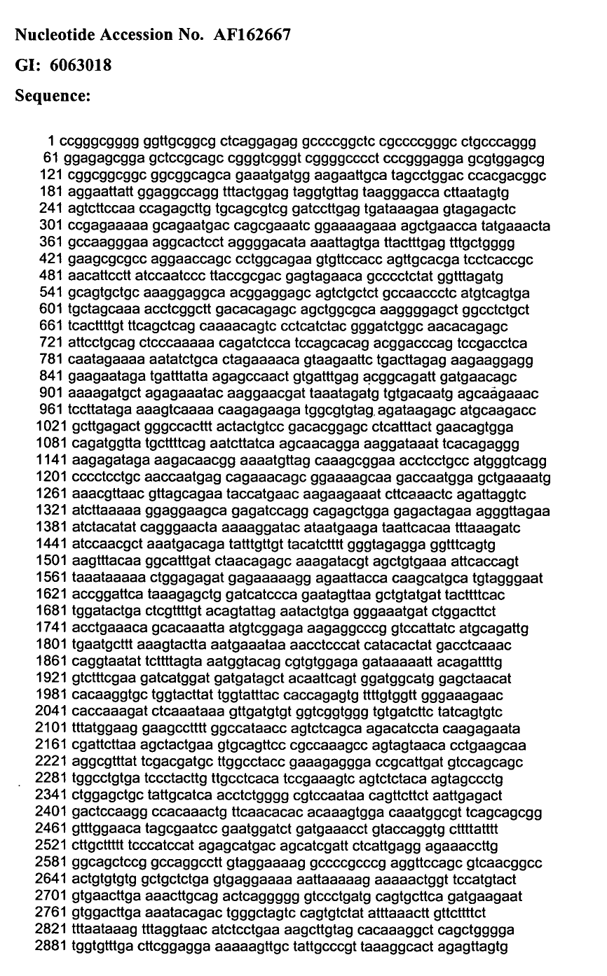

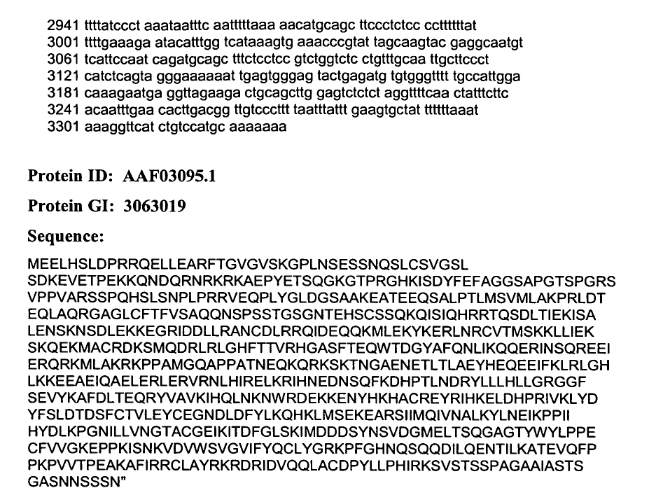

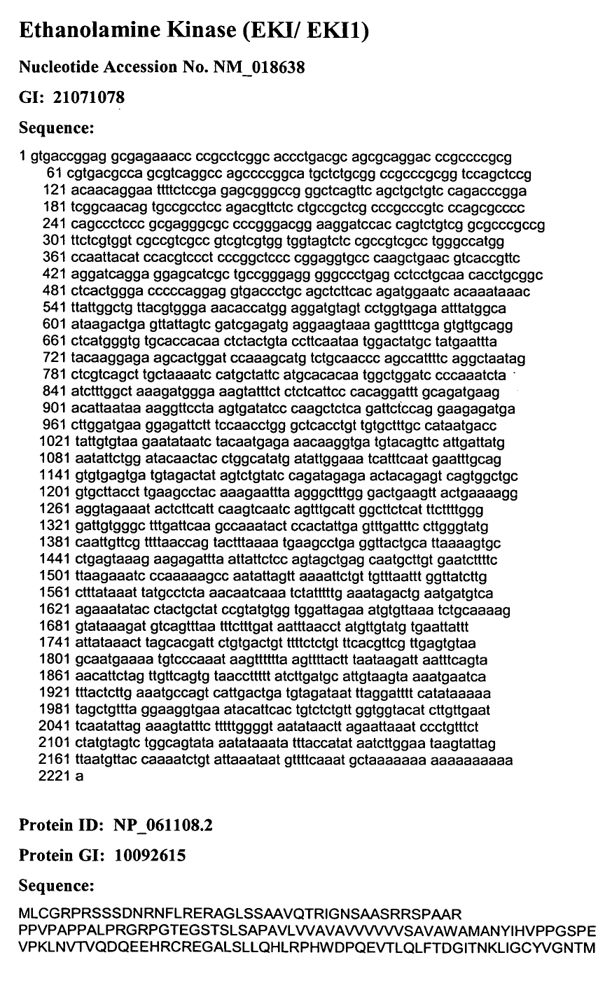

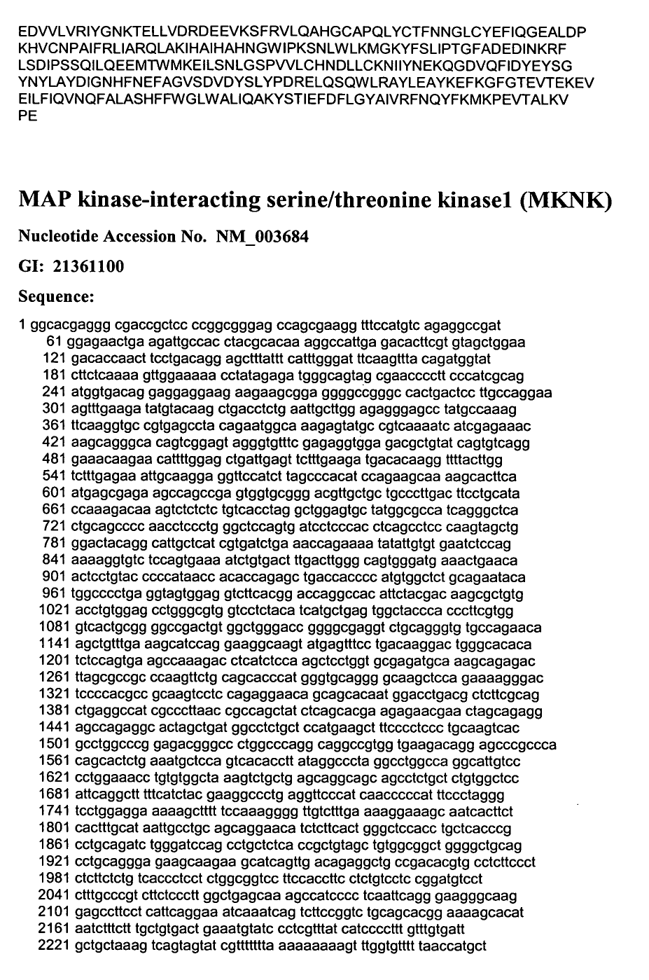

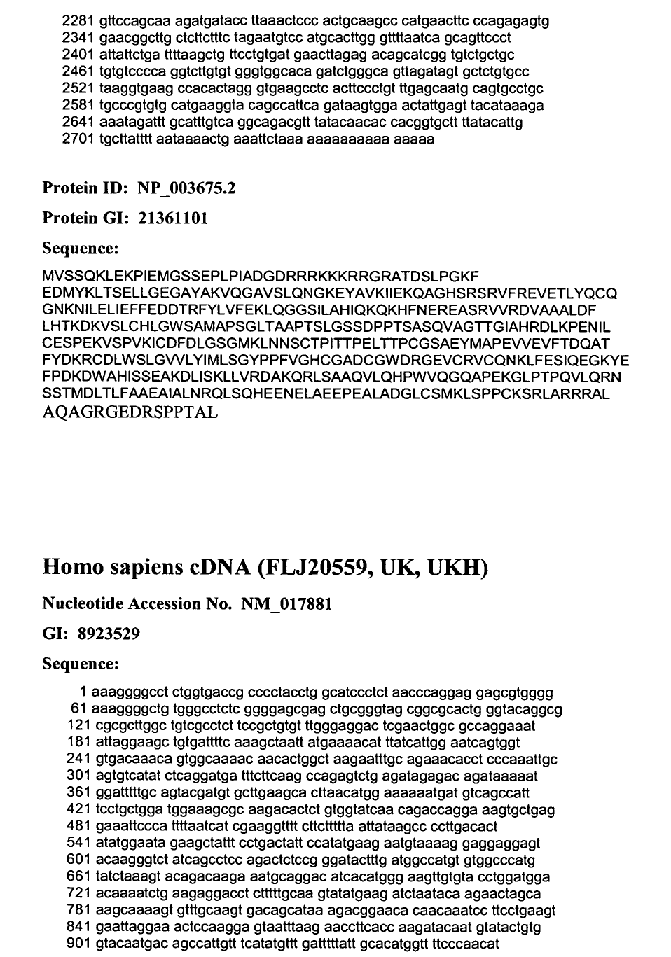

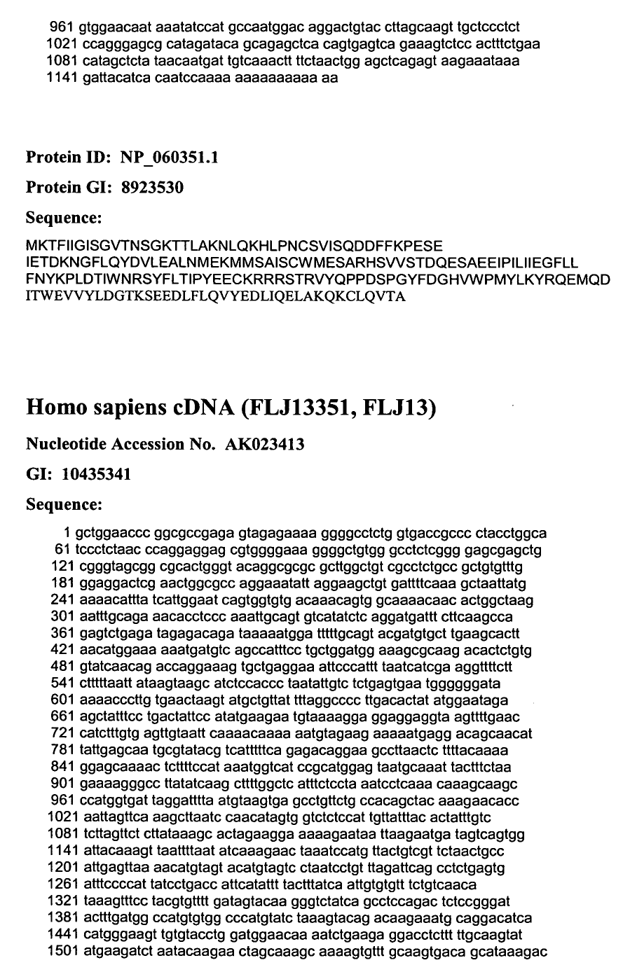

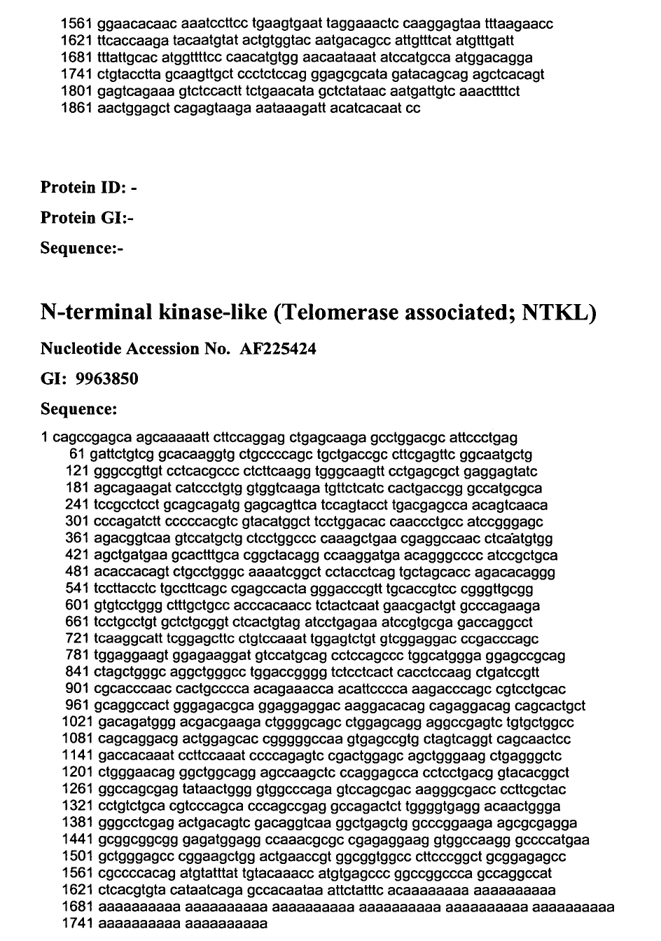

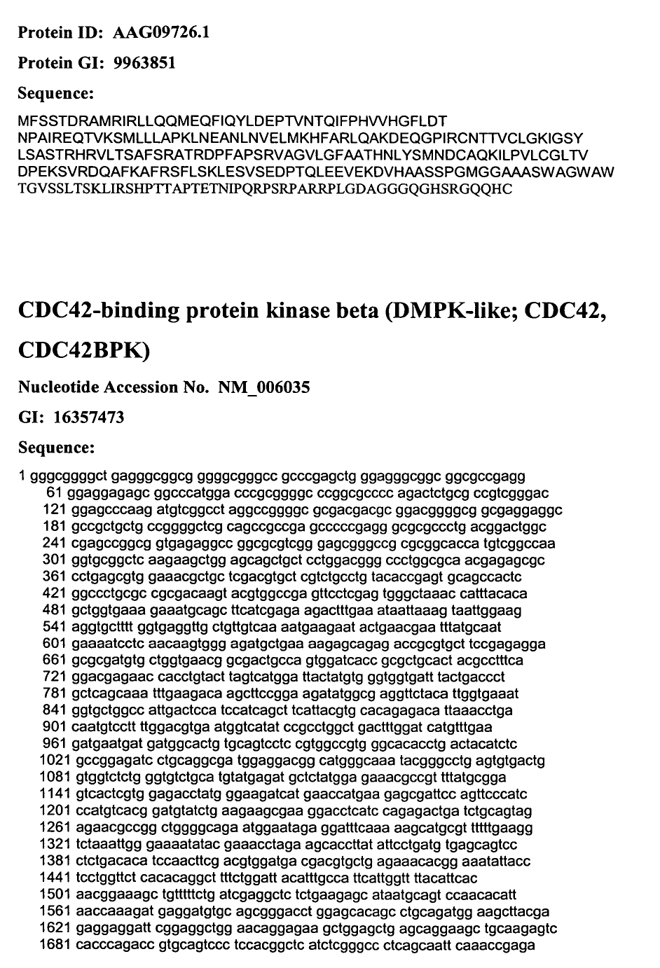

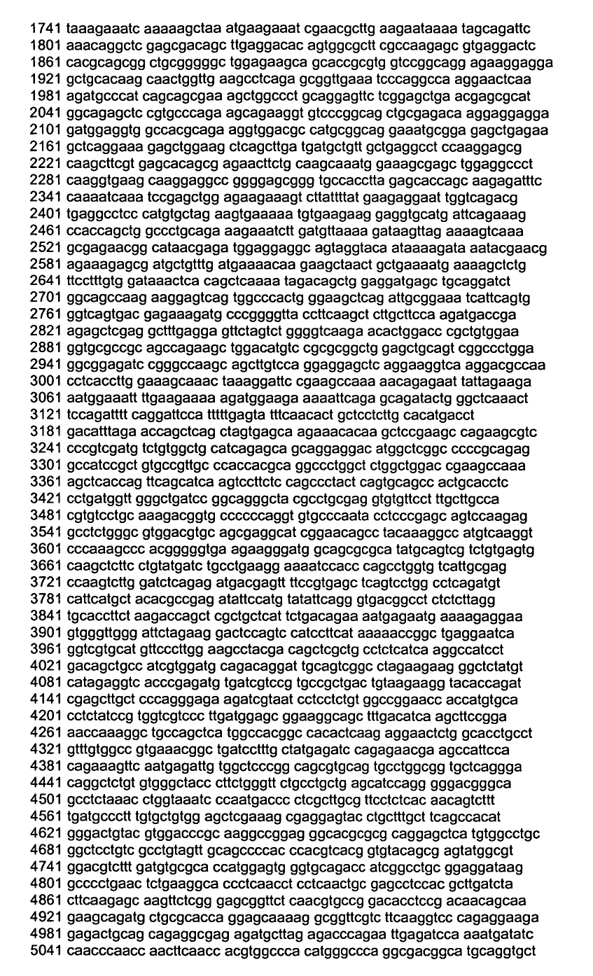

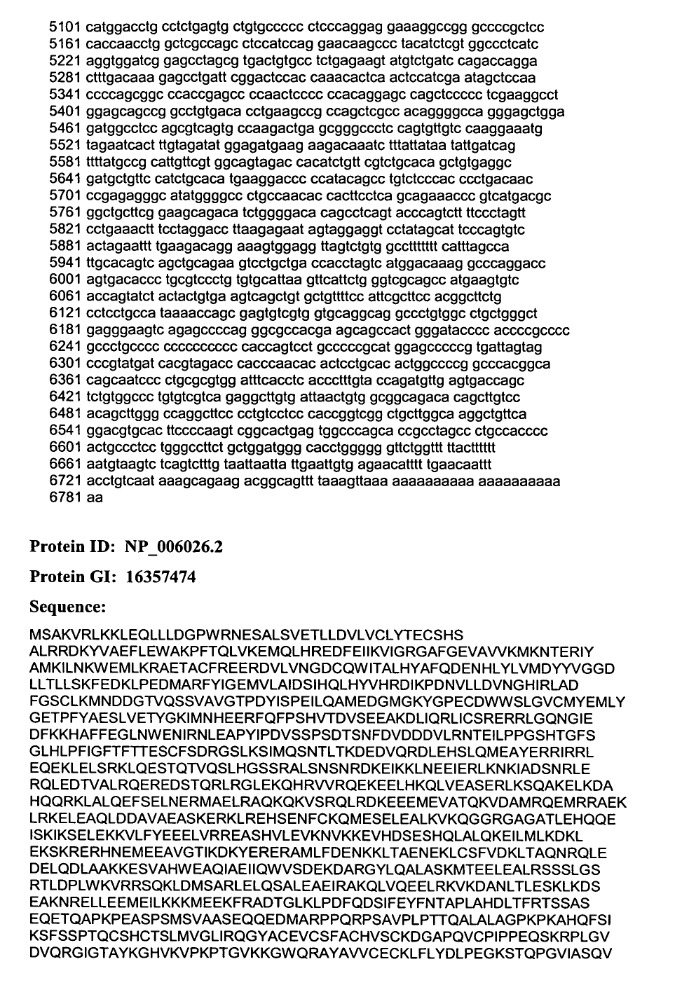

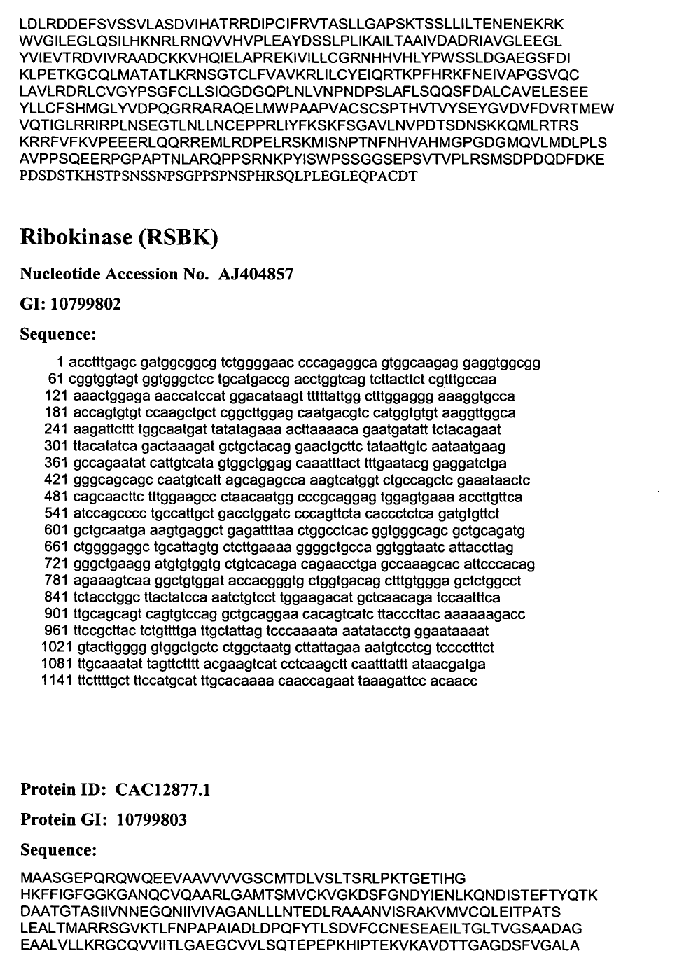

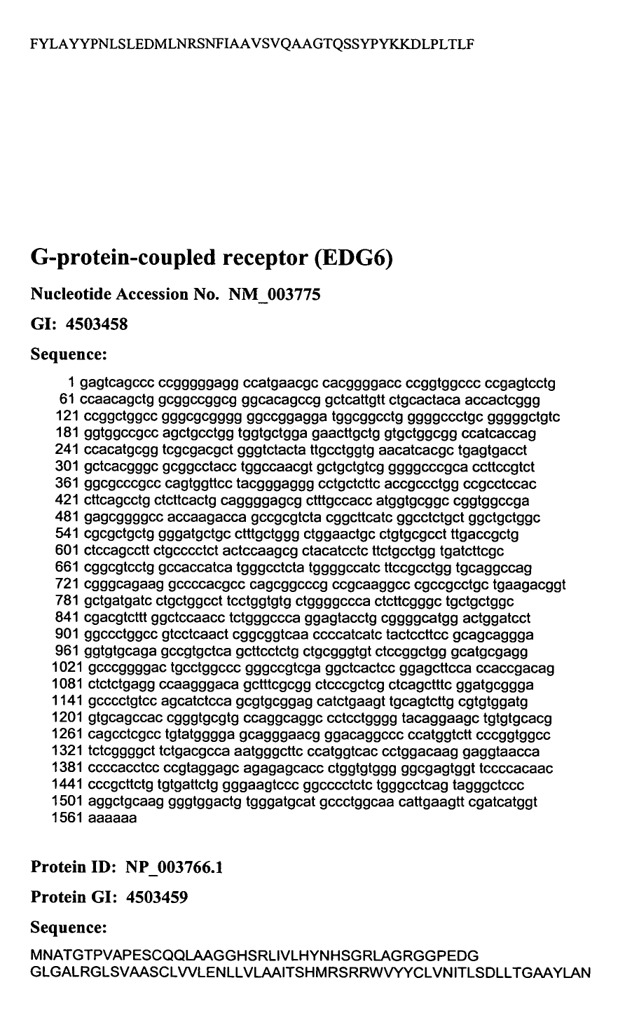

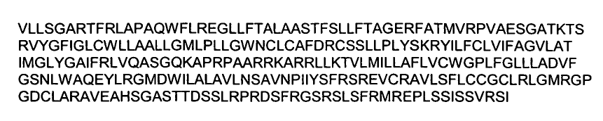

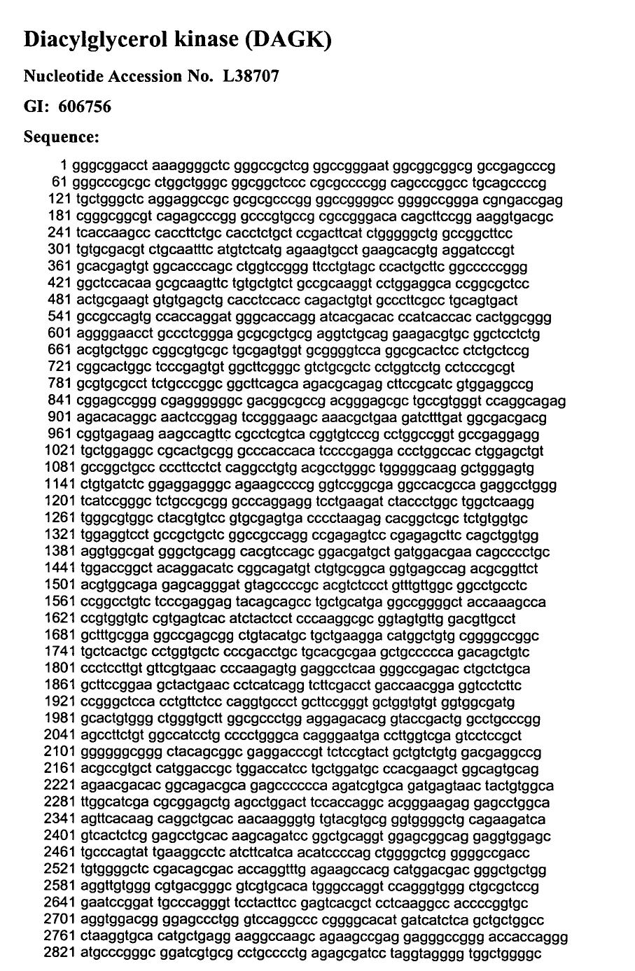

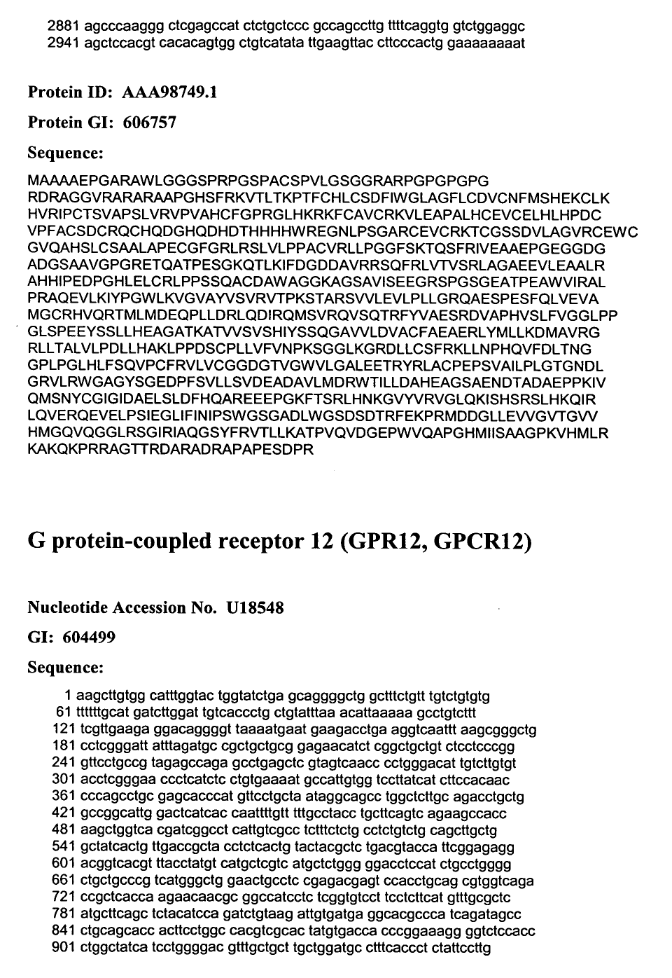

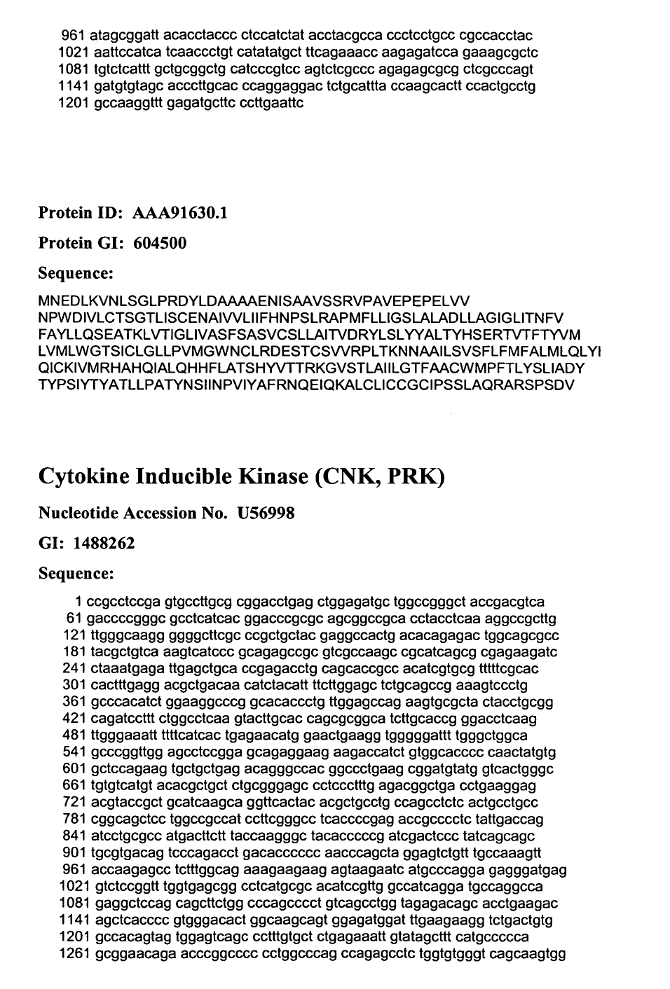

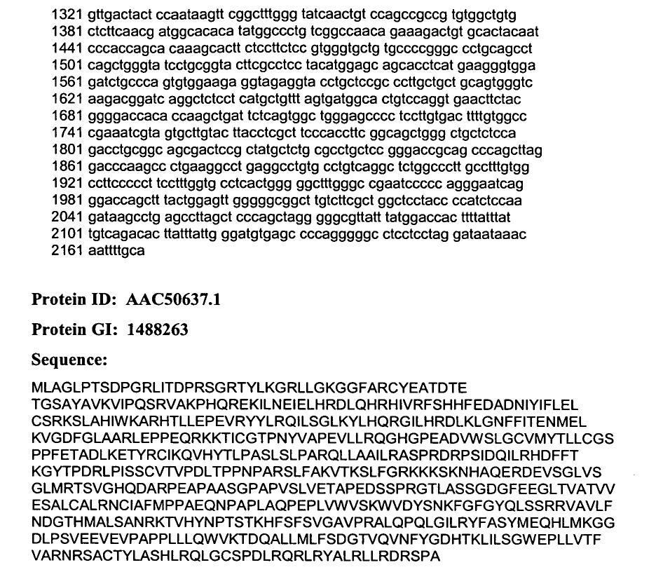

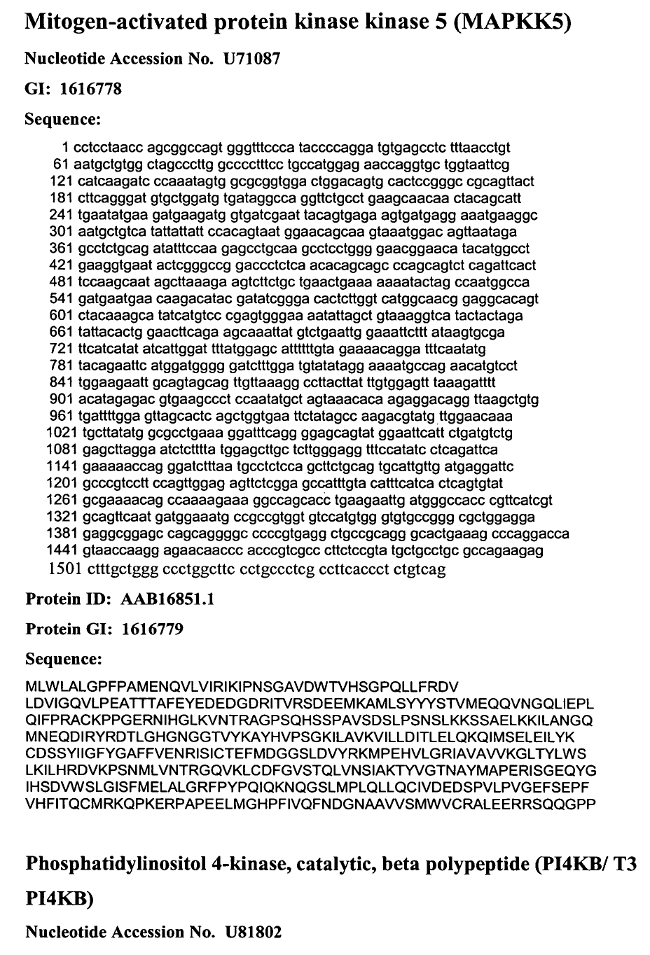

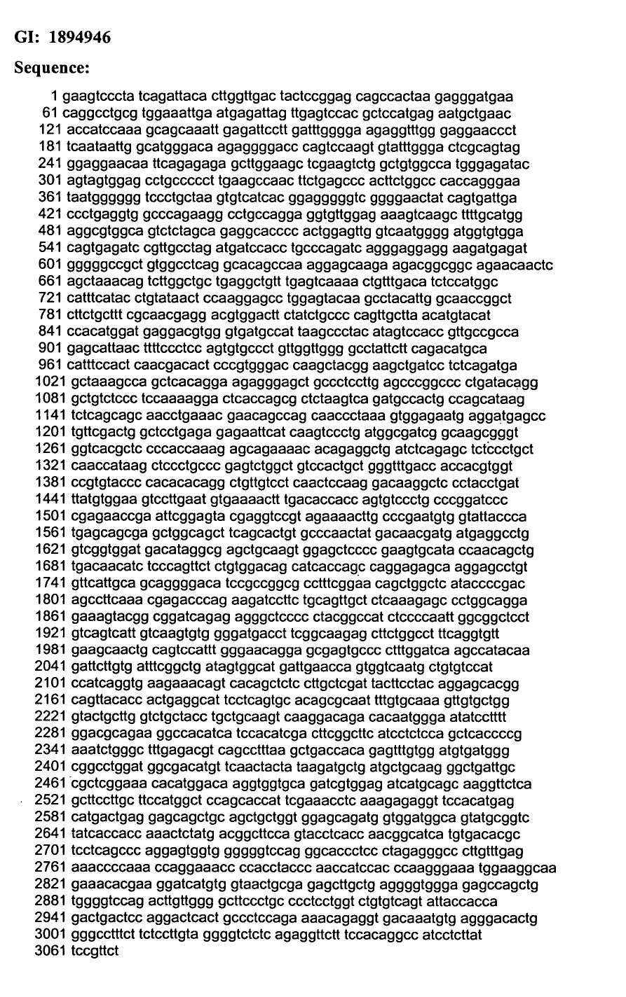

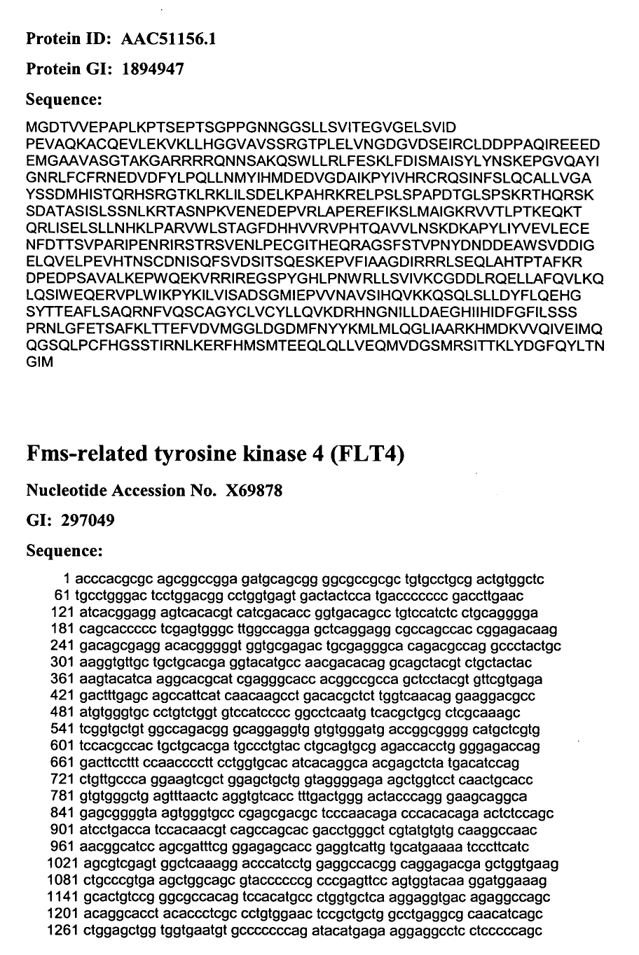

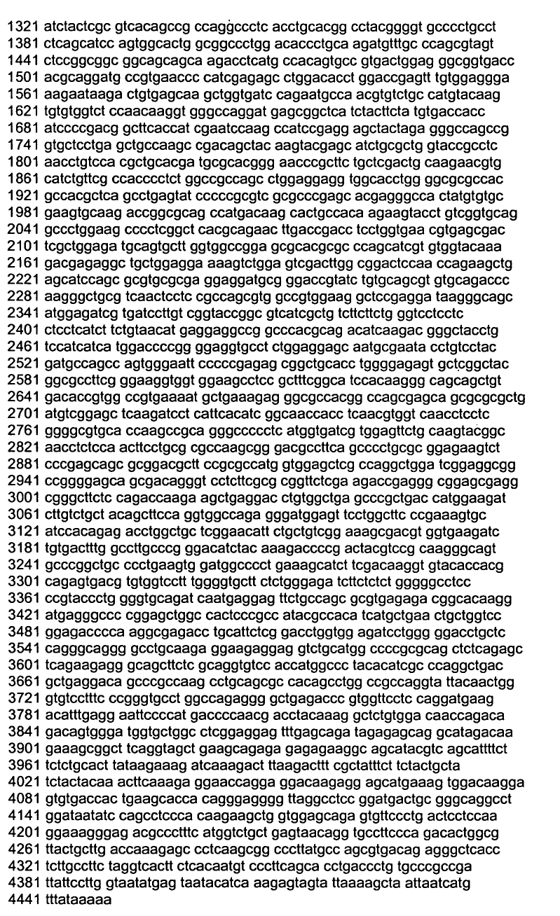

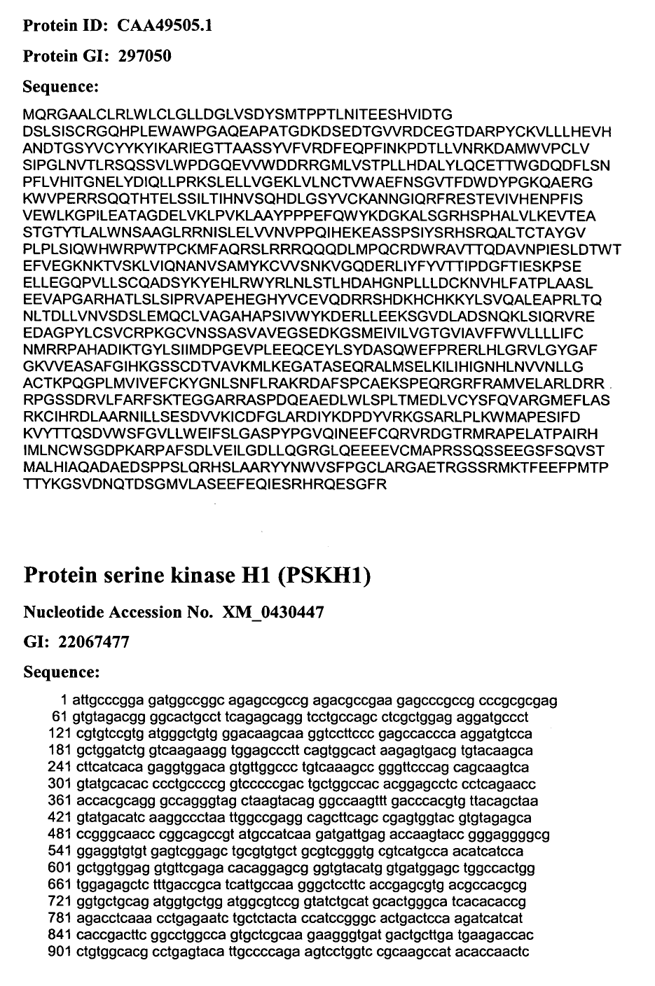

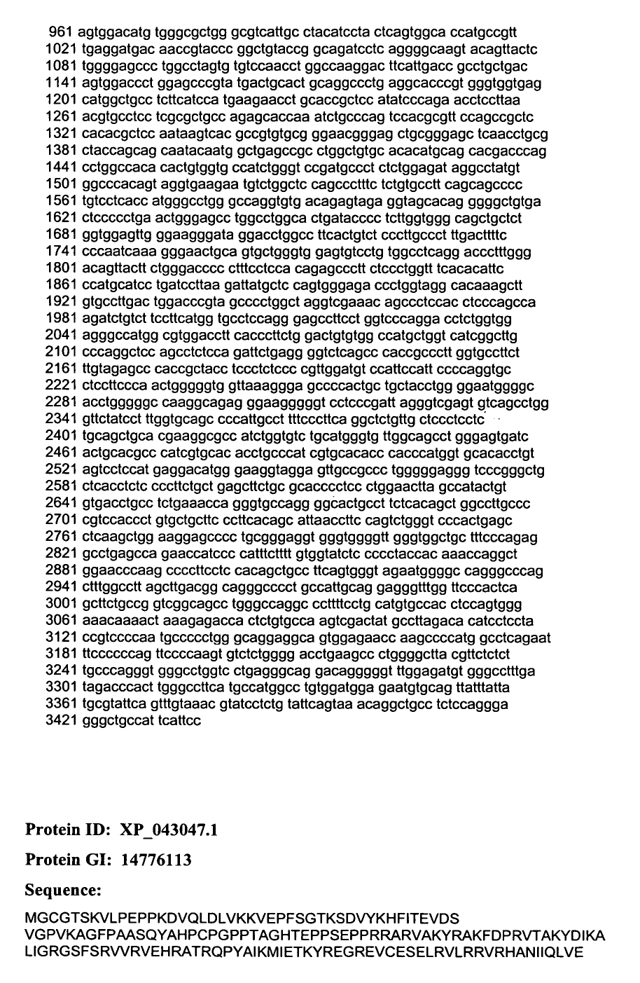

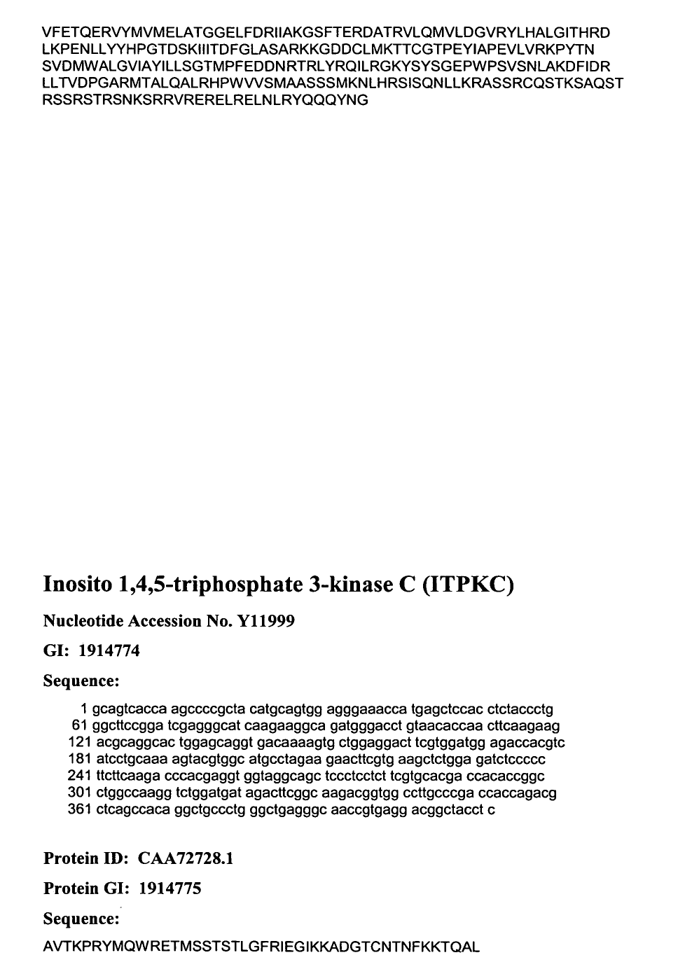

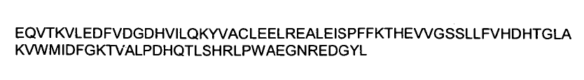

Polynucleotide and polypeptide sequences corresponding to GenBank Accession Nos. provided above are provided in FIG. 42.

TABLE 2
Fold change of gene expression across treatment
groups, referenced to untreated (T0) neutrophils.

| Accession | T4: T0 | G: T0 | M: G | M: T4 |
|---|---|---|---|---|
| AA058851 | −5.9 | −3.5 | −1.9 | −1.3 |
| AA142914 | −1.8 | −2.8 | 3.8 | 2.4 |
| AA535884 | −3.1 | −3.8 | 3.4 | 2.5 |
| AB005298 | 2.2 | 1.6 | −4.2 | −4.8 |
| AB014521 | −7.8 | −3.4 | 3 | 6.8 |
| AF011468 | 2.3 | 14.2 | −14.5 | −2.3 |
| AF045458 | −1.3 | −1.8 | −3 | −3.9 |
| AF060798 | −2.2 | −2.1 | 2.5 | 2.6 |
| AF090421 | 1.2 | 2.2 | −14.2 | −7.3 |
| AF162667 | −1.3 | −1.9 | −1.5 | −2.1 |
| AI003792 | −3.3 | −2.7 | 2.3 | 3 |
| AI368064 | −2.5 | −2.6 | 2.5 | 2.4 |
| AI400203 | 3.5 | 2.8 | −3.3 | −4.5 |
| AI741642 | −4.8 | −5.9 | 1.8 | 1.5 |
| AI761729 | 4.4 | 2.2 | −2.8 | −4.7 |
| AI823980 | 6 | 3.7 | −1.6 | −1.6 |
| AJ000479 | −2.3 | −4.1 | 1 | −2 |
| L38707 | −1.1 | −2.2 | 2 | 1.9 |
| U18548 | 2.8 | 4.4 | −3.7 | −2.1 |
| U56998 | 1.4 | −1.5 | 4.4 | 2.3 |
| U71087 | −6 | −4.5 | 4.4 | 3.5 |
| U81802 | −5.5 | −5.5 | 3.1 | 2.5 |
| X69878 | 3.8 | 3 | −3.1 | −4.2 |
| X71875 | −2.9 | −2.8 | 3.2 | 3.2 |
| Y11999 | −4.4 | −3.4 | 2.5 | 3.2 |
| AI125216 | −2.6 | −2.2 | 2.4 | 2.7 |
| AI274392 | −2.6 | −3.2 | 2.8 | 2.4 |
| AI684957 | −4.2 | −3 | 3.8 | 3.6 |
| U09607 | 2.6 | 2.1 | −2.7 | −3.4 |
| X15573 | −2.6 | −2.8 | 4 | 3.8 |

TABLE 3

| Cell Line | Tissue/histology |
|---|---|
| HL-60 | Acute promyelocytic leukaemia |
| NCI-H460 | Large cell lung carcinoma |
| HCT-15 | Colorectal adenocarcinoma |
| HT29 | Colorectal adenocarcinoma |
| U251 | CNS |
| M14 | Melanoma |
| OVCAR-3 | Ovarian adenocarcinoma |
| SK-OV-3 | Ovarian adenocarcinoma |
| 786-0 | Renal Cell Carcinoma |
| MCF7 | Breast adenocarcinoma |
| PC3 | Prostate Adenocarcinoma |
| DU145 | Prostate Adenocarcinoma |
| LnCAP | Prostate Adenocarcinoma |
| Daudi | B-Cell; Burkitts Lymphoma |
| U937 | Histocytic lymphoma |
| K562 | Chronic myelogenous leukaemia |
| CEM | Acute T lymphoblastic leukeamia |
| Molt4 | T Acute lymphoblastic leukaemia |
| TF1 | Erythroleukaemia |
| Hela | Cervical Carcinoma |

TABLE 4
QPCR primers for Target Genes:

| | | |
|---|---|---|
| Gene | Forward Primer (5′-3′) | Reverse Primer (5′-3′) |

| | | |
|---|---|---|
| MAK | GGGAGCTGGTGGCCATCAAAA | TGGATAAAAGCCAGCCCTTGCA |
| GPR86 | TGAGCGGTGCCCCAGAGACA | CAGGGTGCCAGGTGTGAGTCAGA |
| PCTAIRE | GCCGCTCAGCCGCATGTCC | GGCGCTCCCTCCTCGTGCTC |
| GRAF | CAGCGAAGCGGAAGTTTGCAGA | CTTCCTTGGCAGCCCCGATC |
| MPSK1 | CGCGCTGTGTGTCTGCTCTCG | GCGAAGGATGTTGGGGTGATTG |
| RBS5PK | GCCGCCAAAAAAGTGCCTGC | TCCTTCATCATTGCACTCCTGGC |
| TLK2 | GCAGTTCCCGCCAAAGCCAGTA | GGACGCCCCAGAGGTTGATGC |
| EK1 | CGGGCCGGGCTCAGTTCA | CGGCGGAGACTACCACCACGA |
| MKNK | CAAGCAGGGCACAGTCGGAGTAG | CGGCTGGCTTCTCGCTCATTG |
| NTKL | GGCAGCCCCGTGTCCATCTTC | CCAGCCTCCACTCTCGCCTTGA |
| CDC42 | CAAAGCGAGAACGGCATAACGAG | CCGGGCATCTTTCTCGTCACTG |
| RBSK | GGCGGCGTCTGGGGAACC | AGCCGAGCAGCTTGGACACACTG |
| EDG6 | CGGCGGTCAACCCCATCATCT | CCCGCATCCGAAAGCTGAGC |
| CNK/PRK | CGCGGACCTGAGCTGGAGATG | TGGCGACGCGGCTCTGC |
| MAPKK5 | CGGGCCGCAGTTACTCTTCAGG | CCGGCCCGAGTATTCACCTTCA |
| P14KB | CGGAGGGGGTCGGGGAAC | GCGGCCCCCATCTCATCTTC |
| FLT4 | TGCCGTGAACCCCATCGAGAG | CGTGGACAGGTTGAGGCGGTAC |
| PSKH1 | CCCGAGCCACCCAAGGATGTC | GGCCCTGCGTGGTGGTTCTGA |
| ITPKC | AGCCGGGACAGCAGCGACCT | TTTGCTTGGGCCTCTCGGTCTC |
| ROCK | GTGGGCTTGGGAAACGCTC | TCTGCATTGGAGCTAGTTCTGTTATC |

TABLE 5

| Ac No | Gene | Kidney | Colon | Ovary | Breast | Lung |
|---|---|---|---|---|---|---|
| AA058851 | MAK | 1.75 | −1.41 | 1.07 | | −2.63 |
| AA142914 | GPR86 | 4.80 | 1.60 | not | | −2.50 |
| | | | | ex- | | |
| | | | | pressed | | |
| AA535884 | PCTAIRE | −1.84 | −1.38 | 1.10 | | −2.53 |
| AB014521 | GRAF | 3.41 | 1.98 | 8.01 | | −2.13 |
| AF060798 | MPSKI | 2.11 | −1.29 | 2.77 | | 0.00 |
| AF060798 | TLK2 | 2.11 | −1.29 | 2.77 | | 0.00 |
| AI003792 | EKI | 3.05 | −2.73 | 4.78 | | −1.47 |
| AI125216 | ROCK | 1.40 | 1.50 | | | −2.10 |
| AI741642 | NTKL | 1.56 | −2.15 | 5.68 | | −2.34 |
| AI761729 | CDC42BPK | 3.15 | 1.10 | 3.43 | | −2.70 |
| AI823980 | RBSK | 1.31 | −2.63 | 5.75 | | −2.27 |
| AJ000429 | EDG6 | 4.45 | 1.11 | −1.90 | | −2.50 |
| U56998 | PRK/CNK | 5.4 | 2.5 | −1.7 | 1.5 | −3.3 |
| U71087 | MAPKK5 | 1.75 | −1.41 | 1.07 | | −2.63 |
| U81802 | T3 PI4KB | 1.69 | −1.21 | −1.01 | | −2.16 |
| X69878 | FLT4 | −1.71 | −1.25 | not ex | | −3.68 |
| X71875 | PSKH1 | 1.70 | −1.34 | −1.28 | | −3.46 |

TABLE 6

| Acc. No. | Gene | U251 | SKOV3 | OVCAR3 | MCF7 | PC3 | HCT15 | 786-O | M-14 | HT29 | H460 |
|---|---|---|---|---|---|---|---|---|---|---|---|
| AA058851 | MAK | 0.004 | 0.013 | 0.006 | 0.001 | 0.004 | 0.012 | 0.003 | 0.001 | 0.003 | 0.006 |
| AA142914 | GPR86 | 0.012 | 0.015 | 0.013 | 0.011 | 0.013 | 0.000 | 0.008 | 0.007 | 0.000 | 0.050 |
| AA535884 | PCTAIRE | 1.07 | 0.11 | 1.11 | 0.93 | 0.63 | 1.10 | 2.01 | 1.79 | 1.15 | 1.17 |
| AB014521 | GRAF | 0.123 | 0.091 | 0.015 | 0.000 | 0.019 | 0.015 | 0.050 | 0.012 | 0.047 | 0.004 |
| AF060798 | MPSKI | 3.60 | 1.76 | 0.86 | 0.78 | 1.44 | 1.95 | 1.52 | 2.26 | 1.17 | 1.42 |
| AF060798 | TLK2 | 6.68 | 5.35 | 3.03 | 3.40 | 4.87 | 4.94 | 4.33 | 3.74 | 3.20 | 3.47 |
| AI003792 | EKI | 0.39 | 0.24 | 0.19 | 0.11 | 0.25 | 0.70 | 0.25 | 0.44 | 0.13 | 0.18 |
| AI125216 | ROCK | 7.69 | 12.29 | 2.87 | 2.38 | 5.31 | 17.42 | 11.28 | 5.15 | 5.20 | 4.84 |
| AI368064 | MKNK | 5.99 | 2.69 | 1.70 | 0.96 | 2.56 | 2.89 | 2.78 | 2.90 | 2.63 | 1.06 |
| AI741642 | NTKL | 2.15 | 1.30 | 1.56 | 0.83 | 1.37 | 1.52 | 2.18 | 1.92 | 1.30 | 1.05 |
| AI761729 | CDC42BPk | 3.52 | 4.98 | 5.17 | 0.87 | 2.28 | 3.97 | 2.64 | 1.37 | 1.07 | 2.50 |
| AI823980 | RBSK | 0.36 | 0.49 | 0.42 | 0.20 | 0.15 | 0.13 | 2.31 | 0.06 | 0.16 | 0.34 |
| AJ000429 | EDG6 | 0.000 | 0.007 | 0.000 | 0.000 | 0.000 | 0.000 | 0.000 | 0.000 | 0.000 | 0.000 |
| U56998 | PRK/CNK | 0.04 | | 0.03 | 0.01 | 0.12 | 0.09 | | | | 0.07 |
| U71087 | MAPKK5 | 4.16 | 2.18 | 1.30 | 1.26 | 1.82 | 1.98 | 2.28 | 4.14 | 1.50 | 3.28 |
| U81802 | T3 PI4KB | 11.50 | 4.09 | 3.63 | 2.62 | 5.03 | 3.86 | 2.09 | 4.83 | 2.34 | 3.74 |
| X69878 | FLT4 | 0.007 | 0.009 | 0.009 | 0.023 | 0.003 | 0.003 | 0.000 | 0.000 | 0.000 | 0.195 |
| X71875 | PSKH1 | 1.22 | 1.41 | 1.13 | 0.29 | 1.25 | 1.30 | 0.67 | 1.62 | 0.52 | 0.93 |

| Acc. No. | Gene | DU145 | Daudi | U937 | K562 | CEM | MOLT4 | LNCaP | Hela | TF1 |
|---|---|---|---|---|---|---|---|---|---|---|
| AA058851 | MAK | 0.042 | 0.004 | 0.000 | 0.050 | 0.000 | 0.000 | 0.075 | 0.000 | 0.052 |
| AA142914 | GPR86 | 0.051 | 0.000 | 0.046 | 0.001 | 0.109 | 0.058 | 0.005 | 0.000 | 0.003 |
| AA535884 | PCTAIRE | 5.37 | 1.73 | 4.03 | 0.01 | 1.69 | 2.87 | 0.57 | 0.05 | 0.43 |
| AB014521 | GRAF | 0.109 | 0.021 | 0.048 | 0.002 | 0.041 | 0.088 | | 0.009 | 0.016 |
| AF060798 | MPSKI | 4.142 | 0.847 | 1.142 | 1.002 | 2.205 | 2.535 | 2.16 | 0.307 | 0.621 |
| AF060798 | TLK2 | 9.57 | 4.44 | 3.34 | 3.11 | 4.96 | 7.79 | | 1.51 | 1.49 |
| AI003792 | EKI | 1.34 | 0.40 | 0.52 | 0.71 | 1.51 | 1.28 | 0.36 | 0.59 | 0.31 |
| AI125216 | ROCK | 19.16 | 5.18 | 8.58 | 4.61 | 10.51 | 14.16 | 4.86 | 1.23 | 3.44 |
| AI368064 | MKNK | 4.27 | 1.12 | 3.34 | 2.80 | 1.86 | 3.01 | 2.64 | 0.75 | 2.05 |
| AI741642 | NTKL | 7.90 | 1.59 | 2.46 | 2.89 | 1.78 | 1.89 | 1.34 | 0.65 | 3.59 |
| AI761729 | CDC42BPk | 20.73 | 0.01 | 1.00 | 1.30 | 0.07 | 0.01 | 3.17 | 0.45 | 2.99 |
| AI823980 | RBSK | 4.42 | 0.27 | 0.58 | 0.32 | 0.48 | 0.68 | 0.57 | 0.74 | 0.52 |
| AJ000429 | EDG6 | 0.225 | 0.157 | 0.522 | 0.001 | 0.843 | 1.153 | | 0.000 | 0.438 |
| U56998 | PRK/CNK | | 0.04 | 0.02 | | | 0.02 | 0.05 | 0.06 | |
| U71087 | MAPKK5 | 11.44 | 1.53 | 7.91 | 2.33 | 5.90 | 8.37 | 2.43 | 2.92 | 6.30 |
| U81802 | T3 PI4KB | 17.13 | 3.30 | 3.52 | 4.74 | 4.14 | 5.58 | 6.42 | 0.32 | 2.96 |
| X69878 | FLT4 | 0.677 | 0.009 | 0.000 | 0.076 | 0.215 | 0.019 | 0.036 | 0.000 | 0.020 |
| X71875 | PSKH1 | 3.21 | 0.76 | 0.71 | 0.90 | 1.29 | 1.90 | 1.96 | 0.02 | 0.45 |

TABLE 7
Cancer cell lines used for expression profiling of target genes.

| Cell Line | Tissue of origin |
|---|---|
| MES-SA | Uterine sarcoma |
| H209 | Lung carcinoma |
| C-33 A | Cervical carcinoma |
| COLO 205 | Colorectal adenocarcinoma |
| COLO 201 | Colorectal adenocarcinoma |
| MDA-Pca-2b | Prostate adenocarcinoma |
| HOP92 | Lung carcinoma |
| SK-BR-3 | Breast adenocarcinoma |
| A253 | Submaxillary salivary gland epidermoid |
| | carcinoma |
| Caov-3 | Ovarian adenocarcinoma |
| MDA-MB-175 | Breast carcinoma |
| MDA-MB-231 | Breast adenocarcinoma |
| MCF7 RPI | Breast adenocarcinoma |
| MDA-MB-435-BAG | Breast adenocarcinoma |
| H460 | Lung carcinoma |
| KLE | Uterine adenocarcinoma |
| MSTO 211 | Lung mesothelioma |
| ES-2 | Ovarian carcinoma |
| RWPE-2 | Prostate (normal) |
| A549 | Lung carcinoma |
| MDAH-2774 | Ovarian adenocarcinoma |
| DU145 | Prostate carcinoma |
| MCF7 | Breast adenocarcinoma |
| 22Rv1 | Prostate carcinoma |
| 293 (ATCC) | Kidney (transformed with adenovirus 5) |
| HB4a (C5.2) | Normal Breast transformed with ErbB2 |
| HB4a | Normal Breast |
| MDA-MB-468 | Breast adenocarcinoma |
| PC-3 | Prostate adenocarcinoma |
| HCT 116 | Colorectal carcinoma |
| CAMA-1 | Breast adenocarcinoma |
| AU565 | Breast adenocarcinoma |
| ZR-75-1 | Breast carcinoma |
| HT-29 | Colorectal adenocarcinoma |
| IGROV-1 | Ovarian carcinoma |
| TOV-112D | Ovarian adenocarcinoma |
| SW626 | Ovarian adenocarcinoma |
| TOV-21G | Ovarian adenocarcinoma |
| A2780 | Ovarian carcinoma |
| BT-483 | Breast carcinoma |
| NCI-H69 | Lung carcinoma |
| BT-474 | Breast carcinoma |
| OV-90 | Ovarian adenocarcinoma |
| OVCAR-3 | Ovarian adenocarcinoma |
| SKOV-3 | Ovarian adenocarcinoma |
| MDA-MB-436 | Breast adenocarcinoma |
| SW48 | Colorectal adenocarcinoma |
| A1165 | Pancreatic carcinoma |
| PANC-1 | Pancreatic carcinoma |
| HPAC | Pancreatic carcinoma |
| HeLa | Cervical adenocarcinoma |
| RWPE-1 | Normal Prostate |
| H460a | Lung carcinoma |
| Capan-1 | Pancreatic adenocarcinoma |
| LNCaP | Prostate carcinoma |
| Capan-2 | Pancreatic adenocarcinoma |
| SK-MEL-31 | Melanoma |
| SK-MEL-28 | Melanoma |
| SK-MEL-2 | Melanoma |
| A2058 | Melanoma |
| NCI-H322 | Lung carcinoma |

TABLE 8
Q-PCR primers for target genes and relevant control genes.

| | | | |
|---|---|---|---|
| Gene | Forward Primer (5′ to 3′)¹ | Reverse Primer (5′ to 3′)¹ | Conc.² |

| | | | |
|---|---|---|---|
| GRAF | GATAGTCCGCACTTCCG | GAGTGACTTCCCGTCCTT | 100 nM |
| ULK1 | GACTTCCAGGAAATGGCT | AGAGCCTGATGGTGTCCT | 100 nM |
| EKI | CGTCGTGGTGGTAGTCTC | GATGCTCCTCCTGATCCT | 100 nM |
| ROCK1 | GCATAAATCCACCAGGAA | ATGTCCCTTTCTTCCCAG | 100 nM |
| NTKL | TACCTCAAGGCGAGAGTG | CAGTCGTTGACCAGGAAG | 100 nM |
| RBSK | ATACGGAGGATCTGAGGG | TCCAAAGAAGTTGCTGGA | 100 nM |
| DAGK | GGAAGGTGACGCTCACCAAG | ACATGAAATTGCAGACGTCGC | 200 nM |
| ITPKC | CAGACGGACAGACTGAGC | TCCATTCTAGATGCGTCC | 100 nM |
| UKH | TGCAGTACGATGTGCTTG | CAGCACTTTCCTGGTCTG | 100 nM |
| BAI2 | CCTGCTGAGGCCGATTTG | TTTCACTTTCGGTTCCTCTTCC | 100 nM |
| GPR12 | AAGGTCAATTTAAGCGGGCTG | TCTGGCTCTACGGCAGGAAC | 200 nM |
| GPR86 | AGGTGACACTGGAAGCAA | CACTGTGTAGAGGGCTGG | 100 nM |
| BcI2 | CACGCTGGGAGAACAGGGT | CACATCTCCCGCATCCCA | 100 nM |
| Survivin | TCAAGGACCACCGCATCTCT | CAGTGGATGAAGCCAGCCTC | 100 nM |
| B | | | |
| GAPDH | CGACCACTTTGTCAAGCTCA | GGGTCTTACTCCTTGGAGGC | 100 nM |

¹Primers are synthesised by MWG-Biotech. ²The final concentration of each primer in a Q-PCR reaction.

TABLE 9
Sequence of siRNA oligonucleotides.

| | | |
|---|---|---|
| Gene | Sense Oligonucleotide (5′-3′) | Antisense Oligonucleotide (5′-3′) |

| | | |
|---|---|---|
| GRAF | GCGGAAGUUUGCAGAUUCCtt | GGAAUCUGCAAACUUCCGCtt |
| ULK1¹ | GGAACUGAAACAUGAAAACtt | GUUUUCAUGUUUCAGUUCCtt |
| EKI | GCACUGGAUCCAAAGCAUGtt | CAUGCUUUGGAUCCAGUGCtt |
| ROCK | UACAUGCCUGGUGGAGAUCtt | GAUCUCCACCAGGCAUGUAtt |
| NTKL | UGUGGAGCUGAUGAAGCACtt | GUGCUUCAUCAGCUCCACAtt |
| RBSK | CGUCCUGGAGUGACAAAUGtt | CAUUUGUCACUCCAGGACGtt |
| DAGK¹ | GGCUGCACAACAAGGGUGUtt | ACACCCUUGUUGUGCAGCCtg |
| ITPKC | GUCCUGGGCUGAUAACCUCtt | GAGGUUAUCAGCCCAGGACtt |
| UKH | AGCGCAAGACACUCUGUGGtt | CCACAGAGUGUCUUGCGCUtt |
| BAI2¹ | GGACCUGUUUGGUACCAUCtt | GAUGGUAGGAAAGAGGUCCtg |
| GPR12¹ | GGACGGUCACGUUUACCUAtt | UAGGUAAACGUGACCGUCCtc |
| GPR86 | AAACACUUUGGUGGCCGACtt | GUCGGCCACCAAAGUGUUUtt |

¹siRNA sequences designed and synthesised by Ambion.

TABLE 10
Demonstration of efficacy of siRNA in the PC3 prostate
cancer cell line.

| | Decrease in expression |
|---|---|
| Target Gene | of target gene |
| GRAF | 64.5% |
| ULK1 | 78.9% |
| EKI | 76.0% |
| ROCK1 | 65.0% |
| NTKL | 82.9% |
| RBSK | 77.0% |
| DAGK | 79.9% |
| ITPKC | 83.9% |
| UKH | 75.3% |
| BAI2 | 82.7% |
| GPR12 | 78.5% |
| GPR86 | 86.9% |
| Bcl2 | 70.8% |
| Survivin B | 82.2% |

TABLE 11

| | | |
|---|---|---|
| Gene | Sense Oligonucleotide (5′-3′) | Antisense Oligonucleotide (5′-3′) |

| | | |
|---|---|---|
| MAK | GAAGCCAAGCAUGGGUGUUtt | AACACCCAUGCUUGGCUUCtt |
| GPR86 | AAACACUUUGGUGGCCGACtt | GUCGGCCACCAAAGUGUUUtt |
| PCTAIRE | GUCAGUGCCCACAAAGACUtt | AGUCUUUGUGGGCACUGACtt |
| GRAF | GCGGAAGUUUGCAGAUUCCtt | GGAAUCUGCAAACUUCCGCtt |
| MPSK1 | GGGUUAUGCCCACAGAGACtt | GUCUCUGUGGGCAUAACCCtt |
| MPSK1seq2¹ | GCCGACAUGCAUCGCCUCUtt | AGAGGCGAUGCAUGUCGGCtt |
| RBS6PK | CGUCCUGGAGUGACAAAUGtt | CAUUUGUCACUCCAGGACGtt |
| TLK2A² | GUGUUCCACCAGUUGCACGtt | CGUGCAACUGGUGGAACACtt |
| TLK2B² | GAUGGCGUGUAGAGAUAAGtt | CUUAUCUCUACACGCCAUCtt |
| EKI1 | GCACUGGAUCCAAAGCAUGtt | CAUGCUUUGGAUCCAGUGCtt |
| MKNK | UACAUGGCCCCUGAGGUAGtt | CUACCUCAGGGGCCAUGUAtt |
| MKNKseq2¹ | AUUGCAAGGAGGUUCCAUCtt | GAUGGAACCUCCUUGCAAUtt |
| NTKL | UGUGGAGCUGAUGAAGCACtt | GUGCUUCAUCAGCUCCACAtt |
| CDC42 | GCUCAGCUUGAUGAUGCUGtt | CAGCAUCAUCAAGCUGAGCtt |
| RBSK | GACCUUCCGCUUACUCUGUtt | ACAGAGUAAGCGGAAGGUCtt |
| EDG6 | CAUCACGCUGAGUGACCUGtt | CAGGUCACUCAGCGUGAUGtt |
| CNK/PRK | UCGUAGUGCUUGUACUUACtt | GUAAGUACAAGCACUACGAtt |
| CNK/PRKseq2¹ | CAGAAAGACUGUGCACUACtt | GUAGUGCACAGUCUUUCUGtt |
| MAPKK5 | GAGGACAGGUUAAGCUGUGtt | CACAGCUUAACCUGUCCUCtt |
| P14KB | GCUACGGAAGCUGAUCCUCtt | GAGGAUCAGCUUCCGUAGCtt |
| FLT4 | GUACGGCAACCUCUCCAACtt | GUUGGAGAGGUUGCCGUACtt |
| PSKH1 | GAACCUGCACCGCUCCAUAtt | UAUGGAGCGGUGCAGGUUCtt |
| PSKH1seq2¹ | UUGGCCGAGGCAGCUUCAGtt | CUGAAGCUGCCUCGGCCAAtt |
| ITPKC | GUCCUGGGCUGAUAACCUCtt | GAGGUUAUCAGCCCAGGACtt |
| ROCK | UACAUGCCUGGUGGAGAUCtt | GAUCUCCACCAGGCAUGUAtt |
| BAI2 | GCUCUGCAGUAUGGCUGCCtt | GGCAGCCAUACUGCAGAGCtt |
| ULK1 | UUCUGUCUACCUGGUUAUGtt | CAUAACCAGGUAGACAGAAtt |
| DAGK | GAUCGUGCAGAUGAGUAACtt | GUUACUCAUCUGCACGAUCtt |
| STK6 | GCCGGUUCAGAAUCAGAAGtt CUUCUGAUUCUGAACCGGCtt | |
| FLJ13551 | CACCAAUUAGUUCAAAGCUtt | AGCUUUGAACUAAUUGGUGtt |
| GPR12 | AGCGCUCUGUCUCAUUUGCtt | GCAAAUGAGACAGAGCGCUtt |
| UK | AGCGCAAGACACUCUGUGGtt | CCACAGAGUGUCUUGCGCUtt |

TABLE 12
% Knockdown by RNAi of Kinases and GPCR's Examined
post transfection U251 and DU145 examined 24 hrs post
transfection whereas Hela and TF1 were
examined 48 hrs post transfection

| SiRNA | | | | | | |
|---|---|---|---|---|---|---|
| Oligomer | Accession No. | HELA | TF1 + GMCSF | TF1 − GMCSF | U251 | DU145 |
| GRAF | AB014521 | 75 | 56 | 73 | | 84.4 |
| MAPKK5 | U71087 | 73 | | 70 | | |
| P14KB | U81802 | 88.5 | | 40 | 36 | |
| EKI | AI003792 | 90 | 46 | 58 | | |
| PCTAIRE | AA535884 | 64 | 20 | 70 | | |
| PSKH1 | X71875 | — | | 60 | | |
| ROCK | AI125216 | 60 | | | | |
| MKNK | AI368064 | 40 | 65 | | | |
| EDG6 | AJ000429 | — | | 70 | | |
| MPSK1 | AF060798 | 84 | | | | |
| FLT4 | X69878 | — | | | | 85 |
| CDC42 | AI7617729 | 73 | | | | |
| GPR86 | AA142914 | — | | | | 74 |
| RBSK | AI823980 | 80 | | | | |
| NTKL | AI741642 | 89 | | | 37 | |
| MAK | AA058851 | — | | | | 19 |
| TLK2 | AF162667 | 14 | 10 | 35 | | |
| SURVIVIN | U75285 | 27 | 20 | | 47 | |

TABLE 13
Clonagenicity Assay - This table gives the percentage
survival of the indicated cell lines relative to the missense
in a clonagenicity assay, over 3 independent experiments.
As an internal reference, the known survival gene
‘survivin’ was also targeted by siRNA.

| | | | |
|---|---|---|---|
| siRNA Oligos | n = 1 | n = 2 | n = 3 |

Table (a) U251

| | | | |
|---|---|---|---|
| MS1 | 100 | 100 | 100 |
| SURB | 45.9 | 64.6 | 76.4 |
| LAMIN | 51 | — | 20 |
| BCL2 | 100 | — | 14.4 |
| C-RAF | — | — | — |
| PI3KR1 | 56.3 | — | 21.4 |
| GPR86 | 14.1 | 24.5 | — |
| BAI2 | 51.7 | 64.6 | — |
| GRAF | — | — | — |
| ULK1 | 64.3 | — | — |
| EKI1 | 62.3 | — | — |
| ROCK | 13.8 | 58.2 | 19.8 |
| UK | 47.7 | — | 48.1 |
| NTKL | 9.5 | 20.9 | 17.1 |
| RBSK | — | — | — |
| DAGK | — | — | — |
| GPR12 | — | — | — |
| ITPKC | — | — | — |
| STK6 | 12.8 | 51.5 | 25.8 |
| FLJ13 | — | — | — |
| MAK | 11.5 | 49.1 | 58.7 |
| PCTAIRE | 24.2 | 42.7 | 1.6 |
| RS6PK | 62.5 | — | 20.2 |
| TLK2 | — | — | 16.9 |
| CDC42 | 100 | — | 21.1 |
| EDG6 | — | — | — |
| MAPKK5 | 44.8 | 51.4 | 12.4 |
| P14KB | 12.3 | 65 | 28.3 |
| FLT4 | — | 63 | 30.9 |
| MPSK1 | — | — | — |
| MPSK1 SEQ 2 | — | — | 59.3 |
| CNK/PRK | 48.8 | — | 44.5 |
| CNK/PRK SEQ 2 | 35.2 | — | 57.5 |
| PSKH1 | 60.5 | — | 56.9 |
| PSKH1 SEQ 2 | 52.1 | 50.7 | 34.1 |
| MKNK | 33.6 | 59.5 | 69 |
| MKNK SEQ 2 | — | 43.6 | 52.1 |

Table (b) PC3

| | | | |
|---|---|---|---|
| MS1 | 100 | 100 | 100 |
| SURB | 39.2 | — | 83.6 |
| LAMIN | — | — | — |
| BCL2 | — | — | 24 |
| C-RAF | — | — | — |
| PI3KR1 | — | — | — |
| GPR86 | 73.1 | — | — |
| BAI2 | 67.3 | — | — |
| GRAF | — | — | — |
| ULK1 | 45.2 | 69.6 | 50.9 |
| EKI1 | 62 | 40.4 | 39.7 |
| ROCK | 50.4 | 32.3 | 22.8 |
| UK | 60.6 | — | 54.4 |
| NTKL | 11.6 | 45.7 | 14.4 |
| RBSK | — | — | — |
| DAGK | — | — | — |
| GPR12 | — | — | — |
| ITPKC | 45.1 | 51.3 | — |
| STK6 | — | — | — |
| FLJ13 | — | — | — |
| MAK | 69.6 | — | — |
| PCTAIRE | — | — | — |
| RS6PK | — | — | — |
| TLK2 | — | — | — |
| CDC42 | — | — | — |
| EDG6 | — | — | — |
| MAPKK5 | 30.5 | — | 8.3 |
| P14KB | — | — | 8.4 |
| FLT4 | — | 71.7 | 8.4 |
| MPSK1 | — | — | — |
| MPSK1 SEQ 2 | — | — | 16.7 |
| CNK/PRK | — | — | — |
| CNK/PRK SEQ 2 | — | — | 28.3 |
| PSKH1 | 29.8 | — | — |
| PSKH1 SEQ 2 | — | — | 21.8 |
| MKNK | — | — | 8.4 |
| MKNK SEQ 2 | — | — | 39.5 |

Table (c) A549

| | | | |
|---|---|---|---|
| MS1 | 100 | 100 | 100 |
| SURB | 28.5 | 62.4 | 67.3 |
| LAMIN | — | — | — |
| BCL2 | — | — | 40.8 |
| C-RAF | — | — | 59.1 |
| PI3KR1 | — | — | 38.3 |
| GPR86 | — | — | — |
| BAI2 | — | — | — |
| GRAF | — | — | — |
| ULK1 | — | — | — |
| EKI1 | 27.1 | 30.9 | 49.1 |
| ROCK | 16.3 | 28.4 | 16.8 |
| UK | — | — | — |
| NTKL | 18.9 | 45.3 | 28.1 |
| RBSK | — | — | — |
| DAGK | — | — | — |
| GPR12 | — | — | — |
| ITPKC | — | — | — |
| STK6 | 56.8 | 69.6 | 31.6 |
| FLJ13 | — | — | — |
| MAK | 51.6 | — | 42.8 |
| PCTAIRE | — | — | — |
| RS6PK | 79.8 | — | 71.7 |
| TLK2 | — | — | — |
| CDC42 | — | — | — |
| EDG6 | — | — | — |
| MAPKK5 | 48.5 | — | — |
| P14KB | — | — | 63.4 |
| FLT4 | 45.4 | — | 28.7 |
| MPSK1 | — | — | — |
| MPSK1 SEQ 2 | 39.1 | — | 26.1 |
| CNK/PRK | — | — | — |
| CNK/PRK SEQ 2 | 62.5 | — | 25.6 |
| PSKH1 | 51.7 | — | — |
| PSKH1 SEQ 2 | 52.3 | — | 28 |
| MKNK | 11.5 | 67.9 | 23.3 |
| MKNK SEQ 2 | — | — | 54.8 |

Table (d) DU145

| | | | |
|---|---|---|---|
| MS1 | 100 | 100 | 100 |
| SURB | 102 | 61.8 | 91 |
| LAMIN | — | — | — |
| BCL2 | — | — | — |
| C-RAF | — | 54.9 | — |
| PI3KR1 | — | — | 44.8 |
| GPR86 | — | — | — |
| BAI2 | — | — | — |
| GRAF | — | — | — |
| ULK1 | — | — | — |
| EKI1 | — | 62.2 | — |
| ROCK | 45.6 | 44.4 | — |
| UK | — | — | — |
| NTKL | 27.8 | 35.1 | 25.4 |
| RBSK | — | — | — |
| DAGK | — | — | — |
| GPR12 | — | 57.6 | — |
| ITPKC | — | — | — |
| STK6 | 48.1 | 45.3 | 34.8 |
| FLJ13 | — | — | — |
| MAK | — | 57.2 | 55.4 |
| PCTAIRE | — | — | — |
| RS6PK | — | — | — |
| TLK2 | — | — | — |
| CDC42 | — | — | — |
| EDG6 | — | — | 37.9 |
| MAPKK5 | — | — | 13.6 |
| P14KB | — | — | 26.1 |
| FLT4 | — | 52.7 | 32.6 |
| MPSK1 | — | — | — |
| MPSK1 SEQ 2 | — | — | 63.4 |
| CNK/PRK | 46.1 | 39 | 16.8 |
| CNK/PRK SEQ 2 | — | — | — |
| PSKH1 | — | — | — |
| PSKH1 SEQ 2 | — | — | 35.2 |
| MKNK | — | — | 33.9 |
| MKNK SEQ 2 | — | — | 53.6 |

Table (e) SKOV3

| | | | |
|---|---|---|---|
| MS1 | 100 | 100 | 100 |
| SURB | 43.9 | 90.8 | 85.7 |
| LAMIN | — | — | — |
| BCL2 | — | — | 32.1 |
| C-RAF | 42.5 | — | — |
| PI3KR1 | 55.3 | — | 41.7 |
| GPR86 | 31.1 | 55.8 | — |
| BAI2 | 44.2 | 25.4 | — |
| GRAF | 82.2 | — | — |
| ULK1 | 25.1 | 61.2 | — |
| EKI1 | 10.5 | 19.8 | 20.6 |
| ROCK | 11.7 | 30.4 | 40.7 |
| UK | 80.3 | — | — |
| NTKL | 6.2 | 18.8 | 14.9 |
| RBSK | — | — | — |
| DAGK | — | — | — |
| GPR12 | — | — | — |
| ITPKC | — | — | — |
| STK6 | 10.2 | 35.3 | 37.2 |
| FLJ13 | — | — | — |
| MAK | 25.6 | 40.3 | — |
| PCTAIRE | 68.6 | — | — |
| RS6PK | — | — | — |
| TLK2 | — | — | — |
| CDC42 | — | — | — |
| EDG6 | — | — | — |
| MAPKK5 | 18.5 | — | 53.7 |
| P14KB | 12.3 | 56 | 44.8 |
| FLT4 | — | 61 | 56.5 |
| MPSK1 | 57.2 | — | — |
| MPSK1 SEQ 2 | — | — | 73.8 |
| CNK/PRK | 13.8 | — | 50.1 |
| CNK/PRK SEQ 2 | 65.7 | — | 72.2 |
| PSKH1 | 48 | — | — |
| PSKH1 SEQ 2 | 42.3 | — | 45.1 |
| MKNK | 14 | — | 34.3 |
| MKNK SEQ 2 | — | — | 47.6 |

Table (f) A498

| | | | |
|---|---|---|---|
| MS1 | 100 | 100 | 100 |
| SURB | 35.9 | — | 75.9 |
| LAMIN | 61.9 | — | 8 |
| BCL2 | — | — | 12.3 |
| C-RAF | — | — | 14.8 |
| PI3KR1 | — | — | 10 |
| GPR86 | 112.6 | — | — |
| BAI2 | 66.2 | — | — |
| GRAF | 92.6 | — | 49.9 |
| ULK1 | 15.1 | — | 19.9 |
| EKI1 | 21.6 | — | 15.4 |
| ROCK | 14.3 | — | 8.3 |
| UK | — | — | — |
| NTKL | 12.3 | — | 7.2 |
| RBSK | 43.1 | — | 28.6 |
| DAGK | 25.2 | — | 37.9 |
| GPR12 | 22.2 | — | 45.7 |
| ITPKC | — | — | — |
| STK6 | 18.5 | — | 32.6 |
| FLJ13 | — | — | — |
| MAK | 19.4 | — | 51.6 |
| PCTAIRE | 24.9 | — | 48.4 |
| RS6PK | — | — | — |
| TLK2 | — | — | 42 |
| CDC42 | — | — | 29.2 |
| EDG6 | — | — | 24.6 |
| MAPKK5 | 10.2 | — | 6.9 |
| P14KB | 19.3 | — | 15.1 |
| FLT4 | — | — | 10 |
| MPSK1 | 44.7 | — | 20.9 |
| MPSK1 SEQ 2 | 39.3 | — | 8.9 |
| CNK/PRK | 9.5 | — | 7.2 |
| CNK/PRK SEQ 2 | 20 | — | 8.2 |
| PSKH1 | 54.8 | — | 10 |
| PSKH1 SEQ 2 | 36 | — | 6 |
| MKNK | 15.4 | — | 2.7 |
| MKNK SEQ 2 | 56.2 | — | 6 |

Table (g) HCT15

| | | | |
|---|---|---|---|
| MS1 | 100 | 100 | 100 |
| SURB | 71.3 | 44.5 | — |
| LAMIN | — | — | — |
| BCL2 | — | — | — |
| C-RAF | 83.5 | — | — |
| PI3KR1 | — | — | — |
| GPR86 | 78.5 | — | — |
| BAI2 | 77.2 | 37.5 | — |
| GRAF | — | — | — |
| ULK1 | — | — | — |
| EKI1 | 63.5 | 60.6 | — |
| ROCK | 61.9 | 34.6 | — |
| UK | 78.1 | 71.7 | — |
| NTKL | 59.5 | 55.3 | — |
| RBSK | — | — | — |
| DAGK | — | — | — |
| GPR12 | 71.1 | — | — |
| ITPKC | — | — | — |
| STK6 | 69.6 | — | — |
| FLJ13 | — | — | — |
| MAK | 75.9 | 67.3 | — |
| PCTAIRE | — | — | — |
| RS6PK | 85.2 | — | — |
| TLK2 | — | — | — |
| CDC42 | — | — | — |
| EDG6 | — | — | — |
| MAPKK5 | 65.3 | 61.2 | — |
| P14KB | — | — | — |
| FLT4 | 64.5 | 48.1 | — |
| MPSK1 | 68.3 | — | — |
| MPSK1 SEQ 2 | — | — | — |
| CNK/PRK | 75.9 | — | — |
| CNK/PRK SEQ 2 | — | — | — |
| PSKH1 | 28.2 | — | — |
| PSKH1 SEQ 2 | — | — | — |
| MKNK | 36 | — | — |
| MKNK SEQ 2 | — | — | — |

TABLE 14
PERCENTAGE SURVIVAL RELATIVE TO MS (%)

| | A549 | | HCT15 | | MCF7 | | U251 | PC3 |
|---|---|---|---|---|---|---|---|---|
| | n = 1 | n = 2 | n = 1 | n = 2 | n = 1 | n = 2 | n = 1 | n = 1 |
| PSKHI | 65.8 | 32.5 | 37.2 | 41 | 82.6 | 11.9 | / | / |
| CRAF | 57 | 24.2 | 70 | 65.1 | 89.7 | 98.2 | / | / |
| PRK | 91 | 39.6 | 38.6 | 72.3 | 98.2 | 66.3 | 13.5 | 10 |
| TLK2B | 65.8 | 36.7 | 67.3 | 115.6 | 83.7 | 99.5 | / | / |
| MPSKI | 59.4 | 19.5 | 42.3 | 62.2 | 35.8 | 30.3 | 18 | / |
| MKNK | 85 | 21.6 | 66.3 | 90.9 | 25.9 | 4 | / | / |
| SURB | 53 | 17.7 | 71.5 | 48.6 | 80.3 | 50.3 | / | / |

1. A method of identifying an agent that modulates the function of an apoptosis-associated protein that is encoded by a gene selected from Table 1B, comprising providing a preparation containing said encoded protein; incubating the preparation with a test agent to be screened under conditions to permit binding of the test agent to the protein; determining whether the test agent interacts with the protein by detecting the presence or absence of a signal generated from the interaction of the agent with the protein, and thereby determining whether the test agent modulates the function of the apoptosis-associated protein.

2. The method according to claim 1 wherein the preparation containing the protein comprises a cell expressing the protein.

3. The method according to claim 1 wherein the apoptosis-associated protein that is encoded by a gene selected from Table 1B is a protein kinase and determining whether the test agent interacts with the protein is by detecting a change in the phosphotransferase activity of the protein kinase.

4. The method according to claim 1 wherein the apoptosis-associated protein that is encoded by a gene selected from Table 1B is a cell surface receptor and the preparation containing said encoded protein comprises a cell expressing said encoded protein on its surface, said protein being associated with a second component capable of providing a detectable signal in response to the binding of an agent to said protein.

5. The method according to claim 1 wherein the apoptosis-associated protein that is encoded by a gene selected from Table 1B is a cell surface receptor and the preparation containing said encoded protein comprises a cell expressing said encoded protein on its surface, said protein being associated with a G-protein in response to the binding of an agent to said protein.

6. The method according to claim 1 wherein the apoptosis-associated protein that is encoded by a gene selected from Table 1B is a cell surface receptor and the preparation containing said encoded protein comprises a cell expressing said encoded protein on its surface, said protein being associated with a a Gi, Go, Gs, G₁₆, G₁₅, Gq or G₁₂₋₁₃ G-protein in response to the binding of an agent to said protein.

7. A process for determining whether a chemical compound specifically binds to and inhibits an apoptosis-associated protein that is encoded by a gene selected from Table 1B, which comprises contacting cells producing a second messenger response and expressing the protein that is encoded by a gene selected from Table 1B, wherein such cells do not normally express said protein, with the chemical compound under conditions suitable for inhibition of the protein, and measuring the second messenger response in the presence and in the absence of the chemical compound, a change in the second messenger response in the presence of the chemical compound indicating that the compound inhibits the apoptosis-associated protein that is encoded by a gene selected from Table 1B.

8. The process according to claim 7, wherein the second messenger response comprises chloride channel activation, a change in intracellular calcium ion levels, a release of inositol phosphate, a release of arachidonic acid, GTPγS binding, activation of MAP kinase, cAMP accumulation, a change in intracellular potassium ion levels, or a change in intracellular sodium ion levels.

9. The process according to claim 7 wherein the second messenger response is measured by a change in reporter gene activity.

10. The process according to claim 7 wherein the second messenger response is measured by a change in reporter gene activity, said reporter gene being selected from secreted alkaline phosphatase, luciferase, and β-galactosidase.

11. A method for identifying an agent that inhibits tumor cell proliferation, which comprises determining whether a test agent modulates the activity or expression of a protein that is encoded by a gene selected from Table 1B, and determining whether any agent that modulates said activity or expression is an inhibitor of tumor proliferation.

12. The method according to claim 11, wherein the test agent is selected from a low molecular weight organic molecule, an antibody or antibody fragment, an antisense oligonucleotide, a small inhibitory dsRNA, and a ribozyme.


SEQUENCE LISTING (226 sequences)

SEQ ID NO: 1 - protein, 16 aa, Artificial sequence
  Penetratin
RQIKIWFQNRRMKWKK

SEQ ID NO: 2 - DNA, 21 bp, Artificial sequence
  missense control siRNA oligomer  (MS, sense)
ugagaaugugaugcgcguctt

SEQ ID NO: 3 - DNA, 21 bp, Artificial sequence
  missense control siRNA oligomer (MS, antisense)
gacgcgcaucacauucucatt

SEQ ID NO: 4 - DNA, 21 bp, Artificial sequence
  siRNA oligomer (Survivin (Survivin B, SurB, SURB, SUR), sense)
gaacuggcccuucuuggagtt

SEQ ID NO: 5 - DNA, 21 bp, Artificial sequence
  siRNA oligomer (Survivin (Survivin B, SurB, SURB, SUR), antisense)
cuccaagaagggccaguuctt

SEQ ID NO: 6 - DNA, 21 bp, Artificial sequence
  siRNA oligomer (PI3KR1, sense)
augaucgaugugcacguuutt

SEQ ID NO: 7 - DNA, 21 bp, Artificial sequence
  siRNA oligomer (PI3KR1, antisense)
aaacgugcacaucgaucautt

SEQ ID NO: 8 - DNA, 21 bp, Artificial sequence
  siRNA oligomer (BCL2, sense)
guacauccauuauaagcugtt

SEQ ID NO: 9 - DNA, 21 bp, Artificial sequence
  siRNA oligomer  (BCL2, antisense)
cagcuuauaauggauguactt

SEQ ID NO: 10 - DNA, 21 bp, Artificial sequence
  siRNA oligomer (c-Raf (CRAF), sense)
uaguucagcaguuuggcuatt

SEQ ID NO: 11 - DNA, 21 bp, Artificial sequence
  siRNA oligomer (c-Raf (CRAF), antisense)
uagccaaacugcugaacuatt

SEQ ID NO: 12 - DNA, 20 bp, Artificial sequence
  QPCR Forward Primer OAS1 (NM_002534)
gcgccccaccaagctcaaga

SEQ ID NO: 13 - DNA, 23 bp, Artificial sequence
  QPCR Reverse Primer OAS1 (NM_002534)
gtccgaaatccctgggctgtgtt

SEQ ID NO: 14 - DNA, 23 bp, Artificial sequence
  QPCR Forward Primer GBP1 (NM_002503)
tatggtggtggtggcaattgtgg

SEQ ID NO: 15 - DNA, 19 bp, Artificial sequence
  QPCR Reverse Primer GBP1 (NM_002503)
acggccagggcgaagatcc

SEQ ID NO: 16 - DNA, 21 bp, Artificial sequence
  QPCR forward primer (MAK)
gggagctggtggccatcaaaa

SEQ ID NO: 17 - DNA, 22 bp, Artificial sequence
  QPCR reverse primer (MAK)
tggataaaagccagcccttgca

SEQ ID NO: 18 - DNA, 20 bp, Artificial sequence
  QPCR forward primer (GPR86)
tgagcggtgccccagagaca

SEQ ID NO: 19 - DNA, 23 bp, Artificial sequence
  QPCR reverse primer (GPR86)
cagggtgccaggtgtgagtcaga

SEQ ID NO: 20 - DNA, 19 bp, Artificial sequence
  QPCR forward primer (PCTAIRE)
gccgctcagccgcatgtcc

SEQ ID NO: 21 - DNA, 20 bp, Artificial sequence
  QPCR reverse primer (PCTAIRE)
ggcgctccctcctcgtgctc

SEQ ID NO: 22 - DNA, 22 bp, Artificial sequence
  QPCR forward primer (GRAF)
cagcgaagcggaagtttgcaga

SEQ ID NO: 23 - DNA, 20 bp, Artificial sequence
  QPCR reverse primer (GRAF)
cttccttggcagccccgatc

SEQ ID NO: 24 - DNA, 21 bp, Artificial sequence
  QPCR forward primer (MPSK1)
cgcgctgtgtgtctgctctcg

SEQ ID NO: 25 - DNA, 22 bp, Artificial sequence
  QPCR reverse primer (MPSK1)
gcgaaggatgttggggtgattg

SEQ ID NO: 26 - DNA, 20 bp, Artificial sequence
  QPCR forward primer (RBS5PK)
gccgccaaaaaagtgcctgc

SEQ ID NO: 27 - DNA, 23 bp, Artificial sequence
  QPCR reverse primer (RBS5PK)
tccttcatcattgcactcctggc

SEQ ID NO: 28 - DNA, 22 bp, Artificial sequence
  QPCR forward primer (TLK2)
gcagttcccgccaaagccagta

SEQ ID NO: 29 - DNA, 21 bp, Artificial sequence
  QPCR reverse primer (TLK2)
ggacgccccagaggttgatgc

SEQ ID NO: 30 - DNA, 18 bp, Artificial sequence
  QPCR forward primer (EK1)
cgggccgggctcagttca

SEQ ID NO: 31 - DNA, 21 bp, Artificial sequence
  QPCR reverse primer (EK1)
cggcggagactaccaccacga

SEQ ID NO: 32 - DNA, 23 bp, Artificial sequence
  QPCR forward primer (MKNK)
caagcagggcacagtcggagtag

SEQ ID NO: 33 - DNA, 21 bp, Artificial sequence
  QPCR reverse primer (MKNK)
cggctggcttctcgctcattg

SEQ ID NO: 34 - DNA, 21 bp, Artificial sequence
  QPCR forward primer (NTKL)
ggcagccccgtgtccatcttc

SEQ ID NO: 35 - DNA, 22 bp, Artificial sequence
  QPCR reverse primer  (NTKL)
ccagcctccactctcgccttga

SEQ ID NO: 36 - DNA, 23 bp, Artificial sequence
  QPCR forward primer (CDC42)
caaagcgagaacggcataacgag

SEQ ID NO: 37 - DNA, 22 bp, Artificial sequence
  QPCR reverse primer  (CDC42)
ccgggcatctttctcgtcactg

SEQ ID NO: 38 - DNA, 18 bp, Artificial sequence
  QPCR forward primer (RBSK)
ggcggcgtctggggaacc

SEQ ID NO: 39 - DNA, 23 bp, Artificial sequence
  QPCR reverse primer (RBSK)
agccgagcagcttggacacactg

SEQ ID NO: 40 - DNA, 21 bp, Artificial sequence
  QPCR forward primer (EDG6)
cggcggtcaaccccatcatct

SEQ ID NO: 41 - DNA, 20 bp, Artificial sequence
  QPCR reverse primer (EDG6)
cccgcatccgaaagctgagc

SEQ ID NO: 42 - DNA, 21 bp, Artificial sequence
  QPCR forward primer (CNK/PRK)
cgcggacctgagctggagatg

SEQ ID NO: 43 - DNA, 17 bp, Artificial sequence
  QPCR reverse primer (CNK/PRK)
tggcgacgcggctctgc

SEQ ID NO: 44 - DNA, 22 bp, Artificial sequence
  QPCR forward primer (MAPKK5)
cgggccgcagttactcttcagg

SEQ ID NO: 45 - DNA, 22 bp, Artificial sequence
  QPCR reverse primer (MAPKK5)
ccggcccgagtattcaccttca

SEQ ID NO: 46 - DNA, 18 bp, Artificial sequence
  QPCR forward primer (P14KB)
cggagggggtcggggaac

SEQ ID NO: 47 - DNA, 20 bp, Artificial sequence
  QPCR reverse primer (P14KB)
gcggcccccatctcatcttc

SEQ ID NO: 48 - DNA, 21 bp, Artificial sequence
  QPCR forward primer (FLT4)
tgccgtgaaccccatcgagag

SEQ ID NO: 49 - DNA, 22 bp, Artificial sequence
  QPCR reverse primer (FLT4)
cgtggacaggttgaggcggtac

SEQ ID NO: 50 - DNA, 21 bp, Artificial sequence
  QPCR forward primer (PSKH1)
cccgagccacccaaggatgtc

SEQ ID NO: 51 - DNA, 21 bp, Artificial sequence
  QPCR reverse primer (PSKH1)
ggccctgcgtggtggttctga

SEQ ID NO: 52 - DNA, 20 bp, Artificial sequence
  QPCR forward primer (ITPKC)
agccgggacagcagcgacct

SEQ ID NO: 53 - DNA, 22 bp, Artificial sequence
  QPCR reverse primer (ITPKC)
tttgcttgggcctctcggtctc

SEQ ID NO: 54 - DNA, 19 bp, Artificial sequence
  QPCR forward primer (ROCK)
gtgggcttgggaaacgctc

SEQ ID NO: 55 - DNA, 26 bp, Artificial sequence
  QPCR reverse primer (ROCK)
tctgcattggagctagttctgttatc

SEQ ID NO: 56 - DNA, 17 bp, Artificial sequence
  QPCR forward primer (GRAF)
gatagtccgcacttccg

SEQ ID NO: 57 - DNA, 18 bp, Artificial sequence
  QPCR Reverse Primer (GRAF)
gagtgacttcccgtcctt

SEQ ID NO: 58 - DNA, 18 bp, Artificial sequence
  QPCR Forward Primer (ULK1)
gacttccaggaaatggct

SEQ ID NO: 59 - DNA, 18 bp, Artificial sequence
  QPCR Reverse Primer (ULK1)
agagcctgatggtgtcct

SEQ ID NO: 60 - DNA, 18 bp, Artificial sequence
  QPCR Forward Primer (EKI)
cgtcgtggtggtagtctc

SEQ ID NO: 61 - DNA, 18 bp, Artificial sequence
  QPCR Reverse Primer (EKI)
gatgctcctcctgatcct

SEQ ID NO: 62 - DNA, 18 bp, Artificial sequence
  QPCR Forward Primer (ROCK1)
gcataaatccaccaggaa

SEQ ID NO: 63 - DNA, 18 bp, Artificial sequence
  QPCR Reverse Primer (ROCK1)
atgtccctttcttcccag

SEQ ID NO: 64 - DNA, 18 bp, Artificial sequence
  QPCR Forward Primer (NTKL)
tacctcaaggcgagagtg

SEQ ID NO: 65 - DNA, 18 bp, Artificial sequence
  QPCR Reverse Primer (NTKL)
cagtcgttgaccaggaag

SEQ ID NO: 66 - DNA, 18 bp, Artificial sequence
  QPCR Forward Primer (RBSK)
atacggaggatctgaggg

SEQ ID NO: 67 - DNA, 18 bp, Artificial sequence
  QPCR Reverse Primer (RBSK)
tccaaagaagttgctgga

SEQ ID NO: 68 - DNA, 20 bp, Artificial sequence
  QPCR Forward Primer (DAGK)
ggaaggtgacgctcaccaag

SEQ ID NO: 69 - DNA, 21 bp, Artificial sequence
  QPCR Reverse Primer (DAGK)
acatgaaattgcagacgtcgc

SEQ ID NO: 70 - DNA, 18 bp, Artificial sequence
  QPCR Forward Primer (ITPKC)
cagacggacagactgagc

SEQ ID NO: 71 - DNA, 18 bp, Artificial sequence
  QPCR Reverse Primer (ITPKC)
tccattctagatgcgtcc

SEQ ID NO: 72 - DNA, 18 bp, Artificial sequence
  QPCR Forward Primer (UKH)
tgcagtacgatgtgcttg

SEQ ID NO: 73 - DNA, 18 bp, Artificial sequence
  QCPR Review Primer (UKH)
cagcactttcctggtctg

SEQ ID NO: 74 - DNA, 18 bp, Artificial sequence
  QPCR Forward Primer (BAI2)
cctgctgaggccgatttg

SEQ ID NO: 75 - DNA, 22 bp, Artificial sequence
  QPCR Reverse Primer (BAI2)
tttcactttcggttcctcttcc

SEQ ID NO: 76 - DNA, 21 bp, Artificial sequence
  QPCR Forward Primer (GPR12)
aaggtcaatttaagcgggctg

SEQ ID NO: 77 - DNA, 20 bp, Artificial sequence
  QPCR Reverse Primer (GPR12)
tctggctctacggcaggaac

SEQ ID NO: 78 - DNA, 18 bp, Artificial sequence
  QPCR Forward Primer (GPR86)
aggtgacactggaagcaa

SEQ ID NO: 79 - DNA, 18 bp, Artificial sequence
  QPCR Reverse Primer (GPR86)
cactgtgtagagggctgg

SEQ ID NO: 80 - DNA, 19 bp, Artificial sequence
  QPCR Forward Primer (BcI2)
cacgctgggagaacagggt

SEQ ID NO: 81 - DNA, 18 bp, Artificial sequence
  QPCR  REverse Primer (Bcl2)
cacatctcccgcatccca

SEQ ID NO: 82 - DNA, 20 bp, Artificial sequence
  QPCR Forward Primer (SurvivinB)
tcaaggaccaccgcatctct

SEQ ID NO: 83 - DNA, 20 bp, Artificial sequence
  (SurvivinB)
cagtggatgaagccagcctc

SEQ ID NO: 84 - DNA, 20 bp, Artificial sequence
  QPCR Forward Primer (GAPDH)
cgaccactttgtcaagctca

SEQ ID NO: 85 - DNA, 20 bp, Artificial sequence
  QCPR Reverse Primer (GAPDH)
gggtcttactccttggaggc

SEQ ID NO: 86 - DNA, 21 bp, Artificial sequence
  siRNA sense oligonucleotide (GRAF)
gcggaaguuugcagauucctt

SEQ ID NO: 87 - DNA, 21 bp, Artificial sequence
  siRNA antisense oligonucleotide (GRAF)
ggaaucugcaaacuuccgctt

SEQ ID NO: 88 - DNA, 21 bp, Artificial sequence
  siRNA sense oligonucleotide (ULK1)
ggaacugaaacaugaaaactt

SEQ ID NO: 89 - DNA, 21 bp, Artificial sequence
  siRNA antisense oligonucleotide (ULK1)
guuuucauguuucaguucctt

SEQ ID NO: 90 - DNA, 21 bp, Artificial sequence
  siRNA sense oligonucleotide (EKI)
gcacuggauccaaagcaugtt

SEQ ID NO: 91 - DNA, 21 bp, Artificial sequence
  siRNA antisense oligonucleotide (EKI)
caugcuuuggauccagugctt

SEQ ID NO: 92 - DNA, 21 bp, Artificial sequence
  siRNA sense oligonucleotide (ROCK)
uacaugccugguggagauctt

SEQ ID NO: 93 - DNA, 21 bp, Artificial sequence
  siRNA antisense Oligonucleotide (ROCK)
gaucuccaccaggcauguatt

SEQ ID NO: 94 - DNA, 21 bp, Artificial sequence
  siRNA sense oligonucleotide (NTKL)
uguggagcugaugaagcactt

SEQ ID NO: 95 - DNA, 21 bp, Artificial sequence
  siRNA antisense oligonucleotide (NTKL)
gugcuucaucagcuccacatt

SEQ ID NO: 96 - DNA, 21 bp, Artificial sequence
  siRNA sense oligonucleotide (RBSK)
cguccuggagugacaaaugtt

SEQ ID NO: 97 - DNA, 21 bp, Artificial sequence
  siRNA antisense oligonucleotide (RBSK)
cauuugucacuccaggacgtt

SEQ ID NO: 98 - DNA, 21 bp, Artificial sequence
  siRNA sense oligonucleotide (DAGK)
ggcugcacaacaagggugutt

SEQ ID NO: 99 - DNA, 21 bp, Artificial sequence
  siRNA antisense oligonucleotide (DAGK)
acacccuuguugugcagcctg

SEQ ID NO: 100 - DNA, 21 bp, Artificial sequence
  siRNA sense oligonucleotide (ITPKC)
guccugggcugauaaccuctt

SEQ ID NO: 101 - DNA, 21 bp, Artificial sequence
  siRNA antisense oligonucleotide (ITPKC)
gagguuaucagcccaggactt

SEQ ID NO: 102 - DNA, 21 bp, Artificial sequence
  siRNA sense oligonucleotide (UKH)
agcgcaagacacucuguggtt

SEQ ID NO: 103 - DNA, 21 bp, Artificial sequence
  siRNA antisense oligonucleotide (UKH)
ccacagagugucuugcgcutt

SEQ ID NO: 104 - DNA, 21 bp, Artificial sequence
  siRNA sense oligonucleotide (BAI2)
ggaccuguuugguaccauctt

SEQ ID NO: 105 - DNA, 21 bp, Artificial sequence
  siRNA antisense oligonucleotide (BAI2)
gaugguaggaaagaggucctg

SEQ ID NO: 106 - DNA, 21 bp, Artificial sequence
  siRNA sense oligonucleotide (GPR12)
ggacggucacguuuaccuatt

SEQ ID NO: 107 - DNA, 21 bp, Artificial sequence
  siRNA antisense oligonucleotide (GPR12)
uagguaaacgugaccgucctc

SEQ ID NO: 108 - DNA, 21 bp, Artificial sequence
  siRNA sense oligonucleotide (GPR86)
aaacacuuugguggccgactt

SEQ ID NO: 109 - DNA, 21 bp, Artificial sequence
  siRNA antisense oligonucleotide (GPR86)
gucggccaccaaaguguuutt

SEQ ID NO: 110 - DNA, 21 bp, Artificial sequence
  sense oligonucleotide (MAK)
gaagccaagcauggguguutt

SEQ ID NO: 111 - DNA, 21 bp, Artificial sequence
  antisense oligonucleotide (MAK)
aacacccaugcuuggcuuctt

SEQ ID NO: 112 - DNA, 21 bp, Artificial sequence
  sense oligonucleotide (GPR86)
aaacacuuugguggccgactt

SEQ ID NO: 113 - DNA, 21 bp, Artificial sequence
  antisense oligonucleotide (GPR86)
gucggccaccaaaguguuutt

SEQ ID NO: 114 - DNA, 21 bp, Artificial sequence
  sense oligonucleotide (PCTAIRE)
gucagugcccacaaagacutt

SEQ ID NO: 115 - DNA, 21 bp, Artificial sequence
  antisense oligonucleotide (PCTAIRE)
agucuuugugggcacugactt

SEQ ID NO: 116 - DNA, 21 bp, Artificial sequence
  sense oligonucleotide (GRAF)
gcggaaguuugcagauucctt

SEQ ID NO: 117 - DNA, 21 bp, Artificial sequence
  antisence oligonucleotide (GRAF)
ggaaucugcaaacuuccgctt

SEQ ID NO: 118 - DNA, 21 bp, Artificial sequence
  sene oligonucleotide (MPSK1)
ggguuaugcccacagagactt

SEQ ID NO: 119 - DNA, 21 bp, Artificial sequence
  antisense oligonucleotide (MPSK1)
gucucugugggcauaaccctt

SEQ ID NO: 120 - DNA, 21 bp, Artificial sequence
  sense oligonucleotide (MPSK1seq2)
gccgacaugcaucgccucutt

SEQ ID NO: 121 - DNA, 21 bp, Artificial sequence
  antisense oligonucleotide (MPSK1seq2)
agaggcgaugcaugucggctt

SEQ ID NO: 122 - DNA, 21 bp, Artificial sequence
  sense oligonucleotide (RBS6PK)
cguccuggagugacaaaugtt

SEQ ID NO: 123 - DNA, 21 bp, Artificial sequence
  antisense oligonucleotide (RBS6PK)
cauuugucacuccaggacgtt

SEQ ID NO: 124 - DNA, 21 bp, Artificial sequence
  sense oligonucleotide (TLK2A)
guguuccaccaguugcacgtt

SEQ ID NO: 125 - DNA, 21 bp, Artificial sequence
  antisense oligonucleotide (TLK2A)
cgugcaacugguggaacactt

SEQ ID NO: 126 - DNA, 21 bp, Artificial sequence
  antisense oligonucleotide (TLK2B)
gauggcguguagagauaagtt

SEQ ID NO: 127 - DNA, 21 bp, Artificial sequence
  antisense oligonucleotide (TLK2B)
cuuaucucuacacgccauctt

SEQ ID NO: 128 - DNA, 21 bp, Artificial sequence
  sense oligonucleotide (EKI1)
gcacuggauccaaagcaugtt

SEQ ID NO: 129 - DNA, 21 bp, Artificial sequence
  antisense oligonucleotide (EKI1)
caugcuuuggauccagugctt

SEQ ID NO: 130 - DNA, 21 bp, Artificial sequence
  sense oligonucleotide (MKNK)
uacauggccccugagguagtt

SEQ ID NO: 131 - DNA, 21 bp, Artificial sequence
  anitsense oligonucleotide (MKNK)
cuaccucaggggccauguatt

SEQ ID NO: 132 - DNA, 21 bp, Artificial sequence
  sense oligonucleotide (MKNKseq2)
auugcaaggagguuccauctt

SEQ ID NO: 133 - DNA, 21 bp, Artificial sequence
  antisense oligonucleotide (MKNKseq2)
gauggaaccuccuugcaautt

SEQ ID NO: 134 - DNA, 21 bp, Artificial sequence
  sense oligonucleotide (NTKL)
uguggagcugaugaagcactt

SEQ ID NO: 135 - DNA, 21 bp, Artificial sequence
  antisense oligonucleotide (NTKL)
gugcuucaucagcuccacatt

SEQ ID NO: 136 - DNA, 21 bp, Artificial sequence
  sense oligonucleotide (CDC42)
gcucagcuugaugaugcugtt

SEQ ID NO: 137 - DNA, 21 bp, Artificial sequence
  antisense oligonucleotide (CDC42)
cagcaucaucaagcugagctt

SEQ ID NO: 138 - DNA, 21 bp, Artificial sequence
  sense oligonucleotide (RBSK)
gaccuuccgcuuacucugutt

SEQ ID NO: 139 - DNA, 21 bp, Artificial sequence
  antisense oligonucleotide (RBSK)
acagaguaagcggaagguctt

SEQ ID NO: 140 - DNA, 21 bp, Artificial sequence
  sense oligonucleotide (EDG6)
caucacgcugagugaccugtt

SEQ ID NO: 141 - DNA, 21 bp, Artificial sequence
  antisense oligonucleotide (EDG6)
caggucacucagcgugaugtt

SEQ ID NO: 142 - DNA, 21 bp, Artificial sequence
  sense oligonucleotide (CNK/PRK)
ucguagugcuuguacuuactt

SEQ ID NO: 143 - DNA, 21 bp, Artificial sequence
  antisense oligonucleotide (CNK/PRK)
guaaguacaagcacuacgatt

SEQ ID NO: 144 - DNA, 21 bp, Artificial sequence
  sense oligonucleotide (CNK/PRKseq2)
cagaaagacugugcacuactt

SEQ ID NO: 145 - DNA, 21 bp, Artificial sequence
  antisense oligonucleotide (CNK/PRKseq2)
guagugcacagucuuucugtt

SEQ ID NO: 146 - DNA, 21 bp, Artificial sequence
  sense oligonucleotide (MAPKK5)
gaggacagguuaagcugugtt

SEQ ID NO: 147 - DNA, 21 bp, Artificial sequence
  antisense oligonucleotide (MAPKK5)
cacagcuuaaccuguccuctt

SEQ ID NO: 148 - DNA, 21 bp, Artificial sequence
  sense oligonucleotide (P14KB)
gcuacggaagcugauccuctt

SEQ ID NO: 149 - DNA, 21 bp, Artificial sequence
  antisense oligonucleotide (P14KB)
gaggaucagcuuccguagctt

SEQ ID NO: 150 - DNA, 21 bp, Artificial sequence
  sense oligonucleotide (FLT4)
guacggcaaccucuccaactt

SEQ ID NO: 151 - DNA, 21 bp, Artificial sequence
  antisense oligonucleotide (FLT4)
guuggagagguugccguactt

SEQ ID NO: 152 - DNA, 21 bp, Artificial sequence
  sense oligonucleotide (PSKH1)
gaaccugcaccgcuccauatt

SEQ ID NO: 153 - DNA, 21 bp, Artificial sequence
  antisense oligonucleotide (PSKH1)
uauggagcggugcagguuctt

SEQ ID NO: 154 - DNA, 21 bp, Artificial sequence
  sense oligonucleotide (PSKH1seq2)
uuggccgaggcagcuucagtt

SEQ ID NO: 155 - DNA, 21 bp, Artificial sequence
  antisense oligonucleotide (PSKH1seq2)
cugaagcugccucggccaatt

SEQ ID NO: 156 - DNA, 21 bp, Artificial sequence
  sense oligonucleotide (ITPKC)
guccugggcugauaaccuctt

SEQ ID NO: 157 - DNA, 21 bp, Artificial sequence
  antisense oligonucleotide (ITPKC)
gagguuaucagcccaggactt

SEQ ID NO: 158 - DNA, 21 bp, Artificial sequence
  sense oligonucleotide (ROCK)
uacaugccugguggagauctt

SEQ ID NO: 159 - DNA, 21 bp, Artificial sequence
  antisense oligonucleotide (ROCK)
gaucuccaccaggcauguatt

SEQ ID NO: 160 - DNA, 21 bp, Artificial sequence
  sense oligonucleotide (RAI2)
gcucugcaguauggcugcctt

SEQ ID NO: 161 - DNA, 21 bp, Artificial sequence
  antisense oligonucleotide (BAI2)
ggcagccauacugcagagctt

SEQ ID NO: 162 - DNA, 21 bp, Artificial sequence
  sense oligonucleotide (ULK1)
uucugucuaccugguuaugtt

SEQ ID NO: 163 - DNA, 21 bp, Artificial sequence
  antisense oligonucleotide (ULK1)
cauaaccagguagacagaatt

SEQ ID NO: 164 - DNA, 21 bp, Artificial sequence
  sense oligonucleotide (DAGK)
gaucgugcagaugaguaactt

SEQ ID NO: 165 - DNA, 21 bp, Artificial sequence
  antisense oligonucleotide (DAGK)
guuacucaucugcacgauctt

SEQ ID NO: 166 - DNA, 21 bp, Artificial sequence
  sense oligonucleotide (STK6)
gccgguucagaaucagaagtt

SEQ ID NO: 167 - DNA, 21 bp, Artificial sequence
  antisense oligonucleotide (STK6)
cuucugauucugaaccggctt

SEQ ID NO: 168 - DNA, 21 bp, Artificial sequence
  sense oligonucleotide (FLJ13551)
caccaauuaguucaaagcutt

SEQ ID NO: 169 - DNA, 21 bp, Artificial sequence
  antisense oligonucleotide (FLJ13551)
agcuuugaacuaauuggugtt

SEQ ID NO: 170 - DNA, 21 bp, Artificial sequence
  sense oligonucleotide (GPR12)
agcgcucugucucauuugctt

SEQ ID NO: 171 - DNA, 21 bp, Artificial sequence
  antisense oligonucleotide (GPR12)
gcaaaugagacagagcgcutt

SEQ ID NO: 172 - DNA, 21 bp, Artificial sequence
  sense oligonucleotide (UK)
agcgcaagacacucuguggtt

SEQ ID NO: 173 - DNA, 21 bp, Artificial sequence
  antisense oligonucleotide (UK)
ccacagagugucuugcgcutt

SEQ ID NO: 174 - DNA, 4065 bp, Homo sapiens
atgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaa

SEQ ID NO: 175 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 176 - DNA, 3823 bp, Homo sapiens
ataagaatggaagtgttgtttccttgcccgattccttcatgctatatctcatgaacctctgtaatcttgggggagagactatatttaatgatgacaaacctgtcaccagtgtagcaacaacagtgtgaggacaaaagcaaataaaaattaagaagcgttcaaatttatattcaacaaggaagtcatttcaatcaacaacttctgctgcattatttttccaagatgaaccgatacacaaccatgagacagttgggggacggcacgtatgggagtgtgcttatgggcaagagtaatgaatccggggagctggtggccatcaaaaggatgaagagaaagttctattcttgggatgaatgcatgaacttgagagaagttaagtctctgaagaaacttaatcatgccaatgttattaaattgaaagaagttatcagagaaaatgaccatctttattttatatttgaatatatgaaagaaaacctctatcaattaatgaaagacagaaacaagttgttccctgaatcagtcatcagaaatattatgtatcaaatattgcaagggctggcttttatccataaacatggcttttttcatagggacatgaaaccagaaaacttgctttgtatgggtccagagcttgtgaaaattgctgattttggacttgcaagagaattaaggtcacagccaccatacactgattatgtatctaccagatggtatcgtgcccctgaagttttactgagatcttcagtttatagttctcccattgatgtgtgggctgttggaagtatcatggctgaactctatatgttaaggccacttttcccagggacaagtgaggtcgatgaaatctttaaaatttgccaagttttagggactcccaaaaaaagtgactggccagaaggataccagctggcatcctctatgaacttccgttttccccagtgtgttcctataaacttaaaaactcttattcccaatgccagtaatgaagctattcagctcatgaccgaaatgttgaattgggatccaaagaaacgaccgacagcaagccaggcattgaaacacccatattttcaagttggtcaggtattaggcccttcgtcaaatcatctggaatcaaaacagtctttaaataagcagctgcaaccattagaatcaaagccatctttagttgaggtagagcctaagcctctgccggatataatcgatcaggttgttggacaaccccagccaaaaactagccagcagccactgcagcccattcagccgccacagaacctgagcgtccagcaacctccaaagcaacagagtcaggagaaaccgccacaaacgctattcccgagcatcgtcaaaaacatgccaactaagccaaatggcacactgagtcataaaagtggtaggaggcgttggggtcagactatcttcaagtctggagatagctgggaagagttggaggactatgatttcggagcctcccattccaagaagccaagcatgggtgtttttaaagaaaaaaggaaaaaagattctccatttcggcttccagagccagtaccctcaggctccaaccactcgacaggggaaaacaagagcttacctgctgttacttccctaaaatctgattccgaattgtcaactgctccaacctctaaacagtactacttgaaacaatcaagatatcttccaggtgtgaatcccaagaaggtgtccttgatagccagtggaaaggaaataaacccccacacttggagcaaccagttattccccaagtcactgggacccgttggggcagaacttgctttcaaaaggagcaatgcaggaaatcttggaagttatgctacttacaatcagtcaggatatattccttcctttctcaaaaaagaagtgcagtcagctggccagaggatccacttagcacctctcaatgcaacggcttcagaatatacctggaacacaaaaactggtcgggggcagttttcaggacgtacttataatcctacagcaaaaaacctaaatattgtgaaccgtgcacagcccattccctcagtgcatgggaggacagactgggtggccaagtatggaggccaccggtaggagtctatggtgtgaaaccctacagcattgctccgtagagtacgtgcaagttccttgaccctgggaaatgtctacaaatgtctatttctactgagttctggaagaaatatgcaaaagtgggtacttggaagggcaaaaatcatcccctattttacttatttccaagaaatgcattttcttagcatcattgcccacagtgttgatatatgggtaggatgttacaaagtattgaataaactatttgccaaagtatgaagtatttgatctacaatttaataaatagtaaatccaataagaacccttaaaaaaaaacaacttccagaaaatgtttagagtgttttagttttcatttgttgtatgtgcccaaatggtttagtagttcttcactttgctgtggttggcttaagaggtttgtcttttgtttttgtacagcagttgggccaacctttgctggctgtcagctgtggttcttatttgactaaggctctgcgctgccattttgtcaatgacagaaagattgcaattatttgtttttctactttgaagctatgggagcgatggtaatttcaatcttgagcaggatatctttttttttttttttgaggcgatgtttcactcttgtgtccaggctggagtgcagtggcactatcttggcttactgcaacctccacctcctgggttcaagtgattctcctgcctcagcctcccgagtagctgggattagaggcgtgtgccaccatgcctggctaatttttgtagtattagtagagacagggtttcaccatgttggtcaggctcaggctggtctcaaactcctgacctcaggtgatccacctgcctcggcctcccaaagtgcagggcttacaggtgtgagccacagtgcccagctttttttttttttttttagaaataaatttatctttaacatagagaatatcaaagttatgccacttgattttagtggacggcttgtgttttgatcttgaccagtacagttcccatgaattcaaggtgacaatcaagcccaatggcttgatgacttcaatagtaaccaagtcaaggttctcttggctggacatcattaagaaagttctggaaactgtgtttgtttgatgctggttcattggacttttcaaattgttttgtttctgtgtccctaccagacacaaagatgaagtgtgccagctggttcccccaagccagctcatgctgctgaccactgactcagctctgaccttcacatttgctctgaagcaagtgcgttcagctgctggggcagtgatatcacatagtacatatattatttccttagtttatttccaaactggtattttaaatagacacttcgaactttgggctactctgtttaaatctgccactttctggactggaccttagtactgtaaattctttttaaagaataataatgttaccaactgctgagatttttatgtattttgtgactttgtaacaactgctattgtaataagtgtcatcttgtgggcattatacaaaggcatattataaaataataatgatatttttgtatagaagagtcaactgttcagatgtaagatgttgaaaaatgttaaaatctaaagagtaatttatcctagtggtaatggttatatgtatttgtacagtttaaattaatgtctcaaagctgtgcagtcttttgttactgggacacttttaaactctgaataggcattaaaaaaaatatggctaa

SEQ ID NO: 177 - protein, 623 aa, Homo sapiens
MNRYTTMRQLGDGTYGSVLMGKSNESGELVAIKRMKRKFYSWDECMNLREVKSLKKLNHANVIKLKEVIRENDHLYFIFEYMKENLYQLMKDRNKLFPESVIRNIMYQILQGLAFIHKHGFFHRDMKPENLLCMGPELVKIADFGLARELRSQPPYTDYVSTRWYRAPEVLLRSSVYSSPIDVWAVGSIMAELYMLRPLFPGTSEVDEIFKICQVLGTPKKSDWPEGYQLASSMNFRFPQCVPINLKTLIPNASNEAIQLMTEMLNWDPKKRPTASQALKHPYFQVGQVLGPSSNHLESKQSLNKQLQPLESKPSLVEVEPKPLPDIIDQVVGQPQPKTSQQPLQPIQPPQNLSVQQPPKQQSQEKPPQTLFPSIVKNMPTKPNGTLSHKSGRRRWGQTIFKSGDSWEELEDYDFGASHSKKPSMGVFKEKRKKDSPFRLPEPVPSGSNHSTGENKSLPAVTSLKSDSELSTAPTSKQYYLKQSRYLPGVNPKKVSLIASGKEINPHTWSNQLFPKSLGPVGAELAFKRSNAGNLGSYATYNQSGYIPSFLKKEVQSAGQRIHLAPLNATASEYTWNTKTGRGQFSGRTYNPTAKNLNIVNRAQPIPSVHGRTDWVAKYGGHR

SEQ ID NO: 178 - DNA, 2857 bp, Homo sapiens
aacagtattttccttttcaacacatctattgaaagtgttggataaatgcaggatgttaatatgctataaacataaagtctgtttttaaaaaatagcatttgaaaatcatgaagggctttttgttttcttttgtttgtatatatgtttattggtaacaggtgacactggaagcaatgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatatgatcttgagcaacaaggaagcaacaccatcgtctgtgaaaaagtgtgcttccttaaaggggcctctggggctgaaatggcatcaaatggtaaataacatatgccagtttattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctgacaactgtacatagggttaacttctatttattgatgagacttccgtagataatgtggaaatcaaatttaaccaagaaaaaaagattggaacaaatgctctcttacattttattatcctggtgtacagaaaagattatataaaatttaaatccacatagatctattcataagctgaatgaaccattactaagagaatgcaacaggatacaaatggccactagaggtcattatttctttctttcttttttttttttttaatttcaagagcatttcactttaacattttggaaaagactaaggagaaacgtatatccctacaaacctcccctccaaacaccttctcacattcttttccacaattcacataacactactgcttttgtgccccttaaatgtagatatgtgctgaaagaaaaaaaaaacgcccaactcttgaagtccattgctgaaaactgcagccaggggttgaaagggatgcagacttgaagagtctgaggaactgaagtgggtcagcaagacctctgaaatcctgggtaaaggattttctccttacaattacaaacagcctctttcacattacaataatataccataggaggcacaagcaccattattaagccactttgcttacaccttaagtgtgtacaattcaagtgtgagaatgctgtgttaactattctttggaattctccttctgtccagcaaatactctaatgatggttaaacatggcacctactcagcaatgccttcctggaccacaacccctatccccctgccccaccctcctcattaaaaacaaatacttctactgtttgggtgtgtgatagggttctcaatgcagatctcccttttctagttagctatattcttgactgcatccgctaaaaatgttaaagcttcttgagagacagacatgccagattttcttggtatctcccataatacgacctacagtccatggtctacagatgttttaaatagaattgctattctcgatacatacaaagacgtaattgctgacccacaatcagtaacatccatattgggagatttttcaaaggatggtgaccctgcttgtatttatttaccttggtattttttcttgcatccttctgtgattcaaaaaagtaaaatgtggctttctgaaatgatggataagagtctacatcttctagaaaaaatacataaaggagtagttaagctctgtaaatgtgccacgagctccaacacgaccatcgtagggtgaagcccacgttttcttccatggcctcaaaggccctagaacttgcctacctttctggccttacctcctagctacttatccatctcttgaactttatactcttgtataaatttctaactttcagaaaatgccatactctgttttggcaccacacatgtatatttccccctggtacacttggaagactcttatccatctgtgaaaccctatgttgtcatcacttggtccatgaaatattacctggccaatatcccaccatcacctcaaacccaatcaccccctcctctgtatgctgtcacacctatattattaaacttatcacattgcattgtaattacttcctgacctttgtatctactcttttagtaactgatgtatatatctgaaaggagagattgtttcattgtgcaatcaataaatgtttgataaaataaagccc

SEQ ID NO: 179 - protein, 333 aa, Homo sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWFFLFFISLPNMILSNKEATPSSVKKCASLKGPLGLKWHQMVNNICQFIFWTVFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG

SEQ ID NO: 180 - DNA, 1242 bp, Homo sapiens
aagaggctctctctgcccatggatatccgcctgccccaggaattcctacagaagctacagatggagagcccagatctgcccaagccgctcagccgcatgtcccgccgggcctccctgtcagacattggctttgggaaactggaaacatacgtgaaactggacaaactgggagagggcacctatgccacagtcttcaaagggcgcagcaaactgacggagaaccttgtggccctgaaagagatccggctggagcacgaggagggagcgccctgcactgccatccgagaggtgtctctgctgaagaacctgaagcacgccaatattgtgaccctgcatgacctcatccacacagatcggtccctcaccctggtgtttgagtacctggacagtgacctgaagcagtatctggaccactgtgggaacctcatgagcatgcacaacgtcaagattttcatgttccagctgctccggggcctcgcctactgtcacacccgcaagatcctgcaccgggacctgaagccccagaacctgctcatcaacgagaggggggagctgaagctggccgactttggactggccagggccaagtcagtgcccacaaagacttactccaatgaggtggtgaccctgtggtacaggccccccgatgtgctgctgggatccacagagtactccacccccattgctatgtggggcgtgggctgcatccactacgagatggccacagggaggcccctcttcccgggctccacagtcaaggaggagctgcacctcatctttcgcctcctcgggacccccacagaagagacgtggcccggcgtgaccgccttctctgagttccgcacctacagcttcccctgctacctcccgcagccgctcatcaaccacgcgcccaggttggatacggatggcatccacctcctgagcagcctgctcgtgtatgaatccaagagtcgcatgtcagcagaggctgccctgagtcactcctacttccggtctctgggagagcgtgtgcaccagcttgaagacactgcctccatcttctccctgaaggagatccagctccagaaggacccaggctaccgaggcttggccttccagcagccaggacgagggaagaacaggcggcagagcatcttctgagccacgcccaccttgctgtggccaagggacaagagaccacatggagcacaaattcgggtaggatggagcctgtgtggccctcggaggactgaacaacga

SEQ ID NO: 181 - protein, 380 aa, Homo sapiens
KRLSLPMDIRLPQEFLQKLQMESPDLPKPLSRMSRRASLSDIGFGKLETYVKLDKLGEGTYATVFKGRSKLTENLVALKEIRLEHEEGAPCTAIREVSLLKNLKHANIVTLHDLIHTDRSLTLVFEYLDSDLKQYLDHCGNLMSMHNVKIFMFQLLRGLAYCHTRKILHRDLKPQNLLINERGELKLADFGLARAKSVPTKTYSNEVVTLWYRPPDVLLGSTEYSTPIAMWGVGCIHYEMATGRPLFPGSTVKEELHLIFRLLGTPTEETWPGVTAFSEFRTYSFPCYLPQPLINHAPRLDTDGIHLLSSLLVYESKSRMSAEAALSHSYFRSLGERVHQLEDTASIFSLKEIQLQKDPGYRGLAFQQPGRGKNRRQSIF

SEQ ID NO: 182 - DNA, 5399 bp, Homo sapiens
gccgcgcgggagagcgggagcctcggccctccgcgcggctgcagctacctaccctgcgcccggccaggtccccgacttagggatggcaaacttgcgccccgtggccgcccccgccagcgccggcccccgctcctgctgctgacggcgcccaggaaatccacagcagtgatacatgtgacgtccacactgacagtgccctcctgtgggcatggctcaggttgtgcgcagttcctggcacactggctgtaactccgcccctttctctccctctcagtaaagcaagattacgcggtgacatgcctcacagctgatcacgacacacggggatggagagcaagagttatggagaatacaggttggatgggcaagggacataggatgaccccagcctgtcccctcttactgtctgtgattctgtccctgcgcctggccaccgccttcgaccccgcccccagtgcctgctctgccctggcctcgggtgtgctctacggggccttctcgctgcaggacctctttcctaccatcgcctcgggctgctcctggaccctggagaaccctgaccccaccaagtactccctctacctgcgcttcaaccgccaggagcaggtgtgcgcacactttgccccccgcctgctgcccctggaccactacctggtcaactttacctgcctgcggcctagccccgaggaggcggtggcccaggcggagtcagaggtggggcggccagaagaggaggaggcagaggcggcagcggggttggagctgtgcagcggctcaggcccctttaccttcctgcacttcgacaagaacttcgtgcagctgtgcctgtcggctgagccctccgaggccccgcgcctgctggcgcccgctgccctagccttccgctttgtcgaggtcttgctcatcaacaacaacaactctagccaattcacctgtggtgtgctctgccgctggagtgaggagtgtggccgcgctgccggcagggcctgcggctttgctcagccaggctgcagctgccctggagaggcgggggccggctccaccaccaccacatctccaggccctcctgctgcccacaccctgtccaatgccctggtgcccgggggcccagccccacctgctgaggccgatttgcactcggggagcagcaatgatctgttcacaaccgagatgagatatggtgaggagccggaagaggaaccgaaagtgaaaacccagtggccgaggtctgcagatgagcctgggctatacatggcgcagacaggcgacccggcggctgaggagtggtccccgtggagcgtgtgttccctgacgtgtgggcagggtctgcaggtgcggacccgctcctgtgtgtcctccccctatgggaccctgtgcagcgggcccctgcgggagaccaggccctgcaacaattcagccacctgcccagtgcacggcgtgtgggaggagtgggggtcctggagcctgtgctcccgcagctgcgggcgggggtcccggagccggatgcggacctgcgtgcccccccagcacggcggcaaggcctgcgagggtcctgagctgcagactaagctctgcagtatggctgcctgcccggtggaaggccagtggttagaatggggtccctggggcccatgctccacgtcctgtgccaatgggacccaacagcgcagccggaagtgcagcgtggcgggcccagcctgggccacatgcacgggtgccctcactgacacccgggagtgcagcaacctcgagtgcccggccactgatagcaagtgggggccatggaatgcgtggagcctgtgctctaagacgtgtgacacaggctggcagcgccgcttccgcatgtgccaggccacgggcacgcagggctacccctgcgagggcaccggagaggaggtgaagccttgtagtgagaagaggtgtccagccttccatgagatgtgcagggatgagtacgtgatgctgatgacgtggaagaaggcagctgctggcgagatcatctacaacaagtgccccccgaatgcctcagggtctgccagccgccgctgtctcctcagtgcccaaggcgtggcgtactgggggctgcccagctttgctcgctgcatctcccatgagtaccgctacctgtatctgtcacttagggagcacctggccaaggggcagcgcatgctggcaggcgagggcatgtcgcaggtggtgcgcagcctgcaggagctactggcccggcgcacctactatagtggggacctgctcttctctgtggacattctgaggaatgtcactgacacctttaagagggccacctacgtgccctcggctgatgatgtgcagcgcttcttccaggtggtgagcttcatggtggatgcggaaaacaaggagaagtgggacgatgctcagcaggtgtcccctggctctgtgcacctgctccgtgtcgtggaggacttcattcacctggtgggcgatgctctcaaggccttccagagctctctgattgtcacagataatctagtgatcagcattcagcgagagcccgtctcagctgtgtccagtgacatcacgttccccatgcggggccgccggggcatgaaggactgggtgcggcactcagaggaccgcctcttcctgcccaaggaggtgctcagcctctcctccccagggaagccagccacatctggggcagcaggcagccctggcagggggaggggcccaggaacggtgcctcctggcccaggccactcccaccagcgcctcctcccagcagaccctgatgagtcctcctactttgtgatcggtgctgtactctaccgcacccttggcctcatcctgccgcctcccaggcccccgctggccgtcacatcccgggtgatgacagtgactgtgcgcccccctacccagcctccagctgagcccctcatcactgtggagctctcctacatcatcaatgggaccacggatccccattgcgccagctgggactactccagagcagatgccagctcaggagactgggacactgaaaattgccagaccctggagacccaggcagctcacacccgctgccagtgccagcacctgtccacctttgctgtactagcccagccgcccaaggacctgaccctggagctggcgggctccccctcggtccccctggtgatcggctgtgcagtgtcgtgcatggcgctgctcaccctgctcgccatctatgccgccttttggaggttcataaaatctgaacgctccatcatcttgctgaacttctgcctgtccatcttggcatccaacatcctgatcctcgtgggccagtcccgggtgctgagcaagggcgtgtgcaccatgacggctgccttcctgcacttcttctttctctcctccttttgctgggtgcttaccgaggcctggcagtcctacctggctgtcattgggcggatgcgcacccgcctcgttcgcaagcgcttcctctgcctgggctggggtctgcctgccctggtggtggccgtgtctgttggctttacccgaacgaaaggatacggtacatccagctactgctggctctccctggagggcggcctgctctacgcctttgtgggccctgcagccgtcattgtcctggtgaacatgctcatcggaatcatcgtcttcaacaagctcatggcacgtgatggcatctccgacaaatccaagaagcagagggccgggtcggagcggtgcccctgggccagcctgctcctcccctgctcagcgtgtggagcggtccccagccccctgctcagctcagcctcggccaggaacgccatggcctcactctggagctcctgcgtggtgctgcccctgctggcgctcacctggatgtctgccgtcctggctatgacagaccgccgttccgtcctcttccaggccctctttgctgtcttcaactccgcgcagggctttgtcatcactgctgtgcactgcttcctgcgccgagaggtccaggatgtggtgaagtgccagatgggggtgtgccgggctgatgagagcgaagactcccctgactcgtgtaagaacgggcagctgcagatcctgtcagactttgaaaaggatgtggatctggcttgtcaaacagtgctgttcaaggaggtcaacacttgcaacccgtccaccatcacgggcacactatcccgcctgtccctggatgaggatgaggagcccaagtcctgcctcgtgggccctgagggcagcctcagcttctcaccactgcctgggaatatcctggtgcccatggcagcctcaccagggctgggggagcctccgcccccacaggaggccaaccctgtttacatgtgtggggagggtggcctgcggcagctggacctcacatggctgcggcccactgagccaggctctgagggagactacatggtgctgccccggcggactttgagcctgcagcctggcggtgggggtggaggtggtgaggatgcccccagggcccggcccgaggggaccccccggcgagctgccaagacagtggcccacactgaaggctaccccagcttcctgtccgtggaccactcgggcctggggctgggccctgcctatggatctctccagaatccctatggaatgaccttccaaccgccaccgccgacacccagcgcccgccaagtgcccgagccaggggagcgcagccggaccatgcctcgcaccgtgcccggctctaccatgaagatgggctccctggagcgaaagaaattacggtattcagacctggactttgaggtgatgcacacccggaaacggcattcagaactctaccacgagctcaaccagaagttccacactttcgaccgctaccgcagccagtccacggccaagagggagaagcggtggagtgtgtcctcgggtggggcggccgagcggagcgtgtgcaccgataagcccagccctggggagcgccccagcttgtcccaacatcggcgccatcagagctggagcaccttcaaatctatgacactgggctcgctgccccccaagccccgagaacggctgactctgcaccgggcagcagcctgggagcccacagaaccaccggatggtgacttccagacagaggtgtgagtgccacgctggactgcccactgcatataaatatatatatctctctattttcacactccactttggaactacccaggagccagcgccctctcccctctcccgagggctgggcagggaggcgccgtggactcagccaggctgggggagccggacatggcttggcctggggtcccagggcccttccttgtttctcagaggcccctcagccactggaaccccatcttcagcccagcctgtccgtccctgtcccgggctggggaggggggaggggaactttgttgggaataaacttcactctgtgg

SEQ ID NO: 183 - protein, 1572 aa, Homo sapiens
MTPACPLLLSVILSLRLATAFDPAPSACSALASGVLYGAFSLQDLFPTIASGCSWTLENPDPTKYSLYLRFNRQEQVCAHFAPRLLPLDHYLVNFTCLRPSPEEAVAQAESEVGRPEEEEAEAAAGLELCSGSGPFTFLHFDKNFVQLCLSAEPSEAPRLLAPAALAFRFVEVLLINNNNSSQFTCGVLCRWSEECGRAAGRACGFAQPGCSCPGEAGAGSTTTTSPGPPAAHTLSNALVPGGPAPPAEADLHSGSSNDLFTTEMRYGEEPEEEPKVKTQWPRSADEPGLYMAQTGDPAAEEWSPWSVCSLTCGQGLQVRTRSCVSSPYGTLCSGPLRETRPCNNSATCPVHGVWEEWGSWSLCSRSCGRGSRSRMRTCVPPQHGGKACEGPELQTKLCSMAACPVEGQWLEWGPWGPCSTSCANGTQQRSRKCSVAGPAWATCTGALTDTRECSNLECPATDSKWGPWNAWSLCSKTCDTGWQRRFRMCQATGTQGYPCEGTGEEVKPCSEKRCPAFHEMCRDEYVMLMTWKKAAAGEIIYNKCPPNASGSASRRCLLSAQGVAYWGLPSFARCISHEYRYLYLSLREHLAKGQRMLAGEGMSQVVRSLQELLARRTYYSGDLLFSVDILRNVTDTFKRATYVPSADDVQRFFQVVSFMVDAENKEKWDDAQQVSPGSVHLLRVVEDFIHLVGDALKAFQSSLIVTDNLVISIQREPVSAVSSDITFPMRGRRGMKDWVRHSEDRLFLPKEVLSLSSPGKPATSGAAGSPGRGRGPGTVPPGPGHSHQRLLPADPDESSYFVIGAVLYRTLGLILPPPRPPLAVTSRVMTVTVRPPTQPPAEPLITVELSYIINGTTDPHCASWDYSRADASSGDWDTENCQTLETQAAHTRCQCQHLSTFAVLAQPPKDLTLELAGSPSVPLVIGCAVSCMALLTLLAIYAAFWRFIKSERSIILLNFCLSILASNILILVGQSRVLSKGVCTMTAAFLHFFFLSSFCWVLTEAWQSYLAVIGRMRTRLVRKRFLCLGWGLPALVVAVSVGFTRTKGYGTSSYCWLSLEGGLLYAFVGPAAVIVLVNMLIGIIVFNKLMARDGISDKSKKQRAGSERCPWASLLLPCSACGAVPSPLLSSASARNAMASLWSSCVVLPLLALTWMSAVLAMTDRRSVLFQALFAVFNSAQGFVITAVHCFLRREVQDVVKCQMGVCRADESEDSPDSCKNGQLQILSDFEKDVDLACQTVLFKEVNTCNPSTITGTLSRLSLDEDEEPKSCLVGPEGSLSFSPLPGNILVPMAASPGLGEPPPPQEANPVYMCGEGGLRQLDLTWLRPTEPGSEGDYMVLPRRTLSLQPGGGGGGGEDAPRARPEGTPRRAAKTVAHTEGYPSFLSVDHSGLGLGPAYGSLQNPYGMTFQPPPPTPSARQVPEPGERSRTMPRTVPGSTMKMGSLERKKLRYSDLDFEVMHTRKRHSELYHELNQKFHTFDRYRSQSTAKREKRWSVSSGGAAERSVCTDKPSPGERPSLSQHRRHQSWSTFKSMTLGSLPPKPRERLTLHRAAAWEPTEPPDGDFQTEV

SEQ ID NO: 184 - DNA, 6906 bp, Homo sapiens
gcccgggcaggtggttgcagcattgcagaagagaaaagtagtttgttggcagagaatcaggtattaatggaaagactcaatcaatctgattctatagaagaccctaacagtccagcaggaagaaggcatttgcagctccagactcaattagaacagctccaagaagaaacattcagactagaagcagccaaagatgattatcgaatacgttgtgaagagttagaaaaggagatctctgaacttcggcaacagaatgatgaactgaccactttggcagatgaagctcagtctctgaaagatgagatcgacgtgctgagacattcttctgataaagtatctaaactagaaggtcaagtatcactagaattcagcggccgctgaattctagggaggcgcgccccccggctgggcgccgcgcgcaccatggggctcccagcgctcgagttcagcgactgctgcctcgatagtccgcacttccgagagacgctcaagtcgcacgaagcagagctggacaagaccaacaaattcatcaaggagctcatcaaggacgggaagtcactcataagcgcgctcaagaatttgtcttcagcgaagcggaagtttgcagattccttaaatgaatttaaatttcagtgcataggagatgcagaaacagatgatgagatgtgtatagcaaggtctttgcaggagtttgccactgtcctcaggaatcttgaagatgaacggatacggatgattgagaatgccagcgaggtgctcatcactcccttggagaagtttcgaaaggaacagatcggggctgccaaggaagccaaaaagaaatatgacaaagagacagaaaagtattgtggcatcttagaaaaacacttgaatttgtcttccaaaaagaaagaatctcagcttcaggaggcagacagccaagtggacctggtccggcagcatttctatgaagtatccctggaatatgtcttcaaggtgcaggaagtccaagagagaaagatgtttgagtttgtggagcctctgctggccttcctgcaaggactcttcactttctatcaccatggttacgaactggccaaggatttcggggacttcaagacacagttaaccattagcatacagaacacaagaaatcgctttgaaggcactagatcagaagtggaatcactgatgaaaaagatgaaggagaatccccttgagcacaagaccatcagtccctacaccatggagggatacctctacgtgcaggagaaacgtcactttggaacttcttgggtgaagcactactgtacatatcaacgggattccaaacaaatcaccatggtaccatttgaccaaaagtcaggaggaaaagggggagaagatgaatcagttatcctcaaatcctgcacacggcggaaaacagactccattgagaagaggttttgctttgatgtggaagcagtagacaggccaggggttatcaccatgcaagctttgtcggaagaggaccggaggctctggatggaagccatggatggccgggaacctgtctacaactcgaacaaagacagccagagtgaagggactgcgcagttggacagcattggcttcagcataatcaggaaatgcatccatgctgtggaaaccagagggatcaacgagcaagggctgtatcgaattgtgggggtcaactccagagtgcagaagttgctgagtgtcctgatggaccccaagactgcttctgagacagaaacagatatctgtgctgaatgggagataaagaccatcactagtgctctgaagacctacctaagaatgcttccaggaccactcatgatgtaccagtttcaaagaagtttcatcaaagcagcaaaactggagaaccaggagtctcgggtctctgaaatccacagccttgttcatcggctcccagagaaaaatcggcagatgttacagctgctcatgaaccacttggcaaatgttgctaacaaccacaagcagaatttgatgacggtggcaaaccttggtgtggtgtttggacccactctgctgaggcctcaggaagaaacagtagcagccatcatggacatcaaatttcagaacattgtcattgagatcctaatagaaaaccacgaaaagatatttaacaccgtgcccgatatgcctctcaccaatgcccagctgcacctgtctcggaagaagagcagtgactccaagcccccgtcctgcagcgagaggcccctgacgctcttccacaccgttcagtcaacagagaaacaggaacaaaggaacagcatcatcaactccagtttggaatctgtctcatcaaatccaaacagcatccttaattccagcagcagcttacagcccaacatgaactccagtgacccagacctggctgtggtcaaacccacccggcccaactcactccccccgaatccaagcccaacttcacccctctcgccatcttggcccatgttctcggcgccatccagccctatgcccacctcatccacgtccagcgactcatcccccgtcaggtctgttgcagggtttgtttggttttctgttgctgccgttgttctctcattggctcggtcctctcttcatgcagtgttcagcctcctcgtcaactttgttccctgccatccaaacctgcacttgctttttgacaggccagaagaagcggtacatgaagactccagcacaccgttccggaaggcaaaagccttgtatgcctgcaaagctgaacatgactcagaactttcgttcacagcaggcacggtcttcgataatgttcacccatctcaggagcctggctggttggaggggactctgaacggaaagactggcctcatccctgagaattacgtggagttcctctaaccgtgggccccagcagaactgctgagctttacatggtatccatgacaactgctgattccagtgtcgaggccatttctctttgccactgagaaatgcagcgtgactgactctgttgctacctgtcaacatgaatgtttctgtgagctctggtgtcactcatctccatgatcatctcagccaacatgcatcagtactgcaagaaaagaagtcaatcagcagaggagagcatttgataactaagaggaagacttgcaaagccgttttctcatgagtaccctgaatagggggcactcattttgtttcaacggtccaaacgcccaaccttcagaaagaggaagtcagatagaaatagtccctgagagcacactgtgtagctaagcctgctggggctgggtgaagaaattggcgctgagatccaggctggatccattgcttttgtttacaataggcactctctctaccccacctctcagtacttgagacttaaagtgctacaggcagctggatctgtttgcatgcaggatgaagagggttaaaacactgtttatataagatccaatctctcaccatctctaaagcagccgttggcctgtcatcagtgagatacaatccagtcttctcatgcacgggaacacacacaccctgcgtttctccctcccaggctaggaacctctctgccaccaagggctgccatccatcgcctagtaaccacggcaacccaacctactctaaaaccaaaccaaaaaaataaaataacacatcctctttgcatgacacattttttttctcccctttttggtacactttttttgaatggttttctaacaacttgaagcacaggatcaaggaattagggtggtctacttgaggcagatgggatagtagctgggaactgttccctttctgattaatttcagcagcatcggaatatatttggagcacaccctagtaacctcttgagattaaattacatagtcttaatatttctgttcctccatgcaactgatgtttgttttttaaagggtaagatgctgcctcccaatgggtgatgccatctgactggtttccccatgtcctcccattcacccatctctgctcccacccttgcctgcctctaacccaccactggccagcccccttgccctactctgggctgctgaacactggtgctgtggtggttttcaaggttaattcctaggctaaccgtatggcctatagtttaaaagcacatctatgttcactgccactctgaaaaagggaattatttctcagtctttcaaggcttgagactaatataggccattgtgattcaggaagaaacccaaggttggagggtgggatgagtaccctctgaaaaagggaatttgctggtgaaaagaggctggatcttgtggaagactgtcttggatggggaagtactacctggagatttcaaattcacttggcctgcaaacaacagagttatccgtatcttccacatgtgaatgtcattgcaagggtgactctagacaaactacaaaccgatggaccgtcaagctccccaggagccccttggatggcagcgttgcttcagagtgtttcctgtttctggaattccttgttagggaactttaaagaagaaaagaaaaacttgaattgtgttgaattactgtatcttttactttttttttttgaaaagataaacttgtaaatagagtgatttgaaatactatatggcaaagttttatatttgatattctttaagttagttgctcacacacttaggctttgattgctgaagaagtatgtttaagagggagagaggggaggcaaagctgaagagagtcaaggtcactgtccccgcttcggcctgaaggaaagagaagacatttctatggccttgctctctgctgtcctgttggtgggcacgacacatcagtggtgttcagtctttatgtgtttttaagcatcccttgggctttggatttggagatgggaagagcatctccaggcaatgagtttttcaaagaatgcctacttagtagtaagatgaagctcaggatttaaataagtggggtcaggcattcgagtttttgtctttcttctcaggtgtatttcttggtacccccaagatatcaggccagaaagagatgagtcagttgctgtgctctttacttctttttctccacatcttctgaggctttagaaatgtggacaagctagttttcaaattttgtgtgcgtctgtaagttcttaaagaaccagcttcttagaatgttcagttctcaatgtgctgctgctttcccttctcctaaacattttaaaactcttccctttcacctccaattcccgtgatcccaaaagaagaggaagactccaggaggggtatagattgtgccgtcatagctttacaggtggttttaaagttagcaggggtttgtcatggtgattcactactcagtttatcagctcaaggattatacagctcttttccgggaactcacccaggagcaagcgagacactaccattgaatcagggaatgagaattaagaatggacaggaccaagacagaactcaagaaagccactggggaaaactcgagaagaaagggagtatactagtaggttagatctgtgaacctgaggacaagaagaccttgggaaatggaggcctcaggggatgtgcattcacatactattacgcttctcaaagagagaccaacatcatgcttttaacacatttgatgaggttttttatttgtgtttttgtttgttttttgagatggagtctcactctgtggcccaggctggagtgcagtggcgcggtcttggctcactgcaacctccgcctcccgggttcaagtgattctcctgtctcagcctcccaagtagctgggactgcaggcatgagccatcacacccggctggttttttgtgtttttagtagagatggggttttgccatgtttgccgggctgatctcgaactcctgacctcaagtgatctgcccacttcggacccccaaagtgctgggattccaggtgtgagccgctgcggccgaccacatttgatgtttgaagttgtaatctgtcccatcataaacttacctggagctcatgtggaggaacagaaggccaagatccttgctttgggggtgcctcacgaagcatccctgtagacacttggccccagcttcactgcttggaagcatgtccctccctcttgagttggctctgatttgaaatcgggagaaacagagctgctgccaatgggatcttttaggtaactccctccctagcttccgtgtgtctgtgcagtgcccatgagctgctgccaatgggatctttcaggtaccccctccccagcttccctgtggctgtgcggtgcccttgacagatggcttctctgtttccctttgcccagccaggctcccctccttcctattagctacaaaactggataaacttcagaatatgagccaatgagtaggaaggaacttgaagactaaagattttactctctcccctatccatgccccctacctctgactctctctgtgtgaacaggaaactttagggtagatgaggagaatgaattggttatcagagtggaagaccatggcccaggatccctgagctttcccagtagcctccagtttcctttgtaagacccagggatcacttagccatagcctgaatcttttaggggtattaaggtcagcctctcactcttccttcaggttactaacaaaatttcgtagctaaagaatgccatggccgggtgcagtggctcacgcctataatcccagcactttgggaggccgaggcgggcggatcacgaggtcaggagattgagaccatcctggctacgacggtgaagccccgtctctgctaagagtgcaaaaaattagccgggtgtggtggcgggcgcctgtagtcccagctactctggaggctgaggcaggagaatggcatgagcccgggaggcggagattgcggtgagccaagatcgcgcccctgcactccagcctgggtgacagagccagactccgtctcaaagg

SEQ ID NO: 185 - protein, 814 aa, Homo sapiens
MGLPALEFSDCCLDSPHFRETLKSHEAELDKTNKFIKELIKDGKSLISALKNLSSAKRKFADSLNEFKFQCIGDAETDDEMCIARSLQEFATVLRNLEDERIRMIENASEVLITPLEKFRKEQIGAAKEAKKKYDKETEKYCGILEKHLNLSSKKKESQLQEADSQVDLVRQHFYEVSLEYVFKVQEVQERKMFEFVEPLLAFLQGLFTFYHHGYELAKDFGDFKTQLTISIQNTRNRFEGTRSEVESLMKKMKENPLEHKTISPYTMEGYLYVQEKRHFGTSWVKHYCTYQRDSKQITMVPFDQKSGGKGGEDESVILKSCTRRKTDSIEKRFCFDVEAVDRPGVITMQALSEEDRRLWMEAMDGREPVYNSNKDSQSEGTAQLDSIGFSIIRKCIHAVETRGINEQGLYRIVGVNSRVQKLLSVLMDPKTASETETDICAEWEIKTITSALKTYLRMLPGPLMMYQFQRSFIKAAKLENQESRVSEIHSLVHRLPEKNRQMLQLLMNHLANVANNHKQNLMTVANLGVVFGPTLLRPQEETVAAIMDIKFQNIVIEILIENHEKIFNTVPDMPLTNAQLHLSRKKSSDSKPPSCSERPLTLFHTVQSTEKQEQRNSIINSSLESVSSNPNSILNSSSSLQPNMNSSDPDLAVVKPTRPNSLPPNPSPTSPLSPSWPMFSAPSSPMPTSSTSSDSSPVRSVAGFVWFSVAAVVLSLARSSLHAVFSLLVNFVPCHPNLHLLFDRPEEAVHEDSSTPFRKAKALYACKAEHDSELSFTAGTVFDNVHPSQEPGWLEGTLNGKTGLIPENYVEFL

SEQ ID NO: 186 - DNA, 2253 bp, Homo sapiens
ggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagaggcatcatggaccgatctaaagaaaactgcatttcaggacctgttaaggctacagctccagttggaggtccaaaacgtgttctcgtgactcagcaaattccttgtcagaatccattacctgtaaatagtggccaggctcagcgggtcttgtgtccttcaaattcttcccagcgcgttcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaaaaaaaaaaa

SEQ ID NO: 187 - protein, 403 aa, Homo sapiens
MDRSKENCISGPVKATAPVGGPKRVLVTQQIPCQNPLPVNSGQAQRVLCPSNSSQRVPLQAQKLVSSHKPVQNQKQKQLQATSVPHPVSRPLNNTQKSKQPLPSAPENNPEEELASKQKNEESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 188 - DNA, 5228 bp, Homo sapiens
ggatccggattcggattagcagcccgggaagagtgccgtggcacaggcgccggagggagcgcgaccctcggaccccgcctggcccgcggggctgggacccggccccggcctgcccgatggggcgcgcggccccggagatgcgccctcgcccggccccgcgcccccggccccgcgcccccggcccgcccgccccggcccgcgcctccgcctgagtcccccgcgccttggcccgccaccccccgccccgcgcccccggcccgcctgcgccatggagcccggccgcggcggcacagagaccgtgggcaagttcgagttctcccgcaaggacctgatcggccacggcgccttcgcggtggtcttcaagggccgccaccgcgagaagcacgatttggaggtcgccgtcaagtgcattaacaagaagaacctcgccaagtctcagacgctgctggggaaggaaatcaaaatcctgaaggaactgaaacatgaaaacatcgtggccctgtacgacttccaggaaatggctaattctgtctacctggttatggagtactgcaacggtggggacctggccgactacctgcacgccatgcgcacgctgagcgaggacaccatcaggctcttcctgcagcagatcgcgggcgccatgcggcttctgcacagcaaaggcatcatccaccgcgacctgaaaccgcagaacatcctgctgtccaaccccgccggccgccgcgccaaccccaacagcatccgcgtcaagatcgctgacttcggcttcgcgcggtacctccagagcaacatgatggcggccacactctgcggctcccccatgtacatggcccccgaggtcatcatgtcccagcactacgacgggaaggcggacctgtggagcatcggcaccatcgtctaccagtgcctgacggggaaggcgcccttccaggccagcagcccccaggacctgcgcctgttctacgagaagaacaagacgttggtccccaccatcccccgggagacctcggccccgctgcggcagctgctcctggccctactgcaacgcaaccacaaggaccgcatggacttcgatgagttttttcatcaccctttcctcgatgccagcccctcggtcaggaaatccccacccgtgcctgtgccctcgtacccaagctcggggtccggcagcagctccagcagcagctccacctcccacctggcctccccgccgtccctgggcgagatgcagcagctgcagaagaccctggcctccccggctgacaccgctggcttcctgcacagctcccgggactctggtggcagcaaggactcttcctgtgacacagacgacttcgtcatggtccccgcgcagtttccaggtgacctggtggctgaggcgcccagtgccaaacccccgccagacagcctgatgtgcagtgggagctcactggtggcctctgcgggcttggagagccacggccggaccccatctccatccccaccctgcagcagctcccccagtccctcaggccgggctggcccgttctccagcagcaggtgcggcgcctctgtccccatcccagtccccacgcaggtgcagaactaccagcgcattgagcgaaacctgcagtcacccacccagttccaaacacctcggtcctctgccatccgcaggtcaggcagcaccagccccctgggctttgcaagggccagcccctcgccccctgcccacgctgagcatggaggcgtcctggccaggaagatgtctctgggtggaggccggccctacacgccatctcctcaagttggaaccatccctgagcggccaggctggagcgggacgccctccccacagggagctgagatgcggggtggcaggtcccctcgtccaggctcctctgcacccgagcactctccccgcacttccgggctgggctgccgcctgcacagcgcccccaacctgtctgacttgcacgtcgtccgccccaagctgcccaaaccccccacggaccccctgggagctgtgttcagcccaccacaggccagccctccccagccgtcccacggcctgcagtcctgccggaacctgcggggctcacccaagctgcccgacttcctgcagcgaaaccccctgccccccatcctgggctcccccaccaaggctgtgccctcctttgacttcccgaagacccccagctcccagaacctgctggccctcctagcccggcagggcgtggtgatgacgccccctcgaaaccggacgctgcccgacctctcggaggtgggacccttccatggtcagccgttgggccctggcctgcggccaggcgaggaccccaagggcccctttggccggtctttcagcaccagccgcctcactgacctgctccttaaggcggcgtttgggacacaagccccggacccgggcagcacggagagcctgcaggagaagcccatggagatcgcaccctcagctggctttggagggagcctgcacccaggagcccgtgctgggggcaccagcagcccctccccggtggtcttcaccgtgggctctcccccgagcgggagcacgcccccccagggcccccgcaccaggatgttctcagcgggccccactggctctgccagctcttctgcccgccacctggtgcctgggccctgcagcgaggccccagcccctgagctccctgctccaggacacggctgcagctttgccgaccccattgctgcgaacctggagggggctgtgaccttcgaggcccccgacctccctgaggagaccctcatggagcaagagcacacggagatcctgcgtggcctgcgcttcacgctgctgttcgtgcagcacgtcctggagatcgcagccctgaagggcagcgccagtgaggcggcggggggccctgagtaccagctgcaggagagtgtggtggccgaccagatcagcctgctgagccgagaatggggcttcgcggaacagctggtgctgtacctgaaggtggccgagctactgtcctccggcctgcaaagtgccatcgaccagatccgggccggcaagctctgcctgtcgtccactgtgaagcaggtggtgcgcaggctgaatgagctgtacaaggccagcgtggtgtcctgccagggcctgagcctgcggctgcagcgcttcttcctggacaagcagcggctcctggaccgcattcacagcatcactgccgagaggctcatcttcagccacgctgtgcagatggtgcagtcggctgccctggacgagatgttccagcaccgtgagggctgcgtcccacgctaccacaaggccctgctgctcctggaggggctgcagcacatgctctcggaccaggccgacatcgagaacgtcaccaagtgcaagctgtgcattgagcggagactctcggcgctgctgactggcatctgtgcctgacctttctggcctggctgggccccccgtcctgccgagccctgcagagtgggctctgtgtgctggctggactcctcgggacaagcccatggcgctgatcgctggtgctgagccctgccctgggccccacggacagtcagcctgccggcctccctgcagctcacggggcagaaccagcacatctggagccacacagcttggggggtgtctcccatcttttacaggtggggatcacagaatttctgcccctccagctgcctggctcagcaggcgtgggtgccaccaccctctagccccagggcagccccggaggacaggcaagggcctgagaccactgccgactcaaagccaaagcgagctcctgcttagggcaggtcagcaggcactgtgcccaggaagagcctgcggcctcggcgtcccccagtctccaggagcctctccctccgagatacccacccagctttgtcaatcacccaagcactttatgcatatagagacagaacctggacctcaccagggactgctgggcagcgattcctggcagtggcctggtgtttgtacatacacatatgcagacacatgccagggccccccaagcccgagcaccggaccacgttgctgcccaggtctggacctcagcgggagaactggctccggggggagtggggccctgcgctagaggcagaggcagttctttgttcaagcgttcctctggggaccggcagcagaggcaccgtgttctctcagccctggatacgtcttgtaatctttcacactttattcctaaaacgtgtcttatttttatgcagctcattttttctttaaaggagaaaacttgtaggtgtttaagaattggttttgggagggcgaggactgggccaggttagaggcagatggcacaggggcgtgtggcgggcgggtgaggctgctttgcacacctgtgttggtggctgtcccctgccgcccctccctgtggcagcagcaggacaggtgtgtgcccagcaccctccctacctgggcctggaagcagatgaggggaatacttcatgcaaagaaaaaagtaacatgtgcaaaagctccccgtccagctttgacagtcagttttgatgtcagctcctcggcagggtaggcctgatgacagccctgtccctccctgcctccgccttgcccaaggccacggagggcgtctgcagagaggcctgccttccggattccaggcgggcatgccctgcaaaccccgcctgggcctcccttggtctgcccagccctcggttagccctgcctgaatcagtagatacttgaacgagtccccagtctgcgggaggcagtggtggggccatggacccatgcggggggttccagggtcacacgccacataacagacaaaaatacacacacgtgtgtttttctttgcaatacttgaaatattgccactgtgcttggacttagaagaagaaaatccccgtgacttcttcctcatcaccttgatggctttattctcaccttgtggggcatgtttgaatttattgcttcatggccgactggaatcctgagtcctgggaagctggcactgcggggatcttgcccggtgtcctggtcctcttgcttccgtcgcggccgcatgtgcgtgtgtccaagcaggtcctgggcgcctcaactgctgcccctggttgaatgttctcttgatagtgctggaccctttgtctattttaaagcgaattttgtgtgatttcctgccctttgcgttatattgtataataccaacgtaaggaaataaacctttggaattgttgaaaaaaaaaaaaaaaaa

SEQ ID NO: 189 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPIAANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA

SEQ ID NO: 190 - DNA, 1382 bp, Homo sapiens
  misc_feature (1233)..(1233): n is a, c, g, or t
ctctacactatagtcatgatccgctgggcccccagcgcatctcctggaagagcccactcaccctggacgagctcttcggtagcctcagaccgtccttgaagaggatgactgagacattatgggccacgcgctgtgtgtctgctctcggggaactgtcatcattgacaataagcgctacctcttcatccagaaactgggggagggtgggttcagctatgtggacctagtggaagggttacatgatggacacttctacgccctgaagcgaatcctgtgtcacgagcagcaggaccgggaggaggcccagcgagaagccgacatgcatcgcctcttcaatcaccccaacatccttcgcctcgtggcttactgtctgagggaacggggtgctaagcatgaggcctggctgctgctaccattcttcaagagaggtacgctgtggaatgagatagaaaggctgaaggacaaaggcaacttcctgaccgaggatcaaatcctttggctgctgctggggatctgcagaggccttgaggccattcatgccaagggttatgcccacagagacttgaagcccaccaatatattgcttggagatgaggggcagccagttttaatggacttgggttccatgaatcaagcatgcatccatgtggagggctcccgccaggctctgaccctgcaggactgggcagcccagcggtgcaccatctcctaccgagccccagagctcttctctgtgcagagtcactgtgtcatcggtgagcggactgatgtctggtccctaggctgcgtgctatatgccatgatgtttggggaaggcccttatgacatggtgttccaaaagggtgacagtgtggcccttgctgtgcagaaccaactcagcatcccacaaagccccaggcattcttcagcattgcggcagctcctgaactcgatgatgaccgtggacccgcatcagcgtcctcacattcctctcctcctcagtcagctggaggcgctgcagcccccagctcctggccaacatactacccaaattgaaaaagcagcatgttgagaagatggccccttgtgccttggaaagaggttcccatccccattggaatcaccacccattccaatccatggacttctcttacacttgggggtagcggggcaggacaatcatctcagtcctgcatcttttcttctgctttcttccctccaagagcaaaactgggcaaggggacttactgagtnanggtgggtgggggttgggaaaagggaaactggtggatatggaacatggctctgagcaggactgttgagctcacatagtgttctgactccaaattgggagcaggagaatgtgtaacaagaataaagttggaagcaggttggtgtaaaaa

SEQ ID NO: 191 - protein, 310 aa, Homo sapiens
MGHALCVCSRGTVIIDNKRYLFIQKLGEGGFSYVDLVEGLHDGHFYALKRILCHEQQDREEAQREADMHRLFNHPNILRLVAYCLRERGAKHEAWLLLPFFKRGTLWNEIERLKDKGNFLTEDQILWLLLGICRGLEAIHAKGYAHRDLKPTNILLGDEGQPVLMDLGSMNQACIHVEGSRQALTLQDWAAQRCTISYRAPELFSVQSHCVIGERTDVWSLGCVLYAMMFGEGPYDMVFQKGDSVALAVQNQLSIPQSPRHSSALRQLLNSMMTVDPHQRPHIPLLLSQLEALQPPAPGQHTTQIEKAAC

SEQ ID NO: 192 - DNA, 3831 bp, Homo sapiens
aactcatgcggccagagcgggaaagagactcgtctttgcgtccgagttctggagccgccgcaccccgactcctggggccgcggcagcggctgcgaggggacgggcgtccgctgtctcctgggttcccctcgtagcgacccgcgggatcggaaaaaaaggagaagatggaggaggagggtggcagcagcggcggcgccgcggggaccagcgcggacggcggcgacggaggagagcagctcctcactgtcaagcacgagctgcggactgctaatttgacaggacatgctgagaaggtgggaatagaaaattttgagctcctgaaggtcctaggaactggagcttatggaaaagtatttctagttcgtaaaataagtggccatgatactggaaagctgtatgccatgaaagttttgaaaaaggcaacaatcgttcaaaaggccaaaaccacagagcatacaaggacagaacgacaagtcctggaacacattaggcagtcgccatttttggtaacattacattatgctttccagacagaaaccaaacttcatctcattttagattatataaatggtggtgaactttttactcatctttctcaaagagagcgtttcacagagcatgaggtgcagatttatgttggagagattgtgcttgccctcgaacatctccacaagttggggattatatatcgtgatattaagcttgagaatattctacttgattctaatggccatgtggtgctgacagattttggtctgagtaaggagtttgtggctgatgaaactgaaagagcatattccttttgtggaactattgaatacatggcaccagatattgtcagagggggagattcaggacatgacaaggcagttgactggtggagtttgggtgttctaatgtatgaattactaactggagcatctcctttcactgttgatggagaaaaaaattcccaagctgagatatctaggagaatattaaaaagtgagcctccatatccccaagaaatgagtgctttagcgaaagacctaattcagcgtcttttgatgaaagatcccaagaagagattgggatgtggtccacgtgatgcagatgaaatcaaagaacatctcttctttcagaaaataaattgggatgatttagccgccaaaaaagtgcctgcaccatttaagccagtcattcgagatgaattagatgtgagtaactttgcagaagagttcacagaaatggatcccacttattctcccgcagccctgccccagagttctgagaagctgtttcagggctattcctttgttgctccttccatcctattcaagcgtaatgcagctgtcatagaccctcttcagtttcacatgggagttgaacgtcctggagtgacaaatgttgccaggagtgcaatgatgaaggactctccattctatcaacactatgacctagatttgaaggacaaacccctgggagaaggtagtttttcaatttgtcgaaagtgtgtgcataaaaaaagtaaccaagctttgcaagtcaaaataatcagcaaaaggatggaagccaatactcaaaaggaaataacagctctgaaactctgtgaaggacaccccaatattgtgaagttgcatgaagtttttcatgatcagcttcacacgtttctagtgatggaacttctgaatggaggagaactgtttgacgcattaagaaaaaagaagcacttcagtgagacggaagccagctacatcatgaggaagcttgtttcagctctaagccacatgcatgatcttggagtggtgcacagggatctgaaacctgagaatttattgttcaccgatgaaaatgacaatttggaaattaaaataattgattttggatttgcacggctaaagccaccggataatcagcccctgaagactccatgcttcacccttcattcatgccgcccagagctcttgaatcagaacggctacgatgagtcctgtgacctgtggagcttgggcgtcattttgtacacaatgttgtcaggacaggttcccttccaatctcatgaccgaagtttgacgtgtaccagcgcggtggaaatcatgaagaaaattaaaaagggagatttctcctttgaaggagaagcctggaagaatgtatcccaagaggctaaagatttgatccaaggacttctcacagtagatccaaacaaaaggcttaaaatgtctggcttgaggtacaatgaatggctacaagatggaagtcagctgtcctccaatcctctgatgactccggatattctaggatcttccggagctgccgtgcatacctgtgtgaaagcaaccttccacgcctttaacaaatacaagagagaggggttttgccttcagaatgttgataaggcccctttggctaagagaagaaaaatgaaaaagactagcaccagtaccgagacgcgcagagggtccagtgagagttcccattcttcttcctctcattctcacggtaaaactacacccaccaagacactgcagcccagcaatcctgccgacagcaataacccggagaccctcttccagttctcggactcagagcttaggcatggtaggagtgatcagtgatccattgcacctttattccctcagcatatgcctgagtcgatcttttatgcttttaaaaatgtttcccgttggtctcattggaatctgcctcctaatgatttttttcaggaaaacctgtttggttatcctcattcaaaagcactggacagagaatgttactgtgaatagagcacatattactctttttacgaacctagcatgatgccaacaagactattcttgaaagagcaaaggttcctgtaaatttaattagggctagatttgagctgcttgtaagtcacaggttttccagatgtctgccaacaagaaatgactcatactgtgatgataccttttgctttgccttgtggacaatgtgggtttttgaaatttgcacccttcaaacaatgatttatcagagaaaggggtctgttttcaaaaaagattctgtaatgaattttatgtgtggcatatacttatttcttgagagaagattttaacttattgtttttattttatggttacatatgatgataacctgctattattaaactttttctaaaaagtgaaaaaaaataaaaaagatataagaactcaaggtcccatactctgtattcgggatccatctgagatgcatgctaagctatgtgtatgtttttaattttgcactgctctttcctggcaatttgttttaatggttattgcagaatattaaggtacatgtctctctgttttaagtaatattgcactttataaaaaagtatgaataaagcaaactattttataaagtgcactgtttaaagcatttgcactgtatttttgccatttattttcattttctactttaaatttgtcctcacatgcctcttctactttgtatgcaacaagtagaatggggccttgttgtgtcatgtagtcagccacttatgcaccaatgtgaggaaaacctaaagggaaattaaactaaacactgtgcttcatatttgtacactgtgttgtactacagtgaggaatttcctcctgtagtcatatattatgtacataatattttagaatcatacctatgacttgtttggaaatttttctgttgaattttaaatccagaaagcatattttataaacttatgcagagcacttttattgctcaaaagttctgaattcatacagaaaacaagtactatgtgatgaaaacatttcattgaaagattgcggcatttaaaaatacaattaattcgttccctatgcaaaaaaaaaaaaa

SEQ ID NO: 193 - protein, 809 aa, Homo sapiens
MEEEGGSSGGAAGTSADGGDGGEQLLTVKHELRTANLTGHAEKVGIENFELLKVLGTGAYGKVFLVRKISGHDTGKLYAMKVLKKATIVQKAKTTEHTRTERQVLEHIRQSPFLVTLHYAFQTETKLHLILDYINGGELFTHLSQRERFTEHEVQIYVGEIVLALEHLHKLGIIYRDIKLENILLDSNGHVVLTDFGLSKEFVADETERAYSFCGTIEYMAPDIVRGGDSGHDKAVDWWSLGVLMYELLTGASPFTVDGEKNSQAEISRRILKSEPPYPQEMSALAKDLIQRLLMKDPKKRLGCGPRDADEIKEHLFFQKINWDDLAAKKVPAPFKPVIRDELDVSNFAEEFTEMDPTYSPAALPQSSEKLFQGYSFVAPSILFKRNAAVIDPLQFHMGVERPGVTNVARSAMMKDSPFYQHYDLDLKDKPLGEGSFSICRKCVHKKSNQALQVKIISKRMEANTQKEITALKLCEGHPNIVKLHEVFHDQLHTFLVMELLNGGELFDALRKKKHFSETEASYIMRKLVSALSHMHDLGVVHRDLKPENLLFTDENDNLEIKIIDFGFARLKPPDNQPLKTPCFTLHSCRPELLNQNGYDESCDLWSLGVILYTMLSGQVPFQSHDRSLTCTSAVEIMKKIKKGDFSFEGEAWKNVSQEAKDLIQGLLTVDPNKRLKMSGLRYNEWLQDGSQLSSNPLMTPDILGSSGAAVHTCVKATFHAFNKYKREGFCLQNVDKAPLAKRRKMKKTSTSTETRRGSSESSHSSSSHSHGKTTPTKTLQPSNPADSNNPETLFQFSDSELRHGRSDQ

SEQ ID NO: 194 - DNA, 3327 bp, Homo sapiens
ccgggcggggggttgcggcgctcaggagaggccccggctccgccccgggcctgcccagggggagagcggagctccgcagccgggtcgggtcggggcccctcccgggaggagcgtggagcgcggcggcggcggcggcagcagaaatgatggaagaattgcatagcctggacccacgacggcaggaattattggaggccaggtttactggagtaggtgttagtaagggaccacttaatagtgagtcttccaaccagagcttgtgcagcgtcggatccttgagtgataaagaagtagagactcccgagaaaaagcagaatgaccagcgaaatcggaaaagaaaagctgaaccatatgaaactagccaagggaaaggcactcctaggggacataaaattagtgattactttgagtttgctgggggaagcgcgccaggaaccagccctggcagaagtgttccaccagttgcacgatcctcaccgcaacattccttatccaatcccttaccgcgacgagtagaacagcccctctatggtttagatggcagtgctgcaaaggaggcaacggaggagcagtctgctctgccaaccctcatgtcagtgatgctagcaaaacctcggcttgacacagagcagctggcgcaaaggggagctggcctctgcttcacttttgtttcagctcagcaaaacagtccctcatctacgggatctggcaacacagagcattcctgcagctcccaaaaacagatctccatccagcacagacggacccagtccgacctcacaatagaaaaaatatctgcactagaaaacagtaagaattctgacttagagaagaaggagggaagaatagatgatttattaagagccaactgtgatttgagacggcagattgatgaacagcaaaagatgctagagaaatacaaggaacgattaaatagatgtgtgacaatgagcaagaaactccttatagaaaagtcaaaacaagagaagatggcgtgtagagataagagcatgcaagaccgcttgagactgggccactttactactgtccgacacggagcctcatttactgaacagtggacagatggttatgcttttcagaatcttatcaagcaacaggaaaggataaattcacagagggaagagatagaaagacaacggaaaatgttagcaaagcggaaacctcctgccatgggtcaggcccctcctgcaaccaatgagcagaaacagcggaaaagcaagaccaatggagctgaaaatgaaacgttaacgttagcagaataccatgaacaagaagaaatcttcaaactcagattaggtcatcttaaaaaggaggaagcagagatccaggcagagctggagagactagaaagggttagaaatctacatatcagggaactaaaaaggatacataatgaagataattcacaatttaaagatcatccaacgctaaatgacagatatttgttgttacatcttttgggtagaggaggtttcagtgaagtttacaaggcatttgatctaacagagcaaagatacgtagctgtgaaaattcaccagttaaataaaaactggagagatgagaaaaaggagaattaccacaagcatgcatgtagggaataccggattcataaagagctggatcatcccagaatagttaagctgtatgattacttttcactggatactgactcgttttgtacagtattagaatactgtgagggaaatgatctggacttctacctgaaacagcacaaattaatgtcggagaaagaggcccggtccattatcatgcagattgtgaatgctttaaagtacttaaatgaaataaaacctcccatcatacactatgacctcaaaccaggtaatattcttttagtaaatggtacagcgtgtggagagataaaaattacagattttggtctttcgaagatcatggatgatgatagctacaattcagtggatggcatggagctaacatcacaaggtgctggtacttattggtatttaccaccagagtgttttgtggttgggaaagaaccaccaaagatctcaaataaagttgatgtgtggtcggtgggtgtgatcttctatcagtgtctttatggaaggaagccttttggccataaccagtctcagcaagacatcctacaagagaatacgattcttaaagctactgaagtgcagttcccgccaaagccagtagtaacacctgaagcaaaggcgtttattcgacgatgcttggcctaccgaaagagggaccgcattgatgtccagcagctggcctgtgatccctacttgttgcctcacatccgaaagtcagtctctacaagtagccctgctggagctgctattgcatcaacctctggggcgtccaataacagttcttctaattgagactgactccaaggccacaaactgttcaacacacacaaagtggacaaatggcgttcagcagcgggtttggaacatagcgaatccgaatggatctgatgaaacctgtaccaggtgcttttattttcttgcttttttcccatccatagagcatgacagcatcgattctcattgaggagaaaccttgggcagctccggccaggccttgtaggaaaaggccccgcccgaggttccagcgtcaacggccactgtgtgtggctgctctgagtgaggaaaaaattaaaaagaaaaactggttccatgtactgtgaacttgaaaacttgcagactcaggggggtccctgatgcagtgcttcagatgaagaatgtggacttgaaaatacagactgggctagtccagtgtctatatttaaacttgttcttttcttttaataaagtttaggtaacatctcctgaaaagcttgtagcacaaaggctcagctggggatggtgtttgacttcggaggaaaaaagttgctattgcccgttaaaggcactagagttagtgttttatccctaaataatttcaatttttaaaaacatgcagcttccctctccccttttttatttttgaaagaatacatttggtcataaagtgaaacccgtattagcaagtacgaggcaatgttcattccaatcagatgcagctttctcctccgtctggtctcctgtttgcaattgcttccctcatctcagtagggaaaaaattgagtgggagtactgagatgtgtgggtttttgccattggacaaagaatgaggttagaagactgcagcttggagtctctctaggttttcaactatttcttcacaatttgaacacttgacggttgtcccttttaatttatttgaagtgctatttttttaaataaaggttcatctgtccatgcaaaaaaa

SEQ ID NO: 195 - protein, 749 aa, Homo sapiens
MEELHSLDPRRQELLEARFTGVGVSKGPLNSESSNQSLCSVGSLSDKEVETPEKKQNDQRNRKRKAEPYETSQGKGTPRGHKISDYFEFAGGSAPGTSPGRSVPPVARSSPQHSLSNPLPRRVEQPLYGLDGSAAKEATEEQSALPTLMSVMLAKPRLDTEQLAQRGAGLCFTFVSAQQNSPSSTGSGNTEHSCSSQKQISIQHRRTQSDLTIEKISALENSKNSDLEKKEGRIDDLLRANCDLRRQIDEQQKMLEKYKERLNRCVTMSKKLLIEKSKQEKMACRDKSMQDRLRLGHFTTVRHGASFTEQWTDGYAFQNLIKQQERINSQREEIERQRKMLAKRKPPAMGQAPPATNEQKQRKSKTNGAENETLTLAEYHEQEEIFKLRLGHLKKEEAEIQAELERLERVRNLHIRELKRIHNEDNSQFKDHPTLNDRYLLLHLLGRGGFSEVYKAFDLTEQRYVAVKIHQLNKNWRDEKKENYHKHACREYRIHKELDHPRIVKLYDYFSLDTDSFCTVLEYCEGNDLDFYLKQHKLMSEKEARSIIMQIVNALKYLNEIKPPIIHYDLKPGNILLVNGTACGEIKITDFGLSKIMDDDSYNSVDGMELTSQGAGTYWYLPPECFVVGKEPPKISNKVDVWSVGVIFYQCLYGRKPFGHNQSQQDILQENTILKATEVQFPPKPVVTPEAKAFIRRCLAYRKRDRIDVQQLACDPYLLPHIRKSVSTSSPAGAAIASTSGASNNSSSN

SEQ ID NO: 196 - DNA, 2221 bp, Homo sapiens
gtgaccggaggcgagaaaccccgcctcggcaccctgacgcagcgcaggacccgccccgcgcgtgacgccagcgtcaggccagccccggcatgctctgcggccgcccgcggtccagctccgacaacaggaattttctccgagagcgggccgggctcagttcagctgctgtccagacccggatcggcaacagtgccgcctccagacgttctcctgccgctcgcccgcccgtcccagcgcccccagccctcccgcgagggcgccccgggacggaaggatccaccagtctgtcggcgcccgccgttctcgtggtcgccgtcgccgtcgtcgtggtggtagtctccgccgtcgcctgggccatggccaattacatccacgtccctcccggctccccggaggtgcccaagctgaacgtcaccgttcaggatcaggaggagcatcgctgccgggagggggccctgagcctcctgcaacacctgcggcctcactgggacccccaggaggtgaccctgcagctcttcacagatggaatcacaaataaacttattggctgttacgtgggaaacaccatggaggatgtagtcctggtgagaatttatggcaataagactgagttattagtcgatcgagatgaggaagtaaagagttttcgagtgttgcaggctcatgggtgtgcaccacaactctactgtaccttcaataatggactatgctatgaatttatacaaggagaagcactggatccaaagcatgtctgcaacccagccattttcaggctaatagctcgtcagcttgctaaaatccatgctattcatgcacacaatggctggatccccaaatctaatctttggctaaagatgggaaagtatttctctctcattcccacaggatttgcagatgaagacattaataaaaggttcctaagtgatatcccaagctctcagattctccaggaagagatgacttggatgaaggagattctttccaacctgggctcacctgttgtgctttgccataatgacctattgtgtaagaatataatctacaatgagaaacaaggtgatgtacagttcattgattatgaatattctggatacaactacctggcatatgatattggaaatcatttcaatgaatttgcaggtgtgagtgatgtagactatagtctgtatccagatagagaactacagagtcagtggctgcgtgcttaccttgaagcctacaaagaatttaagggctttgggactgaagttactgaaaaggaggtagaaatactcttcattcaagtcaatcagtttgcattggcttctcatttcttttggggattgtgggctttgattcaagccaaatactccactattgagtttgatttccttgggtatgcaattgttcgttttaaccagtactttaaaatgaagcctgaggttactgcattaaaagtgcctgagtaaagaagagatttaattattctccagtagctgagcaatgcttgtgaatcttttcttaagaaatcccaaaaagccaatattagttaaaattctgttgtttaatttggttatcttgctttataaattatgcctctaaacaatcaaatctatttttgaaatagactgaatgatgtcaagaaatatacctactgctatccgtatgtggtggattagaaatgtgttaaatctgcaaaaggtataaagatgtcagtttaatttctttgataatttaacctatgttgtatgtgaattatttattataaacttagcacgattctgtgactgtttttctctgtttcacgttcgttgagtgtaagcaatgaaaatgtcccaaataagttttttaagttttactttaataagattaatttcagtaaacattctagttgttcagtgtaacctttttatcttgatgcattgtaagtaaaatgaatcatttactcttgaaatgccagtcattgactgatgtagataatttaggattttcatataaaaatagctgtttaggaaggtgaaatacattcactgtctctgttggtggtacatcttgttgaattcaatattagaaagtatttctttttggggtaatataacttagaattaaatccctgtttctctatgtagtctggcagtataaatataaatatttaccatataatcttggaataagtattagttaatgttaccaaaatctgtattaaataatgttttcaaatgctaaaaaaaaaaaaaaaaaa

SEQ ID NO: 197 - protein, 452 aa, Homo sapiens
MLCGRPRSSSDNRNFLRERAGLSSAAVQTRIGNSAASRRSPAARPPVPAPPALPRGRPGTEGSTSLSAPAVLVVAVAVVVVVVSAVAWAMANYIHVPPGSPEVPKLNVTVQDQEEHRCREGALSLLQHLRPHWDPQEVTLQLFTDGITNKLIGCYVGNTMEDVVLVRIYGNKTELLVDRDEEVKSFRVLQAHGCAPQLYCTFNNGLCYEFIQGEALDPKHVCNPAIFRLIARQLAKIHAIHAHNGWIPKSNLWLKMGKYFSLIPTGFADEDINKRFLSDIPSSQILQEEMTWMKEILSNLGSPVVLCHNDLLCKNIIYNEKQGDVQFIDYEYSGYNYLAYDIGNHFNEFAGVSDVDYSLYPDRELQSQWLRAYLEAYKEFKGFGTEVTEKEVEILFIQVNQFALASHFFWGLWALIQAKYSTIEFDFLGYAIVRFNQYFKMKPEVTALKVPE

SEQ ID NO: 198 - DNA, 2745 bp, Homo sapiens
ggcacgagggcgaccgctccccggcgggagccagcgaaggtttccatgtcagaggccgatggagaactgaagattgccacctacgcacaaaggccattgagacacttcgtgtagctggaagacaccaacttcctgacaggagctttatttcatttgggatttcaagtttacagatggtatcttctcaaaagttggaaaaacctatagagatgggcagtagcgaaccccttcccatcgcagatggtgacaggaggaggaagaagaagcggaggggccgggccactgactccttgccaggaaagtttgaagatatgtacaagctgacctctgaattgcttggagagggagcctatgccaaagttcaaggtgccgtgagcctacagaatggcaaagagtatgccgtcaaaatcatcgagaaacaagcagggcacagtcggagtagggtgtttcgagaggtggagacgctgtatcagtgtcagggaaacaagaacattttggagctgattgagttctttgaagatgacacaaggttttacttggtctttgagaaattgcaaggaggttccatcttagcccacatccagaagcaaaagcacttcaatgagcgagaagccagccgagtggtgcgggacgttgctgctgcccttgacttcctgcataccaaagacaaagtctctctctgtcacctaggctggagtgctatggcgccatcagggctcactgcagccccaacctccctgggctccagtgatcctcccacctcagcctcccaagtagctgggactacaggcattgctcatcgtgatctgaaaccagaaaatatattgtgtgaatctccagaaaaggtgtctccagtgaaaatctgtgactttgacttgggcagtgggatgaaactgaacaactcctgtacccccataaccacaccagagctgaccaccccatgtggctctgcagaatacatggcccctgaggtagtggaggtcttcacggaccaggccacattctacgacaagcgctgtgacctgtggagcctgggcgtggtcctctacatcatgctgagtggctacccacccttcgtgggtcactgcggggccgactgtggctgggaccggggcgaggtctgcagggtgtgccagaacaagctgtttgaaagcatccaggaaggcaagtatgagtttcctgacaaggactgggcacacatctccagtgaagccaaagacctcatctccaagctcctggtgcgagatgcaaagcagagacttagcgccgcccaagttctgcagcacccatgggtgcaggggcaagctccagaaaagggactccccacgccgcaagtcctccagaggaacagcagcacaatggacctgacgctcttcgcagctgaggccatcgcccttaaccgccagctatctcagcacgaagagaacgaactagcagaggagccagaggcactagctgatggcctctgctccatgaagctttcccctccctgcaagtcacgcctggcccggagacgggccctggcccaggcaggccgtggtgaagacaggagcccgcccacagcactctgaaatgctccagtcacaccttataggccctaggcctggccaggcattgtcccctggaaacctgtgtggctaaagtctgctgagcaggcagcagcctctgctctgtggctccattcaggctttttcatctacgaaggccctgaggttcccatcaacccccatttccctagggtcctggaggaaaaagctttttccaaaggggttgtctttgaaaaggaaagcaatcacttctcactttgcataattgcctgcagcaggaacatctcttcactgggctccacctgctcacccgcctgcagatctgggatccagcctgctctcaccgctgtagctgtggcggctggggctgcagcctgcagggagaagcaagaagcatcagttgacagaggctgccgacacgtgcctcttccctctcttctctgtcaccctcctctggcggtccttccaccttcctctgtcctccggatgtcctctttgcccgtcttctcccttggctgagcaaagccatcccctcaattcagggaagggcaaggagccttcctcattcaggaaatcaaatcagtcttccggtctgcagcacggaaaagcacataatctttctttgctgtgactgaaatgtatccctcgtttatcatcccctttgtttgtgattgctgctaaagtcagtagtatcgtttttttaaaaaaaaagtttggtgtttttaaccatgctgttccagcaaagatgataccttaaactcccactgcaagcccatgaacttcccagagagtggaacggcttgctcttctttctagaatgtccatgcacttgggttttaatcagcagttccctattattctgattttaagctgttcctgtgatgaacttagagacagcatcggtgtctgctgctgtgtccccaggtcttgtgtgggtggcacagatctgggcagttagatagtgctctgtgcctaaggtgaagccacactagggtgaagcctcacttccctgtttgagcaatgcagtgcctgctgcccgtgtgcatgaaggtacagccattcagataagtggaactattgagttacataaagaaaatagatttgcatttgtcaggcagacgtttatacaacaccacggtgcttttatacattgtgcttattttaataaaactgaaattctaaaaaaaaaaaaaaaaaa

SEQ ID NO: 199 - protein, 465 aa, Homo sapiens
MVSSQKLEKPIEMGSSEPLPIADGDRRRKKKRRGRATDSLPGKFEDMYKLTSELLGEGAYAKVQGAVSLQNGKEYAVKIIEKQAGHSRSRVFREVETLYQCQGNKNILELIEFFEDDTRFYLVFEKLQGGSILAHIQKQKHFNEREASRVVRDVAAALDFLHTKDKVSLCHLGWSAMAPSGLTAAPTSLGSSDPPTSASQVAGTTGIAHRDLKPENILCESPEKVSPVKICDFDLGSGMKLNNSCTPITTPELTTPCGSAEYMAPEVVEVFTDQATFYDKRCDLWSLGVVLYIMLSGYPPFVGHCGADCGWDRGEVCRVCQNKLFESIQEGKYEFPDKDWAHISSEAKDLISKLLVRDAKQRLSAAQVLQHPWVQGQAPEKGLPTPQVLQRNSSTMDLTLFAAEAIALNRQLSQHEENELAEEPEALADGLCSMKLSPPCKSRLARRRALAQAGRGEDRSPPTAL

SEQ ID NO: 200 - DNA, 1172 bp, Homo sapiens
aaaggggcctctggtgaccgcccctacctggcatccctctaacccaggaggagcgtggggaaaggggctgtgggcctctcggggagcgagctgcgggtagcggcgcactgggtacaggcgcgcgcttggctgtcgcctcttccgctgtgtttgggaggactcgaactggcgccaggaaatattaggaagctgtgattttcaaagctaattatgaaaacatttatcattggaatcagtggtgtgacaaacagtggcaaaacaacactggctaagaatttgcagaaacacctcccaaattgcagtgtcatatctcaggatgatttcttcaagccagagtctgagatagagacagataaaaatggatttttgcagtacgatgtgcttgaagcacttaacatggaaaaaatgatgtcagccatttcctgctggatggaaagcgcaagacactctgtggtatcaacagaccaggaaagtgctgaggaaattcccattttaatcatcgaaggttttcttctttttaattataagccccttgacactatatggaatagaagctatttcctgactattccatatgaagaatgtaaaaggaggaggagtacaagggtctatcagcctccagactctccgggatactttgatggccatgtgtggcccatgtatctaaagtacagacaagaaatgcaggacatcacatgggaagttgtgtacctggatggaacaaaatctgaagaggacctctttttgcaagtatatgaagatctaatacaagaactagcaaagcaaaagtgtttgcaagtgacagcataaagacggaacacaacaaatccttcctgaagtgaattaggaaactccaaggagtaatttaagaaccttcaccaagatacaatgtatactgtggtacaatgacagccattgtttcatatgtttgatttttattgcacatggttttcccaacatgtggaacaataaatatccatgccaatggacaggactgtaccttagcaagttgctccctctccagggagcgcatagatacagcagagctcacagtgagtcagaaagtctccactttctgaacatagctctataacaatgattgtcaaacttttctaactggagctcagagtaagaaataaagattacatcacaatccaaaaaaaaaaaaaaaa

SEQ ID NO: 201 - protein, 199 aa, Homo sapiens
MKTFIIGISGVTNSGKTTLAKNLQKHLPNCSVISQDDFFKPESEIETDKNGFLQYDVLEALNMEKMMSAISCWMESARHSVVSTDQESAEEIPILIIEGFLLFNYKPLDTIWNRSYFLTIPYEECKRRRSTRVYQPPDSPGYFDGHVWPMYLKYRQEMQDITWEVVYLDGTKSEEDLFLQVYEDLIQELAKQKCLQVTA

SEQ ID NO: 202 - DNA, 1902 bp, Homo sapiens
gctggaacccggcgccgagagtagagaaaaggggcctctggtgaccgcccctacctggcatccctctaacccaggaggagcgtggggaaaggggctgtgggcctctcggggagcgagctgcgggtagcggcgcactgggtacaggcgcgcgcttggctgtcgcctctgccgctgtgtttgggaggactcgaactggcgccaggaaatattaggaagctgtgattttcaaagctaattatgaaaacatttatcattggaatcagtggtgtgacaaacagtggcaaaacaacactggctaagaatttgcagaaacacctcccaaattgcagtgtcatatctcaggatgatttcttcaagccagagtctgagatagagacagataaaaatggatttttgcagtacgatgtgcttgaagcacttaacatggaaaaaatgatgtcagccatttcctgctggatggaaagcgcaagacactctgtggtatcaacagaccaggaaagtgctgaggaaattcccattttaatcatcgaaggttttcttctttttaattataagtaagcatctccaccctaatattgtctctgagtgaatggggggataaaaacccttgtgaactaagtatgctgttattttaggccccttgacactatatggaatagaagctatttcctgactattccatatgaagaatgtaaaaggaggaggaggtaagttttgaaccatctttgtgagttgtaattcaaaacaaaaaatgtagaagaaaaatgaggacagcaacattattgagcaatgcgtatacgtcatttttcagagacaggaagccttaactcttttacaaaaggagcaaaactcttttccataaatggtcatccgcatggagtaatgcaaattactttctaagaaaagggccttatatcaagcttttggctcatttctcctaaatcctcaaacaaagcaagcccatggtgattaggattttaatgtaagtgagcctgttctgccacagctacaaagaacaccaattagttcaaagcttaatccaacatagtggtctctccattgttatttacactatttgtctcttagttctcttataaagcactagaaggaaaaagaataattaagaatgatagtcagtggattacaaagttaattttaatatcaaagaactaaatccatgttactgtcgttctaactgccattgagttaaaacatgtagtacatgtagtcctaatcctgtttagattcagcctctgagtgatttccccattatcctgaccattcatattttactttatcaattgtgtgtttctgtcaacataaagtttcctacgtgttttgatagtacaagggtctatcagcctccagactctccgggatactttgatggccatgtgtggcccatgtatctaaagtacagacaagaaatgcaggacatcacatgggaagttgtgtacctggatggaacaaaatctgaagaggacctctttttgcaagtatatgaagatctaatacaagaactagcaaagcaaaagtgtttgcaagtgacagcataaagacggaacacaacaaatccttcctgaagtgaattaggaaactccaaggagtaatttaagaaccttcaccaagatacaatgtatactgtggtacaatgacagccattgtttcatatgtttgatttttattgcacatggttttcccaacatgtggaacaataaatatccatgccaatggacaggactgtaccttagcaagttgctccctctccagggagcgcatagatacagcagagctcacagtgagtcagaaagtctccactttctgaacatagctctataacaatgattgtcaaacttttctaactggagctcagagtaagaaataaagattacatcacaatcc

SEQ ID NO: 203 - DNA, 1760 bp, Homo sapiens
cagccgagcaagcaaaaattcttccaggagctgagcaagagcctggacgcattccctgaggattctgtcggcacaaggtgctgccccagctgctgaccgccttcgagttcggcaatgctggggccgttgtcctcacgcccctcttcaaggtgggcaagttcctgagcgctgaggagtatcagcagaagatcatccctgtggtggtcaagatgttctcatccactgaccgggccatgcgcatccgcctcctgcagcagatggagcagttcatccagtaccttgacgagccaacagtcaacacccagatcttcccccacgtcgtacatggcttcctggacaccaaccctgccatccgggagcagacggtcaagtccatgctgctcctggccccaaagctgaacgaggccaacctcaatgtggagctgatgaagcactttgcacggctacaggccaaggatgaacagggccccatccgctgcaacaccacagtctgcctgggcaaaatcggctcctacctcagtgctagcaccagacacagggtccttacctctgccttcagccgagccactagggacccgtttgcaccgtcccgggttgcgggtgtcctgggctttgctgccacccacaacctctactcaatgaacgactgtgcccagaagatcctgcctgtgctctgcggtctcactgtagatcctgagaaatccgtgcgagaccaggccttcaaggcatttcggagcttcctgtccaaattggagtctgtgtcggaggacccgacccagctggaggaagtggagaaggatgtccatgcagcctccagccctggcatgggaggagccgcagctagctgggcaggctgggcctggaccggggtctcctcactcacctccaagctgatccgttcgcacccaaccactgccccaacagaaaccaacattccccaaagacccagccgtcctgcacgcaggccactgggagacgcaggaggaggacaaggacacagcagaggacagcagcactgctgacagatgggacgacgaagactggggcagcctggagcaggaggccgagtctgtgctggcccagcaggacgactggagcaccgggggccaagtgagccgtgctagtcaggtcagcaactccgaccacaaatccttccaaatccccagagtccgactggagcagctgggaagctgagggctcctgggaacagggctggcaggagccaagctcccaggagccacctcctgacggtacacggctggccagcgagtataactggggtggcccagagtccagcgacaagggcgaccccttcgctaccctgtctgcacgtcccagcacccagccgaggccagactcttggggtgaggacaactgggagggcctcgagactgacagtcgacaggtcaaggctgagctggcccggaagaagcgcgaggagcggcggcgggagatggaggccaaacgcgccgagaggaaggtggccaagggccccatgaagctgggagcccggaagctggactgaaccgtggcggtggcccttcccggctgcggagagcccgccccacagatgtatttattgtacaaaccatgtgagcccggccggcccagccaggccatctcacgtgtacataatcagagccacaataaattctatttcacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 204 - protein, 270 aa, Homo sapiens
MFSSTDRAMRIRLLQQMEQFIQYLDEPTVNTQIFPHVVHGFLDTNPAIREQTVKSMLLLAPKLNEANLNVELMKHFARLQAKDEQGPIRCNTTVCLGKIGSYLSASTRHRVLTSAFSRATRDPFAPSRVAGVLGFAATHNLYSMNDCAQKILPVLCGLTVDPEKSVRDQAFKAFRSFLSKLESVSEDPTQLEEVEKDVHAASSPGMGGAAASWAGWAWTGVSSLTSKLIRSHPTTAPTETNIPQRPSRPARRPLGDAGGGQGHSRGQQHC

SEQ ID NO: 205 - DNA, 6782 bp, Homo sapiens
gggcggggctgagggcggcgggggcgggccgcccgagctgggagggcggcggcgccgaggggaggagagcggcccatggacccgcggggcccggcgccccagactctgcgccgtcgggacggagcccaagatgtcggcctaggccggggcgcgacgacgcggacggggcggcgaggaggcgccgctgctgccggggctcgcagccgccgagcccccgagggcgcgccctgacggactggccgagccggcggtgagaggccggcgcgtcgggagcgggccgcgcggcaccatgtcggccaaggtgcggctcaagaagctggagcagctgctcctggacgggccctggcgcaacgagagcgccctgagcgtggaaacgctgctcgacgtgctcgtctgcctgtacaccgagtgcagccactcggccctgcgccgcgacaagtacgtggccgagttcctcgagtgggctaaaccatttacacagctggtgaaagaaatgcagcttcatcgagaagactttgaaataattaaagtaattggaagaggtgcttttggtgaggttgctgttgtcaaaatgaagaatactgaacgaatttatgcaatgaaaatcctcaacaagtgggagatgctgaaaagagcagagaccgcgtgcttccgagaggagcgcgatgtgctggtgaacggcgactgccagtggatcaccgcgctgcactacgcctttcaggacgagaaccacctgtacttagtcatggattactatgtgggtggtgatttactgaccctgctcagcaaatttgaagacaagcttccggaagatatggcgaggttctacattggtgaaatggtgctggccattgactccatccatcagcttcattacgtgcacagagacattaaacctgacaatgtccttttggacgtgaatggtcatatccgcctggctgactttggatcatgtttgaagatgaatgatgatggcactgtgcagtcctccgtggccgtgggcacacctgactacatctcgccggagatcctgcaggcgatggaggacggcatgggcaaatacgggcctgagtgtgactggtggtctctgggtgtctgcatgtatgagatgctctatggagaaacgccgttttatgcggagtcactcgtggagacctatgggaagatcatgaaccatgaagagcgattccagttcccatcccatgtcacggatgtatctgaagaagcgaaggacctcatccagagactgatctgcagtagagaacgccggctggggcagaatggaatagaggatttcaaaaagcatgcgttttttgaaggtctaaattgggaaaatatacgaaacctagaagcaccttatattcctgatgtgagcagtccctctgacacatccaacttcgacgtggatgacgacgtgctgagaaacacggaaatattacctcctggttctcacacaggcttttctggattacatttgccattcattggttttacattcacaacggaaagctgtttttctgatcgaggctctctgaagagcataatgcagtccaacacattaaccaaagatgaggatgtgcagcgggacctggagcacagcctgcagatggaagcttacgagaggaggattcggaggctggaacaggagaagctggagctgagcaggaagctgcaagagtccacccagaccgtgcagtccctccacggctcatctcgggccctcagcaattcaaaccgagataaagaaatcaaaaagctaaatgaagaaatcgaacgcttgaagaataaaatagcagattcaaacaggctcgagcgacagcttgaggacacagtggcgcttcgccaagagcgtgaggactccacgcagcggctgcgggggctggagaagcagcaccgcgtggtccggcaggagaaggaggagctgcacaagcaactggttgaagcctcagagcggttgaaatcccaggccaaggaactcaaagatgcccatcagcagcgaaagctggccctgcaggagttctcggagctgaacgagcgcatggcagagctccgtgcccagaagcagaaggtgtcccggcagctgcgagacaaggaggaggagatggaggtggccacgcagaaggtggacgccatgcggcaggaaatgcggagagctgagaagctcaggaaagagctggaagctcagcttgatgatgctgttgctgaggcctccaaggagcgcaagcttcgtgagcacagcgagaacttctgcaagcaaatggaaagcgagctggaggccctcaaggtgaagcaaggaggccggggagcgggtgccaccttagagcaccagcaagagatttccaaaatcaaatccgagctggagaagaaagtcttattttatgaagaggaattggtcagacgtgaggcctcccatgtgctagaagtgaaaaatgtgaagaaggaggtgcatgattcagaaagccaccagctggccctgcagaaagaaatcttgatgttaaaagataagttagaaaagtcaaagcgagaacggcataacgagatggaggaggcagtaggtacaataaaagataaatacgaacgagaaagagcgatgctgtttgatgaaaacaagaagctaactgctgaaaatgaaaagctctgttcctttgtggataaactcacagctcaaaatagacagctggaggatgagctgcaggatctggcagccaagaaggagtcagtggcccactgggaagctcagattgcggaaatcattcagtgggtcagtgacgagaaagatgcccggggttaccttcaagctcttgcttccaagatgaccgaagagctcgaggctttgaggagttctagtctggggtcaagaacactggacccgctgtggaaggtgcgccgcagccagaagctggacatgtccgcgcggctggagctgcagtcggccctggaggcggagatccgggccaagcagcttgtccaggaggagctcaggaaggtcaaggacgccaacctcaccttggaaagcaaactaaaggattccgaagccaaaaacagagaattattagaagaaatggaaattttgaagaaaaagatggaagaaaaattcagagcagatactgggctcaaacttccagattttcaggattccatttttgagtatttcaacactgctcctcttgcacatgacctgacatttagaaccagctcagctagtgagcaagaaacacaagctccgaagccagaagcgtccccgtcgatgtctgtggctgcatcagagcagcaggaggacatggctcggcccccgcagaggccatccgctgtgccgttgcccaccacgcaggccctggctctggctggaccgaagccaaaagctcaccagttcagcatcaagtccttctccagccctactcagtgcagccactgcacctccctgatggttgggctgatccggcagggctacgcctgcgaggtgtgttcctttgcttgccacgtgtcctgcaaagacggtgccccccaggtgtgcccaatacctcccgagcagtccaagaggcctctgggcgtggacgtgcagcgaggcatcggaacagcctacaaaggccatgtcaaggtcccaaagcccacgggggtgaagaagggatggcagcgcgcatatgcagtcgtctgtgagtgcaagctcttcctgtatgatctgcctgaaggaaaatccacccagcctggtgtcattgcgagccaagtcttggatctcagagatgacgagttttccgtgagctcagtcctggcctcagatgtcattcatgctacacgccgagatattccatgtatattcagggtgacggcctctctcttaggtgcaccttctaagaccagctcgctgctcattctgacagaaaatgagaatgaaaagaggaagtgggttgggattctagaaggactccagtccatccttcataaaaaccggctgaggaatcaggtcgtgcatgttcccttggaagcctacgacagctcgctgcctctcatcaaggccatcctgacagctgccatcgtggatgcagacaggattgcagtcggcctagaagaagggctctatgtcatagaggtcacccgagatgtgatcgtccgtgccgctgactgtaagaaggtacaccagatcgagcttgctcccagggagaagatcgtaatcctcctctgtggccggaaccaccatgtgcacctctatccgtggtcgtcccttgatggagcggaaggcagctttgacatcaagcttccggaaaccaaaggctgccagctcatggccacggccacactcaagaggaactctggcacctgcctgtttgtggccgtgaaacggctgatcctttgctatgagatccagagaacgaagccattccacagaaagttcaatgagattgtggctcccggcagcgtgcagtgcctggcggtgctcagggacaggctctgtgtgggctacccttctgggttctgcctgctgagcatccagggggacgggcagcctctaaacctggtaaatcccaatgacccctcgcttgcgttcctctcacaacagtcttttgatgccctttgtgctgtggagctcgaaagcgaggagtacctgctttgcttcagccacatgggactgtacgtggacccgcaaggccggagggcacgcgcgcaggagctcatgtggcctgcggctcctgtcgcctgtagttgcagccccacccacgtcacggtgtacagcgagtatggcgtggacgtctttgatgtgcgcaccatggagtgggtgcagaccatcggcctgcggaggataaggcccctgaactctgaaggcaccctcaacctcctcaactgcgagcctccacgcttgatctacttcaagagcaagttctcgggagcggttctcaacgtgccggacacctccgacaacagcaagaagcagatgctgcgcaccaggagcaaaaggcggttcgtcttcaaggtcccagaggaagagagactgcagcagaggcgagagatgcttagagacccagaattgagatccaaaatgatatccaacccaaccaacttcaaccacgtggcccacatgggcccaggcgacggcatgcaggtgctcatggacctgcctctgagtgctgtgcccccctcccaggaggaaaggccgggccccgctcccaccaacctggctcgccagcctccatccaggaacaagccctacatctcgtggccctcatcaggtggatcggagcctagcgtgactgtgcctctgagaagtatgtctgatccagaccaggactttgacaaagagcctgattcggactccaccaaacactcaactccatcgaatagctccaaccccagcggcccaccgagccccaactccccccacaggagccagctccccctcgaaggcctggagcagccggcctgtgacacctgaagccgccagctcgccacaggggccagggagctggagatggcctccagcgtcagtgccaagactgagcgggccctccagtgttgtccaaggaaatgtagaatcactttgtagatatggagatgaagaagacaaatctttattataatattgatcagttttatgccgcattgttcgtggcagtagaccacatctgttcgtctgcacagctgtgaggcgatgctgttccatctgcacatgaaggacccccatacagcctgtctcccacccctgacaacccgagagggcatatggggccctgccaacaccacttcctcagcagaaacccgtcatgacgcggctgcttcggaagcagacatctggggacacagcctcagtacccagtcttttccctagttcctgaaactttcctaggaccttaagagaatagtaggaggtcctatagcattcccagtgtcactagaattttgaagacaggaaagtggaggttagtctgtggcctttttttcatttagccattgcacagtcagctgcagaagtcctgctgaccacctagtcatggacaaaggcccaggaccagtgacaccctgcgtccctgtgtgcattaagttcattctgggtcgcagccatgaagtgtcaccagtatctactactgtgaagtcagctgtgctgttttccattcgcttccacggcttctgcctcctgccataaaaccagcgagtgtcgtggtgcaggcaggccctgtggcctgctgggctgagggaagtcagagccccagggcgccacgaagcagccactgggataccccaccccgccccgccctgcccccccccccccccaccagtcctgcccccgcatggagcccccgtgattagtagcccgtatgatcacgtagacccacccaacacactcctgcacactggccccggcccacggcacagcaatcccctgcgcgtggatttcacctcaccctttgtaccagatgttgagtgaccagctctgtggccctgtgtcgtcagaggcttgtgattaactgtggcggcagacacagcttgtccacagcttgggccaggcttcccctgtcctcccaccggtcggctgcttggcaaggctgttcaggacgtgcacttccccaagtcggcactgagtggcccagcaccgcctagccctgccaccccactgccctcctgggccttctgctggatgggcacctggggggttctggtttttacttttttaatgtaagtctcagtctttgtaattaattattgaattgtgagaacatttttgaacaatttacctgtcaataaagcagaagacggcagttttaaagttaaaaaaaaaaaaaaaaaaaaaaaaa

SEQ ID NO: 206 - protein, 1711 aa, Homo sapiens
MSAKVRLKKLEQLLLDGPWRNESALSVETLLDVLVCLYTECSHSALRRDKYVAEFLEWAKPFTQLVKEMQLHREDFEIIKVIGRGAFGEVAVVKMKNTERIYAMKILNKWEMLKRAETACFREERDVLVNGDCQWITALHYAFQDENHLYLVMDYYVGGDLLTLLSKFEDKLPEDMARFYIGEMVLAIDSIHQLHYVHRDIKPDNVLLDVNGHIRLADFGSCLKMNDDGTVQSSVAVGTPDYISPEILQAMEDGMGKYGPECDWWSLGVCMYEMLYGETPFYAESLVETYGKIMNHEERFQFPSHVTDVSEEAKDLIQRLICSRERRLGQNGIEDFKKHAFFEGLNWENIRNLEAPYIPDVSSPSDTSNFDVDDDVLRNTEILPPGSHTGFSGLHLPFIGFTFTTESCFSDRGSLKSIMQSNTLTKDEDVQRDLEHSLQMEAYERRIRRLEQEKLELSRKLQESTQTVQSLHGSSRALSNSNRDKEIKKLNEEIERLKNKIADSNRLERQLEDTVALRQEREDSTQRLRGLEKQHRVVRQEKEELHKQLVEASERLKSQAKELKDAHQQRKLALQEFSELNERMAELRAQKQKVSRQLRDKEEEMEVATQKVDAMRQEMRRAEKLRKELEAQLDDAVAEASKERKLREHSENFCKQMESELEALKVKQGGRGAGATLEHQQEISKIKSELEKKVLFYEEELVRREASHVLEVKNVKKEVHDSESHQLALQKEILMLKDKLEKSKRERHNEMEEAVGTIKDKYERERAMLFDENKKLTAENEKLCSFVDKLTAQNRQLEDELQDLAAKKESVAHWEAQIAEIIQWVSDEKDARGYLQALASKMTEELEALRSSSLGSRTLDPLWKVRRSQKLDMSARLELQSALEAEIRAKQLVQEELRKVKDANLTLESKLKDSEAKNRELLEEMEILKKKMEEKFRADTGLKLPDFQDSIFEYFNTAPLAHDLTFRTSSASEQETQAPKPEASPSMSVAASEQQEDMARPPQRPSAVPLPTTQALALAGPKPKAHQFSIKSFSSPTQCSHCTSLMVGLIRQGYACEVCSFACHVSCKDGAPQVCPIPPEQSKRPLGVDVQRGIGTAYKGHVKVPKPTGVKKGWQRAYAVVCECKLFLYDLPEGKSTQPGVIASQVLDLRDDEFSVSSVLASDVIHATRRDIPCIFRVTASLLGAPSKTSSLLILTENENEKRKWVGILEGLQSILHKNRLRNQVVHVPLEAYDSSLPLIKAILTAAIVDADRIAVGLEEGLYVIEVTRDVIVRAADCKKVHQIELAPREKIVILLCGRNHHVHLYPWSSLDGAEGSFDIKLPETKGCQLMATATLKRNSGTCLFVAVKRLILCYEIQRTKPFHRKFNEIVAPGSVQCLAVLRDRLCVGYPSGFCLLSIQGDGQPLNLVNPNDPSLAFLSQQSFDALCAVELESEEYLLCFSHMGLYVDPQGRRARAQELMWPAAPVACSCSPTHVTVYSEYGVDVFDVRTMEWVQTIGLRRIRPLNSEGTLNLLNCEPPRLIYFKSKFSGAVLNVPDTSDNSKKQMLRTRSKRRFVFKVPEEERLQQRREMLRDPELRSKMISNPTNFNHVAHMGPGDGMQVLMDLPLSAVPPSQEERPGPAPTNLARQPPSRNKPYISWPSSGGSEPSVTVPLRSMSDPDQDFDKEPDSDSTKHSTPSNSSNPSGPPSPNSPHRSQLPLEGLEQPACDT

SEQ ID NO: 207 - DNA, 1196 bp, Homo sapiens
acctttgagcgatggcggcgtctggggaaccccagaggcagtggcaagaggaggtggcggcggtggtagtggtgggctcctgcatgaccgacctggtcagtcttacttctcgtttgccaaaaactggagaaaccatccatggacataagttttttattggctttggagggaaaggtgccaaccagtgtgtccaagctgctcggcttggagcaatgacgtccatggtgtgtaaggttggcaaagattcttttggcaatgattatatagaaaacttaaaacagaatgatatttctacagaatttacatatcagactaaagatgctgctacaggaactgcttctataattgtcaataatgaaggccagaatatcattgtcatagtggctggagcaaatttacttttgaatacggaggatctgagggcagcagccaatgtcattagcagagccaaagtcatggtctgccagctcgaaataactccagcaacttctttggaagccctaacaatggcccgcaggagtggagtgaaaaccttgttcaatccagcccctgccattgctgacctggatccccagttctacaccctctcagatgtgttctgctgcaatgaaagtgaggctgagattttaactggcctcacggtgggcagcgctgcagatgctggggaggctgcattagtgctcttgaaaaggggctgccaggtggtaatcattaccttaggggctgaaggatgtgtggtgctgtcacagacagaacctgagccaaagcacattcccacagagaaagtcaaggctgtggataccacgggtgctggtgacagctttgtgggagctctggccttctacctggcttactatccaaatctgtccttggaagacatgctcaacagatccaatttcattgcagcagtcagtgtccaggctgcaggaacacagtcatcttacccttacaaaaaagaccttccgcttactctgttttgattgctattagtcccaaaataaatatacctgggaataaaatgtacttgggggtggctgctcctggctaatgcttattagaaaatgtcctcgtcccctttctttgcaaatattagttcttttacgaagtcatcctcaagcttcaatttatttataacgatgattcttttgctttccatgcatttgcacaaaacaaccagaattaaagattccacaacc

SEQ ID NO: 208 - protein, 322 aa, Homo sapiens [sequence omitted]

SEQ ID NO: 209 - DNA, 1566 bp, Homo sapiens
gagtcagcccccgggggaggccatgaacgccacggggaccccggtggcccccgagtcctgccaacagctggcggccggcgggcacagccggctcattgttctgcactacaaccactcgggccggctggccgggcgcggggggccggaggatggcggcctgggggccctgcgggggctgtcggtggccgccagctgcctggtggtgctggagaacttgctggtgctggcggccatcaccagccacatgcggtcgcgacgctgggtctactattgcctggtgaacatcacgctgagtgacctgctcacgggcgcggcctacctggccaacgtgctgctgtcgggggcccgcaccttccgtctggcgcccgcccagtggttcctacgggagggcctgctcttcaccgccctggccgcctccaccttcagcctgctcttcactgcaggggagcgctttgccaccatggtgcggccggtggccgagagcggggccaccaagaccagccgcgtctacggcttcatcggcctctgctggctgctggccgcgctgctggggatgctgcctttgctgggctggaactgcctgtgcgcctttgaccgctgctccagccttctgcccctctactccaagcgctacatcctcttctgcctggtgatcttcgccggcgtcctggccaccatcatgggcctctatggggccatcttccgcctggtgcaggccagcgggcagaaggccccacgcccagcggcccgccgcaaggcccgccgcctgctgaagacggtgctgatgatcctgctggccttcctggtgtgctggggcccactcttcgggctgctgctggccgacgtctttggctccaacctctgggcccaggagtacctgcggggcatggactggatcctggccctggccgtcctcaactcggcggtcaaccccatcatctactccttccgcagcagggaggtgtgcagagccgtgctcagcttcctctgctgcgggtgtctccggctgggcatgcgagggcccggggactgcctggcccgggccgtcgaggctcactccggagcttccaccaccgacagctctctgaggccaagggacagctttcgcggctcccgctcgctcagctttcggatgcgggagcccctgtccagcatctccagcgtgcggagcatctgaagttgcagtcttgcgtgtggatggtgcagccaccgggtgcgtgccaggcaggccctcctggggtacaggaagctgtgtgcacgcagcctcgcctgtatggggagcagggaacgggacaggcccccatggtcttcccggtggcctctcggggcttctgacgccaaatgggcttcccatggtcaccctggacaaggaggtaaccaccccacctccccgtaggagcagagagcaccctggtgtgggggcgagtggttccccacaaccccgcttctgtgtgattctggggaagtcccggcccctctctgggcctcagtagggctcccaggctgcaaggggtggactgtgggatgcatgccctggcaacattgaagttcgatcatggtaaaaaa

SEQ ID NO: 210 - protein, 384 aa, Homo sapiens
MNATGTPVAPESCQQLAAGGHSRLIVLHYNHSGRLAGRGGPEDGGLGALRGLSVAASCLVVLENLLVLAAITSHMRSRRWVYYCLVNITLSDLLTGAAYLANVLLSGARTFRLAPAQWFLREGLLFTALAASTFSLLFTAGERFATMVRPVAESGATKTSRVYGFIGLCWLLAALLGMLPLLGWNCLCAFDRCSSLLPLYSKRYILFCLVIFAGVLATIMGLYGAIFRLVQASGQKAPRPAARRKARRLLKTVLMILLAFLVCWGPLFGLLLADVFGSNLWAQEYLRGMDWILALAVLNSAVNPIIYSFRSREVCRAVLSFLCCGCLRLGMRGPGDCLARAVEAHSGASTTDSSLRPRDSFRGSRSLSFRMREPLSSISSVRSI

SEQ ID NO: 211 - DNA, 3000 bp, Homo sapiens
  misc_feature (173)..(173): n is a, c, g, or t
gggcggacctaaaggggctcgggccgctcgggccgggaatggcggcggcggccgagcccggggcccgcgcctggctgggcggcggctccccgcgccccggcagcccggcctgcagccccgtgctgggctcaggaggccgcgcgcgcccggggccggggccggggccgggacgngaccgagcgggcggcgtcagagcccgggcccgtgccgcgccgggacacagcttccggaaggtgacgctcaccaagcccaccttctgccacctctgctccgacttcatctgggggctggccggcttcctgtgcgacgtctgcaatttcatgtctcatgagaagtgcctgaagcacgtgaggatcccgtgcacgagtgtggcacccagcctggtccgggttcctgtagcccactgcttcggcccccgggggctccacaagcgcaagttctgtgctgtctgccgcaaggtcctggaggcaccggcgctccactgcgaagtgtgtgagctgcacctccacccagactgtgtgcccttcgcctgcagtgactgccgccagtgccaccaggatgggcaccaggatcacgacacccatcaccaccactggcgggaggggaacctgccctcgggagcgcgctgcgaggtctgcaggaagacgtgcggctcctctgacgtgctggccggcgtgcgctgcgagtggtgcggggtccaggcgcactccctctgctccgcggcactggctcccgagtgtggcttcgggcgtctgcgctccctggtcctgcctcccgcgtgcgtgcgccttctgcccggcggcttcagcaagacgcagagcttccgcatcgtggaggccgcggagccgggcgaggggggcgacggcgccgacgggagcgctgccgtgggtccaggcagagagacacaggcaactccggagtccgggaagcaaacgctgaagatctttgatggcgacgacgcggtgagaagaagccagttccgcctcgtcacggtgtcccgcctggccggtgccgaggaggtgctggaggccgcactgcgggcccaccacatccccgaggaccctggccacctggagctgtgccggctgcccccttcctctcaggcctgtgacgcctgggctgggggcaaggctgggagtgctgtgatctcggaggagggcagaagccccgggtccggcgaggccacgccagaggcctgggtcatccgggctctgccgcgggcccaggaggtcctgaagatctaccctggctggctcaaggtgggcgtggcctacgtgtccgtgcgagtgacccctaagagcacggctcgctctgtggtgctggaggtcctgccgctgctcggccgccaggccgagagtcccgagagcttccagctggtggaggtggcgatgggctgcaggcacgtccagcggacgatgctgatggacgaacagcccctgctggaccggctacaggacatccggcagatgtctgtgcggcaggtgagccagacgcggttctacgtggcagagagcagggatgtagccccgcacgtctccctgtttgttggcggcctgcctcccggcctgtctcccgaggagtacagcagcctgctgcatgaggccggggctaccaaagccaccgtggtgtccgtgagtcacatctactcctcccaaggcgcggtagtgttggacgttgcctgctttgcggaggccgagcggctgtacatgctgctgaaggacatggctgtgcggggccggctgctcactgccctggtgctccccgacctgctgcacgcgaagctgcccccagacagctgtcccctccttgtgttcgtgaaccccaagagtggaggcctcaagggccgagacctgctctgcagcttccggaagctactgaaccctcatcaggtcttcgacctgaccaacggaggtcctcttcccgggctccacctgttctcccaggtgccctgcttccgggtgctggtgtgtggtggcgatggcactgtgggctgggtgcttggcgccctggaggagacacggtaccgactggcctgcccggagccttctgtggccatcctgcccctgggcacagggaatgaccttggtcgagtcctccgctggggggcgggctacagcggcgaggacccgttctccgtactgctgtctgtggacgaggccgacgccgtgctcatggaccgctggaccatcctgctggatgcccacgaagctggcagtgcagagaacgacacggcagacgcagagccccccaagatcgtgcagatgagtaactactgtggcattggcatcgacgcggagctgagcctggacttccaccaggcacgggaagaggagcctggcaagttcacaagcaggctgcacaacaagggtgtgtacgtgcgggtggggctgcagaagatcagtcactctcggagcctgcacaagcagatccggctgcaggtggagcggcaggaggtggagctgcccagtattgaaggcctcatcttcatcaacatccccagctggggctcgggggccgacctgtggggctccgacagcgacaccaggtttgagaagccacgcatggacgacgggctgctggaggttgtgggcgtgacgggcgtcgtgcacatgggccaggtccagggtgggctgcgctccggaatccggattgcccagggttcctacttccgagtcacgctcctcaaggccaccccggtgcaggtggacggggagccctgggtccaggccccggggcacatgatcatctcagctgctggccctaaggtgcacatgctgaggaaggccaagcagaagccgaggagggccgggaccaccagggatgcccgggcggatcgtgcgcctgcccctgagagcgatcctaggtaggggtggctggggcagcccaagggctcgagccatctctgctcccgccagccttgttttcaggtggtctggaggcagctccacgtcacacagtggctgtcatatattgaagttaccttcccactggaaaaaaaat

SEQ ID NO: 212 - protein, 942 aa, Homo sapiens
MAAAAEPGARAWLGGGSPRPGSPACSPVLGSGGRARPGPGPGPGRDRAGGVRARARAAPGHSFRKVTLTKPTFCHLCSDFIWGLAGFLCDVCNFMSHEKCLKHVRIPCTSVAPSLVRVPVAHCFGPRGLHKRKFCAVCRKVLEAPALHCEVCELHLHPDCVPFACSDCRQCHQDGHQDHDTHHHHWREGNLPSGARCEVCRKTCGSSDVLAGVRCEWCGVQAHSLCSAALAPECGFGRLRSLVLPPACVRLLPGGFSKTQSFRIVEAAEPGEGGDGADGSAAVGPGRETQATPESGKQTLKIFDGDDAVRRSQFRLVTVSRLAGAEEVLEAALRAHHIPEDPGHLELCRLPPSSQACDAWAGGKAGSAVISEEGRSPGSGEATPEAWVIRALPRAQEVLKIYPGWLKVGVAYVSVRVTPKSTARSVVLEVLPLLGRQAESPESFQLVEVAMGCRHVQRTMLMDEQPLLDRLQDIRQMSVRQVSQTRFYVAESRDVAPHVSLFVGGLPPGLSPEEYSSLLHEAGATKATVVSVSHIYSSQGAVVLDVACFAEAERLYMLLKDMAVRGRLLTALVLPDLLHAKLPPDSCPLLVFVNPKSGGLKGRDLLCSFRKLLNPHQVFDLTNGGPLPGLHLFSQVPCFRVLVCGGDGTVGWVLGALEETRYRLACPEPSVAILPLGTGNDLGRVLRWGAGYSGEDPFSVLLSVDEADAVLMDRWTILLDAHEAGSAENDTADAEPPKIVQMSNYCGIGIDAELSLDFHQAREEEPGKFTSRLHNKGVYVRVGLQKISHSRSLHKQIRLQVERQEVELPSIEGLIFINIPSWGSGADLWGSDSDTRFEKPRMDDGLLEVVGVTGVVHMGQVQGGLRSGIRIAQGSYFRVTLLKATPVQVDGEPWVQAPGHMIISAAGPKVHMLRKAKQKPRRAGTTRDARADRAPAPESDPR

SEQ ID NO: 213 - DNA, 1230 bp, Homo sapiens
aagcttgtggcatttggtactggtatctgagcaggggctggctttctgtttgtctgtgtgttttttgcatgatcttggattgtcaccctgctgtatttaaacattaaaaagcctgtcttttcgttgaagaggacaggggttaaaatgaatgaagacctgaaggtcaatttaagcgggctgcctcgggattatttagatgccgctgctgcggagaacatctcggctgctgtctcctcccgggttcctgccgtagagccagagcctgagctcgtagtcaacccctgggacattgtcttgtgtacctcgggaaccctcatctcctgtgaaaatgccattgtggtccttatcatcttccacaaccccagcctgcgagcacccatgttcctgctaataggcagcctggctcttgcagacctgctggccggcattggactcatcaccaattttgtttttgcctacctgcttcagtcagaagccaccaagctggtcacgatcggcctcattgtcgcctctttctctgcctctgtctgcagcttgctggctatcactgttgaccgctacctctcactgtactacgctctgacgtaccattcggagaggacggtcacgtttacctatgtcatgctcgtcatgctctgggggacctccatctgcctggggctgctgcccgtcatgggctggaactgcctccgagacgagtccacctgcagcgtggtcagaccgctcaccaagaacaacgcggccatcctctcggtgtccttcctcttcatgtttgcgctcatgcttcagctctacatccagatctgtaagattgtgatgaggcacgcccatcagatagccctgcagcaccacttcctggccacgtcgcactatgtgaccacccggaaaggggtctccaccctggctatcatcctggggacgtttgctgcttgctggatgcctttcaccctctattccttgatagcggattacacctacccctccatctatacctacgccaccctcctgcccgccacctacaattccatcatcaaccctgtcatatatgctttcagaaaccaagagatccagaaagcgctctgtctcatttgctgcggctgcatcccgtccagtctcgcccagagagcgcgctcgcccagtgatgtgtagcacccttgcacccaggaggactctgcatttaccaagcacttccactgcctggccaaggtttgagatgcttcccttgaattc

SEQ ID NO: 214 - protein, 334 aa, Homo sapiens
MNEDLKVNLSGLPRDYLDAAAAENISAAVSSRVPAVEPEPELVVNPWDIVLCTSGTLISCENAIVVLIIFHNPSLRAPMFLLIGSLALADLLAGIGLITNFVFAYLLQSEATKLVTIGLIVASFSASVCSLLAITVDRYLSLYYALTYHSERTVTFTYVMLVMLWGTSICLGLLPVMGWNCLRDESTCSVVRPLTKNNAAILSVSFLFMFALMLQLYIQICKIVMRHAHQIALQHHFLATSHYVTTRKGVSTLAIILGTFAACWMPFTLYSLIADYTYPSIYTYATLLPATYNSIINPVIYAFRNQEIQKALCLICCGCIPSSLAQRARSPSDV

SEQ ID NO: 215 - DNA, 2169 bp, Homo sapiens
ccgcctccgagtgccttgcgcggacctgagctggagatgctggccgggctaccgacgtcagaccccgggcgcctcatcacggacccgcgcagcggccgcacctacctcaaaggccgcttgttgggcaaggggggcttcgcccgctgctacgaggccactgacacagagactggcagcgcctacgctgtcaaagtcatcccgcagagccgcgtcgccaagccgcatcagcgcgagaagatcctaaatgagattgagctgcaccgagacctgcagcaccgccacatcgtgcgtttttcgcaccactttgaggacgctgacaacatctacattttcttggagctctgcagccgaaagtccctggcccacatctggaaggcccggcacaccctgttggagccagaagtgcgctactacctgcggcagatcctttctggcctcaagtacttgcaccagcgcggcatcttgcaccgggacctcaagttgggaaattttttcatcactgagaacatggaactgaaggtgggggattttgggctggcagcccggttggagcctccggagcagaggaagaagaccatctgtggcacccccaactatgtggctccagaagtgctgctgagacagggccacggccctgaagcggatgtatggtcactgggctgtgtcatgtacacgctgctctgcgggagccctccctttgagacggctgacctgaaggagacgtaccgctgcatcaagcaggttcactacacgctgcctgccagcctctcactgcctgcccggcagctcctggccgccatccttcgggcctcaccccgagaccgcccctctattgaccagatcctgcgccatgacttctttaccaagggctacacccccgatcgactccctatcagcagctgcgtgacagtcccagacctgacaccccccaacccagctaggagtctgtttgccaaagttaccaagagcctctttggcagaaagaagaagagtaagaatcatgcccaggagagggatgaggtctccggtttggtgagcggcctcatgcgcacatccgttggccatcaggatgccaggccagaggctccagcagcttctggcccagcccctgtcagcctggtagagacagcacctgaagacagctcaccccgtgggacactggcaagcagtggagatggatttgaagaaggtctgactgtggccacagtagtggagtcagccctttgtgctctgagaaattgtatagctttcatgcccccagcggaacagaacccggcccccctggcccagccagagcctctggtgtgggtcagcaagtgggttgactactccaataagttcggctttgggtatcaactgtccagccgccgtgtggctgtgctcttcaacgatggcacacatatggccctgtcggccaacagaaagactgtgcactacaatcccaccagcacaaagcacttctccttctccgtgggtgctgtgccccgggccctgcagcctcagctgggtatcctgcggtacttcgcctcctacatggagcagcacctcatgaagggtggagatctgcccagtgtggaagaggtagaggtacctgctccgcccttgctgctgcagtgggtcaagacggatcaggctctcctcatgctgtttagtgatggcactgtccaggtgaacttctacggggaccacaccaagctgattctcagtggctgggagcccctccttgtgacttttgtggcccgaaatcgtagtgcttgtacttacctcgcttcccaccttcggcagctgggctgctctccagacctgcggcagcgactccgctatgctctgcgcctgctccgggaccgcagcccagcttaggacccaagccctgaaggcctgaggcctgtgcctgtcaggctctggcccttgcctttgtggccttcccccttcctttggtgcctcactgggggctttgggccgaatcccccagggaatcagggaccagctttactggagttgggggcggcttgtcttcgctggctcctaccccatctccaagataagcctgagccttagctcccagctagggggcgttatttatggaccacttttatttattgtcagacacttatttattgggatgtgagccccagggggcctcctcctaggataataaacaattttgca

SEQ ID NO: 216 - protein, 607 aa, Homo sapiens
MLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 217 - DNA, 1547 bp, Homo sapiens
cctcctaaccagcggccagtgggtttcccataccccaggatgtgagcctctttaacctgtaatgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatactcgggccggaccctctcaacacagcagcccagcagtctcagattcacttccaagcaatagcttaaagaagtcttctgctgaactgaaaaaaatactagccaatggccagatgaatgaacaagacatacgatatcgggacactcttggtcatggcaacggaggcacagtctacaaagcatatcatgtcccgagtgggaaaatattagctgtaaaggtcatactactagatattacactggaacttcagaagcaaattatgtctgaattggaaattctttataagtgcgattcatcatatatcattggattttatggagcattttttgtagaaaacaggatttcaatatgtacagaattcatggatgggggatctttggatgtatataggaaaatgccagaacatgtccttggaagaattgcagtagcagttgttaaaggccttacttatttgtggagtttaaagattttacatagagacgtgaagccctccaatatgctagtaaacacaagaggacaggttaagctgtgtgattttggagttagcactcagctggtgaattctatagccaagacgtatgttggaacaaatgcttatatggcgcctgaaaggatttcaggggagcagtatggaattcattctgatgtctggagcttaggaatctcttttatggagcttgctcttgggaggtttccatatcctcagattcagaaaaaccagggatctttaatgcctctccagcttctgcagtgcattgttgatgaggattcgcccgtccttccagttggagagttctcggagccatttgtacatttcatcactcagtgtatgcgaaaacagccaaaagaaaggccagcacctgaagaattgatgggccacccgttcatcgtgcagttcaatgatggaaatgccgccgtggtgtccatgtgggtgtgccgggcgctggaggagaggcggagccagcaggggcccccgtgaggctgccgcagggcactgaaagcccaggaccagtaaccaaggagaacaacccacccgtcgcccttctccgtatgctgcctgcgccagaagagctttgctgggccctggcttccctgccctcgccttcaccctctgtcag

SEQ ID NO: 218 - protein, 448 aa, Homo sapiens
MLWLALGPFPAMENQVLVIRIKIPNSGAVDWTVHSGPQLLFRDVLDVIGQVLPEATTTAFEYEDEDGDRITVRSDEEMKAMLSYYYSTVMEQQVNGQLIEPLQIFPRACKPPGERNIHGLKVNTRAGPSQHSSPAVSDSLPSNSLKKSSAELKKILANGQMNEQDIRYRDTLGHGNGGTVYKAYHVPSGKILAVKVILLDITLELQKQIMSELEILYKCDSSYIIGFYGAFFVENRISICTEFMDGGSLDVYRKMPEHVLGRIAVAVVKGLTYLWSLKILHRDVKPSNMLVNTRGQVKLCDFGVSTQLVNSIAKTYVGTNAYMAPERISGEQYGIHSDVWSLGISFMELALGRFPYPQIQKNQGSLMPLQLLQCIVDEDSPVLPVGEFSEPFVHFITQCMRKQPKERPAPEELMGHPFIVQFNDGNAAVVSMWVCRALEERRSQQGPP

SEQ ID NO: 219 - DNA, 3068 bp, Homo sapiens
gaagtccctatcagattacacttggttgactactccggagcagccactaagagggatgaacaggcctgcgtggaaattgaatgagattagttgagtccacgctccatgagaatgctgaacaccatccaaagcagcaaattgagattccttgatttggggaagaggtttgggaggaacccttcaataattggcatgggacaagaggggacccagtccaagtgtatttgggactcgcagtagggaggaacaattcagagagagcttggaagctcgaagtctggctgtggccatgggagatacagtagtggagcctgcccccttgaagccaacttctgagcccacttctggcccaccagggaataatggggggtccctgctaagtgtcatcacggagggggtcggggaactatcagtgattgaccctgaggtggcccagaaggcctgccaggaggtgttggagaaagtcaagcttttgcatggaggcgtggcagtctctagcagaggcaccccactggagttggtcaatggggatggtgtggacagtgagatccgttgcctagatgatccacctgcccagatcagggaggaggaagatgagatgggggccgctgtggcctcaggcacagccaaaggagcaagaagacggcggcagaacaactcagctaaacagtcttggctgctgaggctgtttgagtcaaaactgtttgacatctccatggccatttcatacctgtataactccaaggagcctggagtacaagcctacattggcaaccggctcttctgctttcgcaacgaggacgtggacttctatctgccccagttgcttaacatgtacatccacatggatgaggacgtgggtgatgccattaagccctacatagtccaccgttgccgccagagcattaacttttccctccagtgtgccctgttggttggggcctattcttcagacatgcacatttccactcaacgacactcccgtgggaccaagctacggaagctgatcctctcagatgagctaaagccagctcacaggaagagggagctgccctccttgagcccggcccctgatacagggctgtctccctccaaaaggactcaccagcgctctaagtcagatgccactgccagcataagtctcagcagcaacctgaaacgaacagccagcaaccctaaagtggagaatgaggatgagcctgttcgactggctcctgagagagaattcatcaagtccctgatggcgatcggcaagcgggtggtcacgctccccaccaaagagcagaaaacacagaggctgatctcagagctctccctgctcaaccataagctccctgcccgagtctggctgtccactgctgggtttgaccaccacgtggtccgtgtaccccacacacaggctgttgtcctcaactccaaggacaaggctccctacctgatttatgtggaagtccttgaatgtgaaaactttgacaccaccagtgtccctgcccggatccccgagaaccgaattcggagtacgaggtccgtagaaaacttgcccgaatgtggtattacccatgagcagcgagctggcagcttcagcactgtgcccaactatgacaacgatgatgaggcctggtcggtggatgacataggcgagctgcaagtggagctccccgaagtgcataccaacagctgtgacaacatctcccagttctctgtggacagcatcaccagccaggagagcaaggagcctgtgttcattgcagcaggggacatccgccggcgcctttcggaacagctggctcataccccgacagccttcaaacgagacccagaagatccttctgcagttgctctcaaagagccctggcaggagaaagtacggcggatcagagagggctccccctacggccatctccccaattggcggctcctgtcagtcattgtcaagtgtggggatgaccttcggcaagagcttctggcctttcaggtgttgaagcaactgcagtccatttgggaacaggagcgagtgcccctttggatcaagccatacaagattcttgtgatttcggctgatagtggcatgattgaaccagtggtcaatgctgtgtccatccatcaggtgaagaaacagtcacagctctccttgctcgattacttcctacaggagcacggcagttacaccactgaggcattcctcagtgcacagcgcaattttgtgcaaagttgtgctgggtactgcttggtctgctacctgctgcaagtcaaggacagacacaatgggaatatccttttggacgcagaaggccacatcatccacatcgacttcggcttcatcctctccagctcaccccgaaatctgggctttgagacgtcagcctttaagctgaccacagagtttgtggatgtgatgggcggcctggatggcgacatgttcaactactataagatgctgatgctgcaagggctgattgccgctcggaaacacatggacaaggtggtgcagatcgtggagatcatgcagcaaggttctcagcttccttgcttccatggctccagcaccattcgaaacctcaaagagaggttccacatgagcatgactgaggagcagctgcagctgctggtggagcagatggtggatggcagtatgcggtctatcaccaccaaactctatgacggcttccagtacctcaccaacggcatcatgtgacacgctcctcagcccaggagtggtggggggtccagggcaccctccctagagggcccttgtttgagaaaccccaaaccaggaaaccccacctacccaaccatccacccaagggaaatggaaggcaagaaacacgaaggatcatgtggtaactgcgagagcttgctgaggggtgggagagccagctgtggggtccagacttgttggggcttccctgcccctcctggtctgtgtcagtattaccaccagactgactccaggactcactgccctccagaaaacagaggtgacaaatgtgagggacactggggcctttcttctccttgtaggggtctctcagaggttctttccacaggccatcctcttattccgttct

SEQ ID NO: 220 - protein, 801 aa, Homo sapiens [sequence omitted]

SEQ ID NO: 221 - DNA, 4450 bp, Homo sapiens
acccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggacctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaggggttaggcctccggatgactgcgggcaggcctggataatatccagcctcccacaagaagctggtggagcagagtgttccctgactcctccaaggaaagggagacgccctttcatggtctgctgagtaacaggtgccttcccagacactggcgttactgcttgaccaaagagccctcaagcggcccttatgccagcgtgacagagggctcacctcttgccttctaggtcacttctcacaatgtcccttcagcacctgaccctgtgcccgccgattattccttggtaatatgagtaatacatcaaagagtagtattaaaagctaattaatcatgtttataaaaa

SEQ ID NO: 222 - protein, 1298 aa, Homo sapiens
MQRGAALCLRLWLCLGLLDGLVSDYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGPYLCSVCRPKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRVRDGTRMRAPELATPAIRHIMLNCWSGDPKARPAFSDLVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 223 - DNA, 3437 bp, Homo sapiens
attgcccggagatggccggcagagccgccgagacgccgaagagcccgccgcccgcgcgaggtgtagacggggcactgccttcagagcaggtcctgccagcctcgctggagaggatgccctcgtgtccgtgatgggctgtgggacaagcaaggtccttcccgagccacccaaggatgtccagctggatctggtcaagaaggtggagcccttcagtggcactaagagtgacgtgtacaagcacttcatcacagaggtggacagtgttggccctgtcaaagccgggttcccagcagcaagtcagtatgcacacccctgccccggtcccccgactgctggccacacggagcctccctcagaaccaccacgcagggccagggtagctaagtacagggccaagtttgacccacgtgttacagctaagtatgacatcaaggccctaattggccgaggcagcttcagccgagtggtacgtgtagagcaccgggcaacccggcagccgtatgccatcaagatgattgagaccaagtaccgggaggggcgggaggtgtgtgagtcggagctgcgtgtgctgcgtcgggtgcgtcatgccaacatcatccagctggtggaggtgttcgagacacaggagcgggtgtacatggtgatggagctggccactggtggagagctctttgaccgcatcattgccaagggctccttcaccgagcgtgacgccacgcgggtgctgcagatggtgctggatggcgtccggtatctgcatgcactgggcatcacacaccgagacctcaaacctgagaatctgctctactaccatccgggcactgactccaagatcatcatcaccgacttcggcctggccagtgctcgcaagaagggtgatgactgcttgatgaagaccacctgtggcacgcctgagtacattgccccagaagtcctggtccgcaagccatacaccaactcagtggacatgtgggcgctgggcgtcattgcctacatcctactcagtggcaccatgccgtttgaggatgacaaccgtacccggctgtaccggcagatcctcaggggcaagtacagttactctggggagccctggcctagtgtgtccaacctggccaaggacttcattgaccgcctgctgacagtggaccctggagcccgtatgactgcactgcaggccctgaggcacccgtgggtggtgagcatggctgcctcttcatccatgaagaacctgcaccgctccatatcccagaacctccttaaacgtgcctcctcgcgctgccagagcaccaaatctgcccagtccacgcgttccagccgctccacacgctccaataagtcacgccgtgtgcgggaacgggagctgcgggagctcaacctgcgctaccagcagcaatacaatggctgagccgcctggctgtgcacacatgcagcacgacccagcctggccacacactgtggtgccatctgggtccgatgccctctctggagataggcctatgtggcccacagtaggtgaagaatgtctggctccagccctttctctgtgccttcagcagcccctgtcctcaccatgggcctgggccaggtgtgacagagtagaggtagcacagggggctgtgactccccctgaactgggagcctggcctggcactgatacccctcttggtgggcagctgctctggtggagttgggaagggataggacctggccttcactgtctcccttgccctttgacttttccccaatcaaagggaactgcagtgctgggtggagtgtcctgtggcctcaggaccctttgggacagttacttctgggaccccctttcctccacagagcccttctccctggtttcacacattcccatgcatcctgatccttaagattatgctccagtgggagaccctggtaggcacaaagcttgtgccttgactggacccgtagcccctggctaggtcgaaacagccctccacctcccagccaagatctgtcttccttcatggtgcctccagggagccttcctggtcccaggacctctggtggagggccatggcgtggaccttcacccttctggactgtgtggccatgctggtcatcggcttgcccaggctccagcctctccagattctgaggggtctcagcccaccgcccttggtgccttctttgtagagcccaccgctacctccctctccccgttggatgtccattccattccccaggtgcctccttcccaactgggggtggttaaagggagccccactgctgctacctggggaatggggcacctgggggccaaggcagagggaagggggtcctcccgattagggtcgagtgtcagcctgggttctatcctttggtgcagccccattgccttttcccttcaggctctgttgctccctcctctgcagctgcacgaaggcgccatctggtgtctgcatgggtgttggcagcctgggagtgatcactgcacgcccatcgtgcacacctgcccatcgtgcacacccacccatggtgcacacctgtagtcctccatgaggacatgggaaggtaggagttgccgccctgggggagggtcccgggctgctcacctctccccttctgctgagcttctgcgcacccctccctggaacttagccatactgtgtgacctgcctctgaaaccagggtgccaggggcactgccttctcacagctggccttgccccgtccaccctgtgctgcttcccttcacagcattaaccttccagtctgggtcccactgagcctcaagctggaaggagcccctgcgggaggtgggtggggttgggtggctgctttcccagaggcctgagccagaaccatccccatttcttttgtggtatctccccctaccacaaaccaggctggaacccaagccccttcctccacagctgccttcagtgggtagaatggggccagggcccagctttggccttagcttgacggcagggcccctgccattgcaggagggtttggttcccactcagcttctgccggtcggcagcctgggccaggcccttttcctgcatgtgccacctccagtgggaaacaaaactaaagagaccactctgtgccaagtcgactatgccttagacacatcctcctaccgtccccaatgccccctgggcaggaggcagtggagaaccaagccccatggcctcagaatttccccccagttccccaagtgtctctggggacctgaagccctggggcttacgttctctcttgcccagggtgggcctggtcctgagggcaggacagggggtttggagatgtgggcctttgatagacccacttgggccttcatgccatggcctgtggatggagaatgtgcagttatttattatgcgtattcagtttgtaaacgtatcctctgtattcagtaaacaggctgcctctccagggagggctgccattcattcc

SEQ ID NO: 224 - protein, 424 aa, Homo sapiens
MGCGTSKVLPEPPKDVQLDLVKKVEPFSGTKSDVYKHFITEVDSVGPVKAGFPAASQYAHPCPGPPTAGHTEPPSEPPRRARVAKYRAKFDPRVTAKYDIKALIGRGSFSRVVRVEHRATRQPYAIKMIETKYREGREVCESELRVLRRVRHANIIQLVEVFETQERVYMVMELATGGELFDRIIAKGSFTERDATRVLQMVLDGVRYLHALGITHRDLKPENLLYYHPGTDSKIIITDFGLASARKKGDDCLMKTTCGTPEYIAPEVLVRKPYTNSVDMWALGVIAYILLSGTMPFEDDNRTRLYRQILRGKYSYSGEPWPSVSNLAKDFIDRLLTVDPGARMTALQALRHPWVVSMAASSSMKNLHRSISQNLLKRASSRCQSTKSAQSTRSSRSTRSNKSRRVRERELRELNLRYQQQYNG

SEQ ID NO: 225 - DNA, 411 bp, Homo sapiens
gcagtcaccaagccccgctacatgcagtggagggaaaccatgagctccacctctaccctgggcttccggatcgagggcatcaagaaggcagatgggacctgtaacaccaacttcaagaagacgcaggcactggagcaggtgacaaaagtgctggaggacttcgtggatggagaccacgtcatcctgcaaaagtacgtggcatgcctagaagaacttcgtgaagctctggagatctcccccttcttcaagacccacgaggtggtaggcagctccctcctcttcgtgcacgaccacaccggcctggccaaggtctggatgatagacttcggcaagacggtggccttgcccgaccaccagacgctcagccacaggctgccctgggctgagggcaaccgtgaggacggctacctc

SEQ ID NO: 226 - protein, 137 aa, Homo sapiens
AVTKPRYMQWRETMSSTSTLGFRIEGIKKADGTCNTNFKKTQALEQVTKVLEDFVDGDHVILQKYVACLEELREALEISPFFKTHEVVGSSLLFVHDHTGLAKVWMIDFGKTVALPDHQTLSHRLPWAEGNREDGYL